(19) United States
(12) Patent Application Publication
(10) Pub. No.: US 2025/0270200 A1
(43) Pub. Date: Aug. 28, 2025

(54) CTPS1 INHIBITORS AND USES THEREOF

(71) Applicant: Nimbus Clotho, Inc., Boston, MA (US)

(72) Inventors: Neelu Kaila, Lexington, MA (US); Steven K. Albanese, Brooklyn, NY (US); Sebastien Campos, Hoddesdon (GB); Kevin DeMarco, New York, NY (US); Kevin D. Kreutter, Arlington, MA (US); Andreas Verras, New York, NY (US); Angela V. West, Franklin, MA (US); Lewis Whitehead, Swampscott, MA (US)

(21) Appl. No.: 18/858,211

(22) PCT Filed: Apr. 21, 2023
(86) PCT No.: PCT/US23/19488

§ 371 Date: Oct. 18, 2024

Related U.S. Application Data
(60) Provisional application No. 63/363,374, filed on Apr. 21, 2022.

(51) Int. Cl. C07D 417/14; A61K 31/437; A61K 31/444; A61K 31/4545; A61K 31/497; A61K 31/506; A61K 31/5377; A61K 31/5386; A61K 31/553; C07D 401/12; C07D 401/14; C07D 413/14; C07D 417/04; C07D 471/04; C07D 491/048; C07D 491/107; C07D 498/08; C07D 519/00

(52) U.S. Cl. CPC C07D 417/14; A61K 31/437; A61K 31/444; A61K 31/4545; A61K 31/497; A61K 31/506; A61K 31/5377; A61K 31/5386; A61K 31/553; C07D 401/12; C07D 401/14; C07D 413/14; C07D 417/04; C07D 471/04; C07D 491/048; C07D 491/107; C07D 498/08; C07D 519/00

(57) ABSTRACT

The present invention provides compounds, compositions thereof, and methods of using the same for the inhibition of CPTS1, and the treatment of CPTS1-mediated disorders.

### CROSS-REFERENCE TO RELATED APPLICATIONS

This application claims the benefit of U.S. Provisional Patent Application No. 63/363,374, filed Apr. 21, 2022; the entirety of which is hereby incorporated by reference.

### TECHNICAL FIELD OF THE INVENTION

The present invention relates to compounds and methods useful for antagonizing CTP synthase 1 (CTPS1). The invention also provides pharmaceutically acceptable compositions comprising compounds of the present invention and methods of using the compositions in the treatment of various disorders.

### BACKGROUND OF THE INVENTION

All cells, whether prokaryotic or eukaryotic in origin, utilize nucleotides as key building blocks for cellular metabolic processes, such as deoxyribonucleic acid (DNA) and ribonucleic acid (RNA) synthesis, membrane lipid biosynthesis, and as a cellular store of biochemical energy for many important enzymatic processes. The key cellular metabolic requirements for nucleotides have led to the development of many therapies that target different aspects of nucleotide biosynthesis and which are successfully used for the treatment of diverse diseases, such as cancer or autoimmune diseases.

Cellular pools of the pyrimidine nucleotide CTP (cytidine 5′ triphosphate) are derived from two sources; either a salvage pathway or through a de novo biosynthetic pathway that depends on the enzyme CTP synthase (or synthetase). In humans, there are two isoforms of CTP synthase, CTPS1 and CTPS2. These enzymes catalyze the conversion of the pyrimidine UTP (uridine 5′ triphosphate) into CTP in a series of coupled reactions that utilize the substrates glutamine and ATP (adenosine 5′ triphosphate), converting glutamine to L-glutamate and ATP to ADP (adenosine 5′ diphosphate). The specific biological roles of the two different isoforms of CTP synthase in humans have yet to be well delineated.

The immune system in multi-cellular organisms has evolved to provide protection from a diverse range of infectious pathogens. This process generally requires recognition of the pathogen by various immune cells and is often followed by amplification and long-term propagation of the immune response through the rapid expansion, proliferation, and differentiation of responding immune cells. Within this process, the activity of CTP synthase appears to play a key role in providing sufficient CTP via the de novo biosynthesis pathway for rapid expansion of immune lymphocytes following activation.

In humans, clinical data showing that CTPS1 is the critical enzyme for supporting lymphocyte proliferation has been provided through the identification of a loss-of-function homozygous mutation (rs145092287) in this enzyme that causes a severe immunodeficiency, characterized by a strongly reduced capacity of activated T- or B-cells to proliferate in response to antigen receptor-mediated activation. The absence of any other reported clinical phenotypes outside the immune system in homozygous carriers indicates a specific role for CTPS1 in supporting immune cell expansion and proliferation and suggests that CTPS1 activity may be dispensable or compensated by CTPS2 activity outside the immune system in humans.

Given the known therapeutic benefit of therapies targeting nucleotide synthesis and the key role of CTP synthase for de novo CTP generation to fuel metabolic demands of the cell, CTPS1 represents a target for a new class of agents with therapeutic potential, especially as related to immune dysfunction. Pathogenic immune cells, such as autoreactive T or B-cells, are drivers or components of diverse diseases, such as autoimmune diseases, severe allergic reactions, cardiovascular and metabolic disorders, degenerative neurological diseases, and hematological cancers. Inhibition of CTPS1 could provide therapeutic benefit in these or other diseases, and the specific role of CTPS1 in select immune cell subsets could also highlight the potential for an improved therapeutic index over other clinical therapies targeting nucleotide biosynthesis more broadly.

### SUMMARY OF THE INVENTION

It has now been found that compounds of this invention, and pharmaceutically acceptable compositions thereof, are effective as inhibitors of CTPS1. In certain embodiments, the invention provides compounds of the formulae presented herein.

Compounds of the present invention, and pharmaceutically acceptable compositions thereof, are useful for treating a variety of diseases, disorders or conditions, associated with regulation of CTPS1. Such diseases, disorders, or conditions include those described herein.

### DETAILED DESCRIPTION OF CERTAIN EMBODIMENTS

### 1. General Description of Certain Embodiments of the Invention

In certain aspects, the present invention provides a compound of formula I: A B C

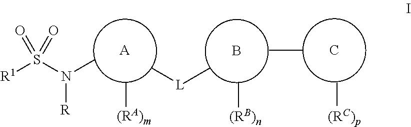

or a pharmaceutically acceptable salt thereof, wherein each of Ring A, Ring B, Ring C, L, R¹, R^{A}, R^{B}, R^{C}, m, n, and p, is as defined below and described in embodiments herein, both singly and in combination.

In some embodiments, the present invention provides a pharmaceutical composition comprising a compound of formula I, and a pharmaceutically acceptable carrier, adjuvant, or diluent.

In some embodiments, the present invention provides a method of treating a CTPS1-mediated disease, disorder, or condition, comprising administering to a patient in need thereof a compound of formula I or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

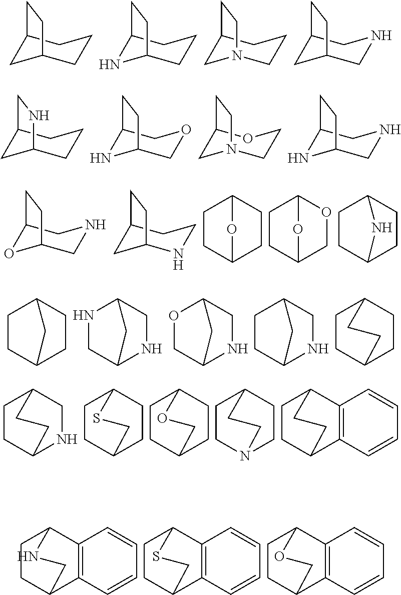

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” or in some embodiments “haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —N(R^{◯})C(NR^{◯})N(R^{◯})₂; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}; —SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; —SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or a 7-12 membered saturated or partially unsaturated bicyclic heterocyclic ring that is optionally bridged bicyclic (having 1-4 heteroatoms independently selected from nitrogen, oxygen, and sulfur), or a 7-12 membered saturated or partially unsaturated spirocyclic heterocyclic ring (having 1-4 heteroatoms independently selected from nitrogen, oxygen, and sulfur), or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR*), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR*, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Unless otherwise indicated, divalent structures or functional groups depicted herein are meant to include either direction at the points of attachment (e.g., the group

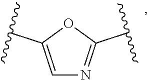 [SMILES: *c1cnc(*)o1]

when part of a Markush group attached to groups “A” and “B”, includes both

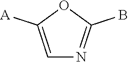

and

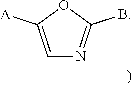

Compound structures shown throughout the present specification and in the Tables herein contain designations at certain stereocenters which indicate the following. “or1” or “or2” is intended to cover stereochemically pure compounds wherein the stereochemistry at the stereocenter marked with “or1” or “or2” is either the stereochemistry shown in the diagram or wherein the marked stereocenter has a configuration opposite to what is shown in the diagram. Stereocenters marked with “abs” intend to cover material wherein the marked stereocenter is of the stereochemistry shown in the diagram. Stereocenters marked with “&1” indicate that the compound material has a mixture of R and S-configured stereoisomers with respect to the marked stereocenter (for example, if the compound only contains one stereocenter and it is marked with “&1,” the material is racemic).

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, a “CTPS1 antagonist” or a “CTPS1 inhibitor” is a molecule that reduces, inhibits, or otherwise diminishes one or more of the biological activities of CTPS1. Antagonism using the CTPS1 antagonist does not necessarily indicate a total elimination of the CTPS1 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of CTPS1 compared to an appropriate control. The presently disclosed compounds bind directly to CTPS1 and inhibit its activity.

By “specific antagonist” is intended an agent that reduces, inhibits, or otherwise diminishes the activity of a defined target greater than that of an unrelated target. For example, a CTPS1 specific antagonist reduces at least one biological activity of CTPS1 by an amount that is statistically greater than the inhibitory effect of the antagonist on any other protein. In some embodiments, the IC₅₀ of the antagonist for the target is about 90%, 80%, 70%, 60%, 50%, 40%, 30%, 20%, 10%, 5%, 1%, 0.1%, 0.01%, 0.001% or less of the IC₅₀ of the antagonist for a non-target.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label,” “fluorescent dye,” and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a CTPS1 activity between a sample comprising a compound of the present invention, or composition thereof, and CTPS1, and an equivalent sample comprising CTPS1, in the absence of the compound, or composition thereof.

### 3. Description of Exemplary Embodiments

As described above, in certain embodiments, the present invention provides a compound of formula I:

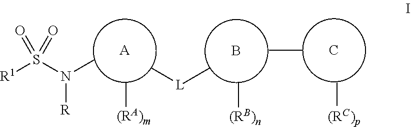

or a pharmaceutically acceptable salt thereof, wherein:
R¹ is selected from C₁₋₆ aliphatic; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; each of which is substituted with q instances of R^{A};
Ring A is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur;
L is

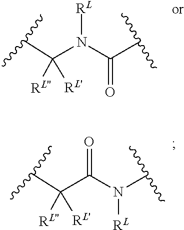

wherein R^{L} is hydrogen or optionally substituted C₁₋₆ aliphatic and one of R^{L′} or R^{L″} is hydrogen and the other of R^{L′} or R^{L″} is -L²-R², wherein:
   R² is hydrogen, halogen, —OR, —NR₂, an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 7-12 membered saturated or partially unsaturated spirocyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and
   L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—; or
R^{L} is hydrogen or optionally substituted C₁₋₆ aliphatic and an R^{L′} and R^{L″} group are taken together with the atom to which they are attached, to form an optionally substituted 3-7 membered saturated or partially unsaturated monocyclic carbocyclic ring or a 3-7 membered optionally substituted saturated or partially unsaturated monocyclic heterocyclic ring having 1-2 heteroatoms independently selected from nitrogen, oxygen, and sulfur;
Ring B is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-3 heteroatoms independently selected from nitrogen, oxygen, and sulfur; a 5-11 membered saturated or partially unsaturated fused, bridged, or spiro, bicyclic carbocyclic ring; a 7-11 membered fused bicyclic aryl ring; a 7-12 membered saturated or partially unsaturated bicyclic heterocyclic ring that is optionally bridged bicyclic (having 1-4 heteroatoms independently selected from nitrogen, oxygen, and sulfur), a 7-12 membered saturated or partially unsaturated spirocyclic heterocyclic ring (having 1-4 heteroatoms independently selected from nitrogen, oxygen, and sulfur); and a 7-11 membered fused bicyclic heteroaryl ring having 1-4 heteroatoms independently selected from nitrogen, oxygen, and sulfur;
Ring C is selected from a phenyl, 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur;
each instance of R^{A}, R^{B}, and R^{C} is independently optionally substituted C₁₋₄ aliphatic, oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂;
each R is independently hydrogen, —CN, halogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 9-12 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring; or
two R groups are taken together with the atoms to which each R is attached, to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and
m is 0, 1, or 2;
n is 0, 1, or 2;
p is 0, 1, or 2; and
q is 0, 1, 2, 3, or 4.

In some embodiments, it is provided that when:
R¹ is C₁₋₆ aliphatic or a 3-7 membered saturated or partially unsaturated monocyclic carbocyclic ring;
the R group of the sulfonamide moiety

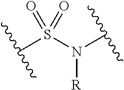

is hydrogen or para-methoxybenzyl;
L is

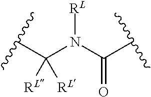

and the R^{L} and R^{L′} or R^{L} and R^{L″} groups are not taken together with the atoms to which each is attached to form an optionally substituted 3-7 membered saturated or partially unsaturated monocyclic heterocyclic ring having 1-2 heteroatoms independently selected from nitrogen, oxygen, and sulfur, or L is

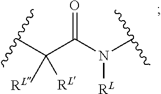

Ring B is phenyl or a 6-membered monocyclic heteroaryl ring having 1-4 heteroatoms independently selected from nitrogen, oxygen, and sulfur; and
Ring C is phenyl or a 6-membered monocyclic heteroaryl ring having 1-4 heteroatoms independently selected from nitrogen, oxygen, and sulfur and is attached to Ring B in the para position relative to the L group;
then Ring A and its R^{A} substituents are other than

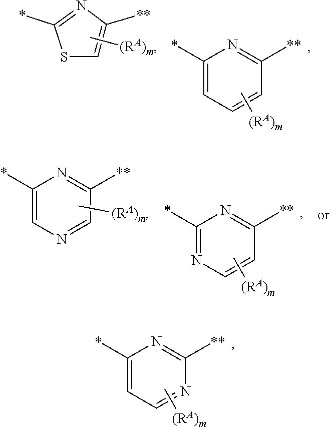

where * denotes attachment to the

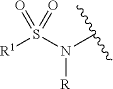

moiety and ** denotes attachment to the

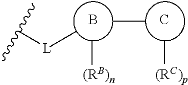

moiety.

In some embodiments, it is provided that when:
R¹ is C₁₋₆ aliphatic or a 3-7 membered saturated or partially unsaturated monocyclic carbocyclic ring;
the R group of the sulfonamide moiety

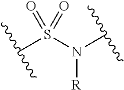

is hydrogen or para-methoxybenzyl;
L is

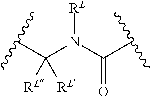

and the R^{L} and R^{L′} or R^{L} and R^{L″} groups are not taken together with the atoms to which each is attached to form an optionally substituted 3-7 membered saturated or partially unsaturated monocyclic heterocyclic ring having 1-2 heteroatoms independently selected from nitrogen, oxygen, and sulfur, or L is

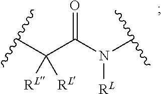

Ring B is phenyl or a 6-membered monocyclic heteroaryl ring having 1-4 heteroatoms independently selected from nitrogen, oxygen, and sulfur; and
Ring C is phenyl or a 6-membered monocyclic heteroaryl ring having 1-4 heteroatoms independently selected from nitrogen, oxygen, and sulfur and is attached to Ring B in the para position relative to the L group;
then Ring A and its R^{A} substituents are other than

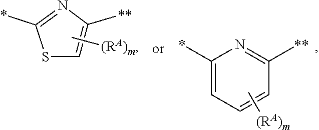

where * denotes attachment to the

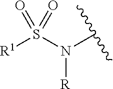

moiety and ** denotes attachment to the

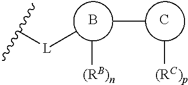

moiety.

As described generally above, R¹ is selected from C₁₋₆ aliphatic; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; each of which is substituted with q instances of R^{A}.

In certain embodiments, R¹ is selected from C₁₋₆ haloalkyl, cyclopropyl, cyclobutyl, cyclopentyl, cyclohexyl, cycloheptyl, aziridinyl, azetidinyl, pyrrolidinyl, piperidinyl, oxiranyl, oxetanyl, tetrahydrofuranyl, and tetrahydropyranyl; each of which is substituted with q instances of R^{A}.

In certain embodiments, R¹ is selected from C₁₋₆ haloalkyl, cyclopropyl, cyclobutyl, aziridinyl, azetidinyl, oxiranyl, and oxetanyl; each of which is substituted with q instances of R^{A}.

In certain embodiments, R¹ is —CF₃, —CHF₂,

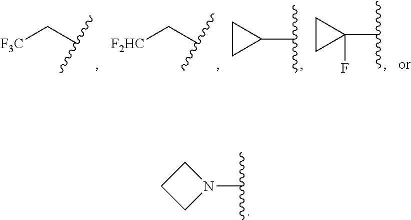

In certain embodiments, R¹ is —CHF₂ or

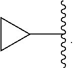 [SMILES: *C1CC1]

In certain embodiments, R¹ is —CHF₂. In certain embodiments, R¹ is

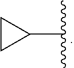 [SMILES: *C1CC1]

In some embodiments, R¹ is selected from those depicted in Table 1, below.

As described generally above, Ring A is selected from phenyl; a 5-6 membered monocyclic heteroaryl ring having 1-4 heteroatoms independently selected from nitrogen, oxygen, and sulfur; and a 7-11 membered fused bicyclic heteroaryl ring having 1-4 heteroatoms independently selected from nitrogen, oxygen, and sulfur.

In some embodiments, Ring A is selected from phenyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, furanyl, furazanyl, imidazolyl, 1H-indazolyl, indolenyl, indolinyl, indolizinyl, indolyl, 3H-indolyl, isoindolinyl, isoindolenyl, isobenzofuranyl, isoindazolyl, isoindolinyl, isoindolyl, isoquinolinyl, isothiazolyl, isoxazolyl, oxadiazolyl, 1,2,3-oxadiazolyl, 1,2,4-oxadiazolyl; 1,2,5oxadiazolyl, 1,3,4-oxadiazolyl, oxazolyl, pyrimidinyl, pteridinyl, purinyl, pyranyl, pyrazinyl, pyrazolidinyl, pyrazolinyl, pyrazolyl, pyridazinyl, pyridinyl, pyrimidinyl, pyrrolinyl, 2H-pyrrolyl, pyrrolyl, quinazolinyl, quinolinyl, quinoxalinyl, thiadiazolyl, 1,2,3-thiadiazolyl, 1,2,4-thiadiazolyl, 1,2,5-thiadiazolyl, 1,3,4-thiadiazolyl, thiazolyl, thienyl, thiophenyl, triazinyl, 1,2,3-triazolyl, 1,2,4-triazolyl, 1,2,5-triazolyl, and 1,3,4-triazolyl.

In some embodiments, Ring A is selected from phenyl, benzimidazolyl, imidazolyl, 1H-indazolyl, indolenyl, indolinyl, indolizinyl, indolyl, 3H-indolyl, isoindolinyl, isoindolenyl, isoindazolyl, isoindolinyl, isoindolyl, isoquinolinyl, isothiazolyl, isoxazolyl, oxadiazolyl, 1,2,3-oxadiazolyl, 1,2,4-oxadiazolyl; 1,2,5oxadiazolyl, 1,3,4-oxadiazolyl, oxazolyl, pyrimidinyl, pyranyl, pyrazinyl, pyrazolidinyl, pyrazolinyl, pyrazolyl, pyridazinyl, pyridinyl, pyrimidinyl, pyrrolinyl, 2H-pyrrolyl, pyrrolyl, thiadiazolyl, 1,2,3-thiadiazolyl, 1,2,4-thiadiazolyl, 1,2,5-thiadiazolyl, 1,3,4-thiadiazolyl, and thiazolyl.

In some embodiments, Ring A is selected from phenyl, pyrazolyl, thiazolyl, thiadiazolyl, pyridinyl, pyridazinyl, pyrimidinyl, and indolyl. In some embodiments, Ring A is selected from phenyl, pyrazinyl, pyrimidinyl, or pyridinyl.

In certain embodiments, Ring A is

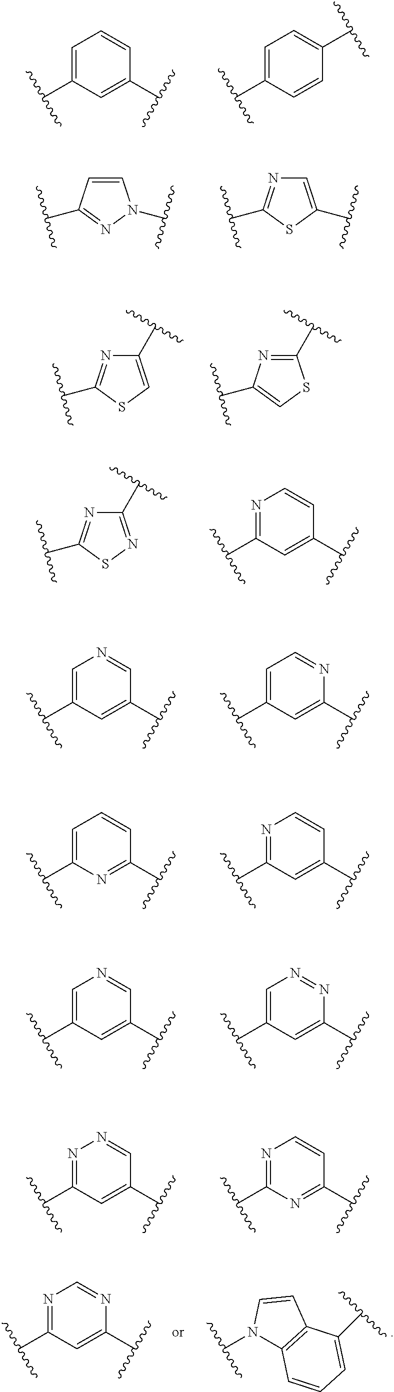

In certain embodiments, Ring A and its R^{A} substituents together form

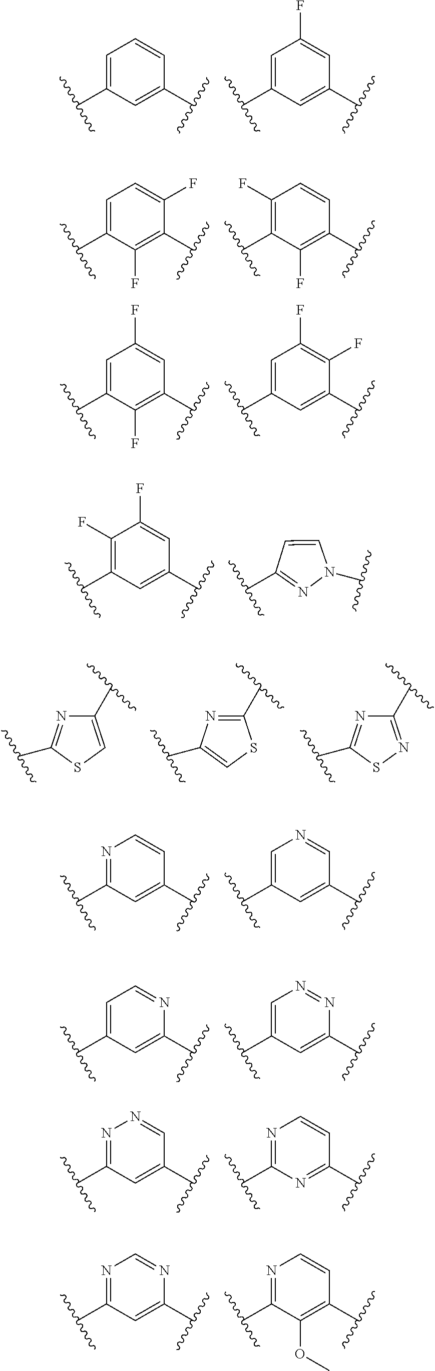

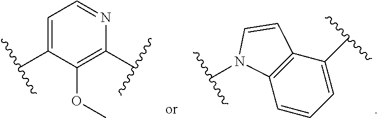

In certain embodiments, Ring A is

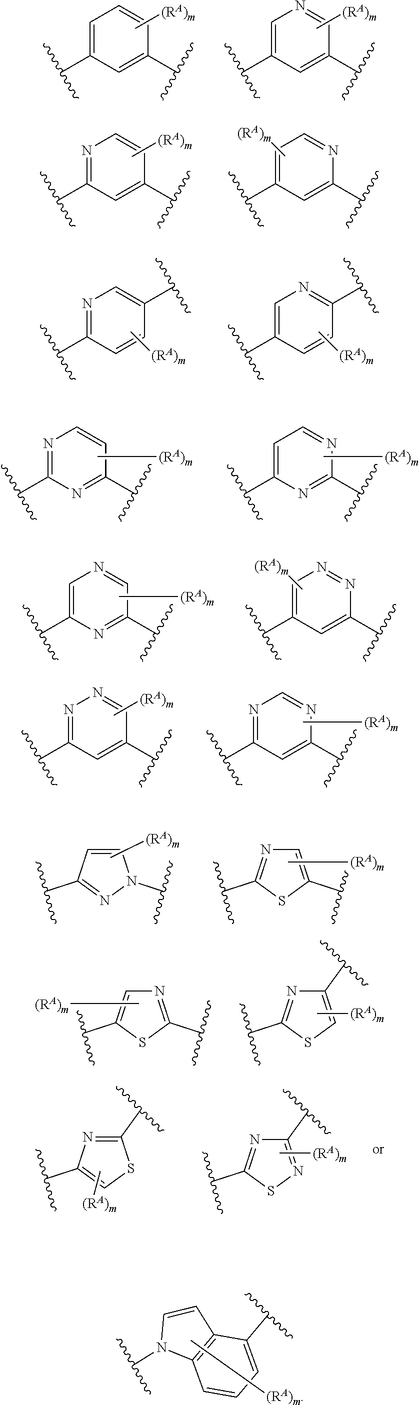

In certain embodiments, Ring A is

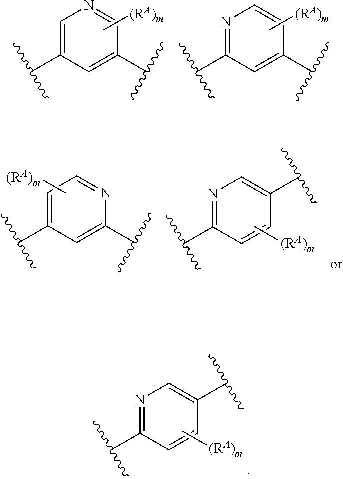

In certain embodiments, Ring A is

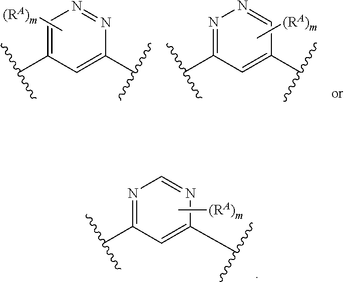

In certain embodiments, Ring A is

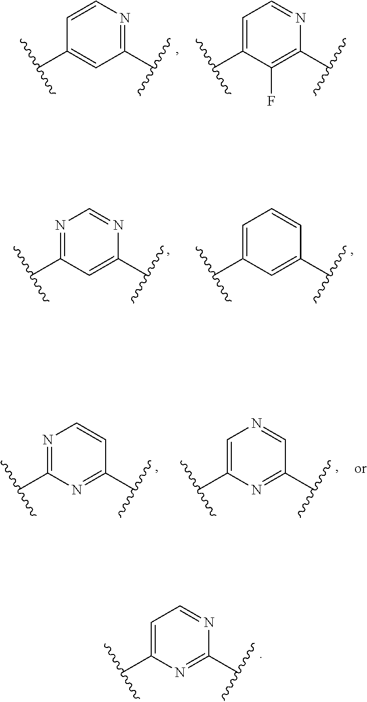

In certain embodiments, Ring A is

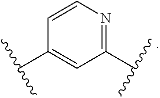 [SMILES: *c1ccnc(*)c1]

In certain embodiments, Ring A is

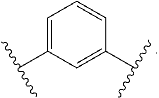 [SMILES: *c1cccc(*)c1]

In some embodiments, Ring A is selected from those depicted in Table 1, below.

In some embodiments, L is

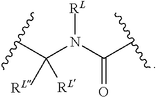

In some embodiments, L is

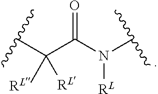

In certain embodiments, L is

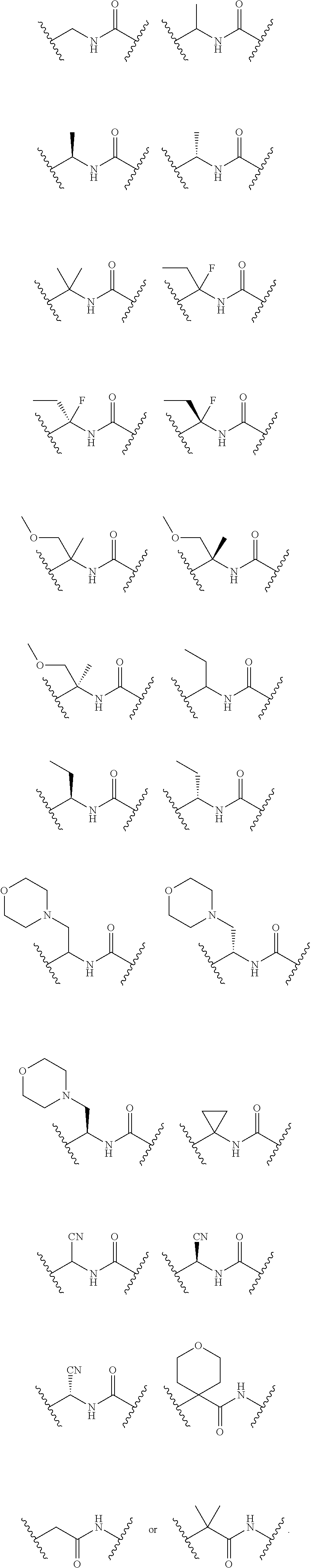

In certain embodiments, L is

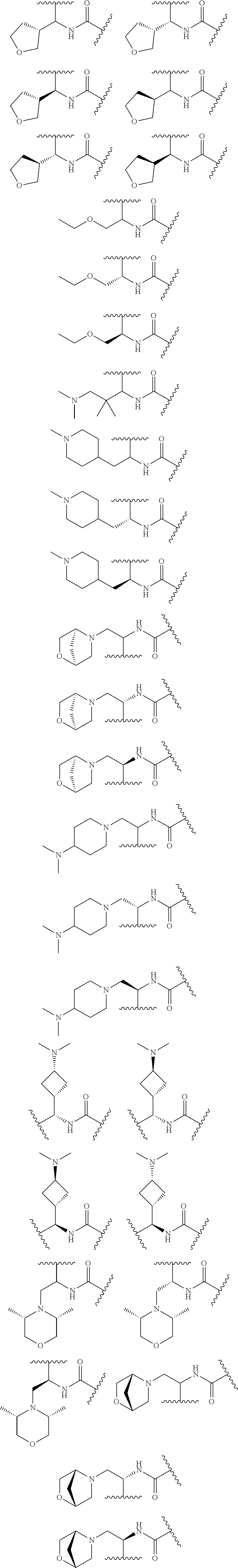

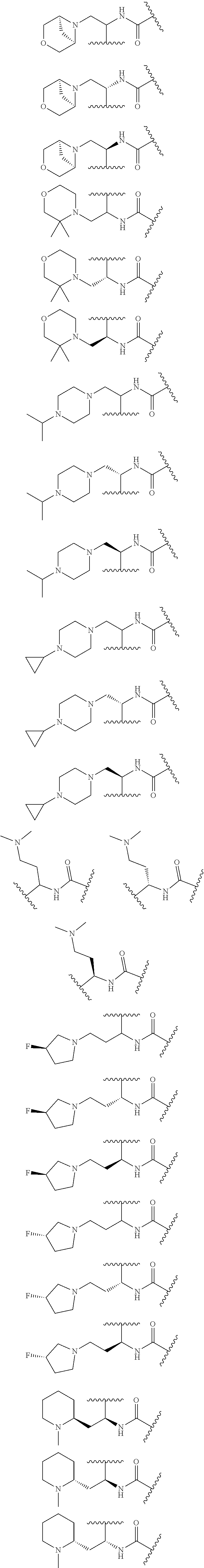

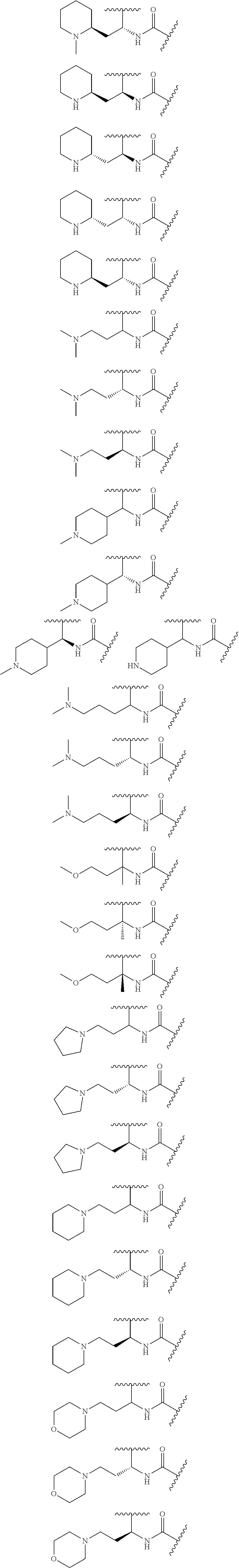

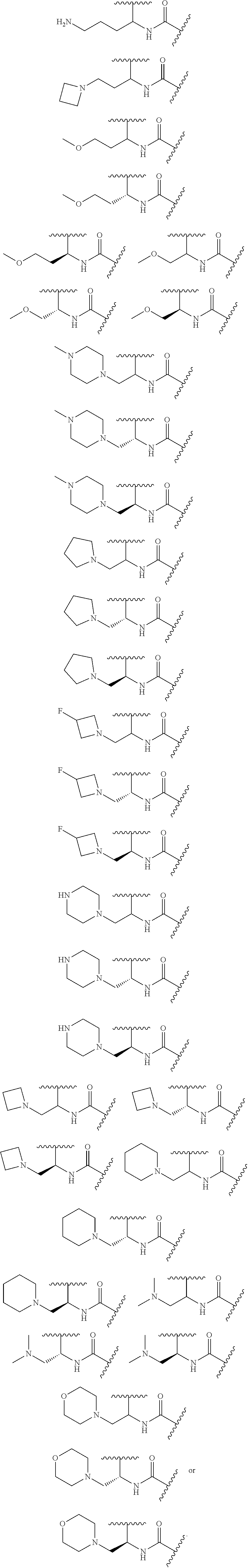

In certain embodiments, L is

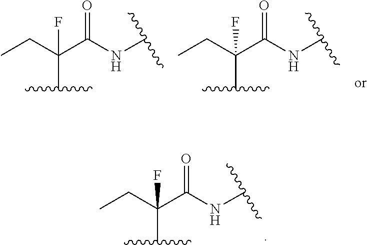

In some embodiments, L is

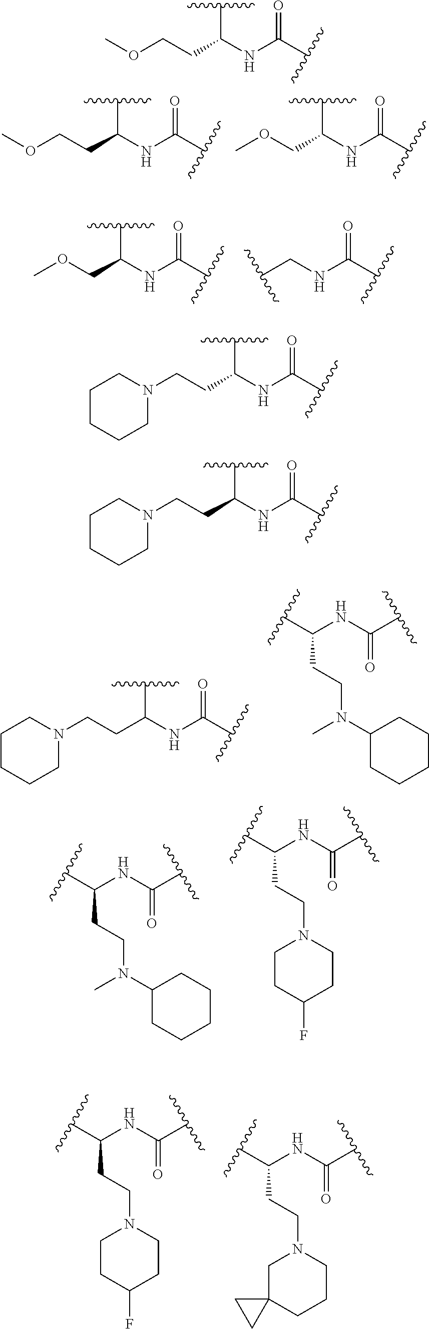

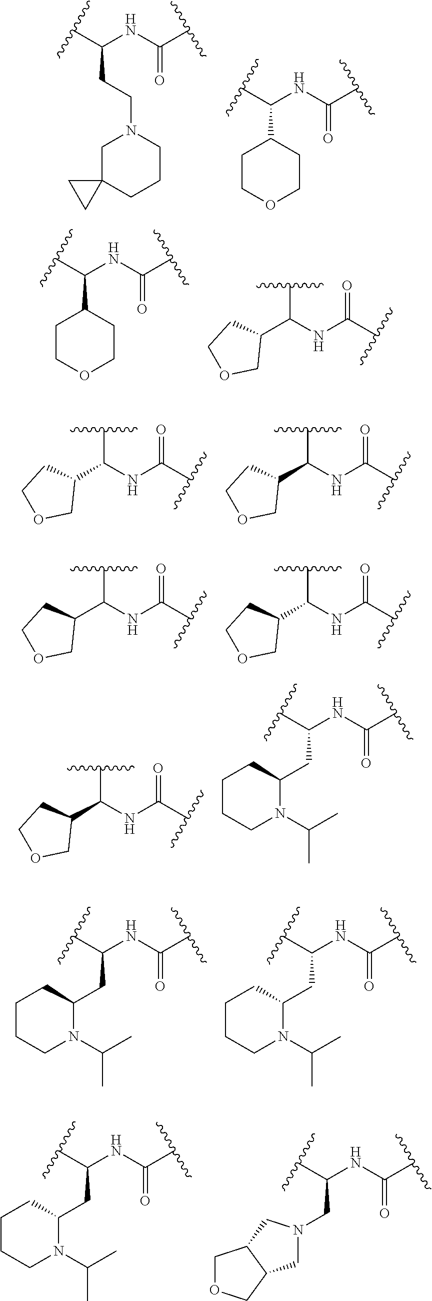

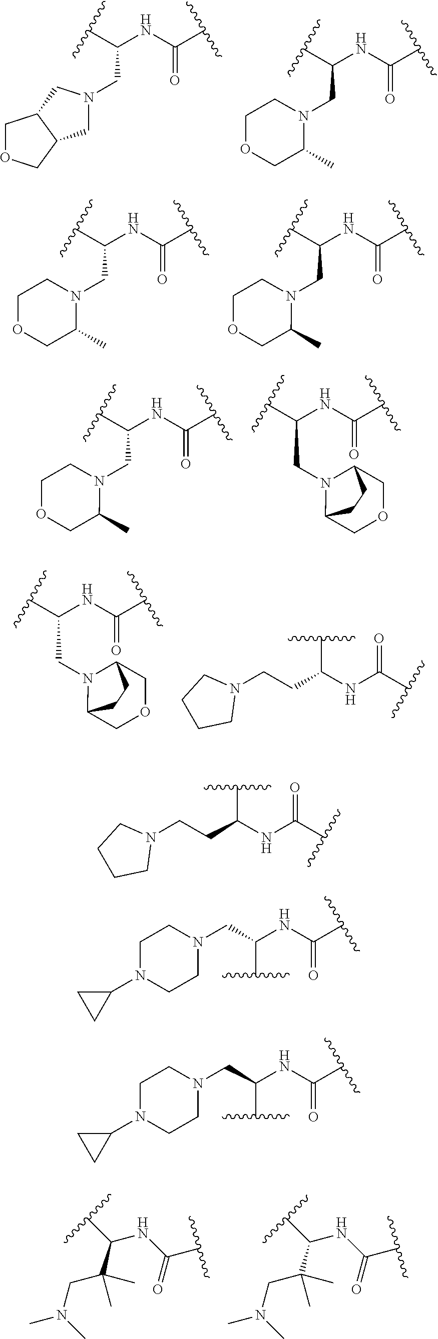

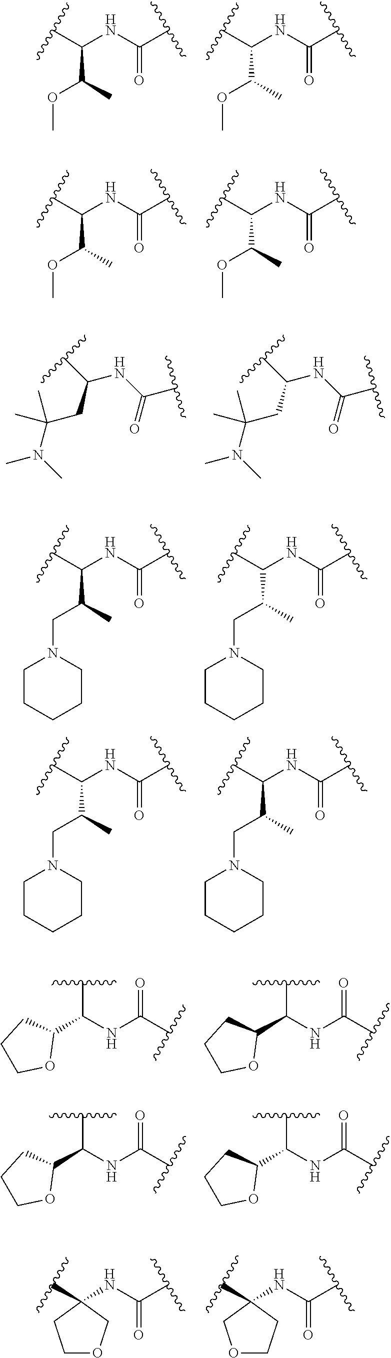

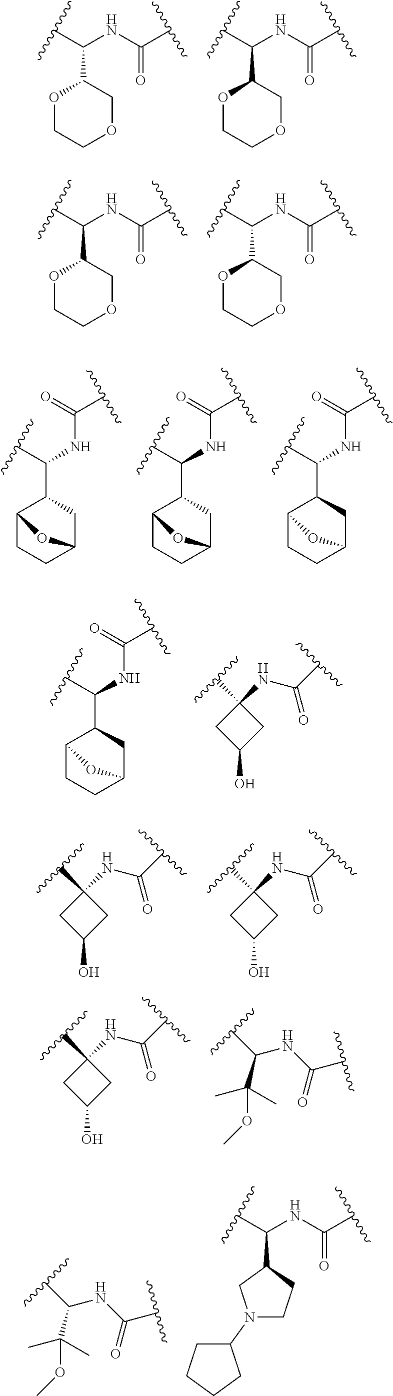

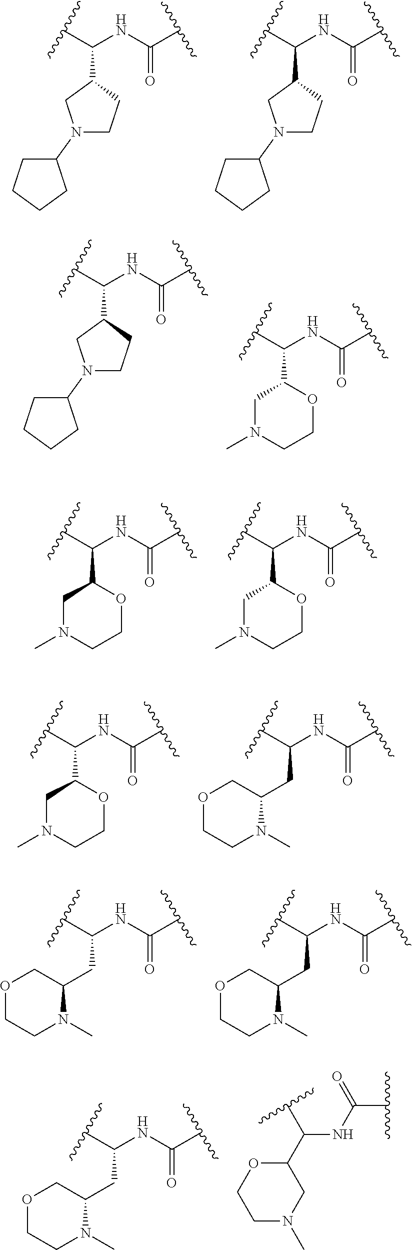

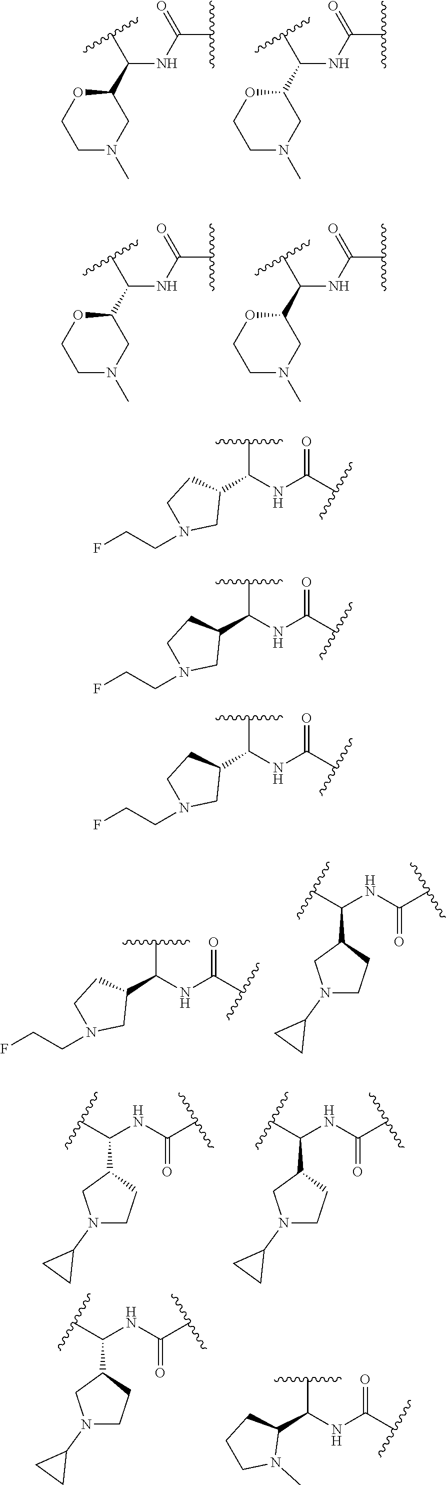

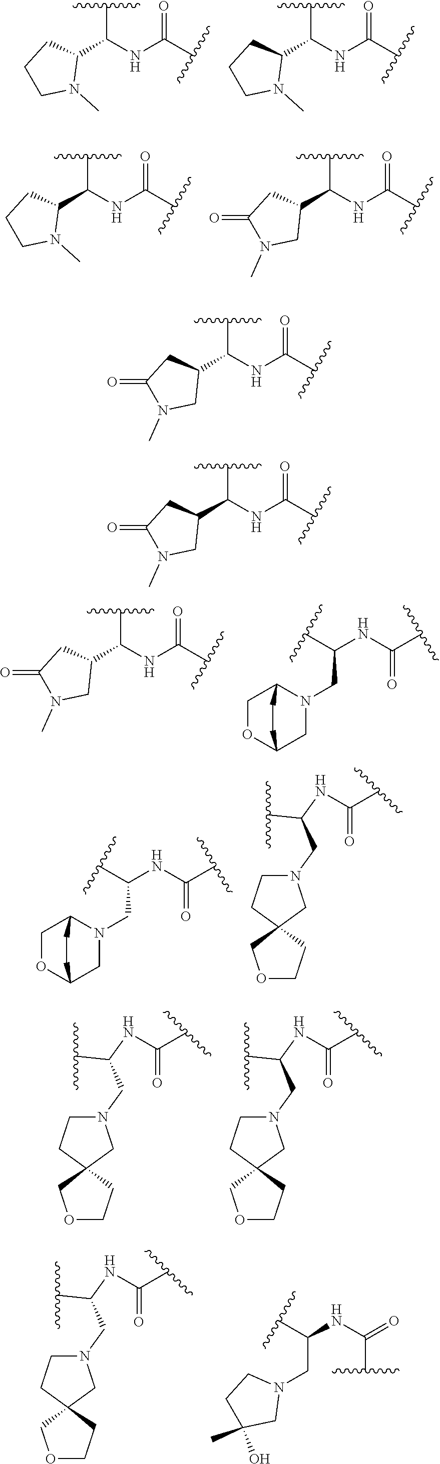

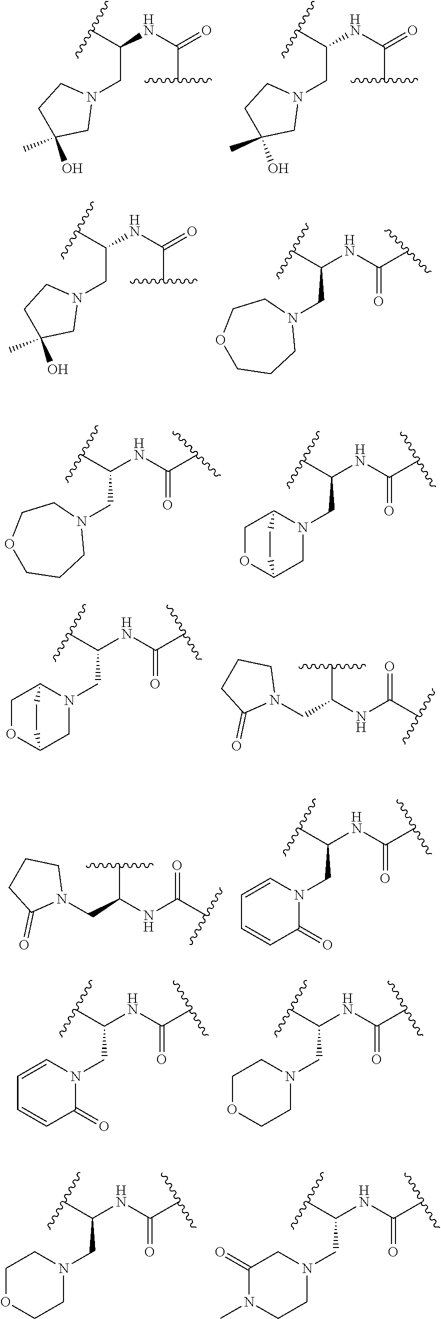

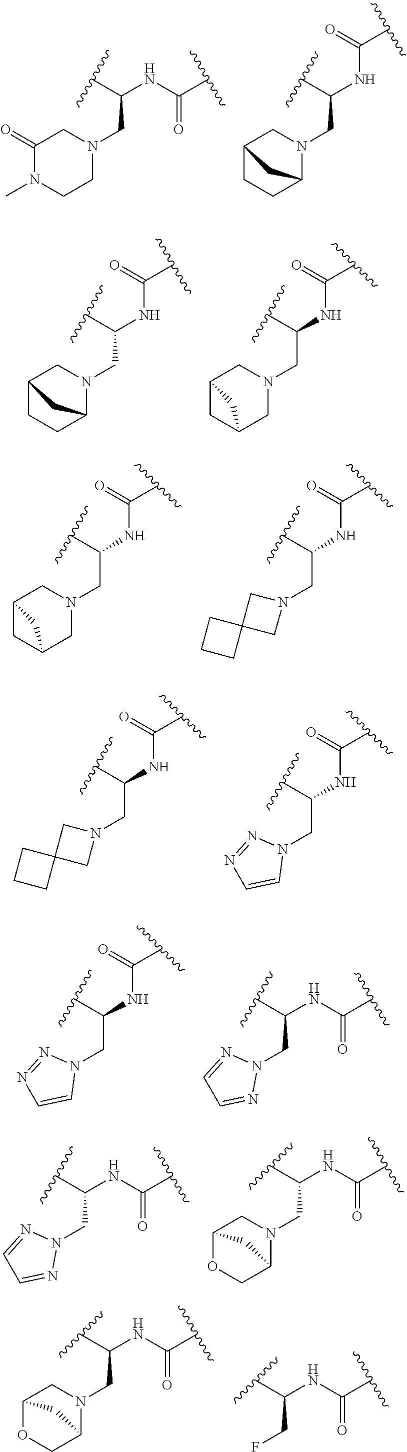

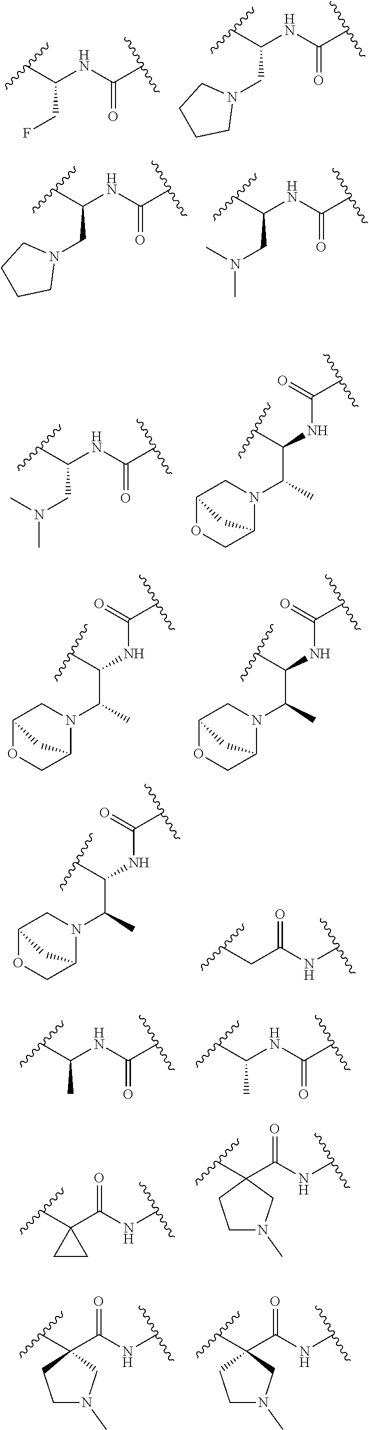

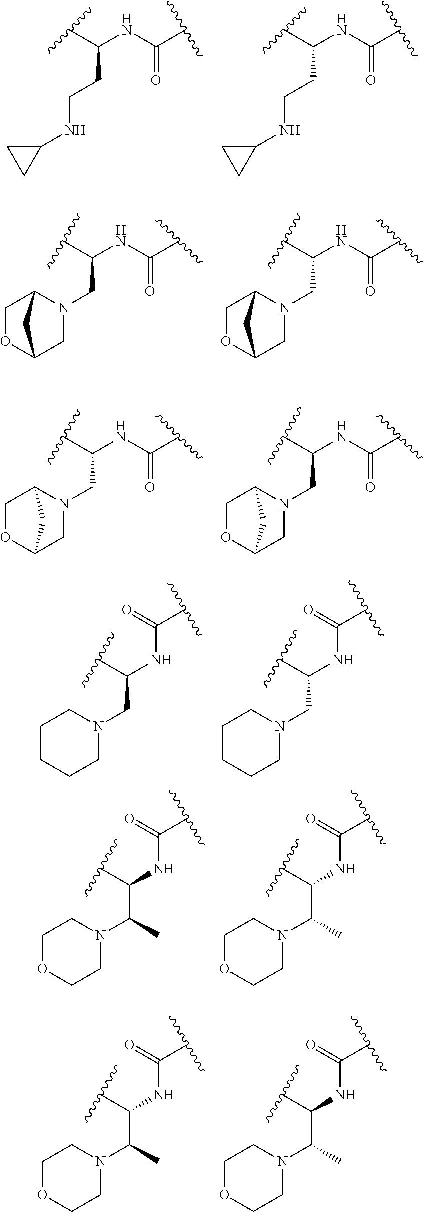

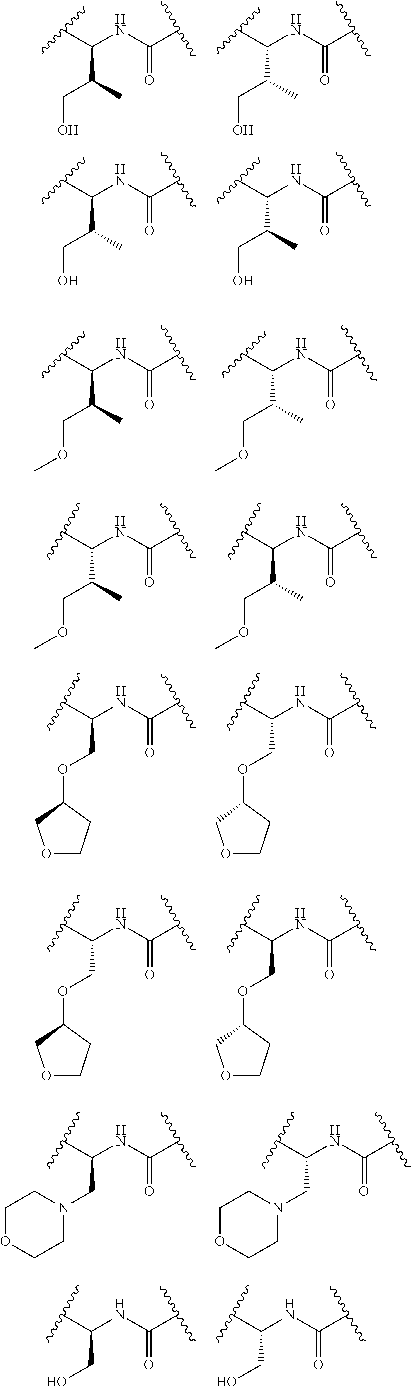

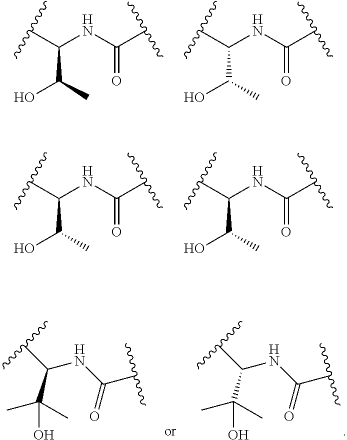

In some embodiments, L is

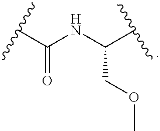 [SMILES: *C(=O)N[C@H](*)COC]

In some embodiments, L is

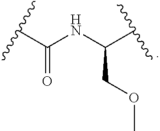 [SMILES: *C(=O)N[C@@H](*)COC]

In some embodiments, L is

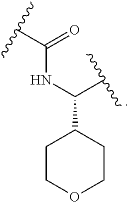 [SMILES: *C(=O)N[C@H](*)C1CCOCC1]

In some embodiments, L is

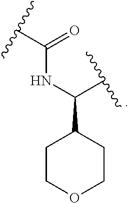 [SMILES: *C(=O)N[C@@H](*)C1CCOCC1]

In some embodiments, each of R^{L′}, and R^{L″} is independently hydrogen,

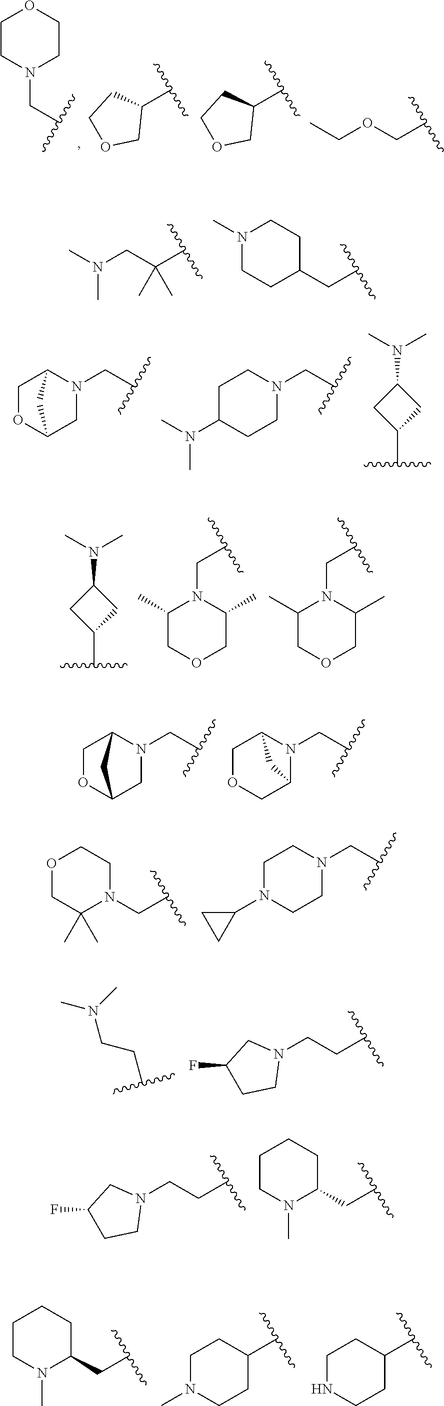

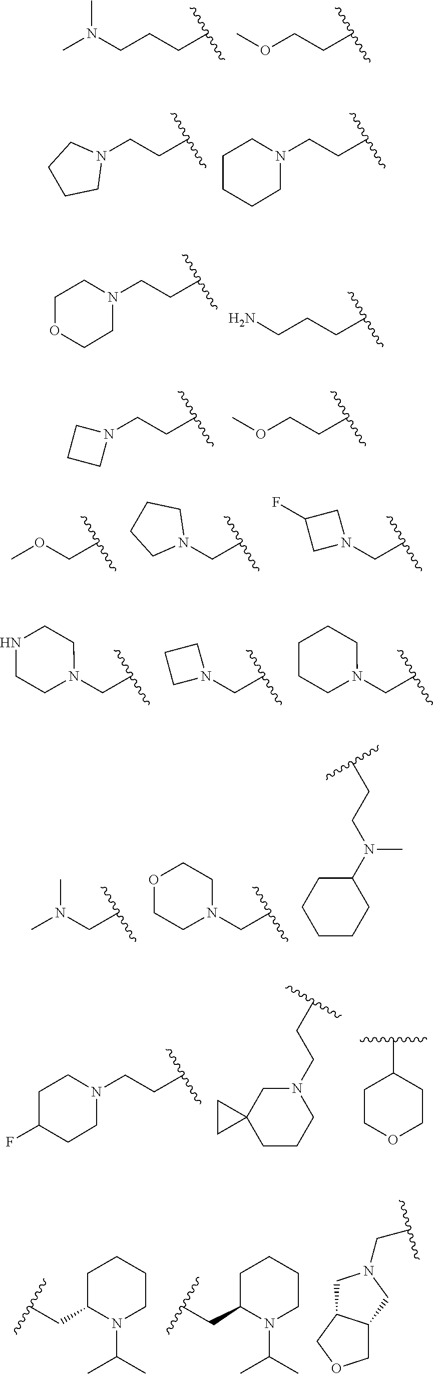

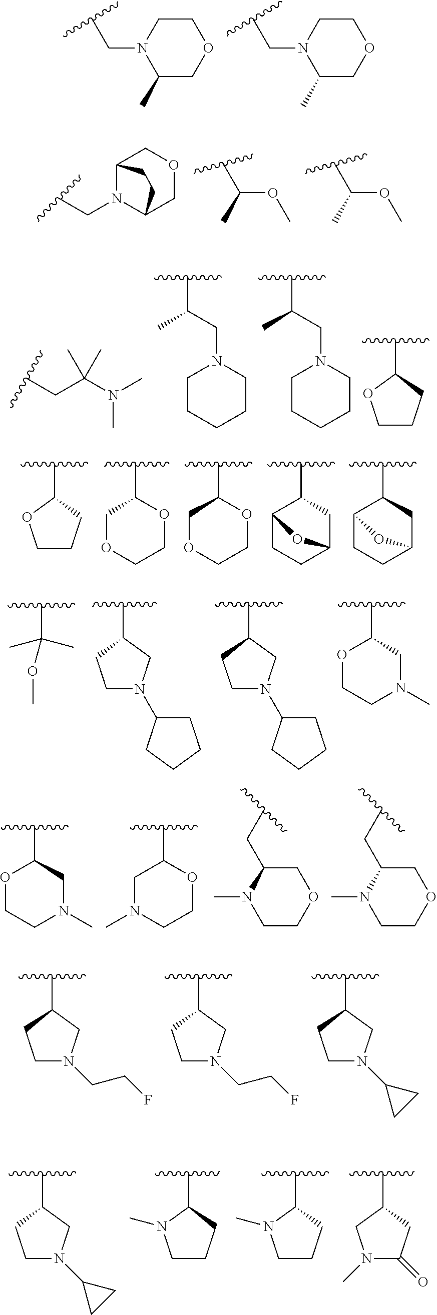

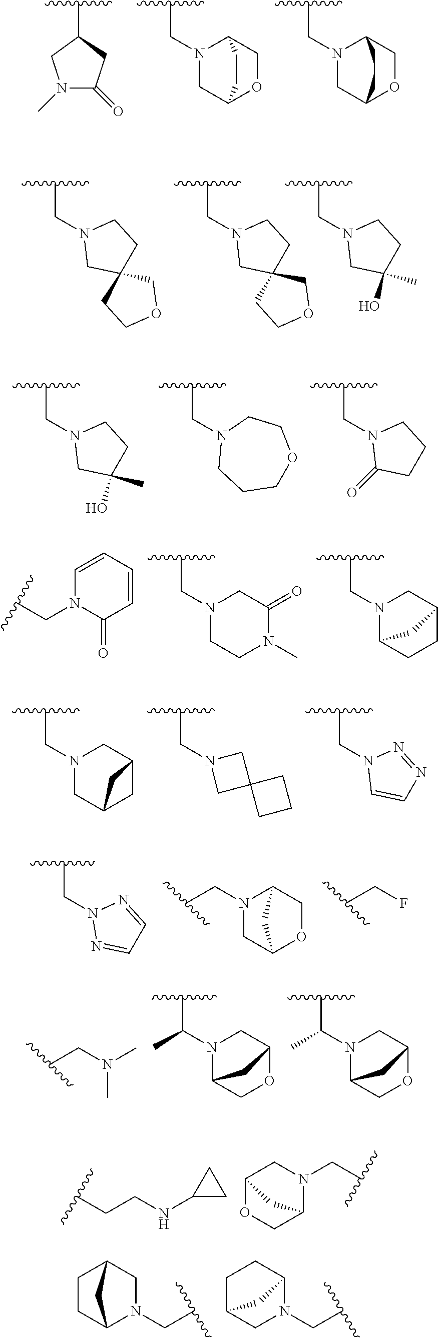

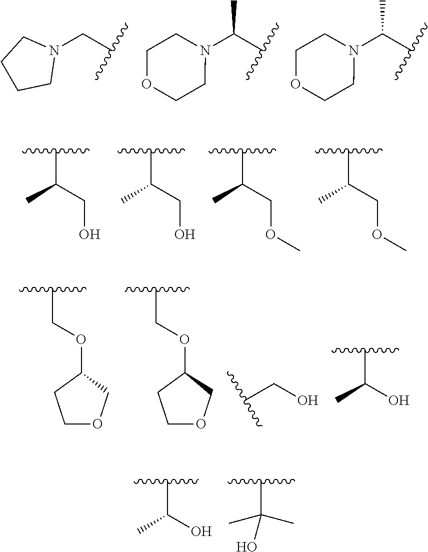

halogen, an optionally substituted C₁₋₆ aliphatic, or methyl.

In some embodiments, each of R^{L′}, and R^{L″} is independently hydrogen,

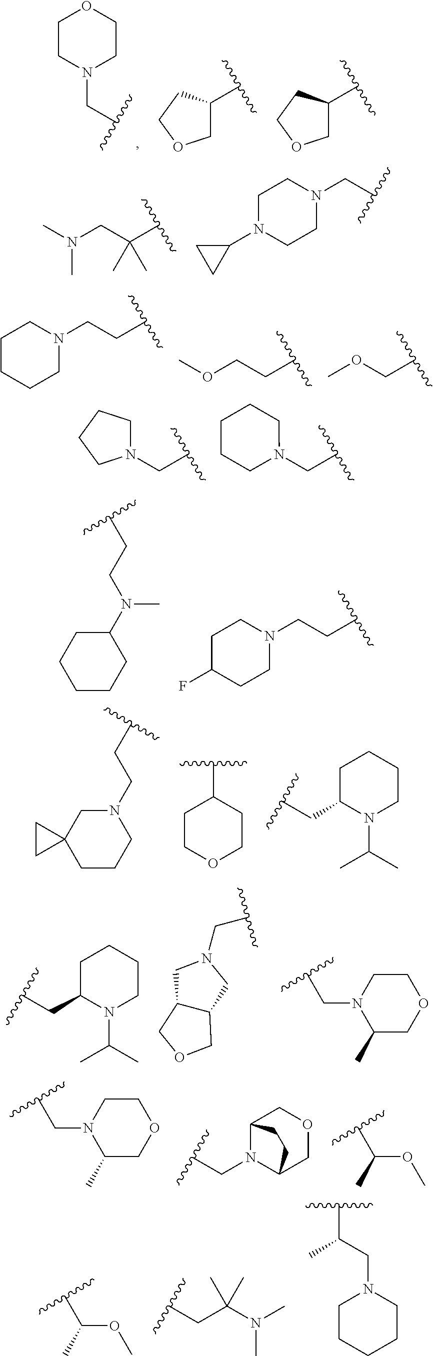

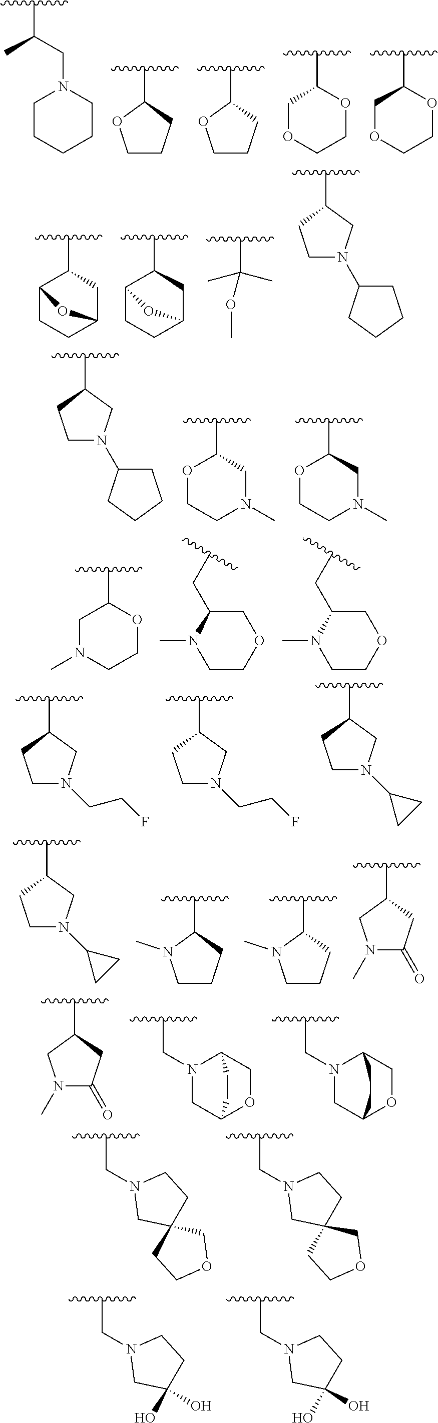

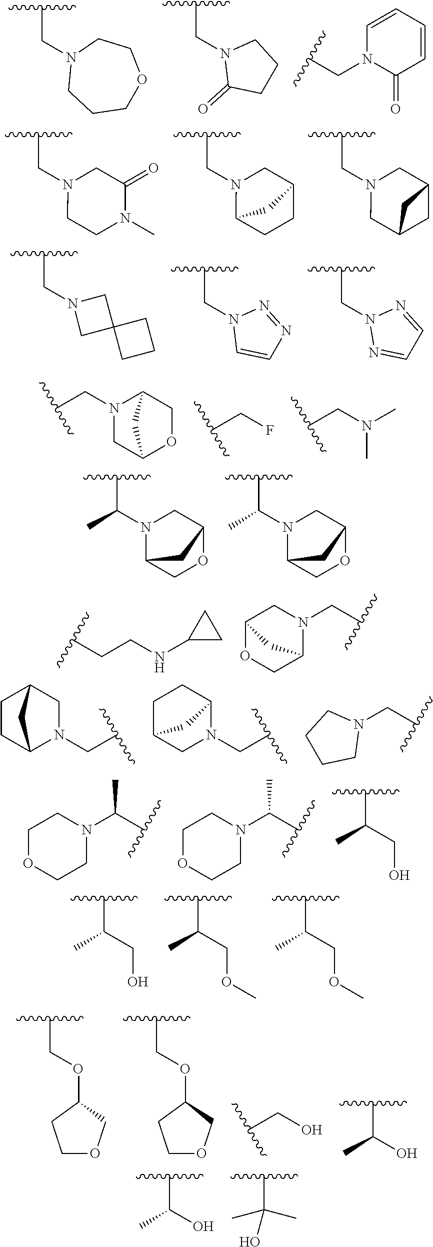

or methyl.

In some embodiments, each of R^{L′}, and R^{L″} is independently hydrogen, F, —CN, -Me or -Et. In some embodiments, R^{L′} and R^{L″} are hydrogen. In some embodiments, one of R^{L′} and R^{L″} is hydrogen.

In some embodiments, R^{L′} and R^{L″} together with the carbon to which they are attached

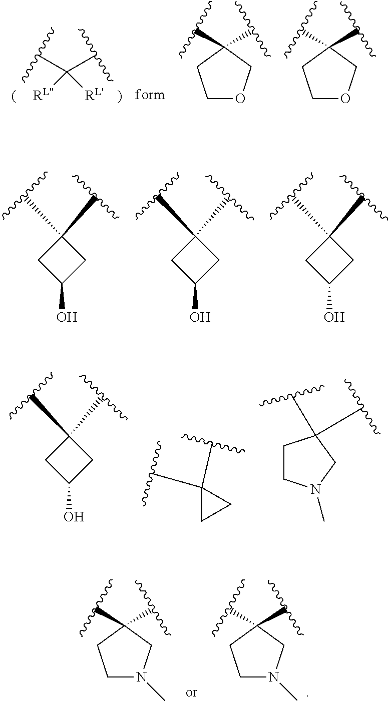

In some embodiments, R^{L} is H or methyl. In some embodiments, R^{L} is H. In some

embodiments, R^{L} is methyl.

In some embodiments, L is selected from those depicted in Table 1, below.

As described generally above, Ring B is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-3 heteroatoms independently selected from nitrogen, oxygen, and sulfur; a 5-11 membered saturated or partially unsaturated fused, bridged, or spiro, bicyclic carbocyclic ring; a 7-11 membered fused bicyclic aryl ring; a 7-11 membered saturated or partially unsaturated fused, bridged, or spiro, bicyclic heterocyclic ring having 1-4 heteroatoms independently selected from nitrogen, oxygen, and sulfur; and a 7-11 membered fused bicyclic heteroaryl ring having 1-4 heteroatoms independently selected from nitrogen, oxygen, and sulfur.

In some embodiments, Ring B is selected from phenyl; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-3 heteroatoms independently selected from nitrogen, oxygen, and sulfur; a 5-11 membered saturated or partially unsaturated fused, bridged, or spiro, bicyclic carbocyclic ring; a 7-11 membered saturated or partially unsaturated fused, bridged, or spiro, bicyclic heterocyclic ring having 1-4 heteroatoms independently selected from nitrogen, oxygen, and sulfur; and a 7-11 membered fused bicyclic heteroaryl ring having 1-4 heteroatoms independently selected from nitrogen, oxygen, and sulfur.

In some embodiments, Ring B is selected from phenyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, decahydroquinolinyl, dihydrofuro [2,3-b]tetrahydrofuran, furanyl, furazanyl, imidazolidinyl, imidazolinyl, isoindolinyl, isoindolenyl, isobenzofuranyl, isoindolinyl, isoindolinonyl, isoquinolinyl, isothiazolyl, isoxazolyl, morpholinyl, octahydroisoquinolinyl, oxadiazolyl, 1,2,3-oxadiazolyl, 1,2,4-oxadiazolyl; 1,2,5oxadiazolyl, 1,3,4-oxadiazolyl, oxazolidinyl, oxazolyl, oxazolidinyl, pyrimidinyl, piperazinyl, piperazinonyl, piperidinyl, pyranyl, pyrazinyl, pyrazolidinyl, pyrazolinyl, pyrazolyl, pyrazolopyridinyl, pyridazinyl, pyridinyl, pyridinonyl, pyrimidinyl, pyrrolidinyl, pyrrolinyl, 2H-pyrrolyl, pyrrolyl, quinazolinyl, quinolinyl, 4H-quinolizinyl, quinoxalinyl, tetrahydrofuranyl, tetrahydropyranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thiazolyl, thienyl, triazinyl, 1,2,3-triazolyl, 1,2,4-triazolyl, 1,2,5-triazolyl, 1,3,4-triazolyl, oxetanyl, azetidinyl, or bicyclo[1.1.1]pentanyl.

In some embodiments, Ring B is selected from phenyl, isoindolinyl, isoindolinonyl, 1,3,4-oxadiazolyl, oxazolyl, piperazinyl, piperazinonly, pyrazinyl, pyrazolyl, pyrazolopyridinyl, pyridinyl, pyridinonyl, tetrahydroisoquinolinyl, tetrahydroquinolinyl, 1,3,4-thiadiazolyl, thiazolyl, or bicyclo[1.1.1]pentanyl.

In certain embodiments, Ring B is

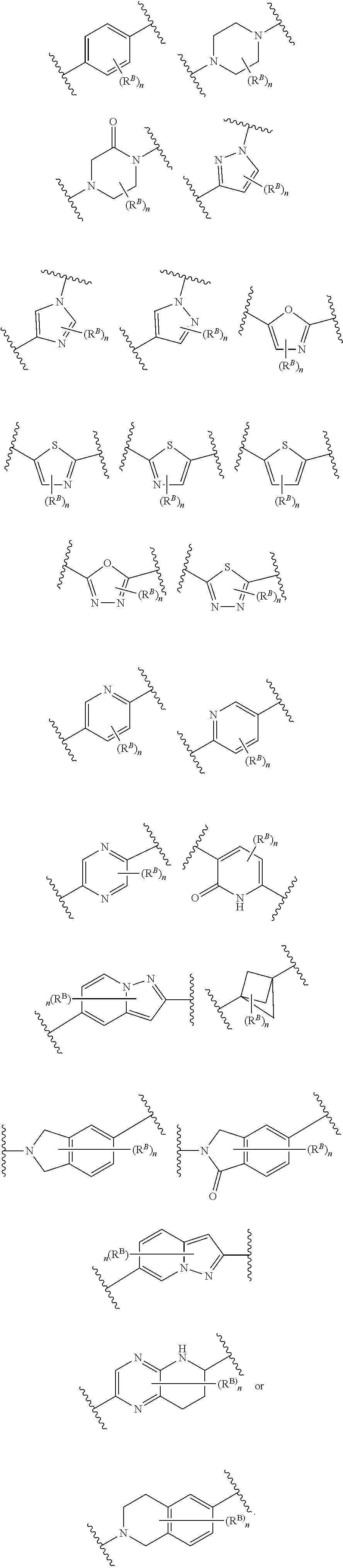

In certain embodiments, Ring B is

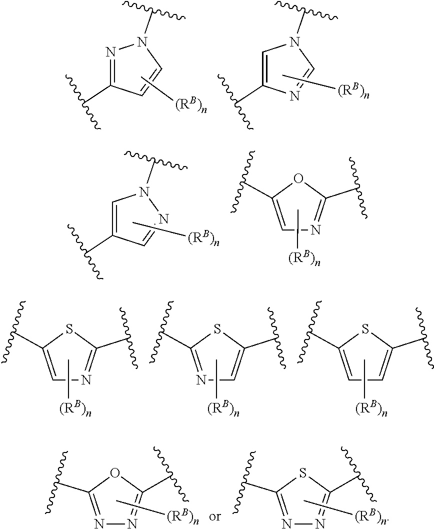

In certain embodiments, Ring B is a 5 membered saturated or partially unsaturated monocyclic carbocyclic ring; a 5 membered monocyclic heteroaryl ring having 1-3 heteroatoms independently selected from nitrogen, oxygen, and sulfur; or a 5 membered saturated or partially unsaturated monocyclic heterocyclic ring having 1-3 heteroatoms independently selected from nitrogen, oxygen, and sulfur. In certain embodiments, Ring B is phenyl, pyridinyl, or thiazolyl.

In certain embodiments, Ring B and its R^{B} substituents together form

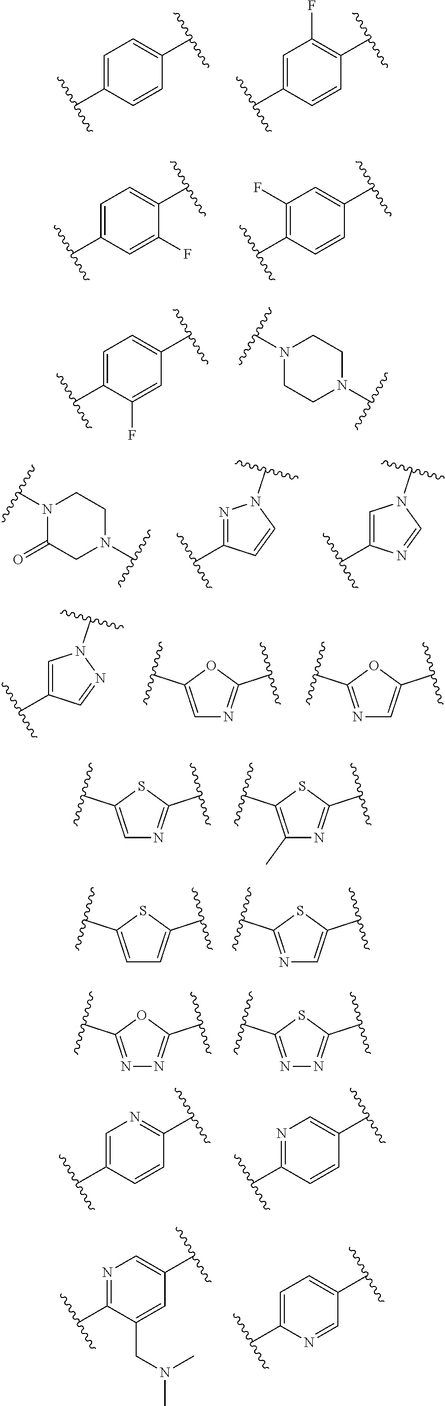

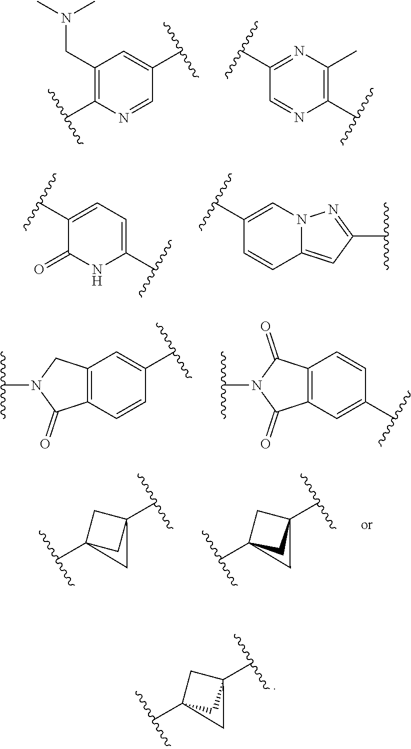

In some embodiments, Ring B is

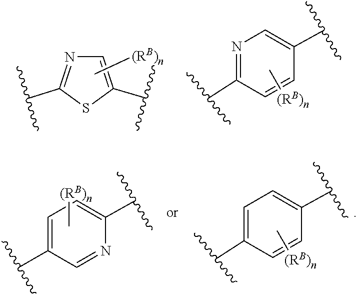

In some embodiments, Ring B and its R^{B} substituents together form

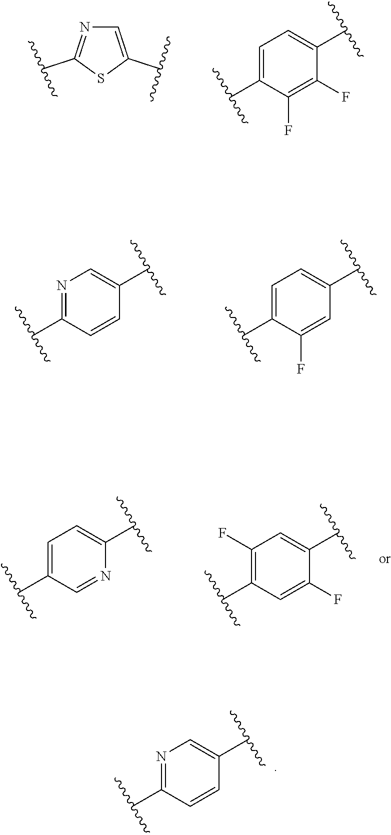

In some embodiments, Ring B and R^{B} together are

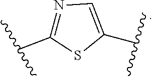 [SMILES: *c1cnc(*)s1]

In some embodiments, Ring B is selected from those depicted in Table 1, below.

As defined generally above, Ring C is selected from a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur.

In certain embodiments, Ring C is phenyl, cyclopropyl, cyclobutyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, furanyl, furazanyl, imidazolidinyl, imidazolinyl, imidazolyl, imidazopyrimidine, 1H-indazolyl, indolenyl, indolinyl, indolizinyl, indolyl, 3H-indolyl, isoindolinyl, isoindolenyl, isobenzofuranyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, oxetanyl, pyrimidinyl, piperazinyl, piperidinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, pyrrolopyridine, quinazolinyl, quinolinyl, 4H-quinolizinyl, tetrahydrofuranyl, tetrahydropyranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, triazinyl, 1,2,3-triazolyl, 1,2,4-triazolyl, 1,2,5-triazolyl, 1,3,4-triazolyl, oxetanyl, or azetidinyl.

In certain embodiments, Ring C is imidazolyl, imidazopyrimidine, oxadiazolyl, 1,2,3-oxadiazolyl, 1,2,4-oxadiazolyl; -1,2,5oxadiazolyl, 1,3,4-oxadiazolyl, piperidinyl, pyrazinyl, pyrrolidinyl, pyrrolopyridine, or azetidinyl. In certain embodiments, Ring C is pyrazinyl or pyrazolo[1,5-a]pyridinyl.

In certain embodiments, Ring C is

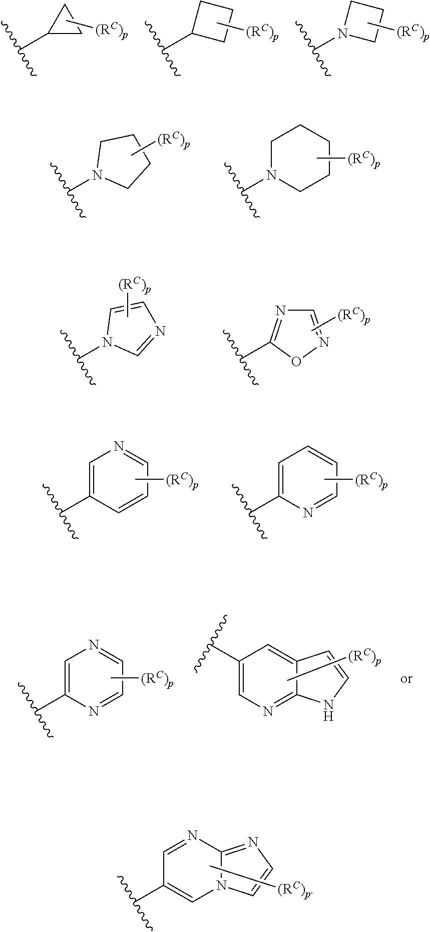

In certain embodiments, Ring C is

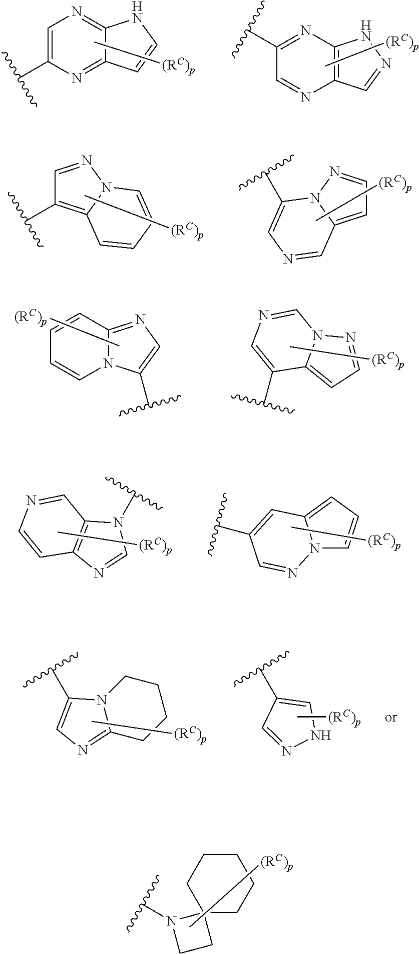

In certain embodiments, Ring C and its R^{C} substituents together form

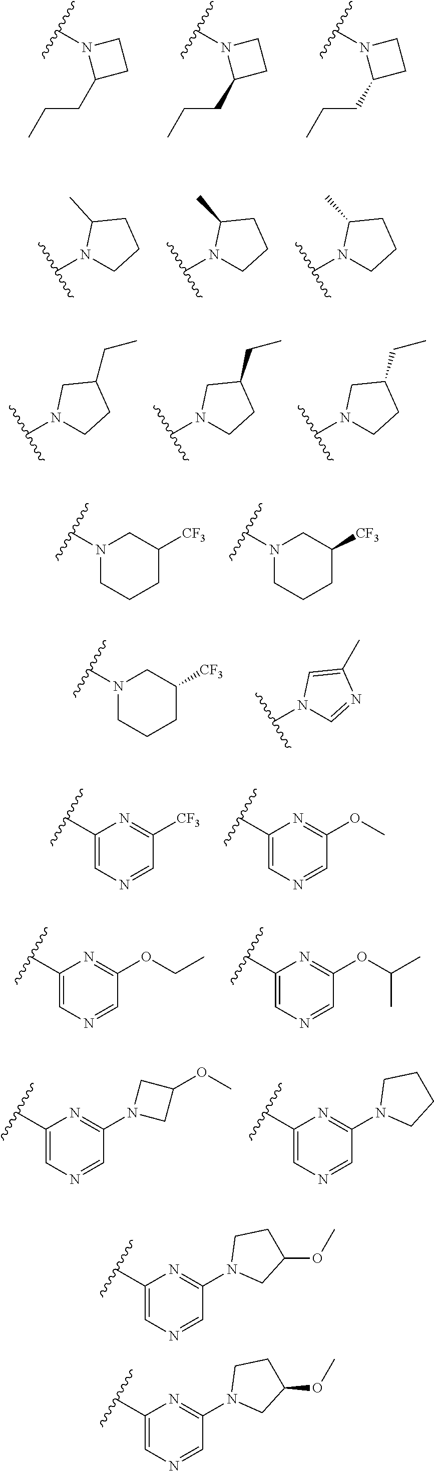

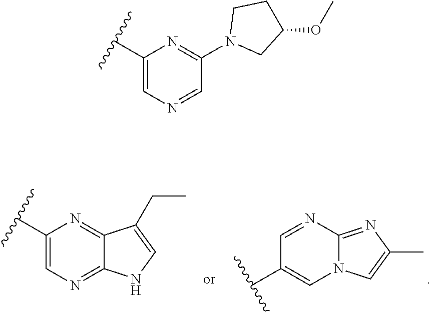 [SMILES: *c1cncc(N2CC[C@H](OC)C2)n1 ; *c1cnc2[nH]cc(CC)c2n1 ; *c1cnc2nc(C)cn2c1]

In certain embodiments, Ring C and its R^{C} substituents together form

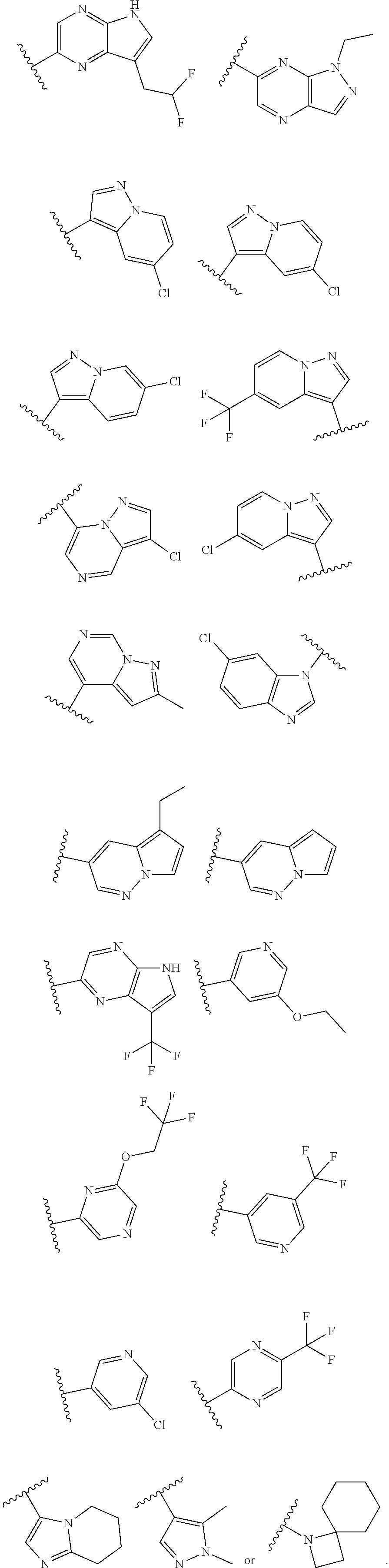

In certain embodiments, Ring C is

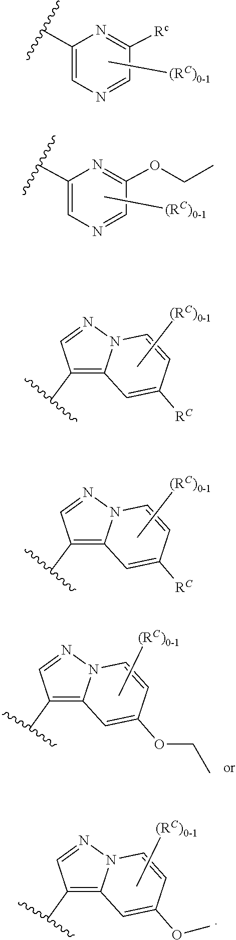

In certain embodiments, Ring C and its Rr substituents together form

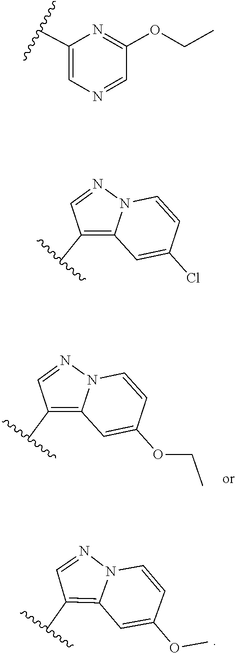

In some embodiments, Ring C is selected from those depicted in Table 1, below.

As defined generally above, each instance of R^{A} is independently optionally substituted C₁₋₄ aliphatic, oxo, halogen, CN, NO₂, OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR, or —P(O)R₂.

In some embodiments, each instance of R^{A} is independently —F or —OCH₃. In some embodiments, R^{A} is —F. In some embodiments, each R^{A} substituted onto R¹ is —F. In some embodiments, the each R^{A} substituted onto R¹ is —F and q is 2.

In some embodiments, each instance of R^{A} is selected from those depicted in Table 1, below.

As defined generally above, each instance of R^{B} is independently optionally substituted C₁₋₄ aliphatic, oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂.

In some embodiments, each instance of R^{B} is halogen or C₁₋₆ aliphatic.

In some embodiments, each instance of R^{B} is —F or -Me. In some embodiments, each instance of R^{B} is —F.

In some embodiments R^{B} is —CH₂N(CH₃)₂.

In some embodiments, each instance of R^{B} is selected from those depicted in Table 1, below.

As defined generally above, each instance of R^{C} is independently optionally substituted C₁₋₄ aliphatic, oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR, or —P(O)R₂.

In some embodiments, each instance of R^{C} is —OR, an optionally substituted C₁₋₆ aliphatic, or an optionally substituted 3-7 membered saturated or partially unsaturated monocyclic heterocyclic ring having 1-2 heteroatoms independently selected from nitrogen, oxygen, phosphorous, silicon and sulfur.

In some embodiments, each instance of R^{C} is -Me, -Et, —Pr, —CF₃, —OMe, -OEt, or -OiPr.

In some embodiments, each instance of R^{C} is —CH₂CHF₂.

In some embodiments, each instance of R^{C} is selected from those depicted in Table 1, below.

As defined generally above, each R is independently hydrogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 9-12 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring; or:

two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur.

In some embodiments, R is selected from those depicted in Table 1, below.

As defined generally above, R² is hydrogen, halogen, —OR, —NR₂, an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 7-12 membered saturated or partially unsaturated spirocyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur).

In some embodiments, R² is hydrogen. In some embodiments, R² is halogen, —OR, —NR₂, an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 7-12 membered saturated or partially unsaturated spirocyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur).

In some embodiments, R² is hydrogen, methyl, halogen, methoxy, hydroxyl, —CHF₂, —N(CH₃)₂,

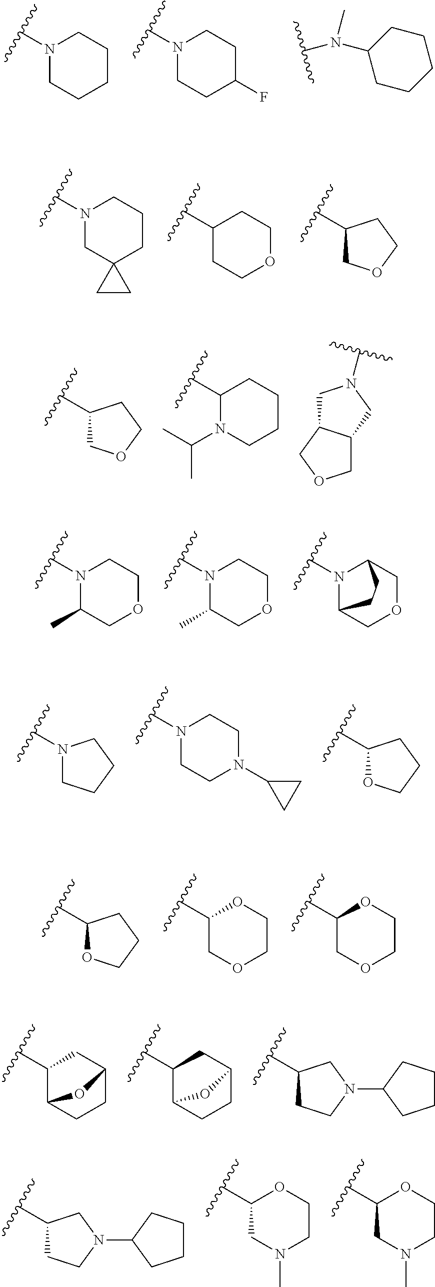

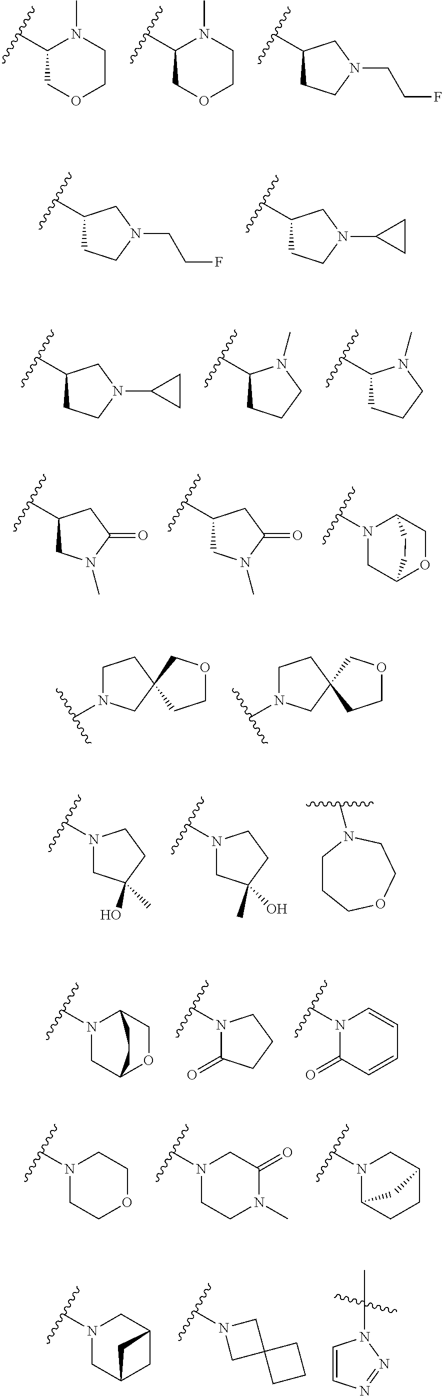

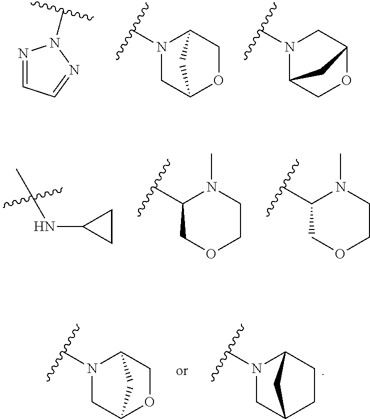

As described generally above, L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—.

In some embodiments, L² is a covalent bond. In some embodiments L² is —CH₂—, —C(CH₃)₂—, —CH₂CH₂—, —CH₂CH₂CH₂—, —C(CH₃)₂CH₂—, —CH₂C(CH₃)₂—, —CH₂O—, —OCH₂—,

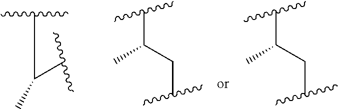

As defined generally above, each hydrogen bound to carbon can be optionally and independently replaced by deuterium.

In some embodiments, a hydrogen bound to carbon is replaced by deuterium.

As defined generally above, m is 0, 1, or 2.

In some embodiments, m is 0. In some embodiments, m is 1. In some embodiments, m is 2.

In some embodiments, m is selected from those depicted in Table 1, below.

In some embodiments, n is 0. In some embodiments, n is 1. In some embodiments, n is 2.

In some embodiments, n is selected from those depicted in Table 1, below.

In some embodiments, p is 0. In some embodiments, p is 1. In some embodiments, p is 2.

In some embodiments, p is selected from those depicted in Table 1, below.

As defined generally above, q is 0, 1, 2, 3, or 4. In some embodiments, q is 0. In some embodiments, q is 1, 2, 3, or 4. In some embodiments, q is 1. In some embodiments, q is 2. In some embodiments, q is 3. In some embodiments, q is 4.

In some embodiments, q is selected from those depicted in Table 1, below.

In some embodiments, the present invention provides a compound of formula I of formula II:

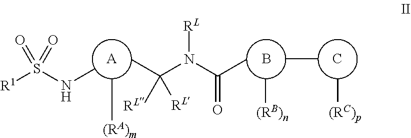

or a pharmaceutically acceptable salt thereof, wherein each of R¹, Ring A, Ring B, Ring C, R^{A}, R^{B}, R^{C}, R^{L}, R^{L′}, R^{L″}, m, n, and, p, is as defined above and described in embodiments herein, both singly and in combination.

In some embodiments, the present invention provides a compound of formula I of formula III:

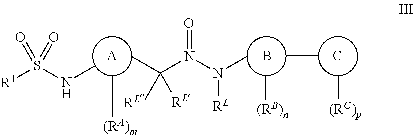

or a pharmaceutically acceptable salt thereof, wherein each of R¹, Ring A, Ring B, Ring C, R^{A}, R^{B}, R^{C}, R^{L}, R^{L′}, R^{L″}, m, n, and, p, is as defined above and described in embodiments herein, both singly and in combination.

In some embodiments, the present invention provides a compound of formula I of formula IV-a, IV-b, or IV-c:

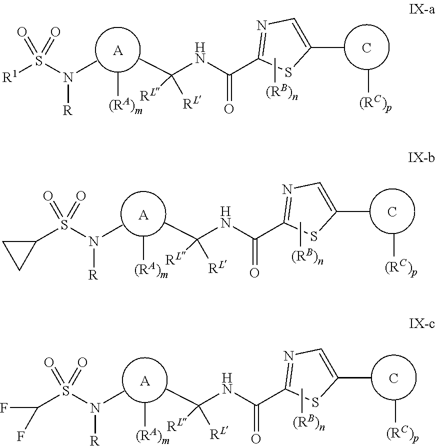

or a pharmaceutically acceptable salt thereof, wherein each of R¹, Ring A, Ring C, R^{A}, R^{B}, R^{C}, R^{L′}, R, m, n, and, p, is as defined above and described in embodiments herein, both singly and in combination. In some embodiments, R is H. In some embodiments, n is 1.

In some embodiments, the present invention provides a compound of formula I of formula V-a, V-b, V-c, V-d, V-e, or V-f:

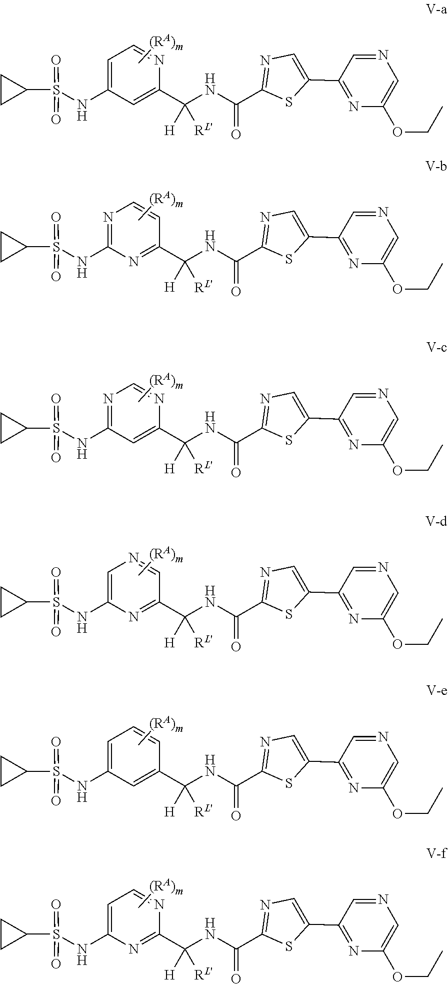

or a pharmaceutically acceptable salt thereof, wherein R^{L′}, R^{A}, and m is as defined above and described in embodiments herein, both singly and in combination.

In some embodiments, the present invention provides a compound of formula I of formula VI-a, VI-b, VI-c, VI-d, VI-e, or VI-f:

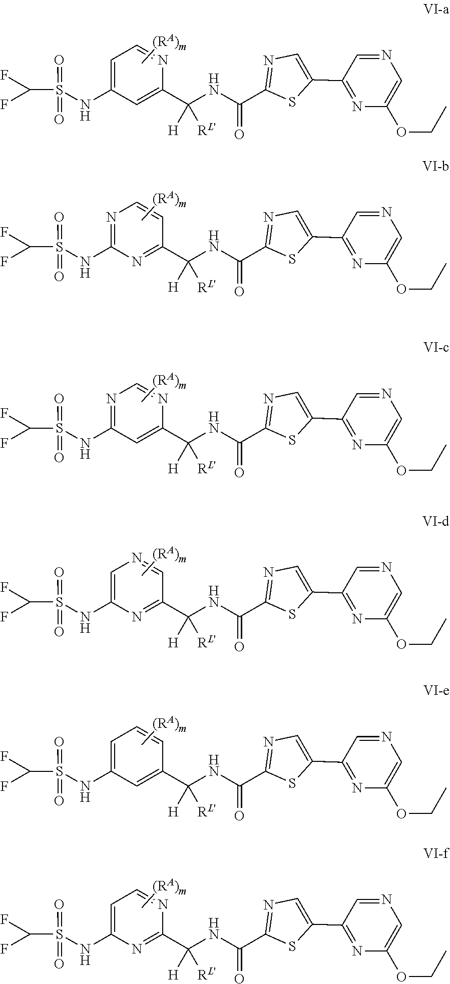

or a pharmaceutically acceptable salt thereof, wherein each of R^{L′}, R^{A}, and m is as defined above and described in embodiments herein, both singly and in combination.

In some embodiments, the present invention provides a compound of formula I of formula VII-a, VII-b, VII-c, VII-d, VII-e, or VII-f:

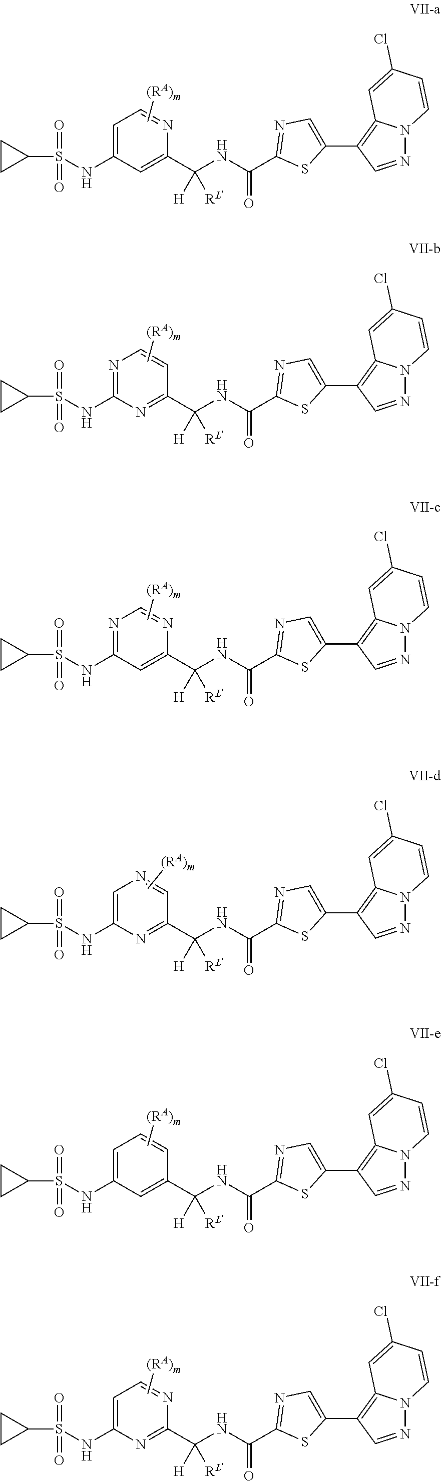

or a pharmaceutically acceptable salt thereof, wherein each of R^{L′}, R^{A}, and m is as defined above and described in embodiments herein, both singly and in combination.

In some embodiments, the present invention provides a compound of formula I of formula VIII-a, VIII-b, VIII-c, VIII-d, VIII-e, or VIII-f:

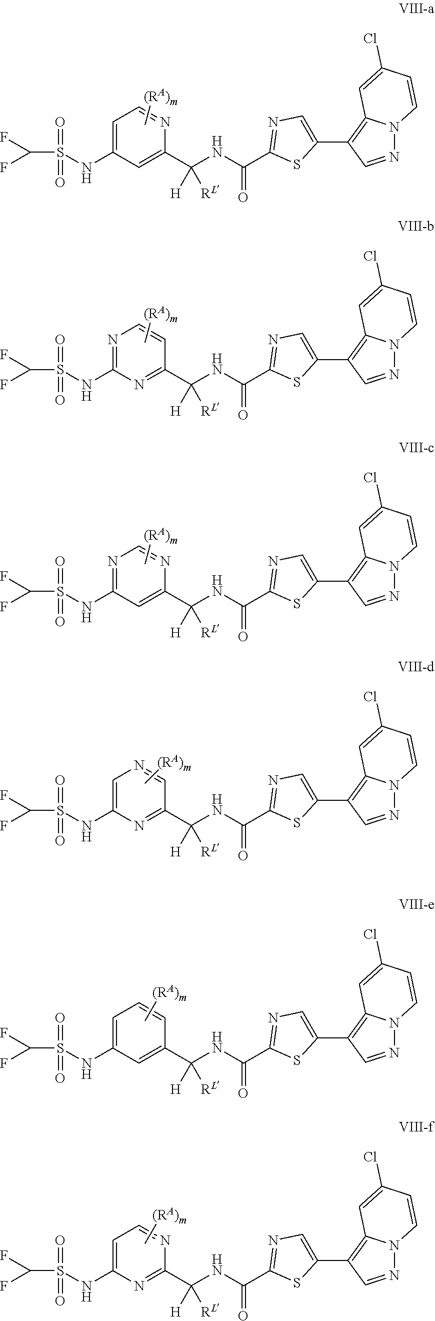

or a pharmaceutically acceptable salt thereof, wherein each of R^{L′}, R^{A}, and m is as defined above and described in embodiments herein, both singly and in combination.

In some embodiments, the present invention provides a compound of formula I of formula IX-a, IX-b, or IX-c:

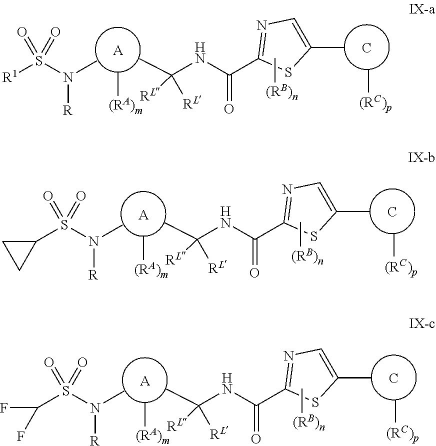

or a pharmaceutically acceptable salt thereof, wherein each of R¹, Ring A, Ring C, R^{A}, R^{B}, R^{C}, R, m, n, and, p, is as defined above and described in embodiments herein, both singly and in combination and wherein the R^{L′} and R^{L″} group are taken together with the atom to which they are attached, to form an optionally substituted 3-7 membered saturated or partially unsaturated monocyclic carbocyclic ring; or a 3-7 membered optionally substituted saturated or partially unsaturated monocyclic heterocyclic ring having 1-2 heteroatoms independently selected from nitrogen, oxygen, and sulfur. In some embodiments, R is H. In some embodiments, n is 1.

Exemplary compounds of the invention are set forth in Table 1, below.

TABLE 1
Selected Compounds

| Compound | Structure |
|---|---|
| I-1 | 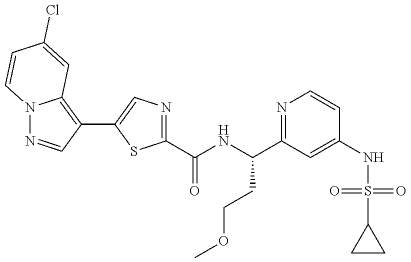 [SMILES: COCC[C@H](NC(=O)c1ncc(-c2cnn3ccc(Cl)cc23)s1)c1cc(NS(=O)(=O)C2CC2)ccn1] |
| I-2 | 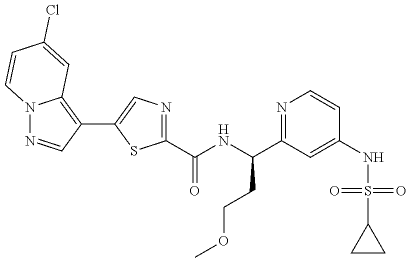 [SMILES: COCC[C@@H](NC(=O)c1ncc(-c2cnn3ccc(Cl)cc23)s1)c1cc(NS(=O)(=O)C2CC2)ccn1] |
| I-3 | 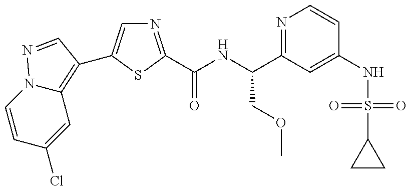 [SMILES: COC[C@H](NC(=O)c1ncc(-c2cnn3ccc(Cl)cc23)s1)c1cc(NS(=O)(=O)C2CC2)ccn1] |
| I-4 | 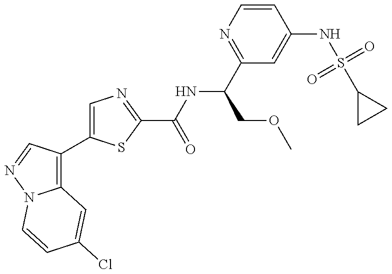 [SMILES: COC[C@@H](NC(=O)c1ncc(-c2cnn3ccc(Cl)cc23)s1)c1cc(NS(=O)(=O)C2CC2)ccn1] |
| I-5 | 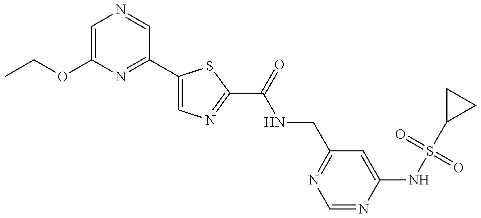 [SMILES: CCOc1cncc(-c2cnc(C(=O)NCc3cc(NS(=O)(=O)C4CC4)ncn3)s2)n1] |
| I-6 | 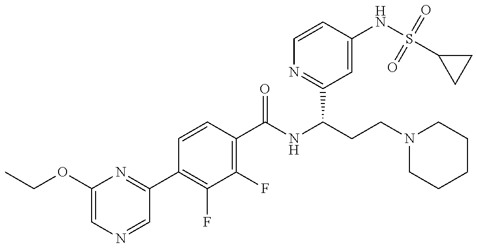 [SMILES: CCOc1cncc(-c2ccc(C(=O)N[C@@H](CCN3CCCCC3)c3cc(NS(=O)(=O)C4CC4)ccn3)c(F)c2F)n1] |
| I-7 | 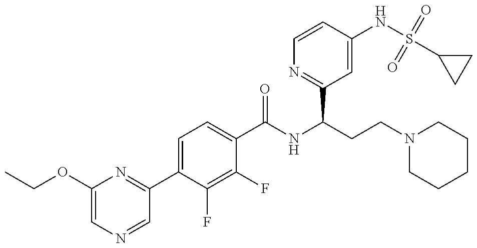 [SMILES: CCOc1cncc(-c2ccc(C(=O)N[C@H](CCN3CCCCC3)c3cc(NS(=O)(=O)C4CC4)ccn3)c(F)c2F)n1] |
| I-8 | 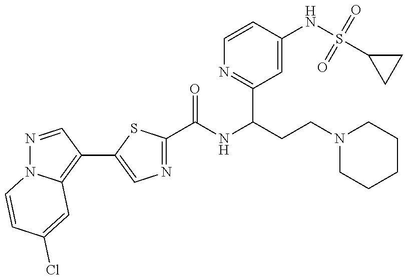 [SMILES: O=C(NC(CCN1CCCCC1)c1cc(NS(=O)(=O)C2CC2)ccn1)c1ncc(-c2cnn3ccc(Cl)cc23)s1] |
| I-9 | 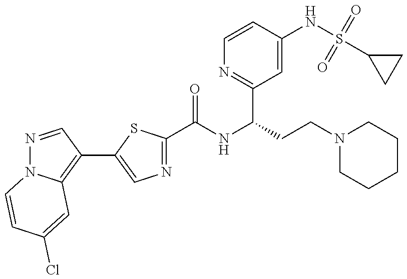 [SMILES: O=C(N[C@@H](CCN1CCCCC1)c1cc(NS(=O)(=O)C2CC2)ccn1)c1ncc(-c2cnn3ccc(Cl)cc23)s1] |
| I-10 | 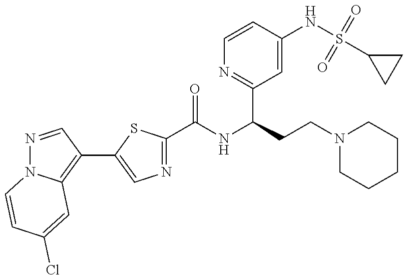 [SMILES: O=C(N[C@H](CCN1CCCCC1)c1cc(NS(=O)(=O)C2CC2)ccn1)c1ncc(-c2cnn3ccc(Cl)cc23)s1] |
| I-11 | 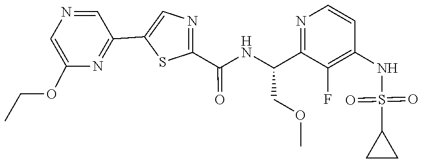 [SMILES: CCOc1cncc(-c2cnc(C(=O)N[C@@H](COC)c3nccc(NS(=O)(=O)C4CC4)c3F)s2)n1] |
| I-12 | 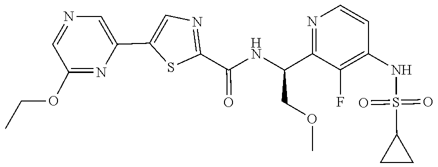 [SMILES: CCOc1cncc(-c2cnc(C(=O)N[C@H](COC)c3nccc(NS(=O)(=O)C4CC4)c3F)s2)n1] |
| I-13 | 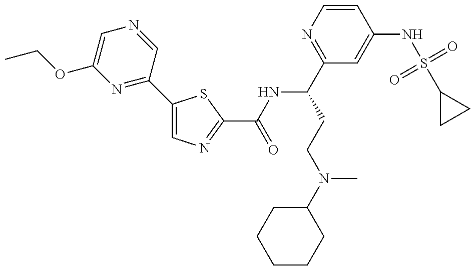 [SMILES: CCOc1cncc(-c2cnc(C(=O)N[C@@H](CCN(C)C3CCCCC3)c3cc(NS(=O)(=O)C4CC4)ccn3)s2)n1] |
| I-14 | 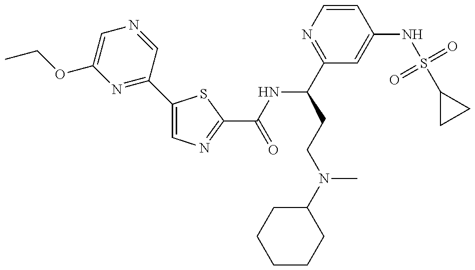 [SMILES: CCOc1cncc(-c2cnc(C(=O)N[C@H](CCN(C)C3CCCCC3)c3cc(NS(=O)(=O)C4CC4)ccn3)s2)n1] |
| I-15 | 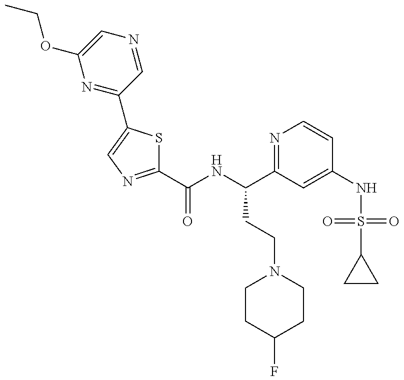 [SMILES: CCOc1cncc(-c2cnc(C(=O)N[C@@H](CCN3CCC(F)CC3)c3cc(NS(=O)(=O)C4CC4)ccn3)s2)n1] |
| I-16 | 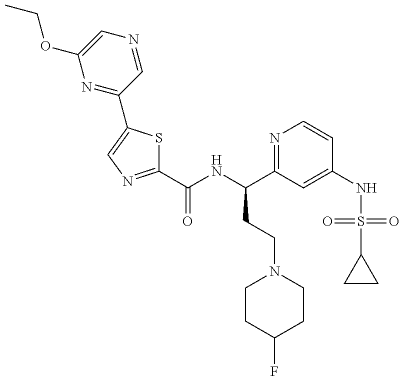 [SMILES: CCOc1cncc(-c2cnc(C(=O)N[C@H](CCN3CCC(F)CC3)c3cc(NS(=O)(=O)C4CC4)ccn3)s2)n1] |
| I-17 | 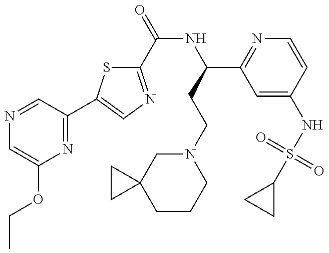 [SMILES: CCOc1cncc(-c2cnc(C(=O)N[C@H](CCN3CCCC4(CC4)C3)c3cc(NS(=O)(=O)C4CC4)ccn3)s2)n1] |
| I-18 | 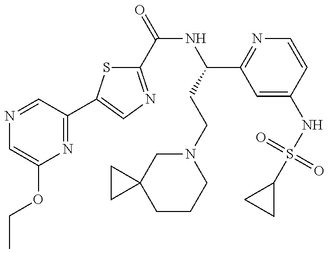 [SMILES: CCOc1cncc(-c2cnc(C(=O)N[C@@H](CCN3CCCC4(CC4)C3)c3cc(NS(=O)(=O)C4CC4)ccn3)s2)n1] |
| I-19 | 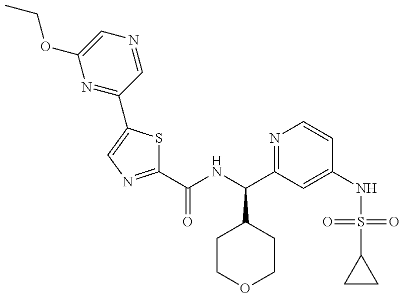 [SMILES: CCOc1cncc(-c2cnc(C(=O)N[C@@H](c3cc(NS(=O)(=O)C4CC4)ccn3)C3CCOCC3)s2)n1] |
| I-20 | 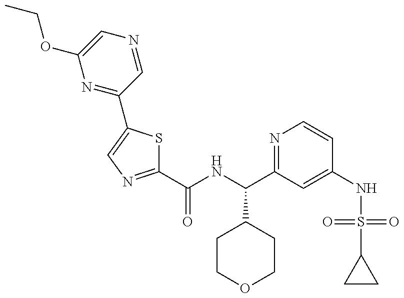 [SMILES: CCOc1cncc(-c2cnc(C(=O)N[C@H](c3cc(NS(=O)(=O)C4CC4)ccn3)C3CCOCC3)s2)n1] |
| I-21 | 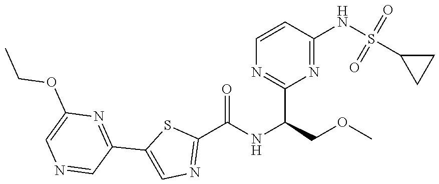 [SMILES: CCOc1cncc(-c2cnc(C(=O)N[C@H](COC)c3nccc(NS(=O)(=O)C4CC4)n3)s2)n1] |
| I-22 | 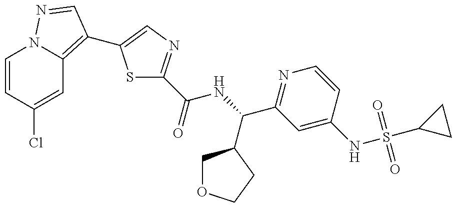 [SMILES: O=C(N[C@H](c1cc(NS(=O)(=O)C2CC2)ccn1)[C@H]1CCOC1)c1ncc(-c2cnn3ccc(Cl)cc23)s1] |
| I-23 | 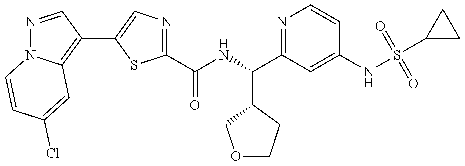 [SMILES: O=C(N[C@H](c1cc(NS(=O)(=O)C2CC2)ccn1)[C@@H]1CCOC1)c1ncc(-c2cnn3ccc(Cl)cc23)s1] |
| I-24 | 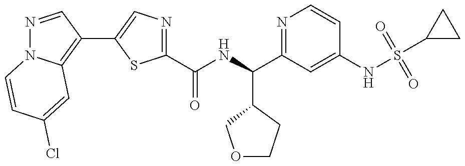 [SMILES: O=C(N[C@@H](c1cc(NS(=O)(=O)C2CC2)ccn1)[C@@H]1CCOC1)c1ncc(-c2cnn3ccc(Cl)cc23)s1] |
| I-25 | 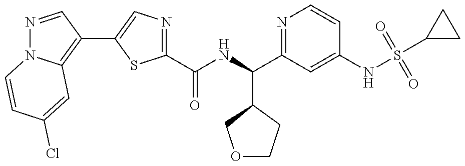 [SMILES: O=C(N[C@@H](c1cc(NS(=O)(=O)C2CC2)ccn1)[C@H]1CCOC1)c1ncc(-c2cnn3ccc(Cl)cc23)s1] |
| I-26 | 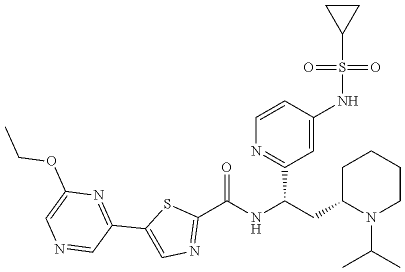 [SMILES: CCOc1cncc(-c2cnc(C(=O)N[C@@H](C[C@@H]3CCCCN3C(C)C)c3cc(NS(=O)(=O)C4CC4)ccn3)s2)n1] |
| I-27 | 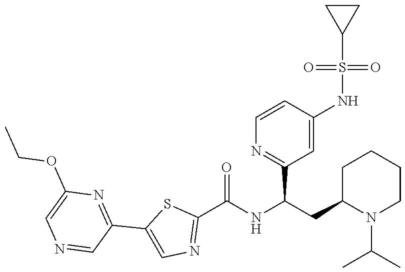 [SMILES: CCOc1cncc(-c2cnc(C(=O)N[C@H](C[C@H]3CCCCN3C(C)C)c3cc(NS(=O)(=O)C4CC4)ccn3)s2)n1] |
| I-28 | 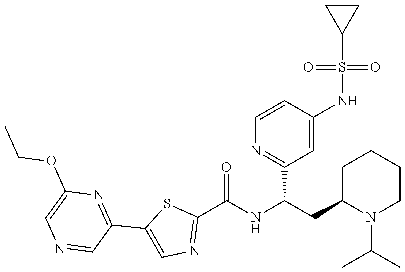 [SMILES: CCOc1cncc(-c2cnc(C(=O)N[C@@H](C[C@H]3CCCCN3C(C)C)c3cc(NS(=O)(=O)C4CC4)ccn3)s2)n1] |
| I-29 | 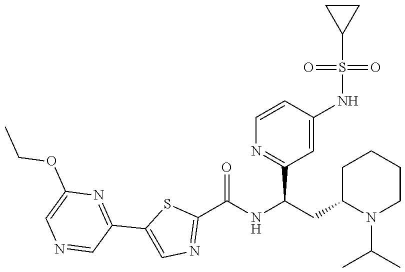 [SMILES: CCOc1cncc(-c2cnc(C(=O)N[C@H](C[C@@H]3CCCCN3C(C)C)c3cc(NS(=O)(=O)C4CC4)ccn3)s2)n1] |
| I-30 | 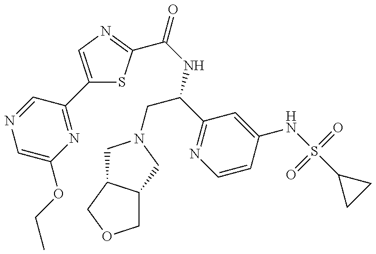 [SMILES: CCOc1cncc(-c2cnc(C(=O)N[C@@H](CN3C[C@H]4COC[C@H]4C3)c3cc(NS(=O)(=O)C4CC4)ccn3)s2)n1] |
| I-31 | 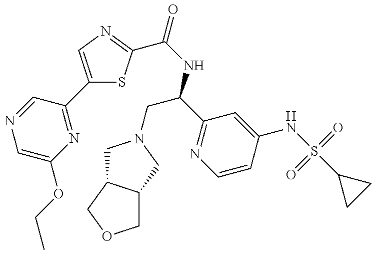 [SMILES: CCOc1cncc(-c2cnc(C(=O)N[C@H](CN3C[C@H]4COC[C@H]4C3)c3cc(NS(=O)(=O)C4CC4)ccn3)s2)n1] |
| I-32 | 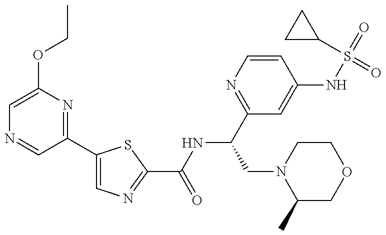 [SMILES: CCOc1cncc(-c2cnc(C(=O)N[C@@H](CN3CCOC[C@H]3C)c3cc(NS(=O)(=O)C4CC4)ccn3)s2)n1] |
| I-33 | 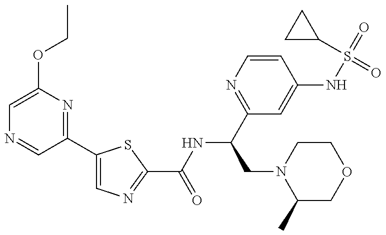 [SMILES: CCOc1cncc(-c2cnc(C(=O)N[C@H](CN3CCOC[C@H]3C)c3cc(NS(=O)(=O)C4CC4)ccn3)s2)n1] |
| I-34 | 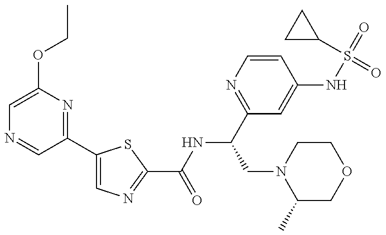 [SMILES: CCOc1cncc(-c2cnc(C(=O)N[C@@H](CN3CCOC[C@@H]3C)c3cc(NS(=O)(=O)C4CC4)ccn3)s2)n1] |
| I-35 | 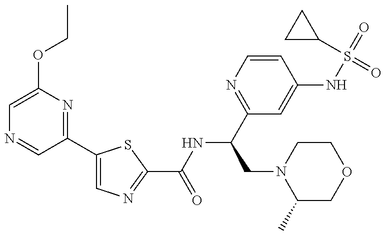 [SMILES: CCOc1cncc(-c2cnc(C(=O)N[C@H](CN3CCOC[C@@H]3C)c3cc(NS(=O)(=O)C4CC4)ccn3)s2)n1] |
| I-36 | 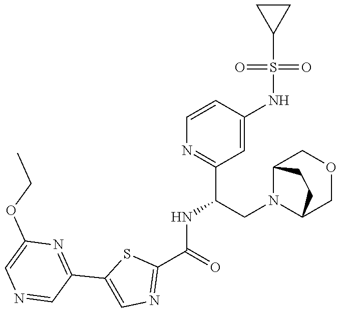 [SMILES: CCOc1cncc(-c2cnc(C(=O)N[C@@H](CN3[C@@H]4CC[C@H]3COC4)c3cc(NS(=O)(=O)C4CC4)ccn3)s2)n1] |
| I-37 | 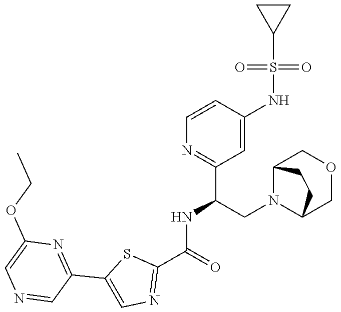 [SMILES: CCOc1cncc(-c2cnc(C(=O)N[C@H](CN3[C@@H]4CC[C@H]3COC4)c3cc(NS(=O)(=O)C4CC4)ccn3)s2)n1] |
| I-38 | 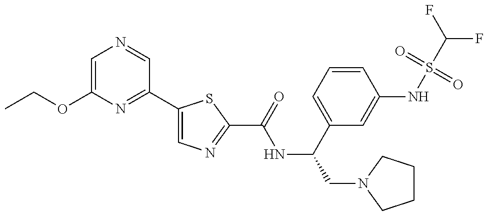 [SMILES: CCOc1cncc(-c2cnc(C(=O)N[C@@H](CN3CCCC3)c3cccc(NS(=O)(=O)C(F)F)c3)s2)n1] |
| I-39 | 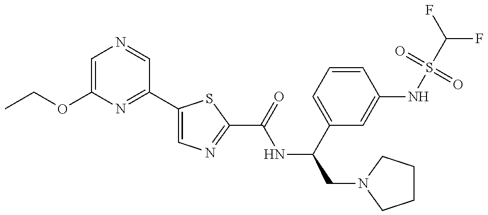 [SMILES: CCOc1cncc(-c2cnc(C(=O)N[C@H](CN3CCCC3)c3cccc(NS(=O)(=O)C(F)F)c3)s2)n1] |
| I-40 | 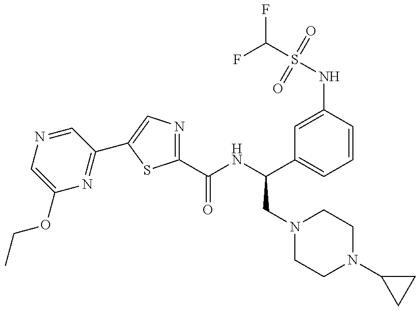 [SMILES: CCOc1cncc(-c2cnc(C(=O)N[C@H](CN3CCN(C4CC4)CC3)c3cccc(NS(=O)(=O)C(F)F)c3)s2)n1] |
| I-41 | 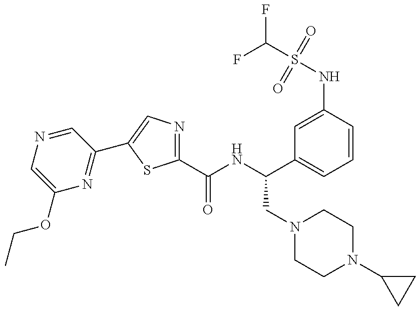 [SMILES: CCOc1cncc(-c2cnc(C(=O)N[C@@H](CN3CCN(C4CC4)CC3)c3cccc(NS(=O)(=O)C(F)F)c3)s2)n1] |
| I-42 | 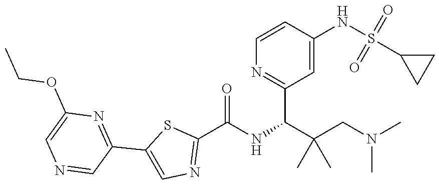 [SMILES: CCOc1cncc(-c2cnc(C(=O)N[C@H](c3cc(NS(=O)(=O)C4CC4)ccn3)C(C)(C)CN(C)C)s2)n1] |
| I-43 | 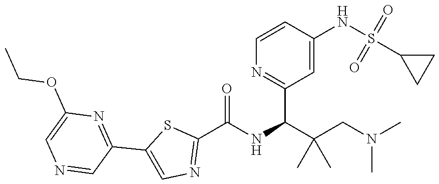 [SMILES: CCOc1cncc(-c2cnc(C(=O)N[C@@H](c3cc(NS(=O)(=O)C4CC4)ccn3)C(C)(C)CN(C)C)s2)n1] |
| I-44 | 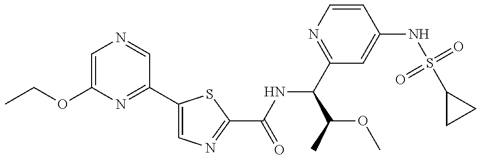 [SMILES: CCOc1cncc(-c2cnc(C(=O)N[C@@H](c3cc(NS(=O)(=O)C4CC4)ccn3)[C@H](C)OC)s2)n1] |
| I-45 | 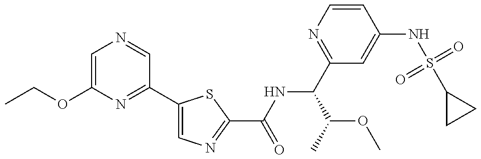 [SMILES: CCOc1cncc(-c2cnc(C(=O)N[C@H](c3cc(NS(=O)(=O)C4CC4)ccn3)[C@@H](C)OC)s2)n1] |
| I-46 | 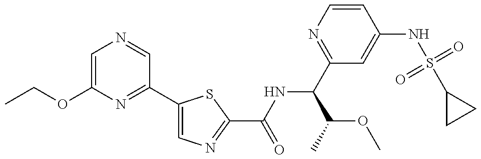 [SMILES: CCOc1cncc(-c2cnc(C(=O)N[C@@H](c3cc(NS(=O)(=O)C4CC4)ccn3)[C@@H](C)OC)s2)n1] |
| I-47 | 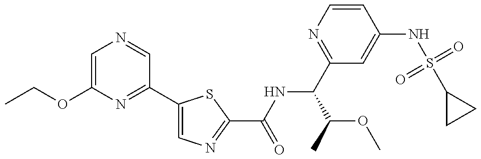 [SMILES: CCOc1cncc(-c2cnc(C(=O)N[C@H](c3cc(NS(=O)(=O)C4CC4)ccn3)[C@H](C)OC)s2)n1] |
| I-48 | 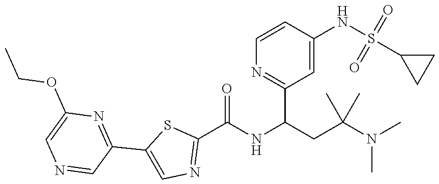 [SMILES: CCOc1cncc(-c2cnc(C(=O)NC(CC(C)(C)N(C)C)c3cc(NS(=O)(=O)C4CC4)ccn3)s2)n1] |
| I-49 | 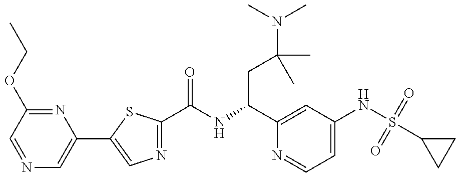 [SMILES: CCOc1cncc(-c2cnc(C(=O)N[C@H](CC(C)(C)N(C)C)c3cc(NS(=O)(=O)C4CC4)ccn3)s2)n1] |
| I-50 | 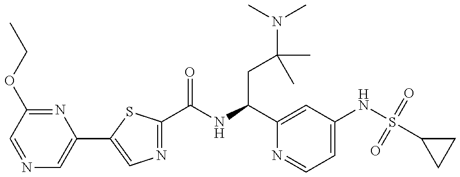 [SMILES: CCOc1cncc(-c2cnc(C(=O)N[C@@H](CC(C)(C)N(C)C)c3cc(NS(=O)(=O)C4CC4)ccn3)s2)n1] |
| I-51 | 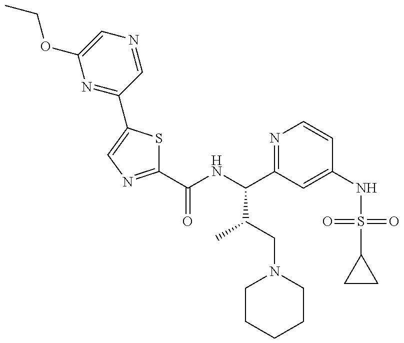 [SMILES: CCOc1cncc(-c2cnc(C(=O)N[C@H](c3cc(NS(=O)(=O)C4CC4)ccn3)[C@@H](C)CN3CCCCC3)s2)n1] |
| I-52 | 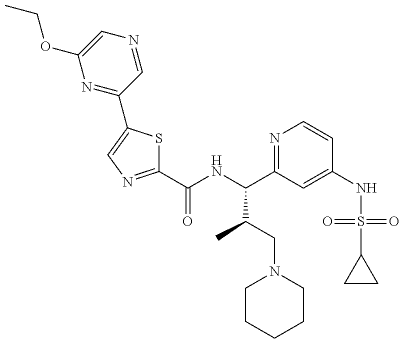 [SMILES: CCOc1cncc(-c2cnc(C(=O)N[C@H](c3cc(NS(=O)(=O)C4CC4)ccn3)[C@H](C)CN3CCCCC3)s2)n1] |
| I-53 | 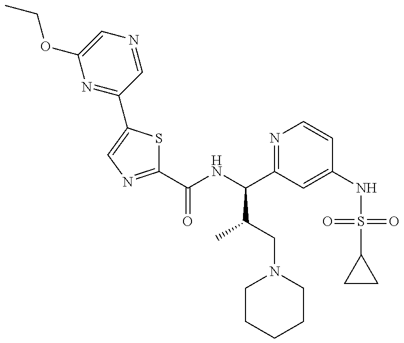 [SMILES: CCOc1cncc(-c2cnc(C(=O)N[C@@H](c3cc(NS(=O)(=O)C4CC4)ccn3)[C@@H](C)CN3CCCCC3)s2)n1] |
| I-54 | 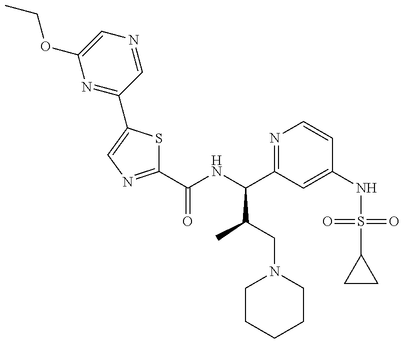 [SMILES: CCOc1cncc(-c2cnc(C(=O)N[C@@H](c3cc(NS(=O)(=O)C4CC4)ccn3)[C@H](C)CN3CCCCC3)s2)n1] |
| I-55 | 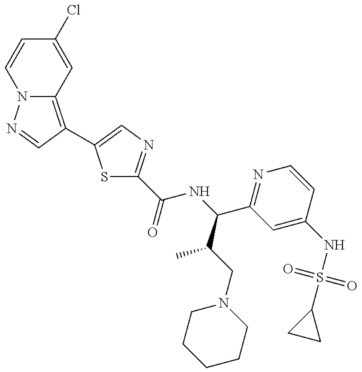 [SMILES: C[C@@H](CN1CCCCC1)[C@@H](NC(=O)c1ncc(-c2cnn3ccc(Cl)cc23)s1)c1cc(NS(=O)(=O)C2CC2)ccn1] |
| I-56 | 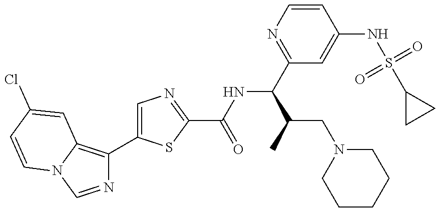 [SMILES: C[C@H](CN1CCCCC1)[C@@H](NC(=O)c1ncc(-c2ncn3ccc(Cl)cc23)s1)c1cc(NS(=O)(=O)C2CC2)ccn1] |
| I-57 | 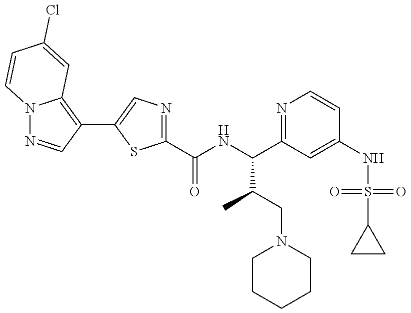 [SMILES: C[C@H](CN1CCCCC1)[C@H](NC(=O)c1ncc(-c2cnn3ccc(Cl)cc23)s1)c1cc(NS(=O)(=O)C2CC2)ccn1] |
| I-58 | 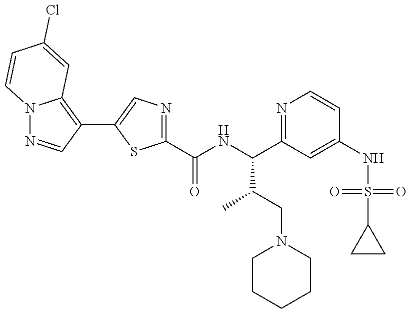 [SMILES: C[C@@H](CN1CCCCC1)[C@H](NC(=O)c1ncc(-c2cnn3ccc(Cl)cc23)s1)c1cc(NS(=O)(=O)C2CC2)ccn1] |
| I-59 | 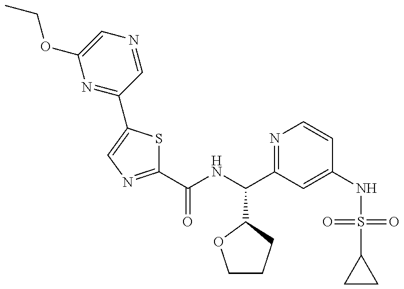 [SMILES: CCOc1cncc(-c2cnc(C(=O)N[C@H](c3cc(NS(=O)(=O)C4CC4)ccn3)[C@H]3CCCO3)s2)n1] |
| I-60 | 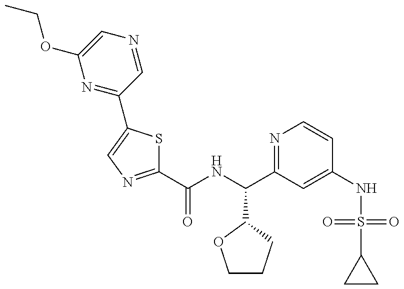 [SMILES: CCOc1cncc(-c2cnc(C(=O)N[C@H](c3cc(NS(=O)(=O)C4CC4)ccn3)[C@@H]3CCCO3)s2)n1] |
| I-61 | 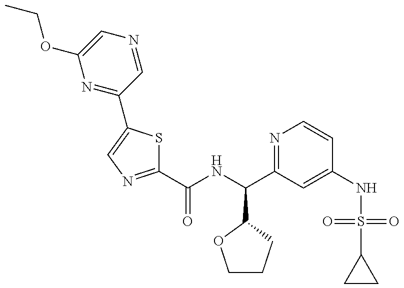 [SMILES: CCOc1cncc(-c2cnc(C(=O)N[C@@H](c3cc(NS(=O)(=O)C4CC4)ccn3)[C@@H]3CCCO3)s2)n1] |
| I-62 | 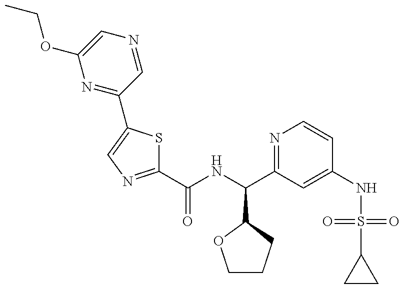 [SMILES: CCOc1cncc(-c2cnc(C(=O)N[C@@H](c3cc(NS(=O)(=O)C4CC4)ccn3)[C@H]3CCCO3)s2)n1] |
| I-63 | 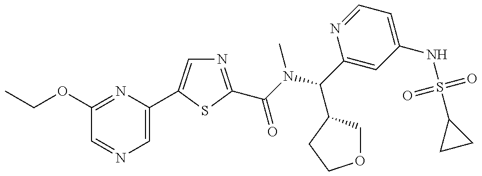 [SMILES: CCOc1cncc(-c2cnc(C(=O)N(C)[C@H](c3cc(NS(=O)(=O)C4CC4)ccn3)[C@H]3CCOC3)s2)n1] |
| I-64 | 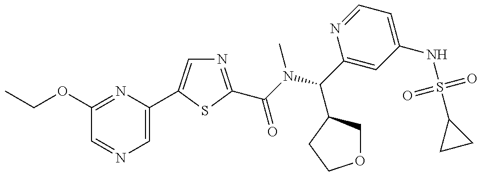 [SMILES: CCOc1cncc(-c2cnc(C(=O)N(C)[C@H](c3cc(NS(=O)(=O)C4CC4)ccn3)[C@@H]3CCOC3)s2)n1] |
| I-65 | 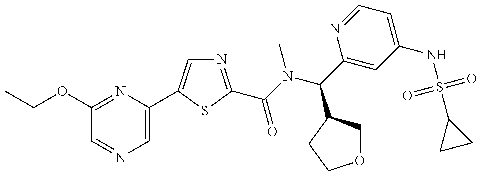 [SMILES: CCOc1cncc(-c2cnc(C(=O)N(C)[C@@H](c3cc(NS(=O)(=O)C4CC4)ccn3)[C@@H]3CCOC3)s2)n1] |
| I-66 | 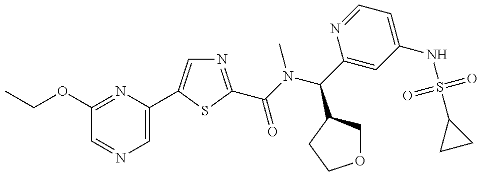 [SMILES: CCOc1cncc(-c2cnc(C(=O)N(C)[C@@H](c3cc(NS(=O)(=O)C4CC4)ccn3)[C@@H]3CCOC3)s2)n1] |
| I-67 | 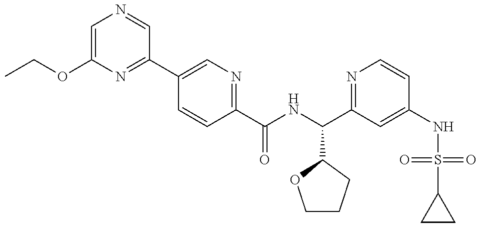 [SMILES: CCOc1cncc(-c2ccc(C(=O)N[C@H](c3cc(NS(=O)(=O)C4CC4)ccn3)[C@H]3CCCO3)nc2)n1] |
| I-68 | 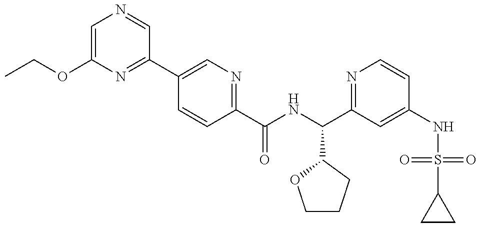 [SMILES: CCOc1cncc(-c2ccc(C(=O)N[C@H](c3cc(NS(=O)(=O)C4CC4)ccn3)[C@@H]3CCCO3)nc2)n1] |
| I-69 | 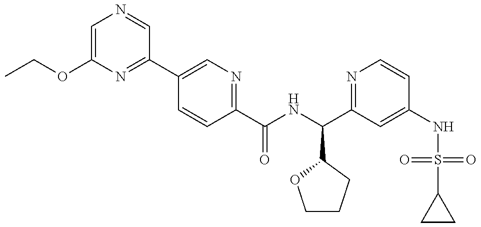 [SMILES: CCOc1cncc(-c2ccc(C(=O)N[C@@H](c3cc(NS(=O)(=O)C4CC4)ccn3)[C@@H]3CCCO3)nc2)n1] |
| I-70 | 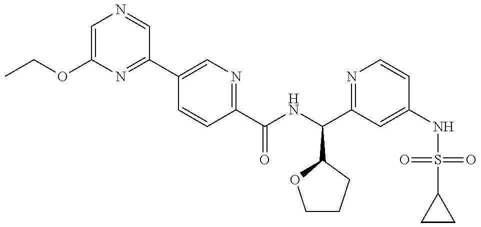 [SMILES: CCOc1cncc(-c2ccc(C(=O)N[C@@H](c3cc(NS(=O)(=O)C4CC4)ccn3)[C@H]3CCCO3)nc2)n1] |
| I-71 | 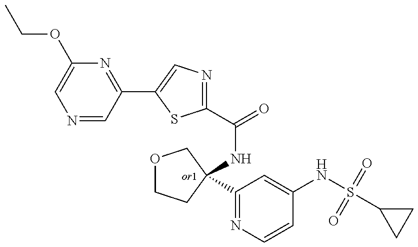 [SMILES: CCOc1cncc(-c2cnc(C(=O)N[C@@]3(c4cc(NS(=O)(=O)C5CC5)ccn4)CCOC3)s2)n1] |
| I-72 | 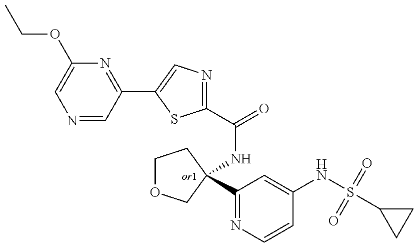 [SMILES: CCOc1cncc(-c2cnc(C(=O)N[C@@]3(c4cc(NS(=O)(=O)C5CC5)ccn4)CCOC3)s2)n1] |
| I-73 | 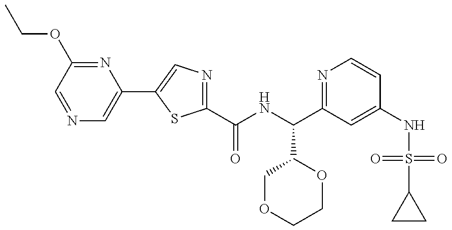 [SMILES: CCOc1cncc(-c2cnc(C(=O)N[C@H](c3cc(NS(=O)(=O)C4CC4)ccn3)[C@H]3COCCO3)s2)n1] |
| I-74 | 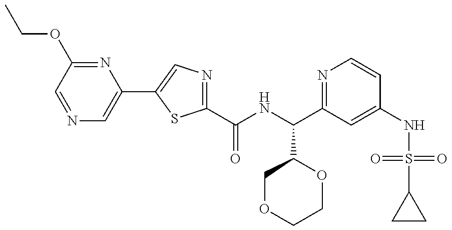 [SMILES: CCOc1cncc(-c2cnc(C(=O)N[C@H](c3cc(NS(=O)(=O)C4CC4)ccn3)[C@@H]3COCCO3)s2)n1] |
| I-75 | 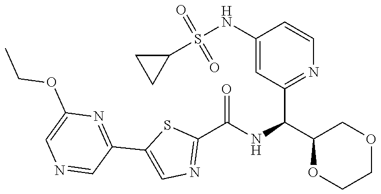 [SMILES: CCOc1cncc(-c2cnc(C(=O)N[C@@H](c3cc(NS(=O)(=O)C4CC4)ccn3)[C@H]3COCCO3)s2)n1] |
| I-76 | 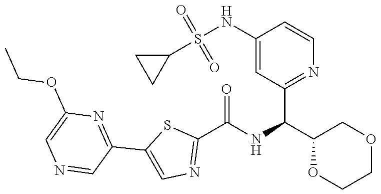 [SMILES: CCOc1cncc(-c2cnc(C(=O)N[C@@H](c3cc(NS(=O)(=O)C4CC4)ccn3)[C@@H]3COCCO3)s2)n1] |
| I-77 | 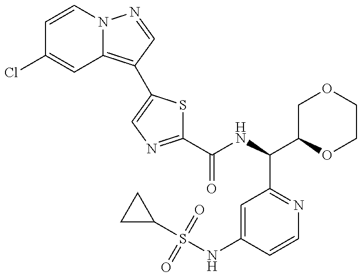 [SMILES: O=C(N[C@H](c1cc(NS(=O)(=O)C2CC2)ccn1)[C@H]1COCCO1)c1ncc(-c2cnn3ccc(Cl)cc23)s1] |
| I-78 | 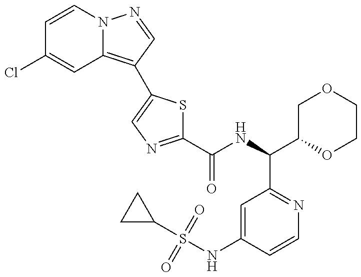 [SMILES: O=C(N[C@H](c1cc(NS(=O)(=O)C2CC2)ccn1)[C@@H]1COCCO1)c1ncc(-c2cnn3ccc(Cl)cc23)s1] |
| I-79 | 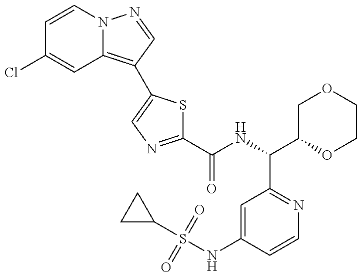 [SMILES: O=C(N[C@@H](c1cc(NS(=O)(=O)C2CC2)ccn1)[C@@H]1COCCO1)c1ncc(-c2cnn3ccc(Cl)cc23)s1] |
| I-80 | 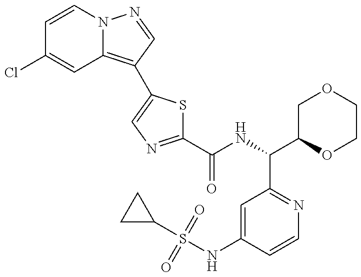 [SMILES: O=C(N[C@@H](c1cc(NS(=O)(=O)C2CC2)ccn1)[C@H]1COCCO1)c1ncc(-c2cnn3ccc(Cl)cc23)s1] |
| I-81 | 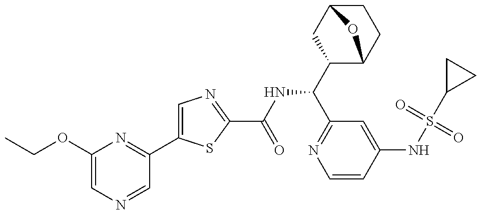 [SMILES: CCOc1cncc(-c2cnc(C(=O)N[C@@H](c3cc(NS(=O)(=O)C4CC4)ccn3)[C@@H]3C[C@@H]4CC[C@H]3O4)s2)n1] |
| I-82 | 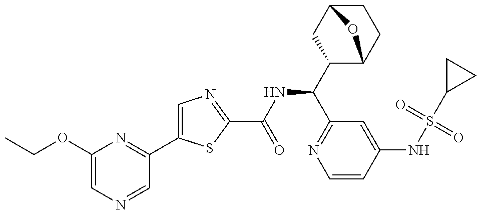 [SMILES: CCOc1cncc(-c2cnc(C(=O)N[C@H](c3cc(NS(=O)(=O)C4CC4)ccn3)[C@@H]3C[C@@H]4CC[C@H]3O4)s2)n1] |
| I-83 | 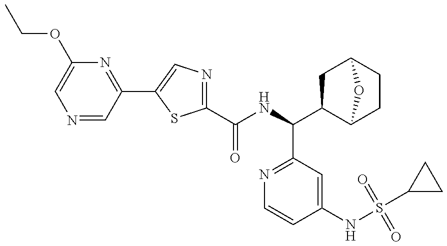 [SMILES: CCOc1cncc(-c2cnc(C(=O)N[C@H](c3cc(NS(=O)(=O)C4CC4)ccn3)[C@H]3C[C@H]4CC[C@@H]3O4)s2)n1] |
| I-84 | 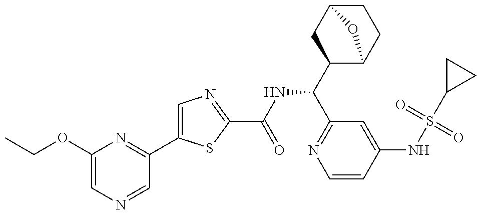 [SMILES: CCOc1cncc(-c2cnc(C(=O)N[C@@H](c3cc(NS(=O)(=O)C4CC4)ccn3)[C@H]3C[C@H]4CC[C@@H]3O4)s2)n1] |
| I-85 | 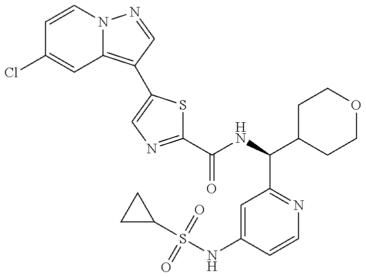 [SMILES: O=C(N[C@H](c1cc(NS(=O)(=O)C2CC2)ccn1)C1CCOCC1)c1ncc(-c2cnn3ccc(Cl)cc23)s1] |
| I-86 | 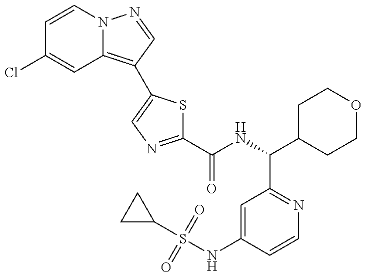 [SMILES: O=C(N[C@@H](c1cc(NS(=O)(=O)C2CC2)ccn1)C1CCOCC1)c1ncc(-c2cnn3ccc(Cl)cc23)s1] |
| I-87 | 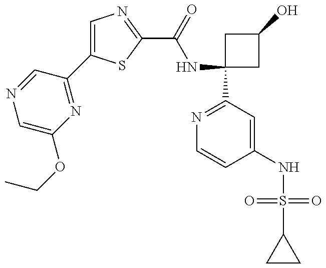 [SMILES: CCOc1cncc(-c2cnc(C(=O)N[C@]3(c4cc(NS(=O)(=O)C5CC5)ccn4)C[C@H](O)C3)s2)n1] |
| I-88 | 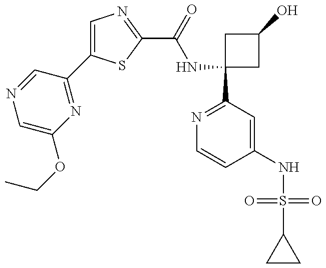 [SMILES: CCOc1cncc(-c2cnc(C(=O)N[C@]3(c4cc(NS(=O)(=O)C5CC5)ccn4)C[C@@H](O)C3)s2)n1] |
| I-89 | 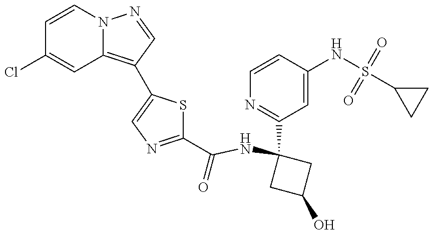 [SMILES: O=C(N[C@]1(c2cc(NS(=O)(=O)C3CC3)ccn2)C[C@H](O)C1)c1ncc(-c2cnn3ccc(Cl)cc23)s1] |
| I-90 | 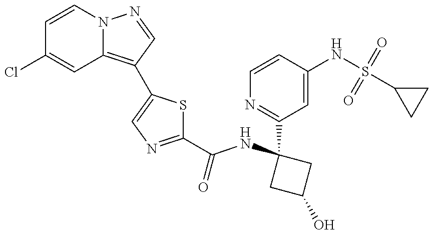 [SMILES: O=C(N[C@]1(c2cc(NS(=O)(=O)C3CC3)ccn2)C[C@@H](O)C1)c1ncc(-c2cnn3ccc(Cl)cc23)s1] |
| I-91 | 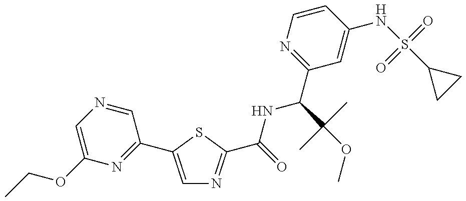 [SMILES: CCOc1cncc(-c2cnc(C(=O)N[C@@H](c3cc(NS(=O)(=O)C4CC4)ccn3)C(C)(C)OC)s2)n1] |
| I-92 | 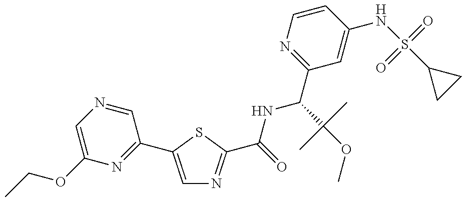 [SMILES: CCOc1cncc(-c2cnc(C(=O)N[C@H](c3cc(NS(=O)(=O)C4CC4)ccn3)C(C)(C)OC)s2)n1] |
| I-93 | 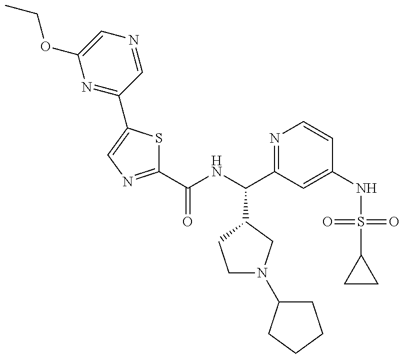 [SMILES: CCOc1cncc(-c2cnc(C(=O)N[C@H](c3cc(NS(=O)(=O)C4CC4)ccn3)[C@H]3CCN(C4CCCC4)C3)s2)n1] |
| I-94 | 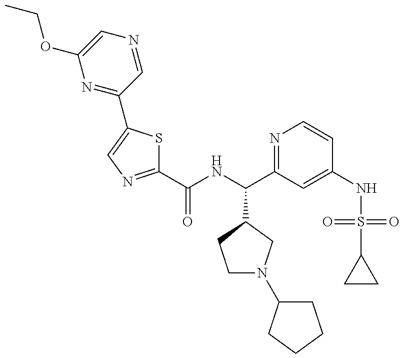 [SMILES: CCOc1cncc(-c2cnc(C(=O)N[C@H](c3cc(NS(=O)(=O)C4CC4)ccn3)[C@@H]3CCN(C4CCCC4)C3)s2)n1] |
| I-95 | 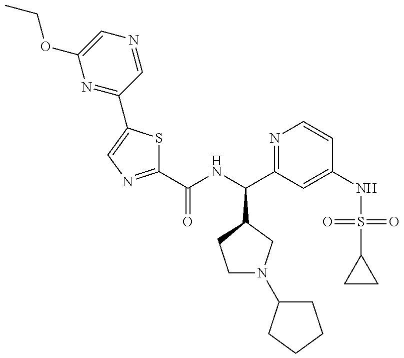 [SMILES: CCOc1cncc(-c2cnc(C(=O)N[C@@H](c3cc(NS(=O)(=O)C4CC4)ccn3)[C@@H]3CCN(C4CCCC4)C3)s2)n1] |
| I-96 | 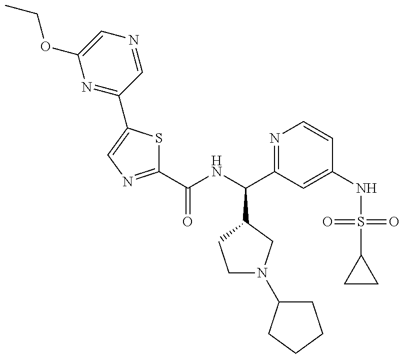 [SMILES: CCOc1cncc(-c2cnc(C(=O)N[C@@H](c3cc(NS(=O)(=O)C4CC4)ccn3)[C@H]3CCN(C4CCCC4)C3)s2)n1] |
| I-97 | 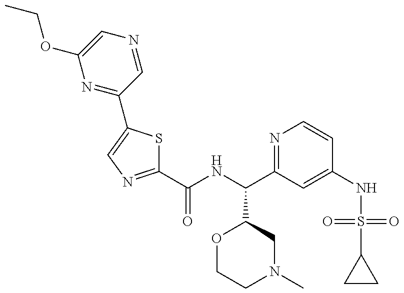 [SMILES: CCOc1cncc(-c2cnc(C(=O)N[C@H](c3cc(NS(=O)(=O)C4CC4)ccn3)[C@H]3CN(C)CCO3)s2)n1] |
| I-98 | 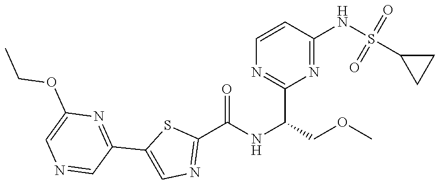 [SMILES: CCOc1cncc(-c2cnc(C(=O)N[C@@H](COC)c3nccc(NS(=O)(=O)C4CC4)n3)s2)n1] |
| I-99 | 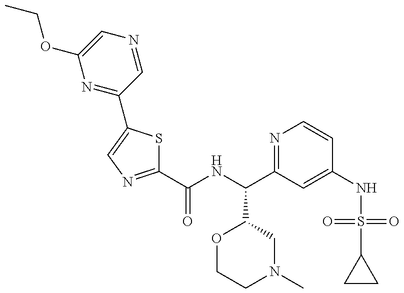 [SMILES: CCOc1cncc(-c2cnc(C(=O)N[C@H](c3cc(NS(=O)(=O)C4CC4)ccn3)[C@@H]3CN(C)CCO3)s2)n1] |
| I-100 | 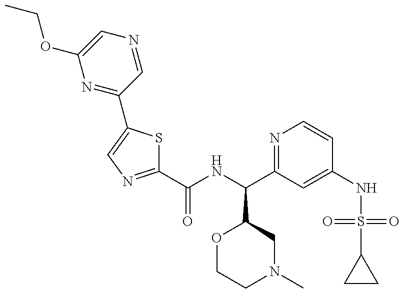 [SMILES: CCOc1cncc(-c2cnc(C(=O)N[C@@H](c3cc(NS(=O)(=O)C4CC4)ccn3)[C@H]3CN(C)CCO3)s2)n1] |
| I-101 | 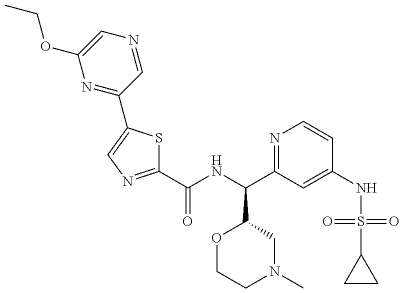 [SMILES: CCOc1cncc(-c2cnc(C(=O)N[C@@H](c3cc(NS(=O)(=O)C4CC4)ccn3)[C@@H]3CN(C)CCO3)s2)n1] |
| I-102 | 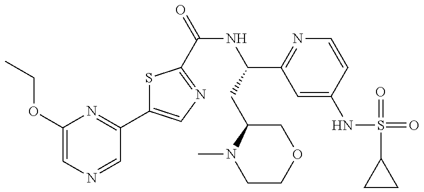 [SMILES: CCOc1cncc(-c2cnc(C(=O)N[C@@H](C[C@H]3COCCN3C)c3cc(NS(=O)(=O)C4CC4)ccn3)s2)n1] |
| I-103 | 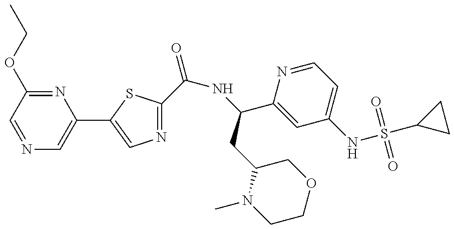 |
| I-104 | 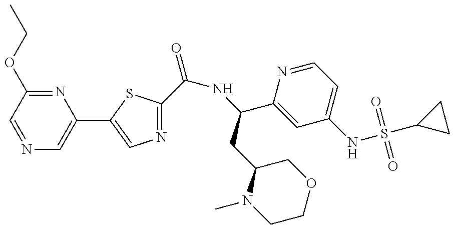 [SMILES: CCOc1cncc(-c2cnc(C(=O)N[C@H](C[C@H]3COCCN3C)c3cc(NS(=O)(=O)C4CC4)ccn3)s2)n1] |
| I-105 | 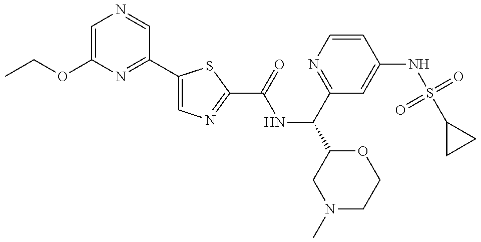 [SMILES: CCOc1cncc(-c2cnc(C(=O)N[C@H](c3cc(NS(=O)(=O)C4CC4)ccn3)C3CN(C)CCO3)s2)n1] |
| I-106 | 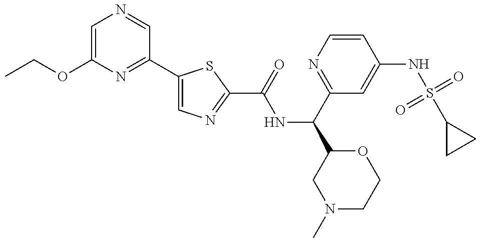 [SMILES: CCOc1cncc(-c2cnc(C(=O)N[C@@H](c3cc(NS(=O)(=O)C4CC4)ccn3)C3CN(C)CCO3)s2)n1] |
| I-107 | 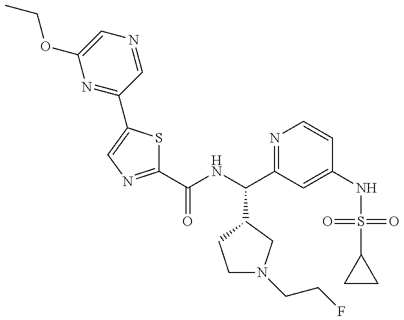 [SMILES: CCOc1cncc(-c2cnc(C(=O)N[C@H](c3cc(NS(=O)(=O)C4CC4)ccn3)[C@H]3CCN(CCF)C3)s2)n1] |
| I-108 | 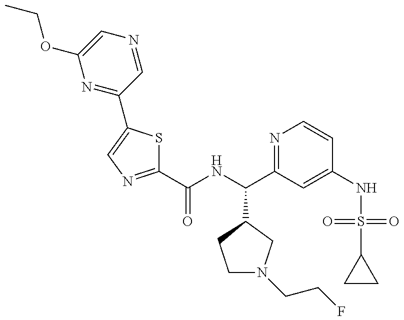 [SMILES: CCOc1cncc(-c2cnc(C(=O)N[C@H](c3cc(NS(=O)(=O)C4CC4)ccn3)[C@@H]3CCN(CCF)C3)s2)n1] |
| I-109 | 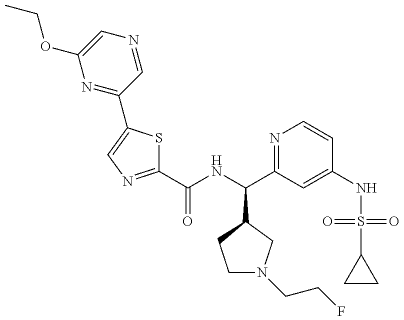 [SMILES: CCOc1cncc(-c2cnc(C(=O)N[C@@H](c3cc(NS(=O)(=O)C4CC4)ccn3)[C@@H]3CCN(CCF)C3)s2)n1] |
| I-110 | 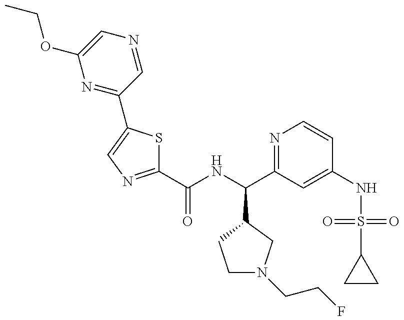 [SMILES: CCOc1cncc(-c2cnc(C(=O)N[C@@H](c3cc(NS(=O)(=O)C4CC4)ccn3)[C@H]3CCN(CCF)C3)s2)n1] |
| I-111 | 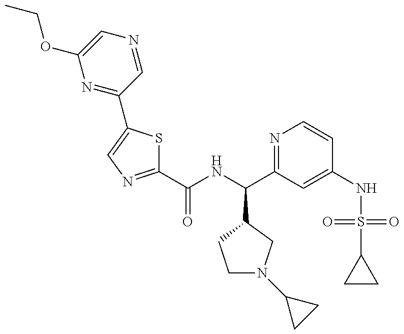 [SMILES: CCOc1cncc(-c2cnc(C(=O)N[C@@H](c3cc(NS(=O)(=O)C4CC4)ccn3)[C@H]3CCN(C4CC4)C3)s2)n1] |
| I-112 | 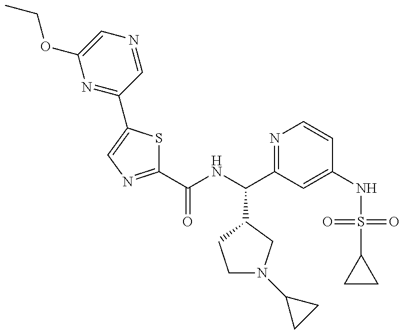 [SMILES: CCOc1cncc(-c2cnc(C(=O)N[C@H](c3cc(NS(=O)(=O)C4CC4)ccn3)[C@H]3CCN(C4CC4)C3)s2)n1] |
| I-113 | 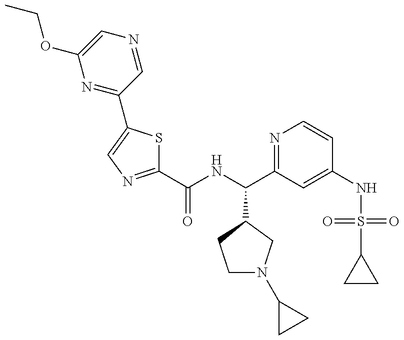 [SMILES: CCOc1cncc(-c2cnc(C(=O)N[C@H](c3cc(NS(=O)(=O)C4CC4)ccn3)[C@@H]3CCN(C4CC4)C3)s2)n1] |
| I-114 | 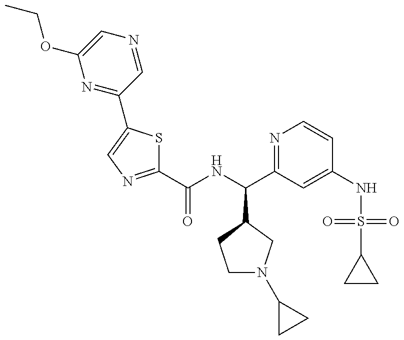 [SMILES: CCOc1cncc(-c2cnc(C(=O)N[C@@H](c3cc(NS(=O)(=O)C4CC4)ccn3)[C@@H]3CCN(C4CC4)C3)s2)n1] |
| I-115 | 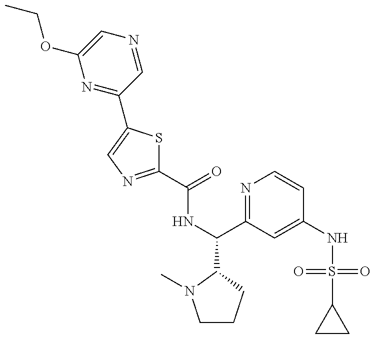 [SMILES: CCOc1cncc(-c2cnc(C(=O)N[C@H](c3cc(NS(=O)(=O)C4CC4)ccn3)[C@@H]3CCCN3C)s2)n1] |
| I-116 | 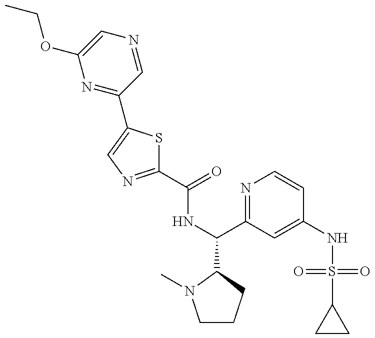 [SMILES: CCOc1cncc(-c2cnc(C(=O)N[C@H](c3cc(NS(=O)(=O)C4CC4)ccn3)[C@H]3CCCN3C)s2)n1] |
| I-117 | 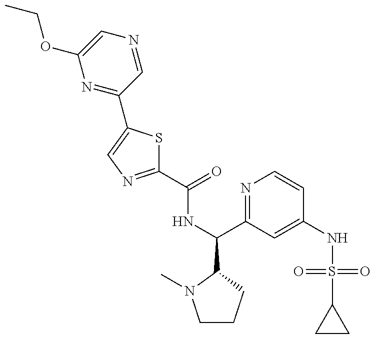 [SMILES: CCOc1cncc(-c2cnc(C(=O)N[C@@H](c3cc(NS(=O)(=O)C4CC4)ccn3)[C@@H]3CCCN3C)s2)n1] |
| I-118 | 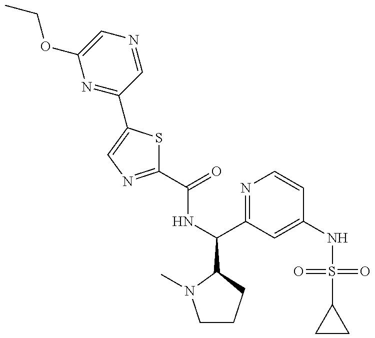 [SMILES: CCOc1cncc(-c2cnc(C(=O)N[C@@H](c3cc(NS(=O)(=O)C4CC4)ccn3)[C@H]3CCCN3C)s2)n1] |
| I-119 | 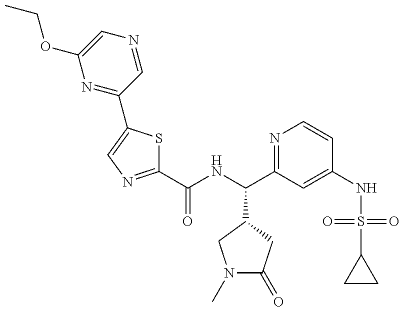 [SMILES: CCOc1cncc(-c2cnc(C(=O)N[C@H](c3cc(NS(=O)(=O)C4CC4)ccn3)[C@@H]3CC(=O)N(C)C3)s2)n1] |
| I-120 | 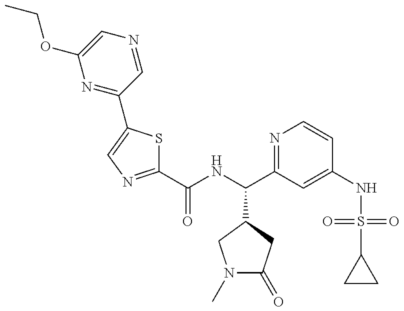 [SMILES: CCOc1cncc(-c2cnc(C(=O)N[C@H](c3cc(NS(=O)(=O)C4CC4)ccn3)[C@H]3CC(=O)N(C)C3)s2)n1] |
| I-121 | 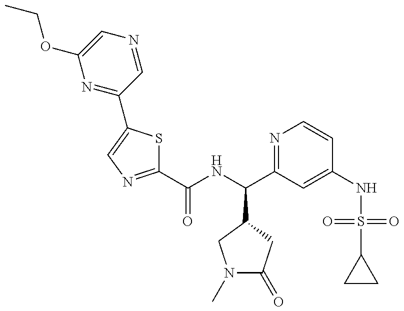 [SMILES: CCOc1cncc(-c2cnc(C(=O)N[C@@H](c3cc(NS(=O)(=O)C4CC4)ccn3)[C@@H]3CC(=O)N(C)C3)s2)n1] |
| I-122 | 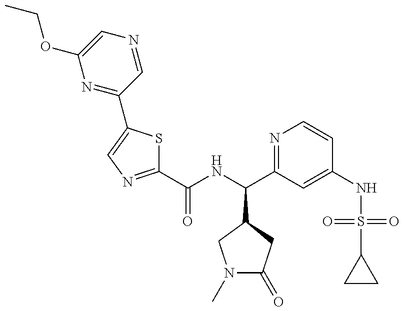 [SMILES: CCOc1cncc(-c2cnc(C(=O)N[C@@H](c3cc(NS(=O)(=O)C4CC4)ccn3)[C@H]3CC(=O)N(C)C3)s2)n1] |
| I-123 | 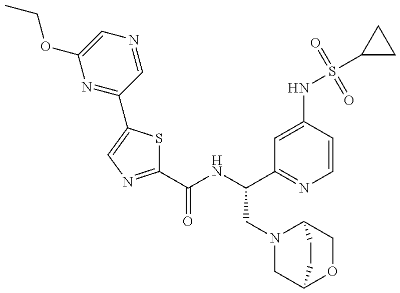 [SMILES: CCOc1cncc(-c2cnc(C(=O)N[C@@H](CN3C[C@H]4CC[C@@H]3CO4)c3cc(NS(=O)(=O)C4CC4)ccn3)s2)n1] |
| I-124 | 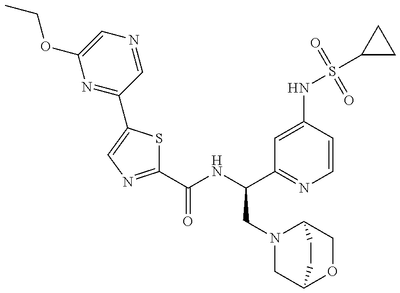 [SMILES: CCOc1cncc(-c2cnc(C(=O)N[C@H](CN3C[C@H]4CC[C@@H]3CO4)c3cc(NS(=O)(=O)C4CC4)ccn3)s2)n1] |
| I-125 | 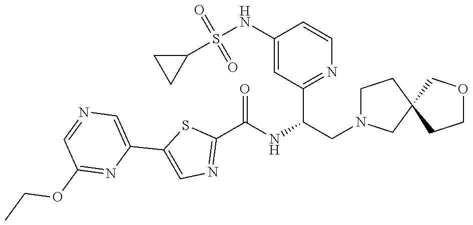 [SMILES: CCOc1cncc(-c2cnc(C(=O)N[C@@H](CN3CC[C@@]4(CCOC4)C3)c3cc(NS(=O)(=O)C4CC4)ccn3)s2)n1] |
| I-126 | 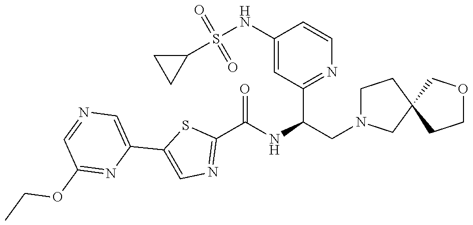 [SMILES: CCOc1cncc(-c2cnc(C(=O)N[C@H](CN3CC[C@@]4(CCOC4)C3)c3cc(NS(=O)(=O)C4CC4)ccn3)s2)n1] |
| I-127 | 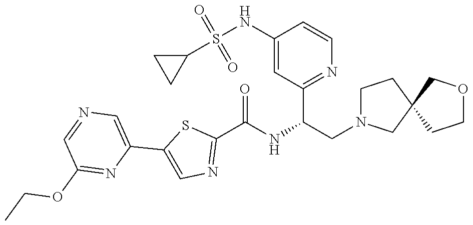 [SMILES: CCOc1cncc(-c2cnc(C(=O)N[C@@H](CN3CC[C@]4(CCOC4)C3)c3cc(NS(=O)(=O)C4CC4)ccn3)s2)n1] |
| I-128 | 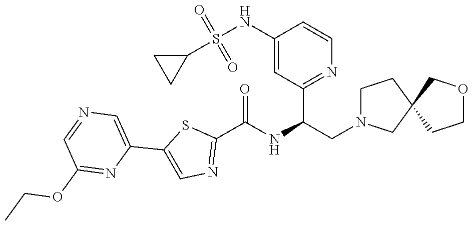 [SMILES: CCOc1cncc(-c2cnc(C(=O)N[C@H](CN3CC[C@]4(CCOC4)C3)c3cc(NS(=O)(=O)C4CC4)ccn3)s2)n1] |
| I-129 | 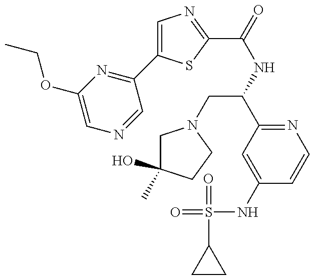 [SMILES: CCOc1cncc(-c2cnc(C(=O)N[C@@H](CN3CC[C@@](C)(O)C3)c3cc(NS(=O)(=O)C4CC4)ccn3)s2)n1] |
| I-130 | 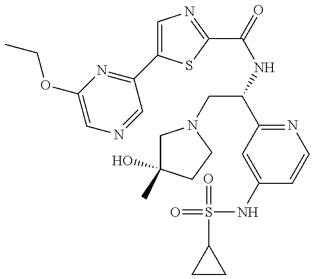 [SMILES: CCOc1cncc(-c2cnc(C(=O)N[C@@H](CN3CC[C@](C)(O)C3)c3cc(NS(=O)(=O)C4CC4)ccn3)s2)n1] |
| I-131 | 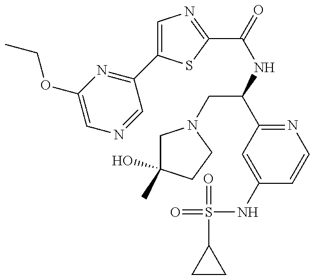 [SMILES: CCOc1cncc(-c2cnc(C(=O)N[C@H](CN3CC[C@](C)(O)C3)c3cc(NS(=O)(=O)C4CC4)ccn3)s2)n1] |
| I-132 | 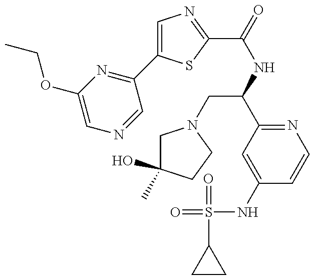 [SMILES: CCOc1cncc(-c2cnc(C(=O)N[C@H](CN3CC[C@@](C)(O)C3)c3cc(NS(=O)(=O)C4CC4)ccn3)s2)n1] |
| I-133 | 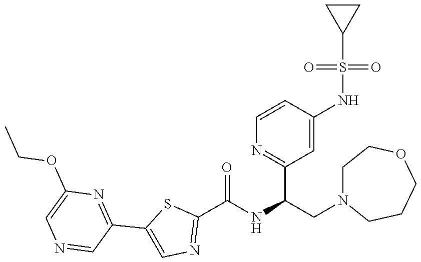 [SMILES: CCOc1cncc(-c2cnc(C(=O)N[C@H](CN3CCCOCC3)c3cc(NS(=O)(=O)C4CC4)ccn3)s2)n1] |
| I-134 | 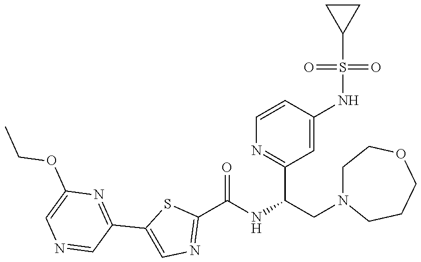 [SMILES: CCOc1cncc(-c2cnc(C(=O)N[C@@H](CN3CCCOCC3)c3cc(NS(=O)(=O)C4CC4)ccn3)s2)n1] |
| I-135 | 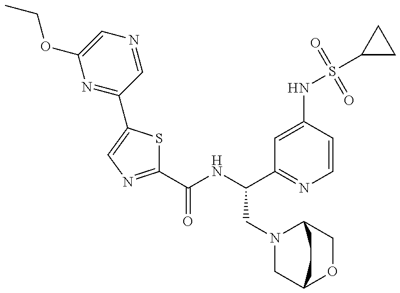 [SMILES: CCOc1cncc(-c2cnc(C(=O)N[C@@H](CN3C[C@@H]4CC[C@H]3CO4)c3cc(NS(=O)(=O)C4CC4)ccn3)s2)n1] |
| I-136 | 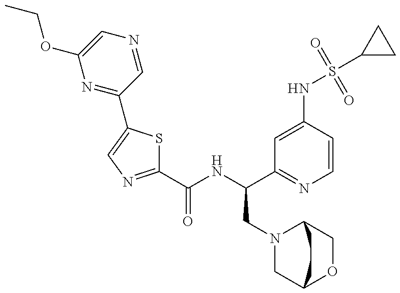 [SMILES: CCOc1cncc(-c2cnc(C(=O)N[C@H](CN3C[C@@H]4CC[C@H]3CO4)c3cc(NS(=O)(=O)C4CC4)ccn3)s2)n1] |
| I-137 | 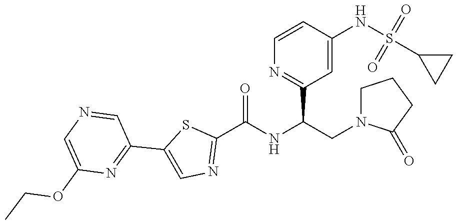 [SMILES: CCOc1cncc(-c2cnc(C(=O)N[C@H](CN3CCCC3=O)c3cc(NS(=O)(=O)C4CC4)ccn3)s2)n1] |
| I-138 | 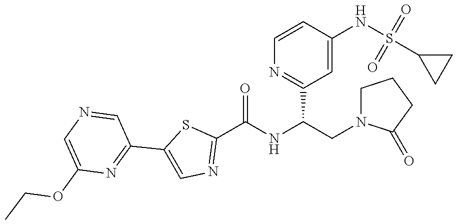 [SMILES: CCOc1cncc(-c2cnc(C(=O)N[C@@H](CN3CCCC3=O)c3cc(NS(=O)(=O)C4CC4)ccn3)s2)n1] |
| I-139 | 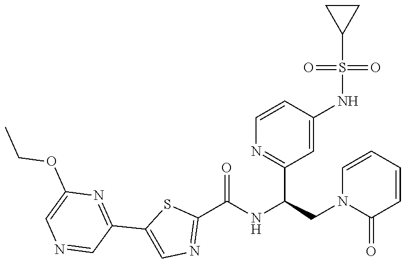 [SMILES: CCOc1cncc(-c2cnc(C(=O)N[C@H](Cn3ccccc3=O)c3cc(NS(=O)(=O)C4CC4)ccn3)s2)n1] |
| I-140 | 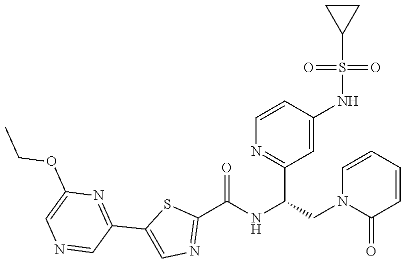 [SMILES: CCOc1cncc(-c2cnc(C(=O)N[C@@H](Cn3ccccc3=O)c3cc(NS(=O)(=O)C4CC4)ccn3)s2)n1] |
| I-141 | 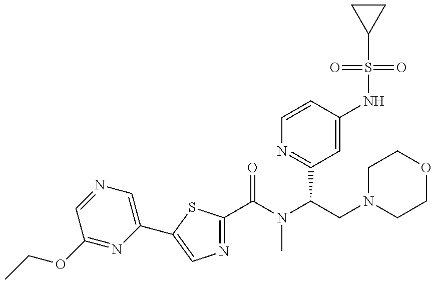 [SMILES: CCOc1cncc(-c2cnc(C(=O)N(C)[C@@H](CN3CCOCC3)c3cc(NS(=O)(=O)C4CC4)ccn3)s2)n1] |
| I-142 | 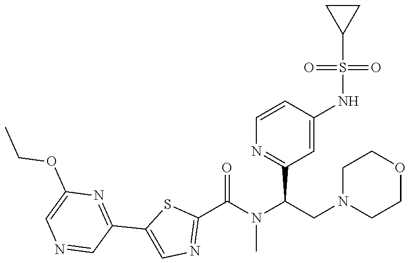 [SMILES: CCOc1cncc(-c2cnc(C(=O)N(C)[C@H](CN3CCOCC3)c3cc(NS(=O)(=O)C4CC4)ccn3)s2)n1] |
| I-143 | 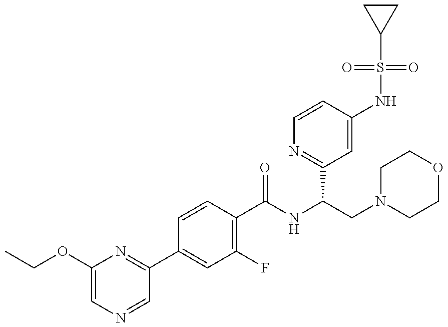 [SMILES: CCOc1cncc(-c2ccc(C(=O)N[C@@H](CN3CCOCC3)c3cc(NS(=O)(=O)C4CC4)ccn3)c(F)c2)n1] |
| I-144 | 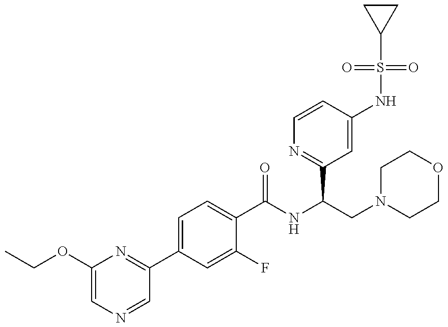 [SMILES: CCOc1cncc(-c2ccc(C(=O)N[C@H](CN3CCOCC3)c3cc(NS(=O)(=O)C4CC4)ccn3)c(F)c2)n1] |
| I-145 | 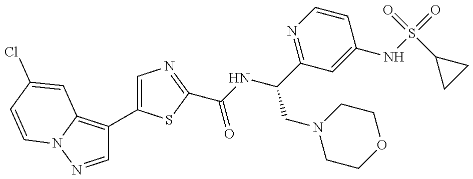 [SMILES: O=C(N[C@@H](CN1CCOCC1)c1cc(NS(=O)(=O)C2CC2)ccn1)c1ncc(-c2cnn3ccc(Cl)cc23)s1] |
| I-146 | 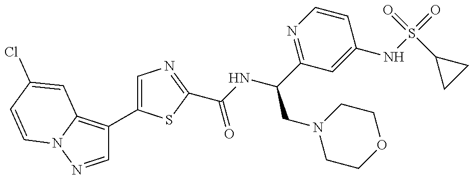 [SMILES: O=C(N[C@H](CN1CCOCC1)c1cc(NS(=O)(=O)C2CC2)ccn1)c1ncc(-c2cnn3ccc(Cl)cc23)s1] |
| I-147 | 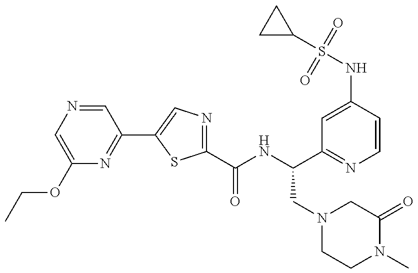 [SMILES: CCOc1cncc(-c2cnc(C(=O)N[C@@H](CN3CCN(C)C(=O)C3)c3cc(NS(=O)(=O)C4CC4)ccn3)s2)n1] |
| I-148 | 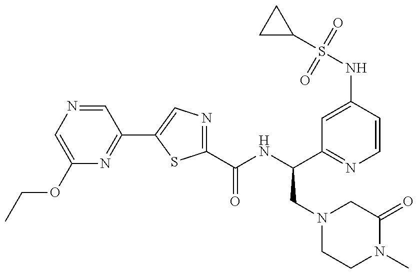 [SMILES: CCOc1cncc(-c2cnc(C(=O)N[C@H](CN3CCN(C)C(=O)C3)c3cc(NS(=O)(=O)C4CC4)ccn3)s2)n1] |
| I-149 | 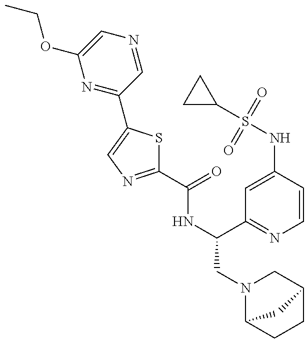 [SMILES: CCOc1cncc(-c2cnc(C(=O)N[C@@H](CN3C[C@H]4CC[C@@H]3C4)c3cc(NS(=O)(=O)C4CC4)ccn3)s2)n1] |
| I-150 | 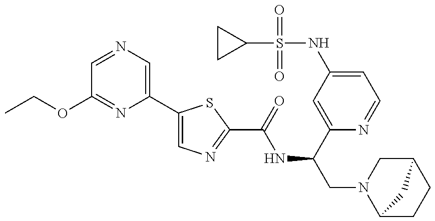 [SMILES: CCOc1cncc(-c2cnc(C(=O)N[C@H](CN3C[C@H]4CC[C@@H]3C4)c3cc(NS(=O)(=O)C4CC4)ccn3)s2)n1] |
| I-151 | 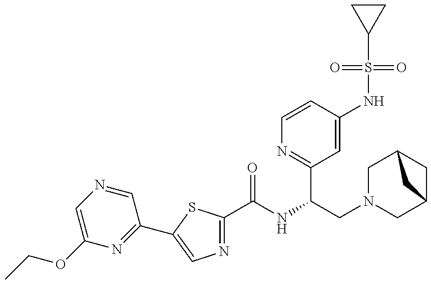 [SMILES: CCOc1cncc(-c2cnc(C(=O)N[C@@H](CN3C[C@H]4C[C@@H](C3)C4)c3cc(NS(=O)(=O)C4CC4)ccn3)s2)n1] |
| I-152 | 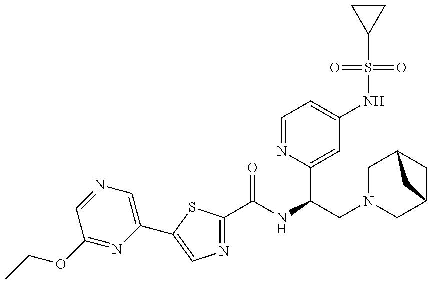 [SMILES: CCOc1cncc(-c2cnc(C(=O)N[C@H](CN3C[C@H]4C[C@@H](C3)C4)c3cc(NS(=O)(=O)C4CC4)ccn3)s2)n1] |
| I-153 | 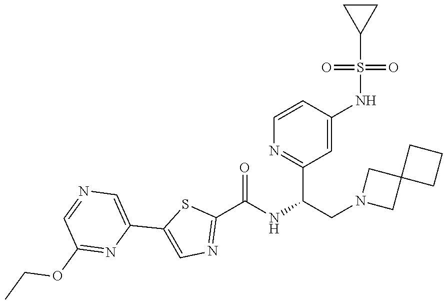 [SMILES: CCOc1cncc(-c2cnc(C(=O)N[C@@H](CN3CC4(CCC4)C3)c3cc(NS(=O)(=O)C4CC4)ccn3)s2)n1] |
| I-154 | 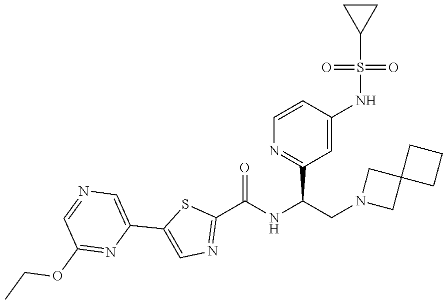 [SMILES: CCOc1cncc(-c2cnc(C(=O)N[C@H](CN3CC4(CCC4)C3)c3cc(NS(=O)(=O)C4CC4)ccn3)s2)n1] |
| I-155 | 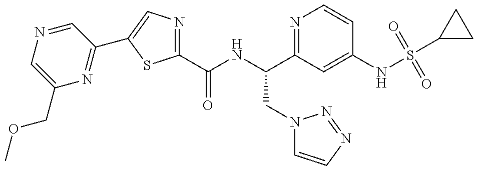 [SMILES: COCc1cncc(-c2cnc(C(=O)N[C@@H](Cn3ccnn3)c3cc(NS(=O)(=O)C4CC4)ccn3)s2)n1] |
| I-156 | 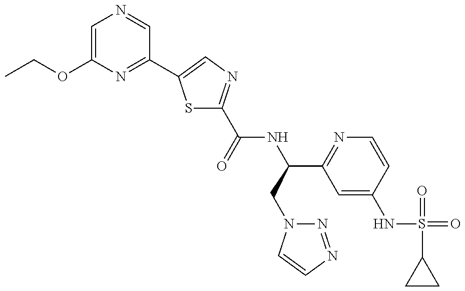 [SMILES: CCOc1cncc(-c2cnc(C(=O)N[C@H](Cn3ccnn3)c3cc(NS(=O)(=O)C4CC4)ccn3)s2)n1] |
| I-157 | 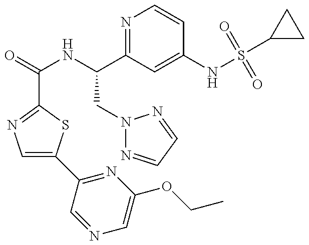 [SMILES: CCOc1cncc(-c2cnc(C(=O)N[C@@H](Cn3nccn3)c3cc(NS(=O)(=O)C4CC4)ccn3)s2)n1] |
| I-158 | 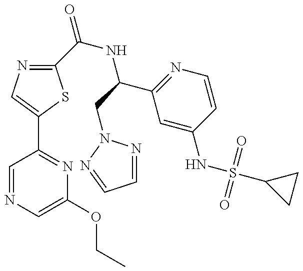 [SMILES: CCOc1cncc(-c2cnc(C(=O)N[C@H](Cn3nccn3)c3cc(NS(=O)(=O)C4CC4)ccn3)s2)n1] |
| I-159 | 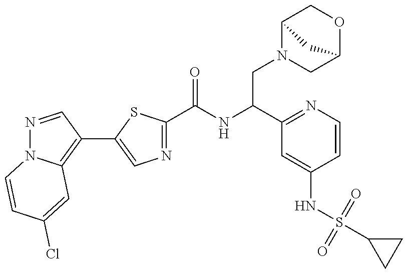 [SMILES: O=C(NC(CN1C[C@H]2C[C@@H]1CO2)c1cc(NS(=O)(=O)C2CC2)ccn1)c1ncc(-c2cnn3ccc(Cl)cc23)s1] |
| I-160 | 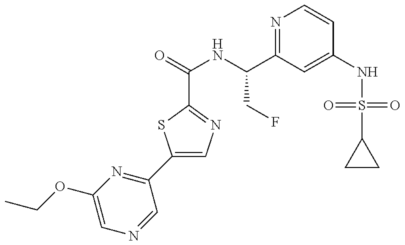 [SMILES: CCOc1cncc(-c2cnc(C(=O)N[C@@H](CF)c3cc(NS(=O)(=O)C4CC4)ccn3)s2)n1] |
| I-161 | 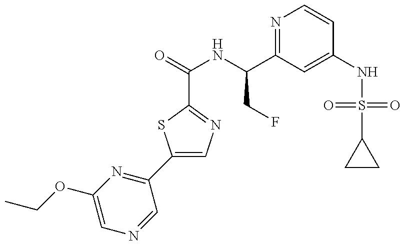 [SMILES: CCOc1cncc(-c2cnc(C(=O)N[C@H](CF)c3cc(NS(=O)(=O)C4CC4)ccn3)s2)n1] |
| I-162 | 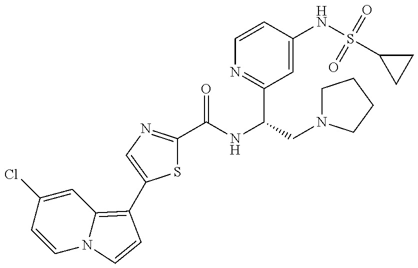 [SMILES: O=C(N[C@@H](CN1CCCC1)c1cc(NS(=O)(=O)C2CC2)ccn1)c1ncc(-c2ccn3ccc(Cl)cc23)s1] |
| I-163 | 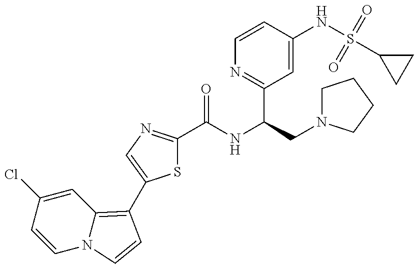 [SMILES: O=C(N[C@H](CN1CCCC1)c1cc(NS(=O)(=O)C2CC2)ccn1)c1ncc(-c2ccn3ccc(Cl)cc23)s1] |
| I-164 | 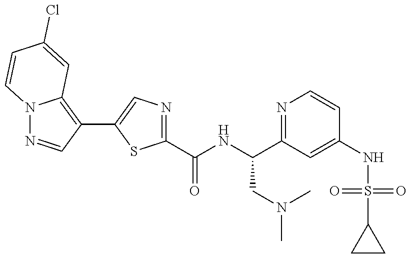 [SMILES: CN(C)C[C@H](NC(=O)c1ncc(-c2cnn3ccc(Cl)cc23)s1)c1cc(NS(=O)(=O)C2CC2)ccn1] |
| I-165 | 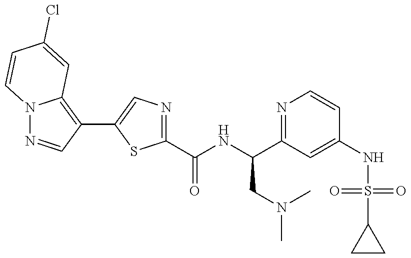 [SMILES: CN(C)C[C@@H](NC(=O)c1ncc(-c2cnn3ccc(Cl)cc23)s1)c1cc(NS(=O)(=O)C2CC2)ccn1] |
| I-166 | 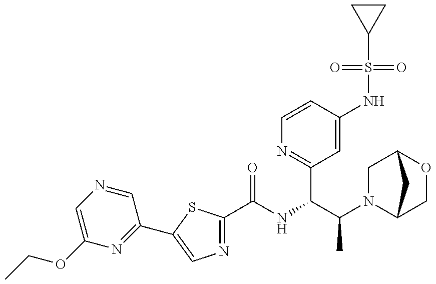 [SMILES: CCOc1cncc(-c2cnc(C(=O)N[C@H](c3cc(NS(=O)(=O)C4CC4)ccn3)[C@H](C)N3C[C@H]4C[C@@H]3CO4)s2)n1] |
| I-167 | 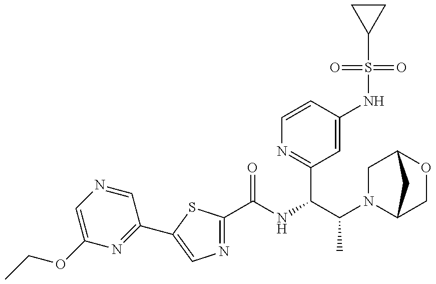 [SMILES: CCOc1cncc(-c2cnc(C(=O)N[C@H](c3cc(NS(=O)(=O)C4CC4)ccn3)[C@@H](C)N3C[C@H]4C[C@@H]3CO4)s2)n1] |
| I-168 | 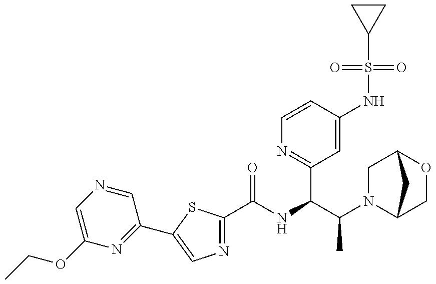 [SMILES: CCOc1cncc(-c2cnc(C(=O)N[C@@H](c3cc(NS(=O)(=O)C4CC4)ccn3)[C@H](C)N3C[C@H]4C[C@@H]3CO4)s2)n1] |
| I-169 | 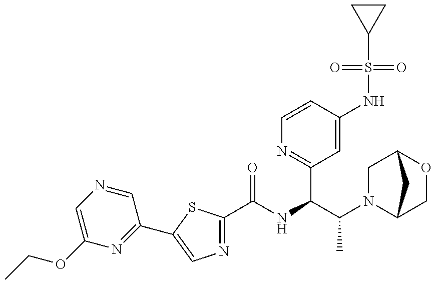 [SMILES: CCOc1cncc(-c2cnc(C(=O)N[C@@H](c3cc(NS(=O)(=O)C4CC4)ccn3)[C@@H](C)N3C[C@H]4C[C@@H]3CO4)s2)n1] |
| I-170 | 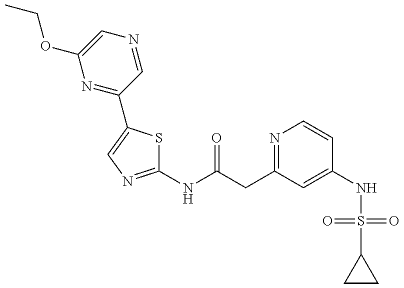 [SMILES: CCOc1cncc(-c2cnc(NC(=O)Cc3cc(NS(=O)(=O)C4CC4)ccn3)s2)n1] |
| I-171 | 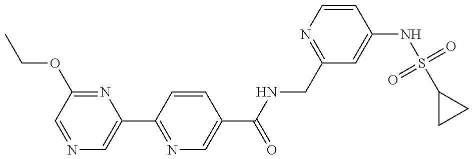 [SMILES: CCOc1cncc(-c2ccc(C(=O)NCc3cc(NS(=O)(=O)C4CC4)ccn3)cn2)n1] |
| I-172 | 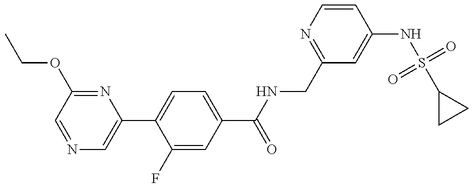 [SMILES: CCOc1cncc(-c2ccc(C(=O)NCc3cc(NS(=O)(=O)C4CC4)ccn3)cc2F)n1] |
| I-173 | 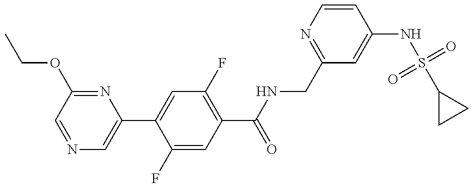 [SMILES: CCOc1cncc(-c2cc(F)c(C(=O)NCc3cc(NS(=O)(=O)C4CC4)ccn3)cc2F)n1] |
| I-174 | 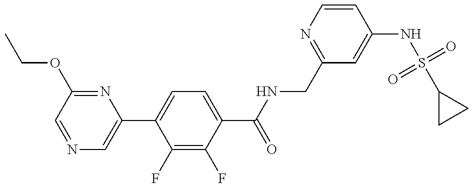 [SMILES: CCOc1cncc(-c2ccc(C(=O)NCc3cc(NS(=O)(=O)C4CC4)ccn3)c(F)c2F)n1] |
| I-175 | 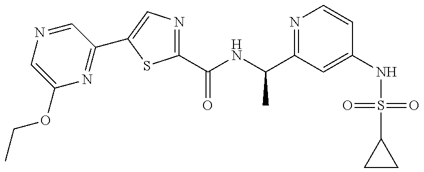 [SMILES: CCOc1cncc(-c2cnc(C(=O)N[C@H](C)c3cc(NS(=O)(=O)C4CC4)ccn3)s2)n1] |
| I-176 | 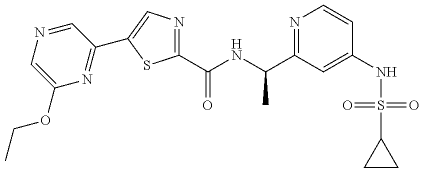 [SMILES: CCOc1cncc(-c2cnc(C(=O)N[C@H](C)c3cc(NS(=O)(=O)C4CC4)ccn3)s2)n1] |
| I-177 | 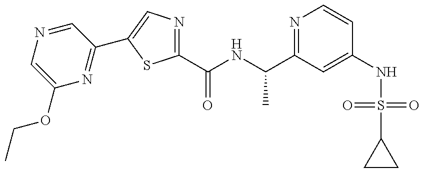 [SMILES: CCOc1cncc(-c2cnc(C(=O)N[C@@H](C)c3cc(NS(=O)(=O)C4CC4)ccn3)s2)n1] |
| I-178 | 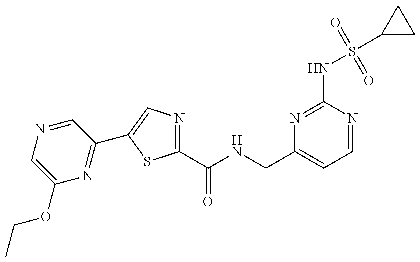 [SMILES: CCOc1cncc(-c2cnc(C(=O)NCc3ccnc(NS(=O)(=O)C4CC4)n3)s2)n1] |
| I-179 | 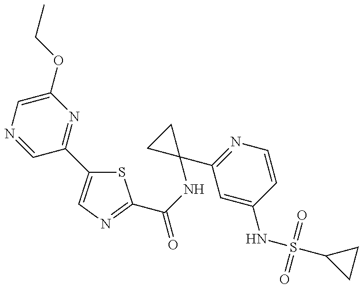 [SMILES: CCOc1cncc(-c2cnc(C(=O)NC3(c4cc(NS(=O)(=O)C5CC5)ccn4)CC3)s2)n1] |
| I-180 | 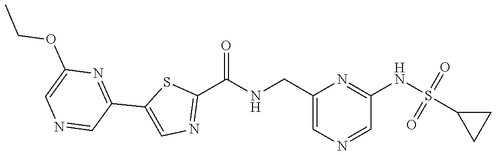 [SMILES: CCOc1cncc(-c2cnc(C(=O)NCc3cncc(NS(=O)(=O)C4CC4)n3)s2)n1] |
| I-181 | 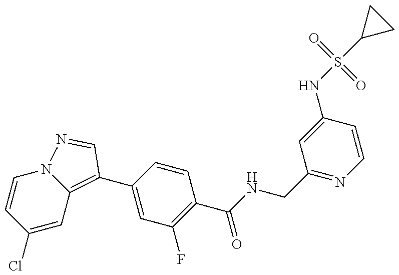 [SMILES: O=C(NCc1cc(NS(=O)(=O)C2CC2)ccn1)c1ccc(-c2cnn3ccc(Cl)cc23)cc1F] |
| I-182 | 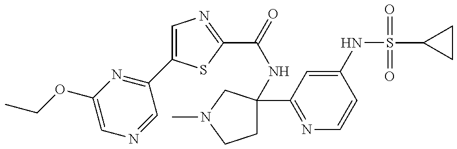 [SMILES: CCOc1cncc(-c2cnc(C(=O)NC3(c4cc(NS(=O)(=O)C5CC5)ccn4)CCN(C)C3)s2)n1] |
| I-183 | 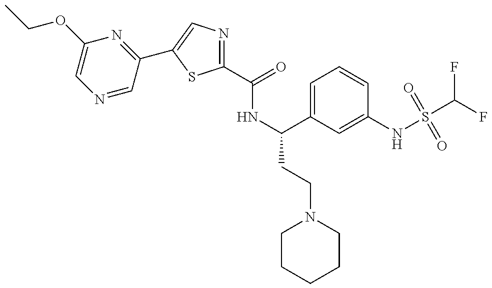 [SMILES: CCOc1cncc(-c2cnc(C(=O)N[C@@H](CCN3CCCCC3)c3cccc(NS(=O)(=O)C(F)F)c3)s2)n1] |
| I-184 | 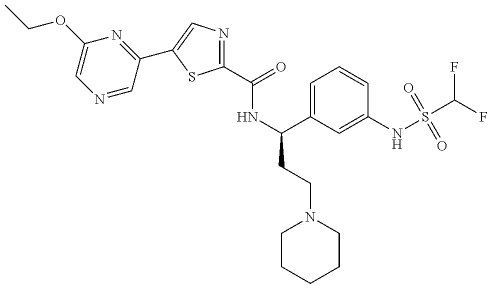 [SMILES: CCOc1cncc(-c2cnc(C(=O)N[C@H](CCN3CCCCC3)c3cccc(NS(=O)(=O)C(F)F)c3)s2)n1] |
| I-185 | 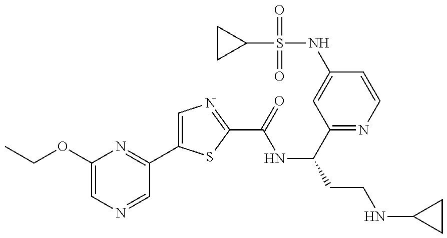 [SMILES: CCOc1cncc(-c2cnc(C(=O)N[C@@H](CCNC3CC3)c3cc(NS(=O)(=O)C4CC4)ccn3)s2)n1] |
| I-186 | 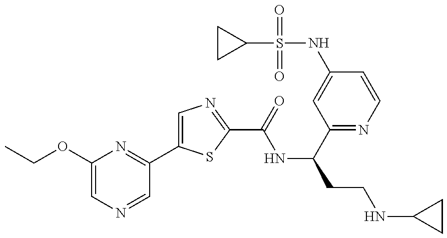 [SMILES: CCOc1cncc(-c2cnc(C(=O)N[C@H](CCNC3CC3)c3cc(NS(=O)(=O)C4CC4)ccn3)s2)n1] |
| I-187 | 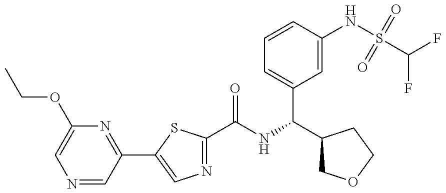 [SMILES: CCOc1cncc(-c2cnc(C(=O)N[C@H](c3cccc(NS(=O)(=O)C(F)F)c3)[C@H]3CCOC3)s2)n1] |
| I-188 | 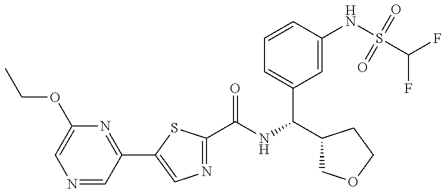 [SMILES: CCOc1cncc(-c2cnc(C(=O)N[C@H](c3cccc(NS(=O)(=O)C(F)F)c3)[C@@H]3CCOC3)s2)n1] |
| I-189 | 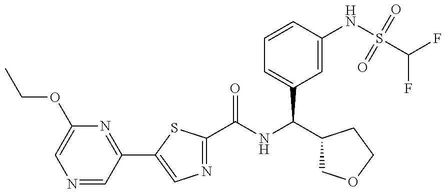 [SMILES: CCOc1cncc(-c2cnc(C(=O)N[C@@H](c3cccc(NS(=O)(=O)C(F)F)c3)[C@@H]3CCOC3)s2)n1] |
| I-190 | 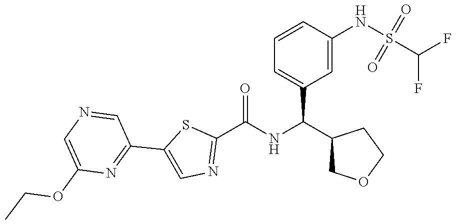 [SMILES: CCOc1cncc(-c2cnc(C(=O)N[C@@H](c3cccc(NS(=O)(=O)C(F)F)c3)[C@H]3CCOC3)s2)n1] |
| I-191 | 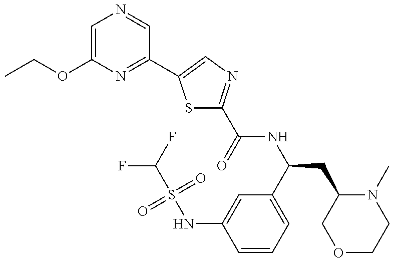 [SMILES: CCOc1cncc(-c2cnc(C(=O)N[C@@H](C[C@@H]3COCCN3C)c3cccc(NS(=O)(=O)C(F)F)c3)s2)n1] |
| I-192 | 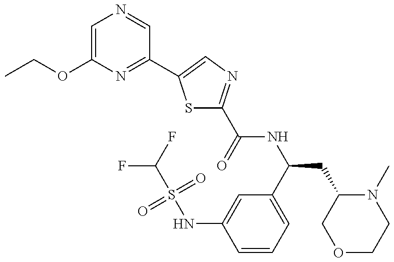 [SMILES: CCOc1cncc(-c2cnc(C(=O)N[C@@H](C[C@H]3COCCN3C)c3cccc(NS(=O)(=O)C(F)F)c3)s2)n1] |
| I-193 | 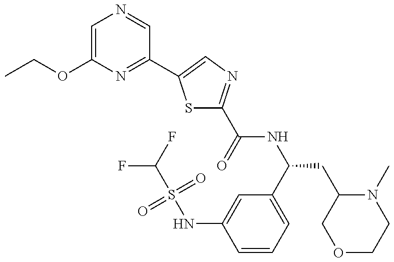 [SMILES: CCOc1cncc(-c2cnc(C(=O)N[C@H](CC3COCCN3C)c3cccc(NS(=O)(=O)C(F)F)c3)s2)n1] |
| I-194 | 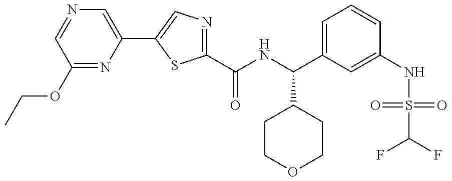 [SMILES: CCOc1cncc(-c2cnc(C(=O)N[C@H](c3cccc(NS(=O)(=O)C(F)F)c3)C3CCOCC3)s2)n1] |
| I-195 | 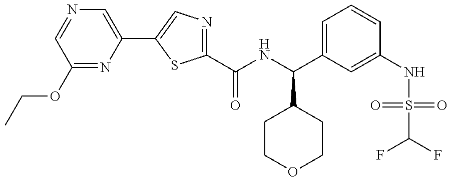 [SMILES: CCOc1cncc(-c2cnc(C(=O)N[C@@H](c3cccc(NS(=O)(=O)C(F)F)c3)C3CCOCC3)s2)n1] |
| I-196 | 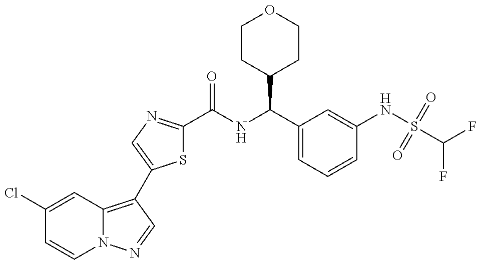 [SMILES: O=C(N[C@H](c1cccc(NS(=O)(=O)C(F)F)c1)C1CCOCC1)c1ncc(-c2cnn3ccc(Cl)cc23)s1] |
| I-197 | 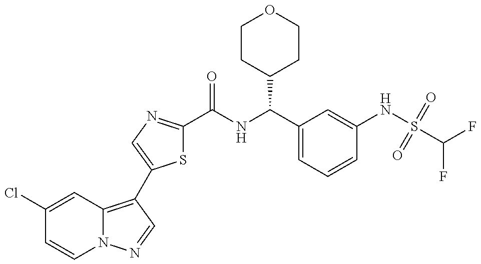 [SMILES: O=C(N[C@@H](c1cccc(NS(=O)(=O)C(F)F)c1)C1CCOCC1)c1ncc(-c2cnn3ccc(Cl)cc23)s1] |
| I-198 | 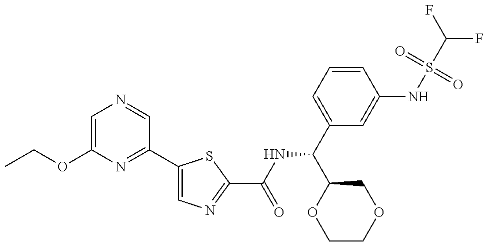 [SMILES: CCOc1cncc(-c2cnc(C(=O)N[C@H](c3cccc(NS(=O)(=O)C(F)F)c3)[C@H]3COCCO3)s2)n1] |
| I-199 | 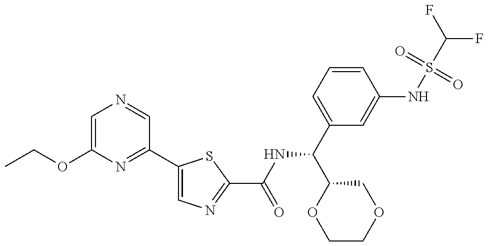 [SMILES: CCOc1cncc(-c2cnc(C(=O)N[C@H](c3cccc(NS(=O)(=O)C(F)F)c3)[C@@H]3COCCO3)s2)n1] |
| I-200 | 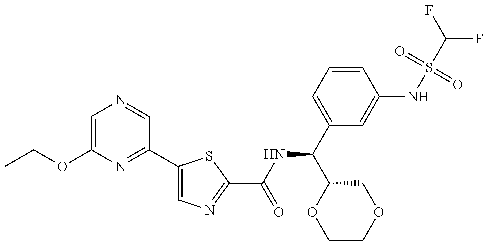 [SMILES: CCOc1cncc(-c2cnc(C(=O)N[C@@H](c3cccc(NS(=O)(=O)C(F)F)c3)[C@@H]3COCCO3)s2)n1] |
| I-201 | 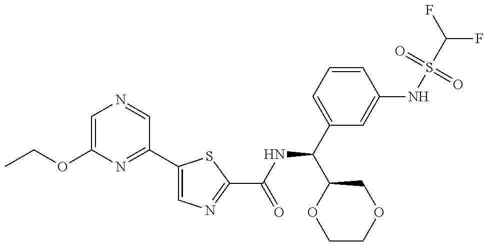 [SMILES: CCOc1cncc(-c2cnc(C(=O)N[C@@H](c3cccc(NS(=O)(=O)C(F)F)c3)[C@H]3COCCO3)s2)n1] |
| I-202 | 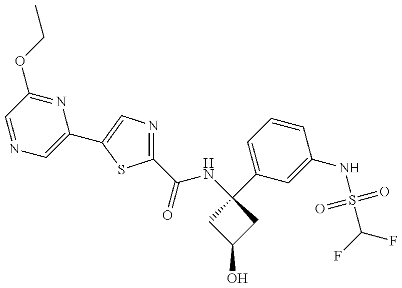 [SMILES: CCOc1cncc(-c2cnc(C(=O)N[C@]3(c4cccc(NS(=O)(=O)C(F)F)c4)C[C@H](O)C3)s2)n1] |
| I-203 | 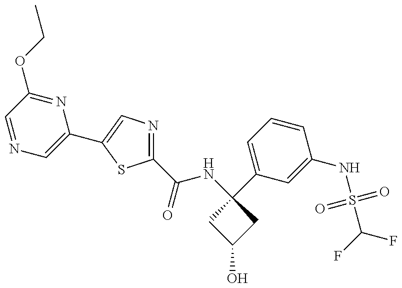 [SMILES: CCOc1cncc(-c2cnc(C(=O)N[C@]3(c4cccc(NS(=O)(=O)C(F)F)c4)C[C@@H](O)C3)s2)n1] |
| I-204 | 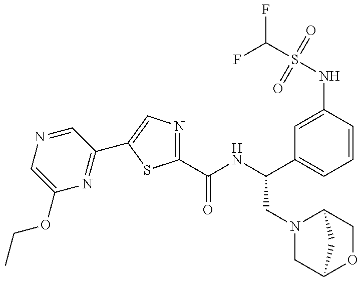 [SMILES: CCOc1cncc(-c2cnc(C(=O)N[C@@H](CN3C[C@H]4C[C@@H]3CO4)c3cccc(NS(=O)(=O)C(F)F)c3)s2)n1] |
| I-205 | 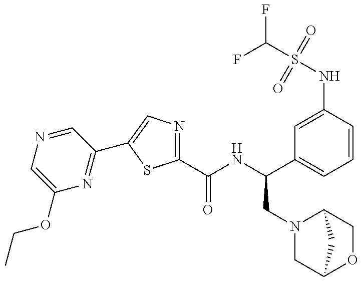 [SMILES: CCOc1cncc(-c2cnc(C(=O)N[C@H](CN3C[C@H]4C[C@@H]3CO4)c3cccc(NS(=O)(=O)C(F)F)c3)s2)n1] |
| I-206 | 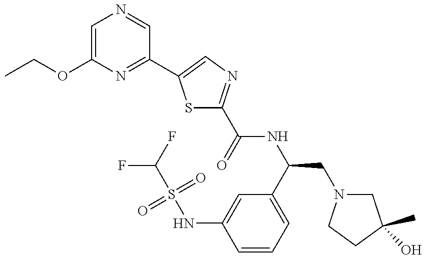 [SMILES: CCOc1cncc(-c2cnc(C(=O)N[C@@H](CN3CC[C@@](C)(O)C3)c3cccc(NS(=O)(=O)C(F)F)c3)s2)n1] |
| I-207 | 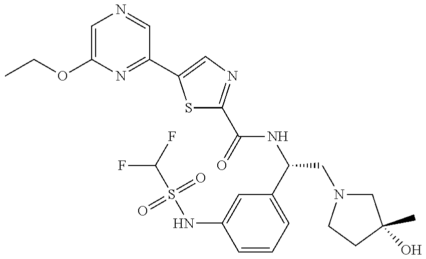 [SMILES: CCOc1cncc(-c2cnc(C(=O)N[C@H](CN3CC[C@@](C)(O)C3)c3cccc(NS(=O)(=O)C(F)F)c3)s2)n1] |
| I-208 | 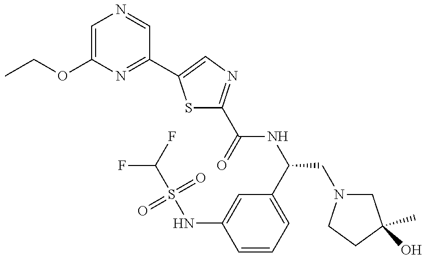 [SMILES: CCOc1cncc(-c2cnc(C(=O)N[C@H](CN3CC[C@](C)(O)C3)c3cccc(NS(=O)(=O)C(F)F)c3)s2)n1] |
| I-209 | 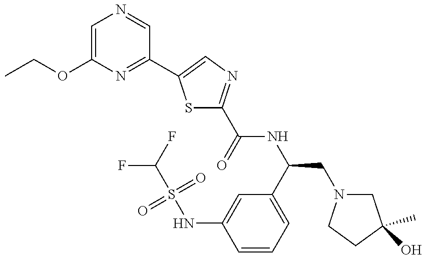 [SMILES: CCOc1cncc(-c2cnc(C(=O)N[C@@H](CN3CC[C@](C)(O)C3)c3cccc(NS(=O)(=O)C(F)F)c3)s2)n1] |
| I-210 | 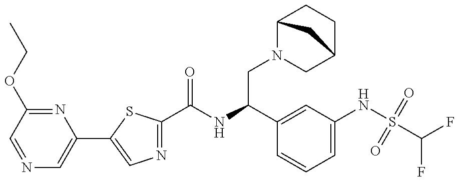 [SMILES: CCOc1cncc(-c2cnc(C(=O)N[C@@H](CN3C[C@H]4CC[C@@H]3C4)c3cccc(NS(=O)(=O)C(F)F)c3)s2)n1] |
| I-211 | 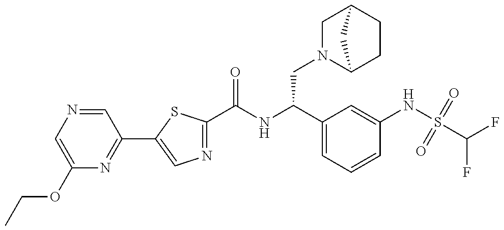 [SMILES: CCOc1cncc(-c2cnc(C(=O)N[C@H](CN3C[C@H]4CC[C@@H]3C4)c3cccc(NS(=O)(=O)C(F)F)c3)s2)n1] |
| I-212 | 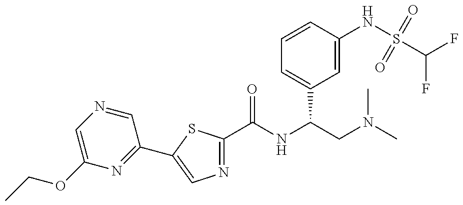 [SMILES: CCOc1cncc(-c2cnc(C(=O)N[C@@H](CN(C)C)c3cccc(NS(=O)(=O)C(F)F)c3)s2)n1] |
| I-213 | 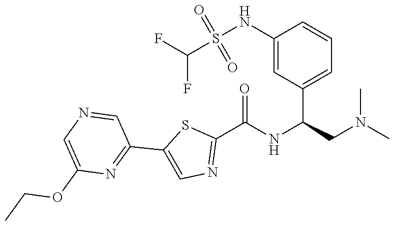 [SMILES: CCOc1cncc(-c2cnc(C(=O)N[C@H](CN(C)C)c3cccc(NS(=O)(=O)C(F)F)c3)s2)n1] |
| I-214 | 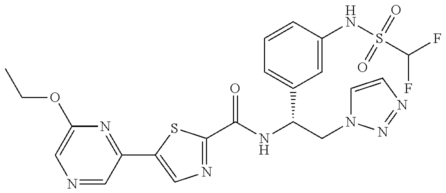 [SMILES: CCOc1cncc(-c2cnc(C(=O)N[C@@H](Cn3ccnn3)c3cccc(NS(=O)(=O)C(F)F)c3)s2)n1] |
| I-215 | 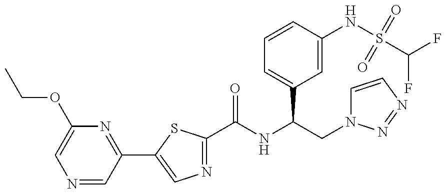 [SMILES: CCOc1cncc(-c2cnc(C(=O)N[C@H](Cn3ccnn3)c3cccc(NS(=O)(=O)C(F)F)c3)s2)n1] |
| I-216 | 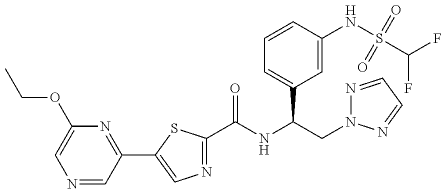 [SMILES: CCOc1cncc(-c2cnc(C(=O)N[C@H](Cn3nccn3)c3cccc(NS(=O)(=O)C(F)F)c3)s2)n1] |
| I-217 | 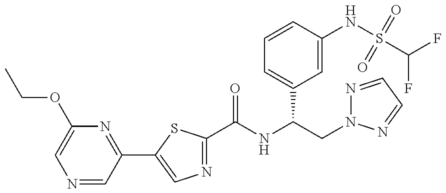 [SMILES: CCOc1cncc(-c2cnc(C(=O)N[C@@H](Cn3nccn3)c3cccc(NS(=O)(=O)C(F)F)c3)s2)n1] |
| I-218 | 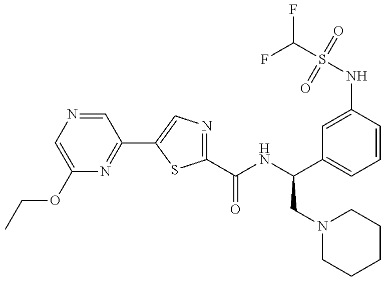 [SMILES: CCOc1cncc(-c2cnc(C(=O)N[C@H](CN3CCCCC3)c3cccc(NS(=O)(=O)C(F)F)c3)s2)n1] |
| I-219 | 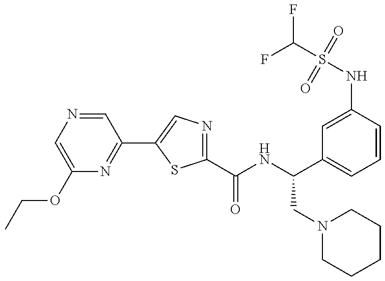 [SMILES: CCOc1cncc(-c2cnc(C(=O)N[C@@H](CN3CCCCC3)c3cccc(NS(=O)(=O)C(F)F)c3)s2)n1] |
| I-220 | 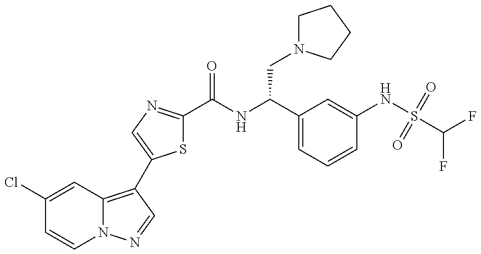 [SMILES: O=C(N[C@H](CN1CCCC1)c1cccc(NS(=O)(=O)C(F)F)c1)c1ncc(-c2cnn3ccc(Cl)cc23)s1] |
| I-221 | 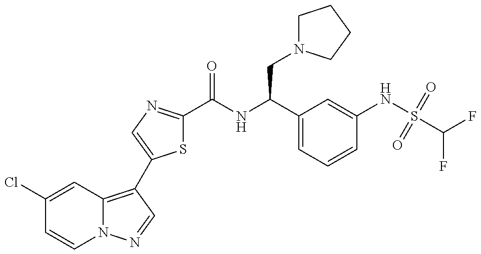 [SMILES: O=C(N[C@@H](CN1CCCC1)c1cccc(NS(=O)(=O)C(F)F)c1)c1ncc(-c2cnn3ccc(Cl)cc23)s1] |
| I-222 | 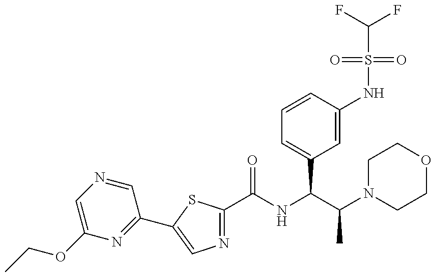 [SMILES: CCOc1cncc(-c2cnc(C(=O)N[C@@H](c3cccc(NS(=O)(=O)C(F)F)c3)[C@H](C)N3CCOCC3)s2)n1] |
| I-223 | 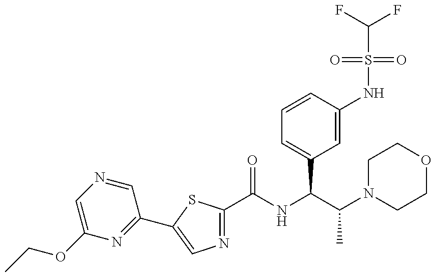 [SMILES: CCOc1cncc(-c2cnc(C(=O)N[C@@H](c3cccc(NS(=O)(=O)C(F)F)c3)[C@@H](C)N3CCOCC3)s2)n1] |
| I-224 | 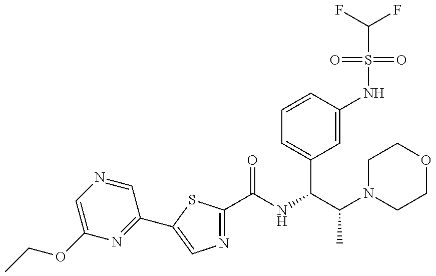 [SMILES: CCOc1cncc(-c2cnc(C(=O)N[C@H](c3cccc(NS(=O)(=O)C(F)F)c3)[C@@H](C)N3CCOCC3)s2)n1] |
| I-225 | 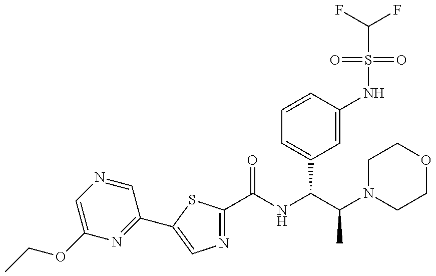 [SMILES: CCOc1cncc(-c2cnc(C(=O)N[C@H](c3cccc(NS(=O)(=O)C(F)F)c3)[C@H](C)N3CCOCC3)s2)n1] |
| I-226 | 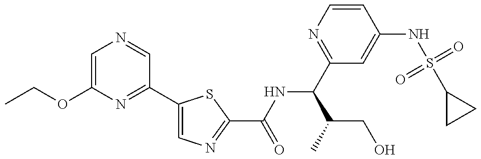 [SMILES: CCOc1cncc(-c2cnc(C(=O)N[C@@H](c3cc(NS(=O)(=O)C4CC4)ccn3)[C@@H](C)CO)s2)n1] |
| I-227 | 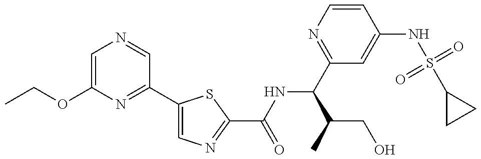 [SMILES: CCOc1cncc(-c2cnc(C(=O)N[C@@H](c3cc(NS(=O)(=O)C4CC4)ccn3)[C@H](C)CO)s2)n1] |
| I-228 | 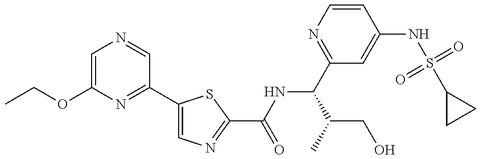 [SMILES: CCOc1cncc(-c2cnc(C(=O)N[C@H](c3cc(NS(=O)(=O)C4CC4)ccn3)[C@@H](C)CO)s2)n1] |
| I-229 | 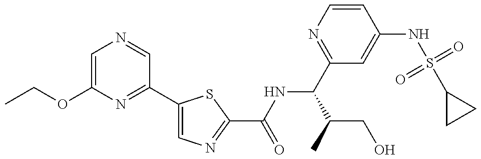 [SMILES: CCOc1cncc(-c2cnc(C(=O)N[C@H](c3cc(NS(=O)(=O)C4CC4)ccn3)[C@H](C)CO)s2)n1] |
| I-230 | 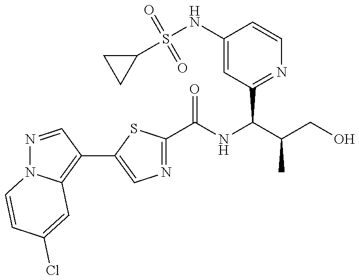 [SMILES: C[C@H](CO)[C@@H](NC(=O)c1ncc(-c2cnn3ccc(Cl)cc23)s1)c1cc(NS(=O)(=O)C2CC2)ccn1] |
| I-231 | 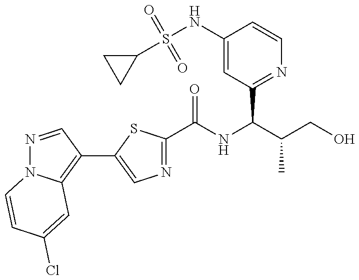 [SMILES: C[C@@H](CO)[C@@H](NC(=O)c1ncc(-c2cnn3ccc(Cl)cc23)s1)c1cc(NS(=O)(=O)C2CC2)ccn1] |
| I-232 | 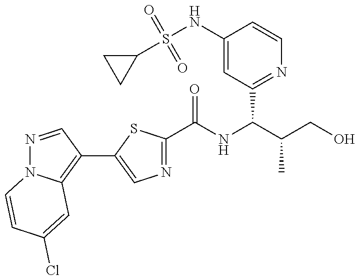 [SMILES: C[C@@H](CO)[C@H](NC(=O)c1ncc(-c2cnn3ccc(Cl)cc23)s1)c1cc(NS(=O)(=O)C2CC2)ccn1] |
| I-233 | 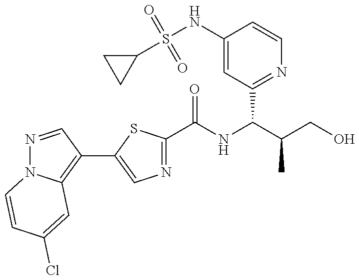 [SMILES: C[C@H](CO)[C@H](NC(=O)c1ncc(-c2cnn3ccc(Cl)cc23)s1)c1cc(NS(=O)(=O)C2CC2)ccn1] |
| I-234 | 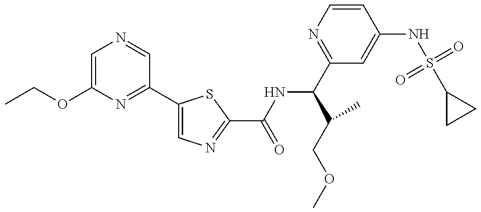 [SMILES: CCOc1cncc(-c2cnc(C(=O)N[C@@H](c3cc(NS(=O)(=O)C4CC4)ccn3)[C@H](C)COC)s2)n1] |
| I-235 | 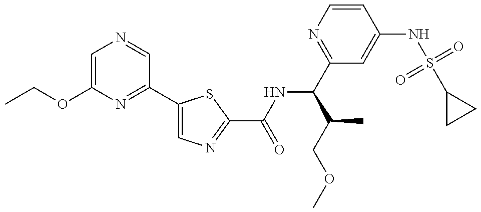 [SMILES: CCOc1cncc(-c2cnc(C(=O)N[C@@H](c3cc(NS(=O)(=O)C4CC4)ccn3)[C@@H](C)COC)s2)n1] |
| I-236 | 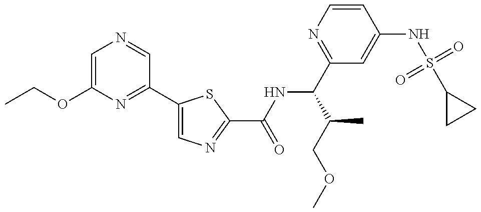 [SMILES: CCOc1cncc(-c2cnc(C(=O)N[C@H](c3cc(NS(=O)(=O)C4CC4)ccn3)[C@@H](C)COC)s2)n1] |
| I-237 | 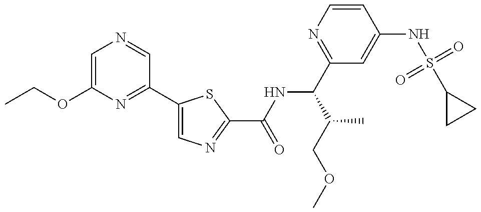 [SMILES: CCOc1cncc(-c2cnc(C(=O)N[C@H](c3cc(NS(=O)(=O)C4CC4)ccn3)[C@H](C)COC)s2)n1] |
| I-238 | 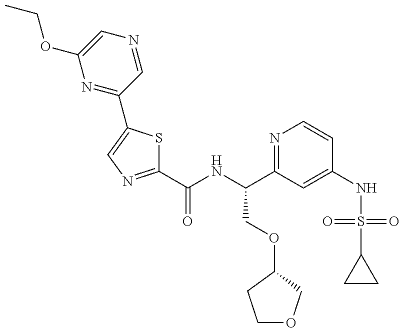 [SMILES: CCOc1cncc(-c2cnc(C(=O)N[C@@H](CO[C@H]3CCOC3)c3cc(NS(=O)(=O)C4CC4)ccn3)s2)n1] |
| I-239 | 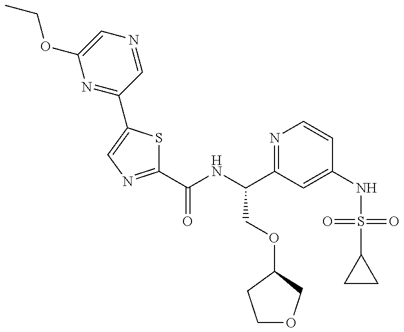 [SMILES: CCOc1cncc(-c2cnc(C(=O)N[C@@H](CO[C@@H]3CCOC3)c3cc(NS(=O)(=O)C4CC4)ccn3)s2)n1] |
| I-240 | 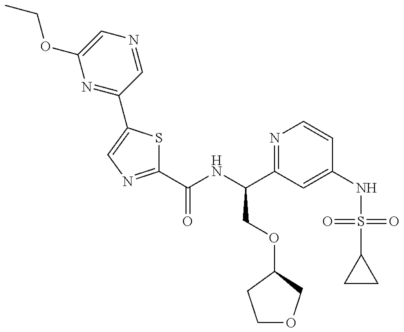 [SMILES: CCOc1cncc(-c2cnc(C(=O)N[C@H](CO[C@@H]3CCOC3)c3cc(NS(=O)(=O)C4CC4)ccn3)s2)n1] |
| I-241 | 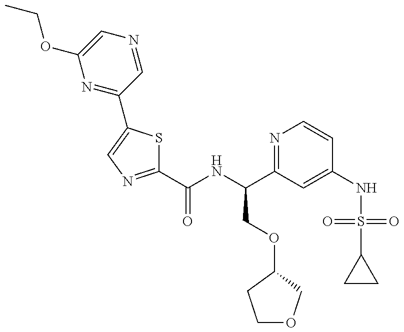 [SMILES: CCOc1cncc(-c2cnc(C(=O)N[C@H](CO[C@H]3CCOC3)c3cc(NS(=O)(=O)C4CC4)ccn3)s2)n1] |
| I-242 | 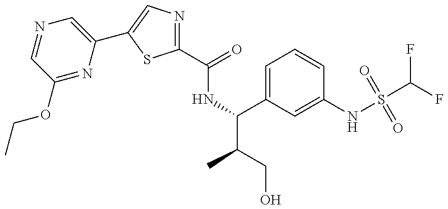 [SMILES: CCOc1cncc(-c2cnc(C(=O)N[C@H](c3cccc(NS(=O)(=O)C(F)F)c3)[C@H](C)CO)s2)n1] |
| I-243 | 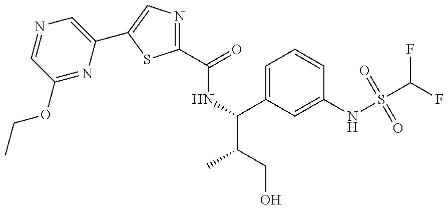 [SMILES: CCOc1cncc(-c2cnc(C(=O)N[C@H](c3cccc(NS(=O)(=O)C(F)F)c3)[C@@H](C)CO)s2)n1] |
| I-244 | 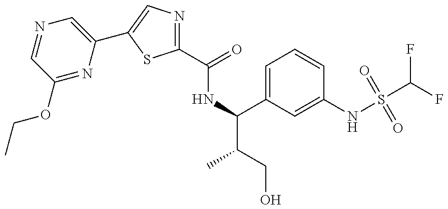 [SMILES: CCOc1cncc(-c2cnc(C(=O)N[C@@H](c3cccc(NS(=O)(=O)C(F)F)c3)[C@@H](C)CO)s2)n1] |
| I-245 | 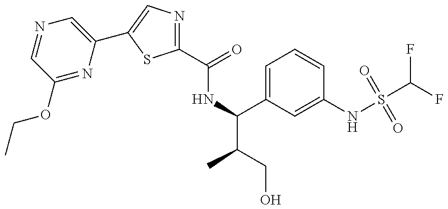 [SMILES: CCOc1cncc(-c2cnc(C(=O)N[C@@H](c3cccc(NS(=O)(=O)C(F)F)c3)[C@H](C)CO)s2)n1] |
| I-246 | 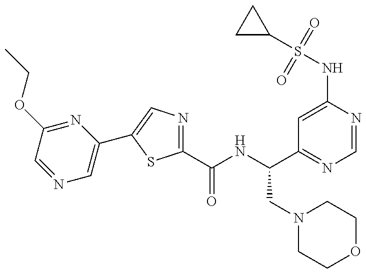 [SMILES: CCOc1cncc(-c2cnc(C(=O)N[C@@H](CN3CCOCC3)c3cc(NS(=O)(=O)C4CC4)ncn3)s2)n1] |
| I-247 | 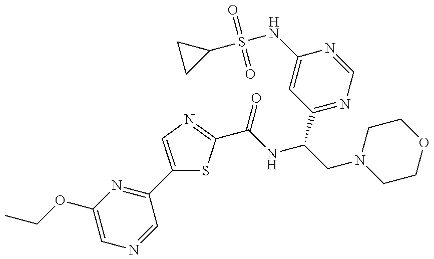 [SMILES: CCOc1cncc(-c2cnc(C(=O)N[C@@H](CN3CCOCC3)c3cc(NS(=O)(=O)C4CC4)ncn3)s2)n1] |
| I-248 | 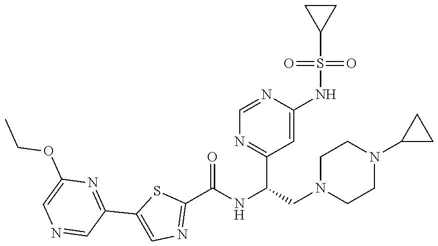 [SMILES: CCOc1cncc(-c2cnc(C(=O)N[C@@H](CN3CCN(C4CC4)CC3)c3cc(NS(=O)(=O)C4CC4)ncn3)s2)n1] |
| I-249 | 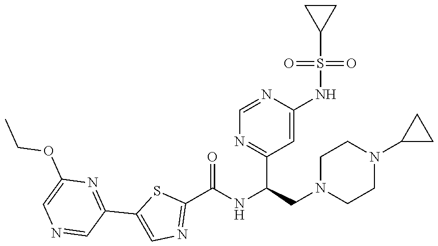 [SMILES: CCOc1cncc(-c2cnc(C(=O)N[C@H](CN3CCN(C4CC4)CC3)c3cc(NS(=O)(=O)C4CC4)ncn3)s2)n1] |
| I-250 | 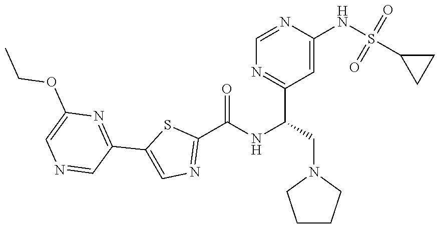 [SMILES: CCOc1cncc(-c2cnc(C(=O)N[C@@H](CN3CCCC3)c3cc(NS(=O)(=O)C4CC4)ncn3)s2)n1] |
| I-251 | 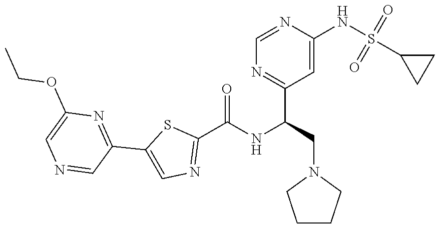 [SMILES: CCOc1cncc(-c2cnc(C(=O)N[C@H](CN3CCCC3)c3cc(NS(=O)(=O)C4CC4)ncn3)s2)n1] |
| I-252 | 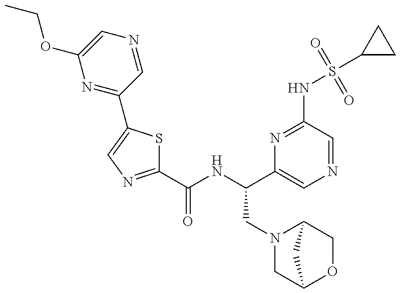 [SMILES: CCOc1cncc(-c2cnc(C(=O)N[C@@H](CN3C[C@H]4C[C@@H]3CO4)c3cncc(NS(=O)(=O)C4CC4)n3)s2)n1] |
| I-253 | 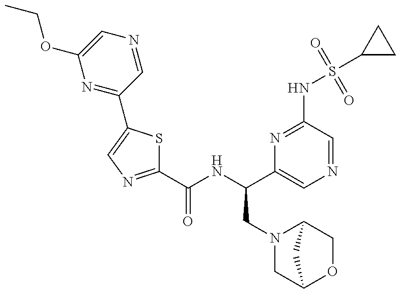 [SMILES: CCOc1cncc(-c2cnc(C(=O)N[C@H](CN3C[C@H]4C[C@@H]3CO4)c3cncc(NS(=O)(=O)C4CC4)n3)s2)n1] |
| I-254 | 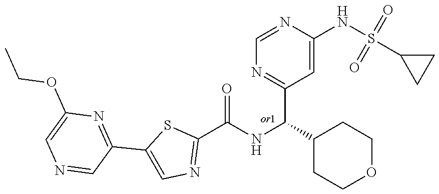 [SMILES: CCOc1cncc(-c2cnc(C(=O)N[C@H](c3cc(NS(=O)(=O)C4CC4)ncn3)C3CCOCC3)s2)n1] |
| I-255 | 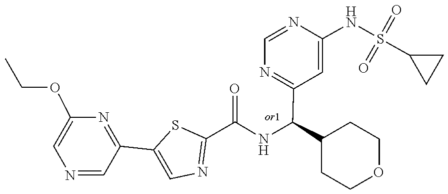 [SMILES: CCOc1cncc(-c2cnc(C(=O)N[C@@H](c3cc(NS(=O)(=O)C4CC4)ncn3)C3CCOCC3)s2)n1] |
| I-256 | 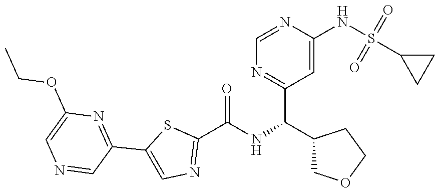 [SMILES: CCOc1cncc(-c2cnc(C(=O)N[C@H](c3cc(NS(=O)(=O)C4CC4)ncn3)[C@@H]3CCOC3)s2)n1] |
| I-257 | 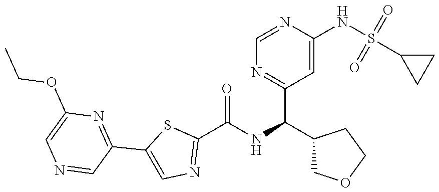 [SMILES: CCOc1cncc(-c2cnc(C(=O)N[C@@H](c3cc(NS(=O)(=O)C4CC4)ncn3)[C@@H]3CCOC3)s2)n1] |
| I-258 | 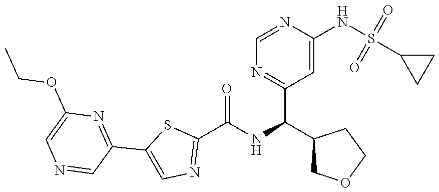 [SMILES: CCOc1cncc(-c2cnc(C(=O)N[C@@H](c3cc(NS(=O)(=O)C4CC4)ncn3)[C@H]3CCOC3)s2)n1] |
| I-259 | 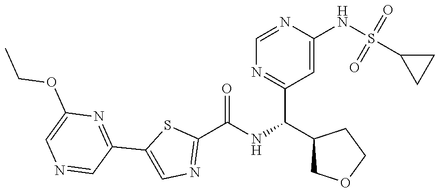 [SMILES: CCOc1cncc(-c2cnc(C(=O)N[C@H](c3cc(NS(=O)(=O)C4CC4)ncn3)[C@H]3CCOC3)s2)n1] |
| I-260 | 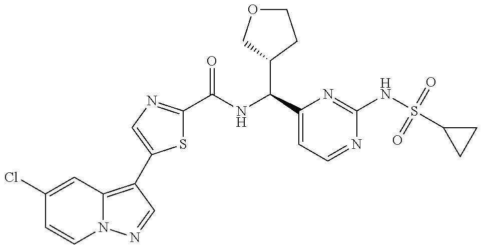 [SMILES: O=C(N[C@H](c1ccnc(NS(=O)(=O)C2CC2)n1)[C@H]1CCOC1)c1ncc(-c2cnn3ccc(Cl)cc23)s1] |
| I-261 | 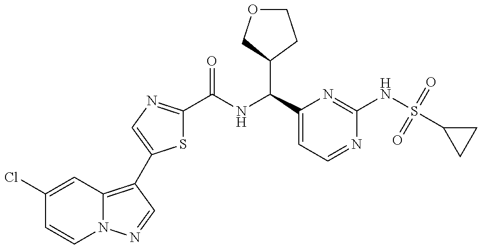 [SMILES: O=C(N[C@H](c1ccnc(NS(=O)(=O)C2CC2)n1)[C@@H]1CCOC1)c1ncc(-c2cnn3ccc(Cl)cc23)s1] |
| I-262 | 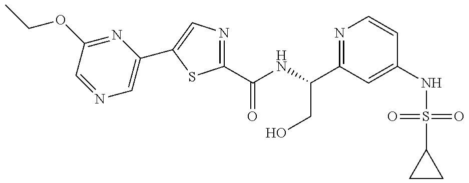 [SMILES: CCOc1cncc(-c2cnc(C(=O)N[C@@H](CO)c3cc(NS(=O)(=O)C4CC4)ccn3)s2)n1] |
| I-263 | 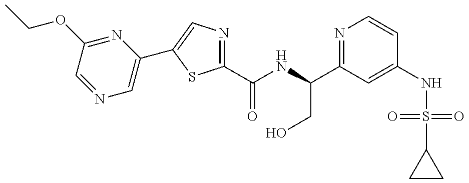 [SMILES: CCOc1cncc(-c2cnc(C(=O)N[C@H](CO)c3cc(NS(=O)(=O)C4CC4)ccn3)s2)n1] |
| I-264 | 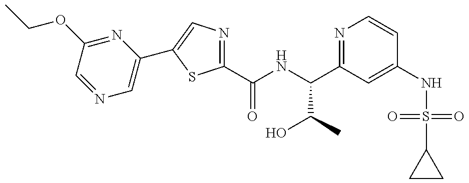 [SMILES: CCOc1cncc(-c2cnc(C(=O)N[C@H](c3cc(NS(=O)(=O)C4CC4)ccn3)[C@@H](C)O)s2)n1] |
| I-265 | 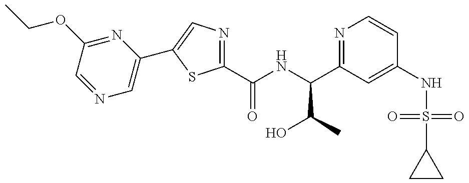 [SMILES: CCOc1cncc(-c2cnc(C(=O)N[C@@H](c3cc(NS(=O)(=O)C4CC4)ccn3)[C@@H](C)O)s2)n1] |
| I-266 | 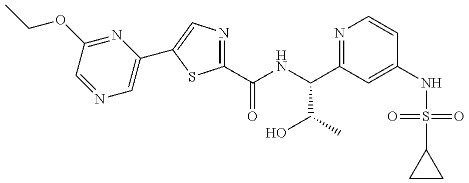 [SMILES: CCOc1cncc(-c2cnc(C(=O)N[C@H](c3cc(NS(=O)(=O)C4CC4)ccn3)[C@H](C)O)s2)n1] |
| I-267 | 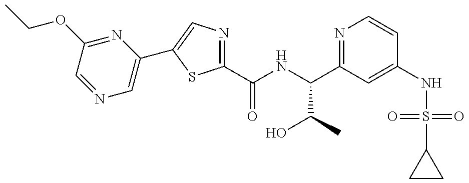 [SMILES: CCOc1cncc(-c2cnc(C(=O)N[C@H](c3cc(NS(=O)(=O)C4CC4)ccn3)[C@@H](C)O)s2)n1] |
| I-268 | 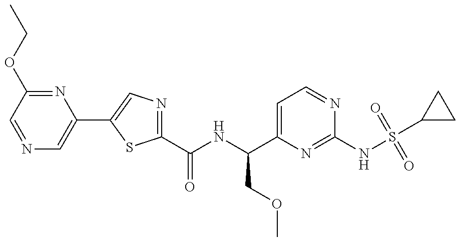 [SMILES: CCOc1cncc(-c2cnc(C(=O)N[C@H](COC)c3ccnc(NS(=O)(=O)C4CC4)n3)s2)n1] |
| I-269 | 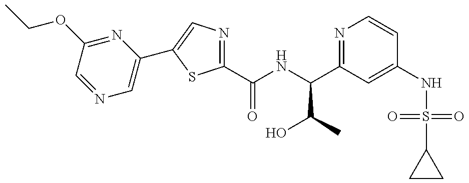 [SMILES: CCOc1cncc(-c2cnc(C(=O)N[C@@H](c3cc(NS(=O)(=O)C4CC4)ccn3)[C@@H](C)O)s2)n1] |
| I-270 | 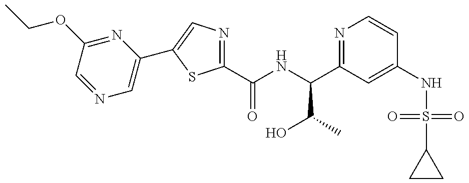 [SMILES: CCOc1cncc(-c2cnc(C(=O)N[C@@H](c3cc(NS(=O)(=O)C4CC4)ccn3)[C@H](C)O)s2)n1] |
| I-271 | 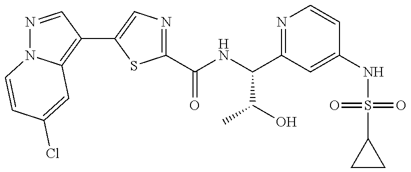 [SMILES: C[C@@H](O)[C@H](NC(=O)c1ncc(-c2cnn3ccc(Cl)cc23)s1)c1cc(NS(=O)(=O)C2CC2)ccn1] |
| I-272 | 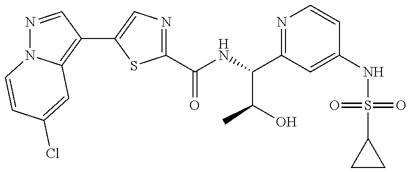 [SMILES: C[C@H](O)[C@H](NC(=O)c1ncc(-c2cnn3ccc(Cl)cc23)s1)c1cc(NS(=O)(=O)C2CC2)ccn1] |
| I-273 | 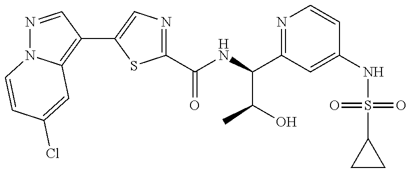 [SMILES: C[C@H](O)[C@@H](NC(=O)c1ncc(-c2cnn3ccc(Cl)cc23)s1)c1cc(NS(=O)(=O)C2CC2)ccn1] |
| I-274 | 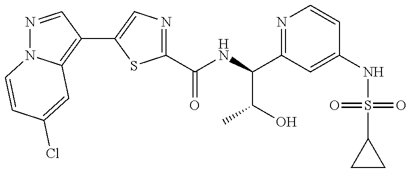 [SMILES: C[C@@H](O)[C@@H](NC(=O)c1ncc(-c2cnn3ccc(Cl)cc23)s1)c1cc(NS(=O)(=O)C2CC2)ccn1] |
| I-275 | 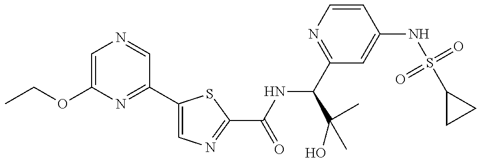 [SMILES: CCOc1cncc(-c2cnc(C(=O)N[C@@H](c3cc(NS(=O)(=O)C4CC4)ccn3)C(C)(C)O)s2)n1] |
| I-276 | 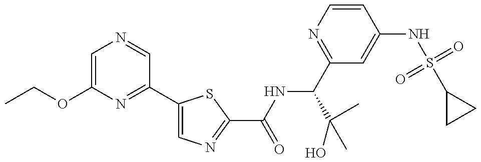 [SMILES: CCOc1cncc(-c2cnc(C(=O)N[C@H](c3cc(NS(=O)(=O)C4CC4)ccn3)C(C)(C)O)s2)n1] |
| I-277 | 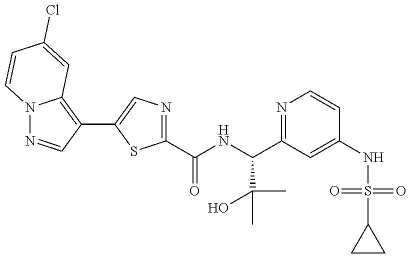 [SMILES: CC(C)(O)[C@H](NC(=O)c1ncc(-c2cnn3ccc(Cl)cc23)s1)c1cc(NS(=O)(=O)C2CC2)ccn1] |
| I-278 | 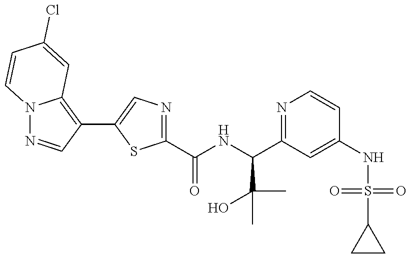 [SMILES: CC(C)(O)[C@@H](NC(=O)c1ncc(-c2cnn3ccc(Cl)cc23)s1)c1cc(NS(=O)(=O)C2CC2)ccn1] |
| I-279 | 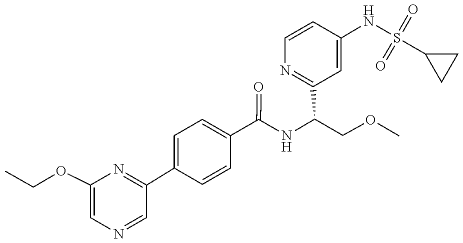 [SMILES: CCOc1cncc(-c2ccc(C(=O)N[C@@H](COC)c3cc(NS(=O)(=O)C4CC4)ccn3)cc2)n1] |
| I-280 | 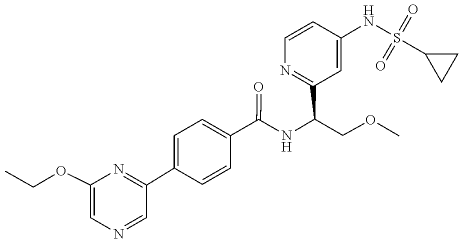 [SMILES: CCOc1cncc(-c2ccc(C(=O)N[C@H](COC)c3cc(NS(=O)(=O)C4CC4)ccn3)cc2)n1] |
| I-281 | 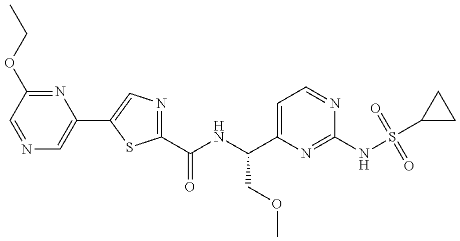 [SMILES: CCOc1cncc(-c2cnc(C(=O)N[C@@H](COC)c3ccnc(NS(=O)(=O)C4CC4)n3)s2)n1] |
| I-282 | 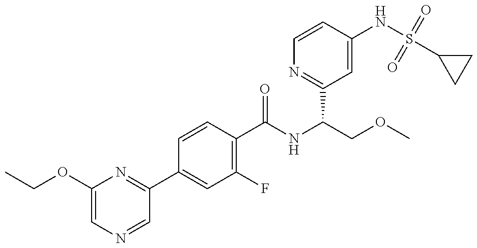 [SMILES: CCOc1cncc(-c2ccc(C(=O)N[C@@H](COC)c3cc(NS(=O)(=O)C4CC4)ccn3)c(F)c2)n1] |
| I-283 | 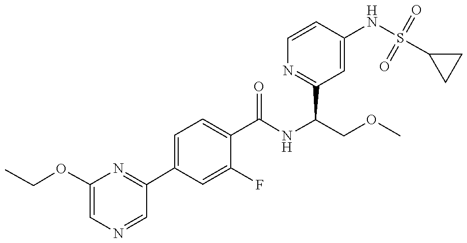 [SMILES: CCOc1cncc(-c2ccc(C(=O)N[C@H](COC)c3cc(NS(=O)(=O)C4CC4)ccn3)c(F)c2)n1] |
| I-284 | 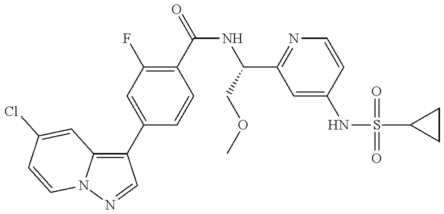 [SMILES: COC[C@H](NC(=O)c1ccc(-c2cnn3ccc(Cl)cc23)cc1F)c1cc(NS(=O)(=O)C2CC2)ccn1] |
| I-285 | 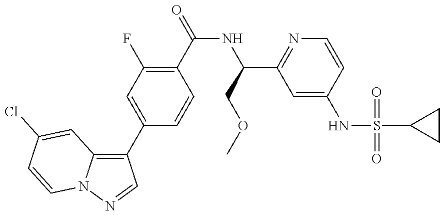 [SMILES: COC[C@@H](NC(=O)c1ccc(-c2cnn3ccc(Cl)cc23)cc1F)c1cc(NS(=O)(=O)C2CC2)ccn1] |
| I-286 | 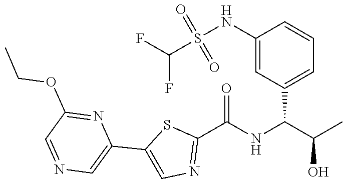 [SMILES: CCOc1cncc(-c2cnc(C(=O)N[C@H](c3cccc(NS(=O)(=O)C(F)F)c3)[C@@H](C)O)s2)n1] |
| I-287 | 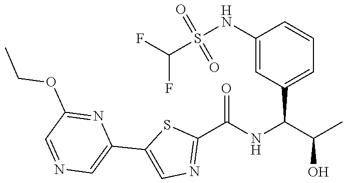 [SMILES: CCOc1cncc(-c2cnc(C(=O)N[C@@H](c3cccc(NS(=O)(=O)C(F)F)c3)[C@@H](C)O)s2)n1] |
| I-288 | 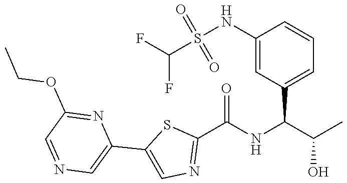 [SMILES: CCOc1cncc(-c2cnc(C(=O)N[C@@H](c3cccc(NS(=O)(=O)C(F)F)c3)[C@H](C)O)s2)n1] |
| I-289 | 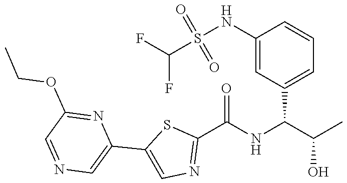 [SMILES: CCOc1cncc(-c2cnc(C(=O)N[C@H](c3cccc(NS(=O)(=O)C(F)F)c3)[C@H](C)O)s2)n1] |
| I-290 | 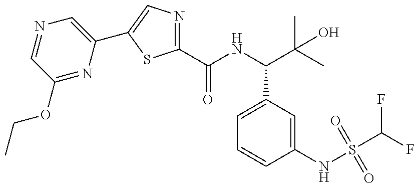 [SMILES: CCOc1cncc(-c2cnc(C(=O)N[C@@H](c3cccc(NS(=O)(=O)C(F)F)c3)C(C)(C)O)s2)n1] |
| I-291 | 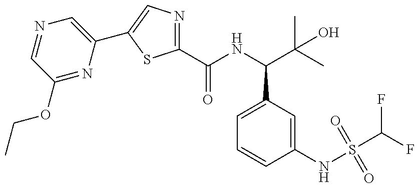 [SMILES: CCOc1cncc(-c2cnc(C(=O)N[C@H](c3cccc(NS(=O)(=O)C(F)F)c3)C(C)(C)O)s2)n1] |
| I-292 | 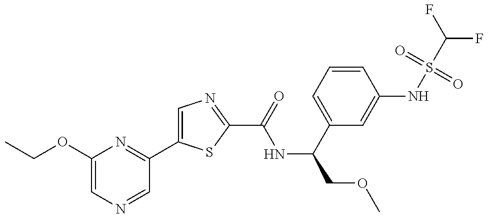 [SMILES: CCOc1cncc(-c2cnc(C(=O)N[C@H](COC)c3cccc(NS(=O)(=O)C(F)F)c3)s2)n1] |
| I-293 | 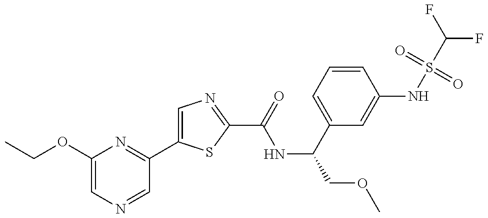 [SMILES: CCOc1cncc(-c2cnc(C(=O)N[C@@H](COC)c3cccc(NS(=O)(=O)C(F)F)c3)s2)n1] |
| II-1 | 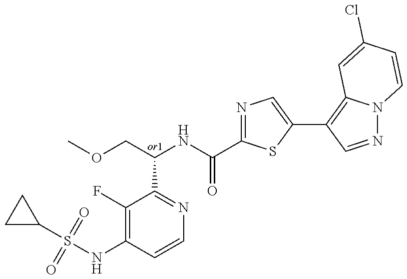 [SMILES: COC[C@H](NC(=O)c1ncc(-c2cnn3ccc(Cl)cc23)s1)c1nccc(NS(=O)(=O)C2CC2)c1F] |
| II-2 | 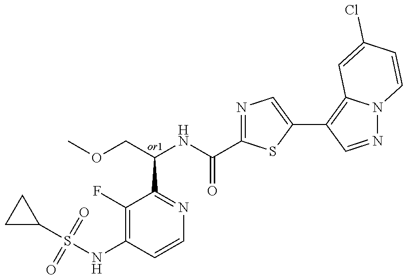 [SMILES: COC[C@@H](NC(=O)c1ncc(-c2cnn3ccc(Cl)cc23)s1)c1nccc(NS(=O)(=O)C2CC2)c1F] |
| II-3 | 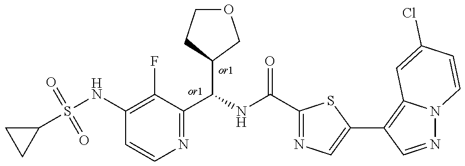 [SMILES: O=C(N[C@H](c1nccc(NS(=O)(=O)C2CC2)c1F)[C@H]1CCOC1)c1ncc(-c2cnn3ccc(Cl)cc23)s1] |
| II-4 | 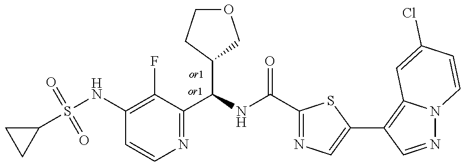 [SMILES: O=C(N[C@@H](c1nccc(NS(=O)(=O)C2CC2)c1F)[C@@H]1CCOC1)c1ncc(-c2cnn3ccc(Cl)cc23)s1] |
| II-5 | 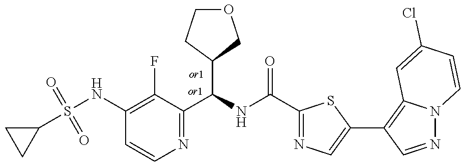 [SMILES: O=C(N[C@@H](c1nccc(NS(=O)(=O)C2CC2)c1F)[C@H]1CCOC1)c1ncc(-c2cnn3ccc(Cl)cc23)s1] |
| II-6 | 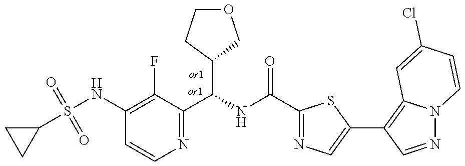 [SMILES: O=C(N[C@H](c1nccc(NS(=O)(=O)C2CC2)c1F)[C@@H]1CCOC1)c1ncc(-c2cnn3ccc(Cl)cc23)s1] |
| II-7 | 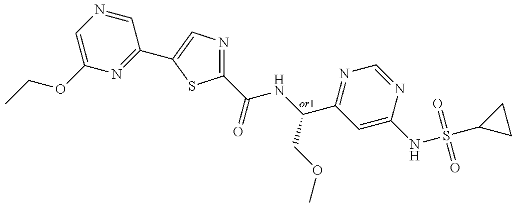 [SMILES: CCOc1cncc(-c2cnc(C(=O)N[C@@H](COC)c3cc(NS(=O)(=O)C4CC4)ncn3)s2)n1] |
| II-8 | 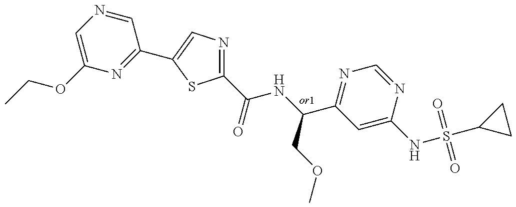 [SMILES: CCOc1cncc(-c2cnc(C(=O)N[C@H](COC)c3cc(NS(=O)(=O)C4CC4)ncn3)s2)n1] |
| II-9 | 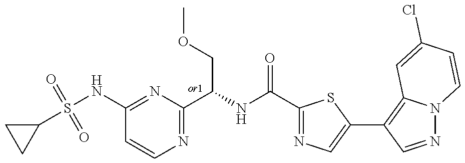 [SMILES: COC[C@H](NC(=O)c1ncc(-c2cnn3ccc(Cl)cc23)s1)c1nccc(NS(=O)(=O)C2CC2)n1] |
| II-10 | 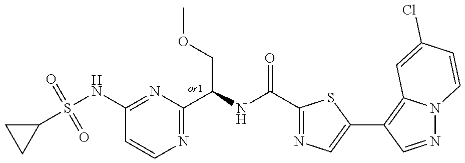 [SMILES: COC[C@@H](NC(=O)c1ncc(-c2cnn3ccc(Cl)cc23)s1)c1nccc(NS(=O)(=O)C2CC2)n1] |
| II-11 | 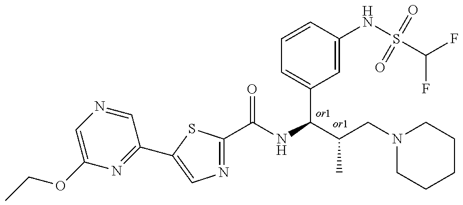 [SMILES: CCOc1cncc(-c2cnc(C(=O)N[C@@H](c3cccc(NS(=O)(=O)C(F)F)c3)[C@@H](C)CN3CCCCC3)s2)n1] |
| II-12 | 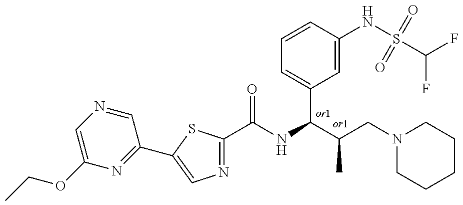 [SMILES: CCOc1cncc(-c2cnc(C(=O)N[C@@H](c3cccc(NS(=O)(=O)C(F)F)c3)[C@H](C)CN3CCCCC3)s2)n1] |
| II-13 | 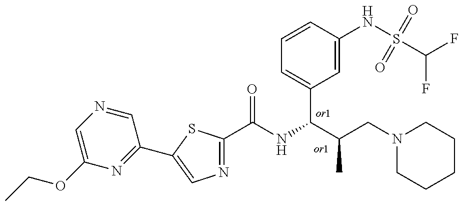 [SMILES: CCOc1cncc(-c2cnc(C(=O)N[C@H](c3cccc(NS(=O)(=O)C(F)F)c3)[C@H](C)CN3CCCCC3)s2)n1] |
| II-14 | 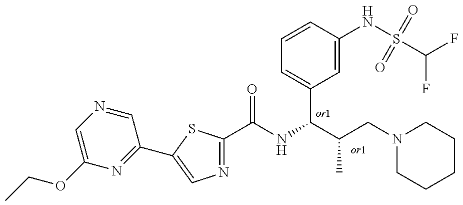 [SMILES: CCOc1cncc(-c2cnc(C(=O)N[C@H](c3cccc(NS(=O)(=O)C(F)F)c3)[C@@H](C)CN3CCCCC3)s2)n1] |
| II-15 | 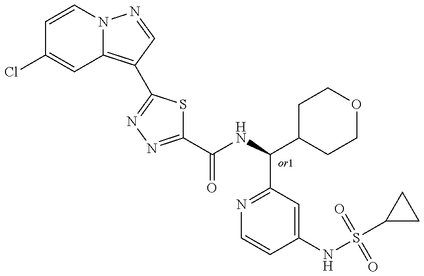 [SMILES: O=C(N[C@H](c1cc(NS(=O)(=O)C2CC2)ccn1)C1CCOCC1)c1nnc(-c2cnn3ccc(Cl)cc23)s1] |
| II-16 | 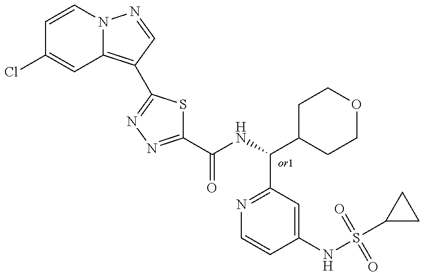 [SMILES: O=C(N[C@@H](c1cc(NS(=O)(=O)C2CC2)ccn1)C1CCOCC1)c1nnc(-c2cnn3ccc(Cl)cc23)s1] |
| II-17 | 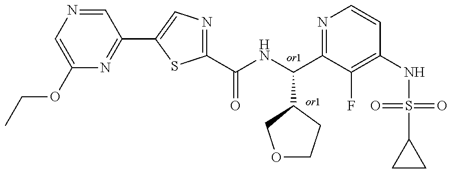 [SMILES: CCOc1cncc(-c2cnc(C(=O)N[C@H](c3nccc(NS(=O)(=O)C4CC4)c3F)[C@H]3CCOC3)s2)n1] |
| II-18 | 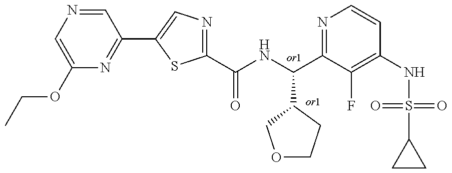 [SMILES: CCOc1cncc(-c2cnc(C(=O)N[C@H](c3nccc(NS(=O)(=O)C4CC4)c3F)[C@@H]3CCOC3)s2)n1] |
| II-19 | 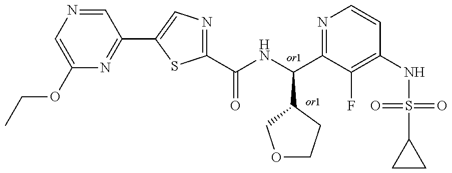 [SMILES: CCOc1cncc(-c2cnc(C(=O)N[C@@H](c3nccc(NS(=O)(=O)C4CC4)c3F)[C@@H]3CCOC3)s2)n1] |
| II-20 | 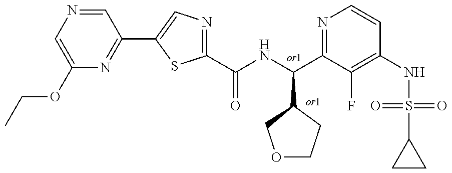 [SMILES: CCOc1cncc(-c2cnc(C(=O)N[C@@H](c3nccc(NS(=O)(=O)C4CC4)c3F)[C@H]3CCOC3)s2)n1] |
| II-21 | 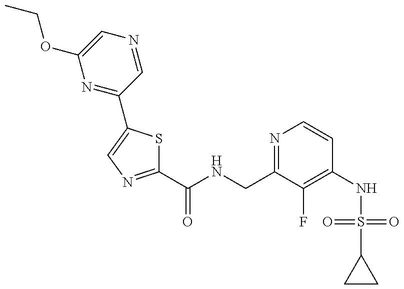 [SMILES: CCOc1cncc(-c2cnc(C(=O)NCc3nccc(NS(=O)(=O)C4CC4)c3F)s2)n1] |

In some embodiments, the compound is:

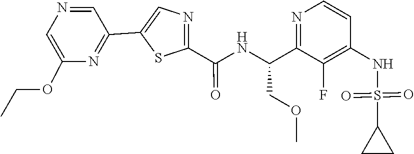 [SMILES: CCOc1cncc(-c2cnc(C(=O)N[C@@H](COC)c3nccc(NS(=O)(=O)C4CC4)c3F)s2)n1]

or a pharmaceutically acceptable salt thereof.

In some embodiments, the compound is:

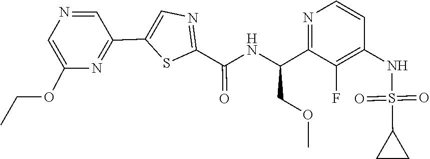 [SMILES: CCOc1cncc(-c2cnc(C(=O)N[C@H](COC)c3nccc(NS(=O)(=O)C4CC4)c3F)s2)n1]

or a pharmaceutically acceptable salt thereof.

In some embodiments, the compound is:

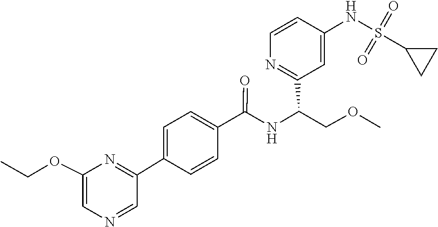 [SMILES: CCOc1cncc(-c2ccc(C(=O)N[C@@H](COC)c3cc(NS(=O)(=O)C4CC4)ccn3)cc2)n1]

or a pharmaceutically acceptable salt thereof.

In some embodiments, the compound is:

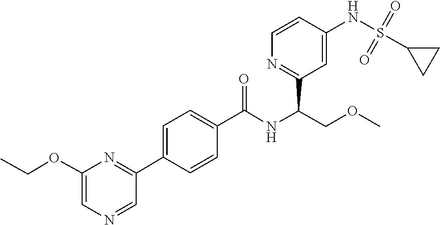 [SMILES: CCOc1cncc(-c2ccc(C(=O)N[C@H](COC)c3cc(NS(=O)(=O)C4CC4)ccn3)cc2)n1]

or a pharmaceutically acceptable salt thereof.

In some embodiments, the compound is:

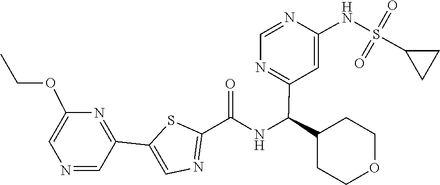 [SMILES: CCOc1cncc(-c2cnc(C(=O)N[C@@H](c3cc(NS(=O)(=O)C4CC4)ncn3)C3CCOCC3)s2)n1]

or a pharmaceutically acceptable salt thereof.

In some embodiments, the compound is:

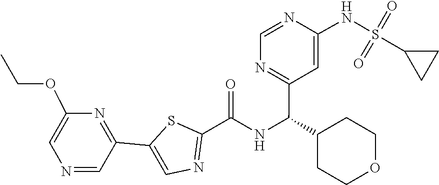 [SMILES: CCOc1cncc(-c2cnc(C(=O)N[C@H](c3cc(NS(=O)(=O)C4CC4)ncn3)C3CCOCC3)s2)n1]

or a pharmaceutically acceptable salt thereof.

In some embodiments, the present invention provides a compound set forth in Table 1, above, or a pharmaceutically acceptable salt thereof. In some embodiments, the present invention provides a compound set forth in Table 1, above. In some embodiments, the present invention provides a pharmaceutical composition comprising a compound set forth in Table 1 above, or a pharmaceutically acceptable salt thereof, together with a pharmaceutically acceptable carrier, excipient, or diluent.

In some embodiments, the present invention provides a compound of formula I as defined above, or a pharmaceutically acceptable salt thereof, or a pharmaceutical composition comprising a compound of formula I as defined above, or a pharmaceutically acceptable salt thereof, and a pharmaceutically acceptable carrier, adjuvant, or vehicle for use as a medicament.

In some embodiments, the invention also provides compounds of formula I described herein or pharmaceutical compositions described herein for use in a method for inhibiting CTPS1 as described herein, in a method for enhancing an immune response in a subject in need thereof as described herein and/or in a method for treating a CTPS1-dependent disorder as described herein.

In some embodiments, the invention also provides compounds of formula I described herein or pharmaceutical compositions described herein for use in a method for inhibiting CTPS1 as described herein.

In some embodiments, the invention also provides compounds of formula I described herein or pharmaceutical compositions described herein for use in a method for enhancing an immune response in a subject in need thereof as described herein.

In some embodiments, the invention also provides compounds of formula I described herein or pharmaceutical compositions described herein for use in a method for treating a CTPS1-dependent disorder as described herein.

In some embodiments, the invention also provides the use of a compound of formula I described herein or a pharmaceutical composition described herein for the manufacture of a medicament for inhibiting CTPS1, a medicament for enhancing an immune response in a subject in need thereof and/or a medicament for treating a CTPS1-dependent disorder.

In some embodiments, the invention also provides the use of a compound of formula I described herein or a pharmaceutical composition described herein for the manufacture of a medicament for inhibiting CTPS1.

In some embodiments, the invention also provides the use of a compound of formula I described herein or a pharmaceutical composition described herein for the manufacture of a medicament for enhancing an immune response in a subject in need thereof.

In some embodiments, the invention also provides the use of a compound of formula I described herein or a pharmaceutical composition described herein for the manufacture of a medicament treating a CTPS1-dependent disorder.

In some embodiments, the invention also provides the use of compounds of formula I described herein or pharmaceutical compositions described herein in a method for inhibiting CTPS1 as described herein, in a method for enhancing an immune response in a subject in need thereof as described herein and/or in a method for treating a CTPS1-dependent disorder as described herein.

In some embodiments, the invention also provides the use of compounds of formula I described herein or pharmaceutical compositions described herein in a method for inhibiting CTPS1 as described herein.

In some embodiments, the invention also provides the use of compounds of formula I described herein or pharmaceutical compositions described herein in a method for enhancing an immune response in a subject in need thereof as described herein.

In some embodiments, the invention also provides the use of compounds of formula I described herein or pharmaceutical compositions described herein in a method for treating a CTPS1-dependent disorder as described herein.

### 4. General Methods of Providing the Present Compounds

The compounds of this invention may be prepared or isolated in general by synthetic and/or semi-synthetic methods known to those skilled in the art for analogous compounds and by the General Methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit CTPS1, or a mutant thereof, in a biological sample or in a patient. In certain embodiments, the amount of compound in compositions of this invention is such that is effective to measurably inhibit CTPS1, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylccllulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of CTPS1, or a mutant thereof.

The subject matter disclosed herein includes prodrugs, metabolites, derivatives, and pharmaceutically acceptable salts of compounds of the invention. Metabolites include compounds produced by a process comprising contacting a compound of the invention with a mammal for a period of time sufficient to yield a metabolic product thereof. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A compound of the invention can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried comstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

All cells utilize nucleotides as key building blocks for cellular metabolic processes, such as deoxyribonucleic acid (DNA) and ribonucleic acid (RNA) synthesis, membrane lipid biosynthesis, and as a cellular store of biochemical energy for many important enzymatic processes. There are two classes of nucleotides which contain either purine (such as guanine or adenine) or pyrimidine (such as cytosine or thymine) bases, and both classes are important for diverse metabolic processes. Nucleotides can be further phosphorylated by kinases to provide mono, di-, or tri-phosphate forms, which are also utilized in biosynthetic processes. Perhaps one of the most well-known uses of nucleotide triphosphates are as the building blocks of DNA within the cell, encoding the information necessary for RNA and protein biosynthesis. Based on the critical role of nucleotides within the cell, many therapies have been developed to target different aspects of nucleotide synthesis, with some inhibiting the generation of purine nucleotides, some pyrimidine nucleotides, or both classes simultaneously. This includes compounds such as leflunomide/teriflunomide, methotrexate, azathioprine, and others.

The pyrimidine nucleotide CTP (cytidine 5′ triphosphate) is a precursor required not just for the anabolic generation of both DNA and RNA, but also phospholipids and sialyation of proteins. CTP originates from two sources: a salvage pathway and a de novo synthesis pathway that depends on the enzyme CTP synthase (Evans and Guy 2004; Higgins, et al. 2007; Ostrander, et al. 1998). In humans, there are two highly-homologous isoforms of CTP synthase (CTPS1 and CTPS2), both of which perform the same enzymatic reaction, although evidence suggests that the cellular and metabolic regulation of the two isoforms may be distinct. Although CTP synthase exists as two isozymes in humans and other eukaryotic organisms, detailed functional differences of the two isozymes in cellular or tissue biology are not yet fully delineated (van Kuilenburg, et al. 2000).

CTPS1 and CTPS2 catalyze the conversion of uridine triphosphate (UTP) and glutamine into cytidine triphosphate (CTP) and L-glutamate, with the concurrent hydrolyzation of ATP to ADP:

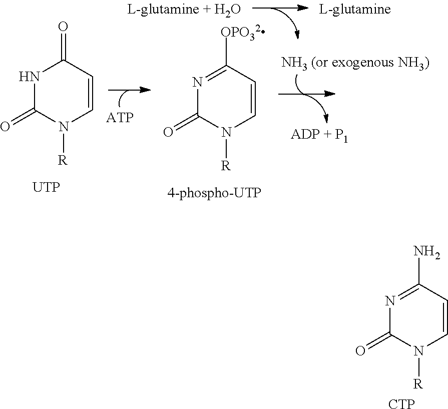

CTP synthase has two functional activities, an N-terminal synthetase domain and a C-terminal glutaminase domain, and the active enzyme is made up of a homotetramer (Kursula, et al. 2006). The synthetase domain of the enzyme transfers a phosphate from adenosine triphosphate (ATP) to the 4-position of UTP to create an activated intermediate, 4-phospho-UTP. The glutaminase domain of the enzyme generates ammonia from glutamine via a covalent thioester intermediate with a conserved active site cysteine, generating glutamate. This ammonium that is generated is transferred from the glutaminase domain to the synthetase domain via a tunnel in the enzyme or can be derived from external ammonium. This ammonium is then used in the synthetase domain to generate CTP from the 4-phospho-UTP (Lieberman, 1956).

Many studies have highlighted a key role of nucleotide synthesis and CTP synthase activity specifically in several aspects of normal and disease cell biology, especially in the cellular context of the high metabolic demands of replication and division where activation of de novo nucleotide synthesis is necessary. For instance, CTPS activity has been shown to be upregulated in a range of tumor types of both hematological and non-hematological origin, suggesting de novo pyrimidine biosynthesis is necessary to support the aggressive growth and division of cancer cells. The mechanistic drivers, cell type, and tissue origin of cancers are obviously diverse, but the underlying result is a breakdown in the control of cell division allowing inappropriate proliferation.

The process of tumorigenesis is highly complex, requiring careful coordination of multiple pathways, many of which remain to be fully characterized. Ultimately any cell division requires the effective replication of the cell's DNA and other constituents and is a metabolically-demanding process. Interfering with a cell's ability to replicate by targeting nucleic acid synthesis has been a core approach in cancer therapy for many years, and examples of therapies acting in this way are 6-thioguanine, 6-mecaptopurine, 5-fluorouracil, cytarabine, gemcitabine, and methotrexate.

Currently, the precise roles that CTPS1 and/or CTPS2 may play in cancer is not well defined. Several non-selective inhibitors of CTP synthase have been previously developed for oncology indications up to phase I/II clinical trials but were likely stopped due to toxicity, poor pharmacokinetic characteristics, or limited efficacy. Most of these early developed inhibitors are nucleoside-analogue prodrugs (3-deazauridine, cyclopentenyl cytosine, carbodine), which are converted by the kinases involved in pyrimidine synthesis into the active tri-phosphorylated metabolite. Other inhibitors (such as acivicin or 6-Diazo-5-oxo-L-norleucine) are reactive analogues of glutamine and irreversibly inhibit the glutaminase domain of CTPS and other glutamine-utilizing enzymes.

Given the high metabolic demands of the proliferating cancer cell and the data suggesting CTP synthase activity in a number of tumor types, selective CTPS inhibitors could offer an attractive alternative approach for the treatment of tumors. Compounds with different activity against CTPS1 and/or CTPS2 may offer important opportunities to target different tumors depending upon their relative dependence on these two enzymes.

In addition to cancer biology, extensive literature highlights the role of nucleotide synthesis and CTP synthase activity in immune biology and disease. The immune system in multi-cellular organisms has evolved to provide protection from a diverse range of infectious pathogens. This process generally requires recognition of the pathogen by various immune cells and is often followed by amplification and long-term propagation of the immune response through the rapid expansion, proliferation, and differentiation of responding immune cells. Within this process, CTP synthase activity appears to play an important role in DNA synthesis and the rapid expansion of lymphocytes following activation (Fairbanks, et al. 1995; van den Berg, et al. 1995).

Direct clinical validation that CTPS1 is the critical enzyme in human lymphocyte proliferation came from the genetic finding that a rare loss-of-function homozygous mutation (rs145092287) in this enzyme causes a severe immunodeficiency, which is characterized by a severely reduced capacity of patient activated T- and B-cells to proliferate in response to antigen receptor-mediated activation. In addition, activated CTPS1-deficient cells from patients were shown to have decreased levels of intracellular CTP compared to normal controls, and normal T-cell proliferation could be restored in CTPS1-deficient cells by expressing wild-type CTPS1 or by the addition of exogenous cytidine. CTPS1 mRNA and protein expression was found to be very low in resting lymphocytes, but rapidly upregulated following activation. The expression of CTPS1 in other tissues was generally low, and it is not known whether expression in other tissues is similarly inducible. CTPS2 seems to be ubiquitously expressed in a range of cells and tissues but at low levels, and the failure of normal levels of CTPS2 to compensate for the mutated CTPS1 in immune cells supports the critical role of CTPS1 in the immune populations affected in the homozygous patients (Martin, et al. 2014). In sum, these findings suggest that CTPS1 activity is critical to meet the metabolic demands of CTP required by several important immune cell populations when they are activated and required to proliferate.

Normally the immune response is tightly regulated to ensure sufficient activity for protection from infection while preventing overactivity or inappropriate recognition of host proteins and cells. In certain diseases or conditions, the control of this process is not effective and can lead to immune-mediated pathology. A wide range of human diseases and pathologies are believed to be due to these types of inappropriate immune responses and are commonly classified as autoimmune diseases or autoinflammatory conditions.

Given the role that pathogenic immune cells, such as autoreactive T and B lymphocytes, are believed to play in a wide range of autoimmune and other diseases, CTPS1 represents a potential therapeutic target for a new class of immunosuppressive agents. Specific CTPS1 inhibitors could therefore provide a novel approach to the functional inhibition of activated lymphocytes and specific other immune cell populations shown to be defective in CTPS1-deficient patients, such as NK (natural killer), MAIT (Mucosal-Associated Invariant T), and iNK (invariant natural killer) cells (Martin, et al. 2014).

In addition to roles in cancer and immune biology, CTPS1 has also been suggested to play a role in vascular smooth muscle cell proliferation (restenosis) following vascular injury or surgery (Tang, et al. 2013).

To date, no specific CTP synthase inhibitors have been described in detail or tested clinically. Available data strongly suggest that inhibitors of CTPS1 could reduce the proliferation of pathogenic immune and cancer cell populations, and potentially other targeted cell populations. Inhibitors of CTPS1 may therefore be expected to have utility for treatment or prophylaxis in a wide range of indications where the pathology is driven by these populations. CTPS1 inhibitors may represent a unique approach for inhibiting selected components of the immune system, such as proliferative pathogenic autoreactive lymphocytes. This could have utility in a number of diseases, such as immune-mediated rejection of transplanted cells, organs or tissues, graft-versus-host disease or reactions, and immune-driven allergies and autoimmune diseases. Additionally, the apparent specificity of the function of CTPS1 in immune cells may suggest that CTPS1 inhibitors could exhibit an improved therapeutic index over other clinically used non-specific nucleotide inhibitors, such as leflunomide or azathioprine. Finally, CTPS1 inhibitors may offer therapeutic potential in a range of cancer indications, especially of hematopoietic origin, and could aid in improving recovery from vascular injury or surgery and reducing morbidity and mortality associated with neointima formation and restenosis.

The present disclosure provides methods of modulating (e.g., inhibiting) CTPS1 activity, the method comprising administering to a patient a compound of the invention, or a pharmaceutically acceptable salt, hydrate, solvate, or prodrug thereof. In certain embodiments, the compounds and compositions described herein are useful for the inhibition of CTPS1.

In one embodiment, the subject matter disclosed herein is directed to a method of inhibiting CTPS1, the method comprising contacting CTPS1 with an effective amount of a compound of the invention, or a pharmaceutically acceptable salt, hydrate, solvate, or prodrug thereof described herein. Such methods comprise contacting CTPS1 with an effective amount of a presently disclosed compound. The compound can be contacted with CTPS1 in vitro or in vivo via administration of the compound to a subject.

In one aspect, the invention provides a method of treating a CTPS1-mediated disease, disorder, or condition in a subject, comprising administering a therapeutically effective amount of a compound of the invention, or a pharmaceutically acceptable salt, hydrate, solvate, or prodrug thereof, to the subject.

In another aspect, the invention provides a method of treating a disease, disorder, or condition related to CTPS1 regulation in a subject, comprising administering a therapeutically effective amount of a compound of the invention, or a pharmaceutically acceptable salt, hydrate, solvate, or prodrug thereof, to the subject.

In certain embodiments, the disease, disorder, or condition is selected from rejection of transplanted cells and tissues, Graft-related diseases or disorders, allergies and autoimmune diseases.

In one embodiment the disease, disorder, or condition is the rejection of transplanted cells and tissues. In certain embodiments, the subject may have been transplanted with a graft selected from the group consisting of heart, kidney, lung, liver, pancreas, pancreatic islets, brain tissue, stomach, large intestine, small intestine, cornea, skin, trachea, bone, bone marrow (or any other source of hematopoietic precursor cells and stem cells including hematopoietic cells mobilized from bone marrow into peripheral blood or umbilical cord blood cells), muscle, and bladder. In certain embodiments, the compounds of the invention are used in preventing or suppressing an immune response associated with rejection of a donor tissue, cell, graft or organ transplant in a subject.

In certain embodiments, the disease, disorder, or condition is a graft-related disease or disorder. In some embodiments, graft-related diseases or disorders include graft versus host disease (GVHD), such as GVHD associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc., and Host-Versus-Graft-Disease (HVGD). In certain embodiments, the compounds of the invention are used in preventing or suppressing acute rejection of such transplant in the recipient and/or for long-term maintenance therapy to prevent rejection of such transplant in the recipient (e.g., inhibiting rejection of insulin-producing islet cell transplant from a donor in the subject recipient suffering from diabetes). In some embodiments, the compounds of the invention prevent Host-Versus-Graft-Disease (HVGD) and Graft-Versus-Host-Disease (GVHD).

In certain embodiments, the compound of the invention is administered to the subject before, after transplantation and/or during transplantation. In some embodiments, the compound of the invention is administered to the subject on a periodic basis before and/or after transplantation.

In another embodiment, the condition, disease, or disorder is an allergy.

In certain embodiments, the autoimmune disease treated by the compound of the invention is Addison's Disease, Adult-onset Still's disease, Alopecia Arcata, Alzheimer's disease, Anti-neutrophil Cytoplasmic Antibodies (ANCA)-Associated Vasculitis, Ankylosing Spondylitis, Anti-phospholipid Syndrome (Hughes Syndrome), Aplastic Anemia, Arthritis, Asthma, Atherosclerosis, Atherosclerotic plaque, Atopic Dermatitis, Autoimmune Hemolytic Anemia, Autoimmune Hepatitis, Autoimmune Hypophysitis (Lymphocytic Hypophysitis), Autoimmune Inner Ear Disease, Autoimmune Lymphoproliferative Syndrome, Autoimmune Myocarditis, Autoimmune Neutropenia, Autoimmune Oophoritis, Autoimmune Orchitis, Auto-Inflammatory Diseases requiring an immunosuppressive treatment, Azoospermia, Bechet's Disease, Berger's Disease, Bullous Pemphigoid, Cardiomyopathy, Cardiovascular disease, Celiac disease including Refractory Celiac Disease (type I and type II), Chronic Fatigue Immune Dysfunction Syndrome (CFIDS), Chronic Idiopathic Polyneuritis, Chronic Inflammatory Demyelinating Polyneuropathy (CIPD), Chronic Relapsing Polyneuropathy (Guillain-Barre syndrome), Churg-Strauss Syndrome (CSS), Cicatricial Pemphigoid, Cold Agglutinin Disease (CAD), chronic obstructive pulmonary disease (COPD), CREST Syndrome, Cryoglobulin Syndromes, Cutaneous Lupus, Dermatitis Herpetiformis, Dermatomyositis, Eczema, Epidermolysis Bullosa Acquisita, Essential Mixed Cryoglobulinemia, Evan's Syndrome, Exophthalmos, Fibromyalgia, Goodpasture's Syndrome, Grave's disease, Hemophagocytic Lymphohistiocytosis (HLH) (including Type 1 Hemophagocytic Lymphohistiocytosis), Histiocytosis/Histiocytic Disorders, Hashimoto's Thyroiditis, Idiopathic Pulmonary Fibrosis, Idiopathic Thrombocytopenia Purpura (ITP), IgA Nephropathy, Immunoproliferative Diseases or Disorders, Inflammatory Bowel Disease (IBD), Interstitial Lung Disease, Juvenile Arthritis, Juvenile Idiopathic Arthritis (JIA), Kawasaki's Disease, Lambert-Eaton Myasthenic Syndrome, Lichen Planus, Localized Scleroderma, Lupus Nephritis, Meniere's Disease, Microangiopathic Hemoytic Anemia, Microscopic Polyangitis, Miller Fischer Syndrome/Acute Disseminated Encephalomyeloradiculopathy, Mixed Connective Tissue Disease, Multiple Sclerosis (MS), Muscular Rheumatism, Myalgic Encephalomyelitis (ME), Myasthenia Gravis, Ocular Inflammation, Pemphigus Foliaceus, Pemphigus Vulgaris, Pernicious Anemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune Cholangiopathy, Primary Glomerulonephritis, Primary Sclerosing Cholangitis, Psoriasis, Psoriatic Arthritis, Pure Red Cell Anemia, Raynaud's Phenomenon, Reiter's Syndrome/Reactive Arthritis, Relapsing Polychondritis, Restenosis, Rheumatic Fever, Rheumatic Disease, Rheumatoid Arthritis, Sarcoidosis, Schmidt's Syndrome, Scleroderma/Systemic Sclerosis, Sjorgen's Syndrome, Stiff-Man Syndrome, The Sweet Syndrome (Febrile Neutrophilic Dermatosis), Systemic Lupus Erythematosus (SLE), Systemic Scleroderma, Takayasu Arteritis, Temporal Arteritis/Giant Cell Arteritis, Thyroiditis, Type 1 diabetes, Type 2 diabetes, Uveitis, Vasculitis, Vitiligo, Wegener's Granulomatosis, or X-linked lymphoproliferative disease.

In some embodiments, the disease treated by the compound of the invention is driven by T-cell activation and proliferation, selected from alopecia areata, atopic dermatitis, eczema, psoriasis, lichen planus, psoriatic arthritis, vitiligo, uveitis, ankylosing spondylitis, Reiter's syndrome/reactive arthritis, aplastic anemia, autoimmune lymphoproliferative syndrome/disorders, hemophagocytic lymphohistiocytosis, type 1 diabetes, and refractory celiac disease. In some embodiments, the disease is acute rejection of grafted tissues and transplanted organs, acute graft versus host disease (GVHD) after transplantation of bone marrow cells or any other source of allogenic cells including hematopoietic precursors cells and/or stem cells.

In certain embodiments, the disease treated by the compound of the invention is driven by both T- and B-cell activation and proliferation, selected from allergy, cicatricial pemphigoid, bullous pemphigoid, epidermolysis bullosa acquisita, pemphigus foliaceus, pemphigus vulgaris, dermatitis herpetiformis, ANCA-associated vasculitis and microscopic polyangitis, vasculitis, Wegener's granulomatosis; Churg-Strauss syndrome (CSS), polyarteritis nodosa, cryoglobulin syndromes and essential mixed cryglobulinemia, Systemic lupus erythematosus (SLE), antiphospholipid syndrome (Hughes Syndrome), cutaneous lupus, lupus nephritis, mixed connective tissue disease, Thyroiditis, Hashimoto thyroiditis, Grave's disease, exophthalmos, autoimmune hemolytic anemia, autoimmune neutropenia, ITP, pernicious anaemia, pure red cell anaemia, micro-angiopathic hemolytic anemia, primary glomerulonephritis, Berger's disease, Goodpasture's syndrome, IgA nephropathy, chronic idiopathic polyneuritis, chronic inflammatory demyelinating polyneuropathy (CIPD), chronic relapsing polyneuropathy (Guillain-Barre syndrome), Miller Fischer syndrome, Stiff man syndrome, Lambert-Eaton myasthenic syndrome, and myasthenia gravis.

In certain embodiments, the disease treated by the compound of the invention is Addison's disease, autoimmune oophoritis and azoospermia, polyglandular syndromes (Whitaker's syndrome), Schmidt's syndrome, autoimmune myocarditis, cardiomyopathy, Kawasaki's disease, rheumatoid arthritis, Sjogren's syndrome, mixed connective tissue disease, polymyositis and dermatomyositis, polychondritis, primary glomerulonephritis, Multiple sclerosis, autoimmune hepatitis, primary biliary cirrhosis/autoimmune cholangiopathy, hyper acute rejection of transplanted organs, chronic rejection of graft or transplants, and Chronic Graft versus Host reaction/disease after transplantation of bone marrow cells or hematopoietic precursor cells.

In certain embodiments, the disease treated by the compound of the invention is COPD, idiopathic pulmonary fibrosis, interstitial lung disease, sarcoidosis, adult onset Still's disease, juvenile idiopathic arthritis, Systemic sclerosis, CREST syndrome where B cells and pathogen antibodies may also play a role, the Sweet syndrome; Takayasu arteritis, temporal arteritis/giant cell arteritis, ulcerative cholangitis, inflammatory bowel disease (IBD) including Crohn's disease and ulcerative colitis, primary sclerosing cholangitis, Alzheimer's disease, cardiovascular syndrome, type 2 diabetes, restenosis, chronic fatigue immune dysfunction syndrome (CFIDS), Autoimmune Lymphoproliferative Syndrome and X-linked lymphoproliferative disease.

In certain embodiments, the disease treated by the compound of the invention is inflammatory skin diseases such as psoriasis or lichen planus; acute and/or chronic GVHD such as steroid resistant acute GVHD; acute lymphoproliferative syndrome; systemic lupus erythematosus, lupus nephritis or cutaneous lupus; or transplantation. In addition, the disease or disorder may be selected from myasthenia gravis, multiple sclerosis, and scleroderma/systemic sclerosis.

In certain aspects, the invention provides a method of treating cell proliferation disorders, including cancers, benign papillomatosis, gestational trophoblastic diseases, and benign neoplastic diseases, such as skin papilloma (warts) and genital papilloma.

In one aspect, provided herein is a method for treating of cancer in a subject in need thereof comprising administering to the subject an effective amount of a compound of the invention or a pharmaceutically acceptable salt, prodrug, metabolite, or derivative thereof.

In the methods described herein, a compound of the invention or a pharmaceutical composition thereof is administered to a subject that has cancer.

In certain embodiments, the cancer is selected from the group consisting of colorectal cancer, melanoma, non-small cell lung cancer, ovarian cancer, breast cancer, pancreatic cancer, a hematological malignancy, and a renal cell carcinoma.

In certain embodiments, the cancer is a haematological cancer, selected from Acute myeloid leukemia, Angioimmunoblastic T-cell lymphoma, B-cell acute lymphoblastic leukemia, Sweet Syndrome, T-cell Non-Hodgkins lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell Non-Hodgkins lymphoma (including Burkitt lymphoma, diffuse large B-cell lymphoma, Follicular lymphoma, Mantle cell lymphoma, Marginal Zone lymphoma), Hairy Cell Leukemia, Hodgkin lymphoma, Lymphoblastic lymphoma, Lymphoplasmacytic lymphoma, Mucosa-associated lymphoid tissue lymphoma, Multiple myeloma, Myelodysplastic syndrome, Plasma cell myeloma, Primary mediastinal large B-cell lymphoma, chronic myeloproliferative disorders (such as chronic myeloid leukemia, primary myelofibrosis, essential thrombocytemia, polycytemia vera) and chronic lymphocytic leukemia.

In some embodiments, the cancer is a non-haematological cancer, selected from bladder cancer, breast, melanoma, neuroblastoma, malignant pleural mesothelioma, and sarcoma.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In certain embodiments, the cancer is brain cancer, leukemia, skin cancer, prostate cancer, thyroid cancer, colon cancer, lung cancer or sarcoma. In another embodiment the cancer is selected from the group consisting of glioma, glioblastoma multiforme, paraganglioma, suprantentorial primordial neuroectodermal tumors, acute myeloid leukemia, myelodysplastic syndrome, chronic myelogenous leukemia, melanoma, breast, prostate, thyroid, colon, lung, central chondrosarcoma, central and periosteal chondroma tumors, fibrosarcoma, and cholangiocarcinoma.

In certain embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In a further embodiment, the cancer is selected from brain and spinal cancers. In particular embodiments, the cancer is selected from the group consisting of anaplastic astrocytomas, glioblastomas, astrocytomas, and estheosioneuroblastomas (olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astrocytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, the cancer is selected from cancers of the head and neck, including nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute mycloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (hepatocellular carcinoma). In particular embodiments, the cancer is bile duct cancer (cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urethelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, and chordoma.

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Stemberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, and primary central nervous system lymphomas.

In specific embodiments, the cancer is selected from glandular cancers. In particular embodiments, the cancer is selected from the group consisting of adrenocortical cancer, pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosarcoma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In some embodiments, the subject has melanoma. The melanoma may be at early stage or at late stage. In some embodiments, the subject has colorectal cancer. The colorectal cancer may be at early stage or at late stage. In some embodiments, the subject has non-small cell lung cancer. The non-small cell lung cancer may be at early stage or at late stage. In some embodiments, the subject has pancreatic cancer. The pancreatic cancer may be at early stage or late state. In some embodiments, the subject has a hematological malignancy. The hematological malignancy may be at early stage or late stage. In some embodiments, the subject has ovarian cancer. The ovarian cancer may be at early stage or at late stage. In some embodiments, the subject has breast cancer. The breast cancer may be at early stage or at late stage. In some embodiments, the subject has renal cell carcinoma. The renal cell carcinoma may be at early stage or at late stage. In some embodiments, the cancer has elevated levels of T-cell infiltration.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer and lung cancer (e.g. non-small cell lung cancer and small cell lung cancer). Additionally, the disclosure includes refractory or recurrent malignancies whose growth may be inhibited using the compounds of the disclosure.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myclogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL), multiple myeloma, cutaneous T-cell lymphoma, Waldenstrom's Macroglobulinemia, hairy cell lymphoma, chronic myclogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers.

In some embodiments, CTPS 1 inhibitors may be used to treat tumors producing PGE2 (e.g. Cox-2 overexpressing tumors) and/or adenosine (CD73 and CD39 over-expressing tumors). Overexpression of Cox-2 has been detected in a number of tumors, such as colorectal, breast, pancreatic and lung cancers, where it correlates with a poor prognosis. Overexpression of COX-2 has been reported in hematological cancer models such as RAJI (Burkitt's lymphoma) and U937 (acute promonocytic leukemia) as well as in patient's blast cells. CD73 is up-regulated in various human carcinomas including those of colon, lung, pancreas and ovary. Importantly, higher expression levels of CD73 are associated with tumor neovascularization, invasiveness, and metastasis and with shorter patient survival time in breast cancer.

In certain embodiments, the invention provides a method of treating a CTPS1-mediated disease or disorder in a subject, wherein the treatment reduces T-cell and/or B-cell proliferation, comprising administering a therapeutically effective amount of a compound of the invention, or a pharmaceutically acceptable salt, hydrate, solvate, or prodrug thereof, to the subject.

In certain embodiments, the invention provides a method of reducing T-cell and/or B-cell proliferation, comprising administering a therapeutically effective amount of a compound of the invention, or a pharmaceutically acceptable salt, hydrate, solvate, or prodrug thereof, to the subject.

In certain embodiments, the invention provides for the use of a compound of the invention, or a pharmaceutically acceptable salt, solvate, or derivative thereof, in the manufacture of a medicament for the reduction of T-cell and/or B-cell proliferation in a subject.

In certain embodiments, the compounds of the invention are used in enhancing recovery from vascular injury or surgery and reducing morbidity and mortality associated with neointima and restenosis in a subject. For example, the compounds of formula (I) may be used in preventing, reducing, or inhibiting neointima formation. A medical device may be treated prior to insertion or implantation with an effective amount of a composition comprising a compound of formula (I) in order to prevent, reduce, or inhibit neointima formation following insertion or implantation of the device or graft into the subject. The device can be a device that is inserted into the subject transiently, or a device that is implanted permanently. In some embodiments, the device is a surgical device. Examples of medical devices include, but are not limited to, needles, cannulas, catheters, shunts, balloons, and implants such as stents and valves.

In some embodiments, the invention provides a pharmaceutical composition comprising an effective amount of a compound of the invention, or a pharmaceutically acceptable salt, hydrate, solvate, or prodrug thereof, and a pharmaceutically acceptable carrier.

In certain embodiments, the invention provides a compound of the invention, or a pharmaceutically acceptable salt, hydrate, solvate, or prodrug thereof, for use in the treatment or prophylaxis of a disease or disorder provided herein.

In certain embodiments, the invention provides a compound of the invention, or a pharmaceutically acceptable salt, hydrate, solvate, or prodrug thereof, for use as a medicament, for the treatment or prophylaxis of a disease or disorder provided herein.

In certain embodiments, the invention provides a compound of the invention, or a pharmaceutically acceptable salt, hydrate, solvate, or prodrug thereof, in the manufacture of a medicament for the inhibition of CTPS1 in a subject.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

The presently disclosed compounds may be administered in any suitable manner known in the art. In some embodiments, the compound of the invention or a pharmaceutically acceptable salt, prodrug, metabolite, or derivative thereof is administered intravenously, intramuscularly, subcutaneously, topically, orally, transdermally, intraperitoneally, intraorbitally, by implantation, by inhalation, intrathecally, intraventricularly, intratumorally, or intranasally.

In some embodiments, the CTPS1 antagonist is administered continuously. In other embodiments, the CTPS1 antagonist is administered intermittently. Moreover, treatment of a subject with an effective amount of a CTPS1 antagonist can include a single treatment or can include a series of treatments.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the invention or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the CTPS1 antagonist is administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 μg/kg, 1 μg/kg, 10 μg/kg, 25 g/kg, 50 μg/kg, 100 g/kg, 250 μg/kg, 500 μg/kg, 1 mg/kg, 5 mg/kg, 10 mg/kg, 25 mg/kg, 50 mg/kg, 100 mg/kg, and 200 mg/kg.

In the methods described herein, the method can further comprise administering a chemotherapeutic agent to the subject. In certain aspects of this embodiment, the chemotherapeutic agent is administered to the subject simultaneously with the compound or the composition. In certain aspects of this embodiment, the chemotherapeutic agent is administered to the subject prior to administration of the compound or the composition. In certain aspects of this embodiment, the chemotherapeutic agent is administered to the subject after administration of the compound or the composition.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “prophylaxis” or “preventing” is used to mean preventing symptoms of a disease or disorder in a subject or preventing recurrence of symptoms of a disease or disorder in an afflicted subject and is not limited to complete prevention of an affliction.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “subject” refers to animals such as mammals, including, but not limited to, primates (e.g., humans), cows, sheep, goats, horses, dogs, cats, rabbits, rats, mice and the like. In certain embodiments, the subject is a human.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 R^{A}, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a compound of formula I and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvise® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Acrobid®), Afviar®, Symbicort®, Dulcra®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating rheumatoid arthritis comprising administering to a patient in need thereof a compound of formula I and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actcmra®).

In some embodiments, the present invention provides a method of treating osteoarthritis comprising administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating cutaneous lupus erythematosus or systemic lupus erythematosus comprising administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating Crohn's disesase, ulcerative colitis, or inflammatory bowel disease comprising administering to a patient in need thereof a compound of formula I and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a compound of formula I and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a compound of formula I and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, the present invention provides a method of treating a hematological malignancy comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PT3K inhibitor, a SYK inhibitor, and combinations thereof.

In another embodiment, the present invention provides a method of treating a hematological malignancy comprising administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple mycloma comprising administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a compound of formula I and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behect's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom's macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, myasthenia gravis, Hashimoto's thyroiditis, atopic dermatitis, degenerative joint disease, vitiligo, autoimmune hypopituitarism, Guillain-Barre syndrome, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a compound of formula I and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALT), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, crythema multiforma, dermatitis hcrpctiformis, sclcroderma, vitiligo, hyperscnsitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoinmmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments the present invention provides a method of treating or lessening the severity of a disease comprising administering to a patient in need thereof a compound of formula I and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, the disease is an autoimmune disorder, an inflammatory disorder, a proliferative disorder, an endocrine disorder, a neurological disorder, or a disorder associated with transplantation. In some embodiments the JH2 binding compound is a compound of formula I. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

The compounds and compositions, according to the method of the present invention, may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity in a biological sample comprising the step of contacting said biological sample with a compound of this invention, or a composition comprising said compound.

According to another embodiment, the invention relates to a method of inhibiting CTPS 1, or a mutant thereof, activity in a biological sample comprising the step of contacting said biological sample with a compound of this invention, or a composition comprising said compound. In certain embodiments, the invention relates to a method of irreversibly inhibiting CTPS 1, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of CTPS 1 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of inhibiting protein kinase activity in a patient comprising the step of administering to said patient a compound of the present invention, or a composition comprising said compound.

According to another embodiment, the invention relates to a method of inhibiting activity of CTPS1, or a mutant thereof, in a patient comprising the step of administering to said patient a compound of the present invention, or a composition comprising said compound. According to certain embodiments, the invention relates to a method of reversibly or irreversibly inhibiting one or more of CTPS1, or a mutant thereof, activity in a patient comprising the step of administering to said patient a compound of the present invention, or a composition comprising said compound. In other embodiments, the present invention provides a method for treating a disorder mediated by CTPS1, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal*); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SUl01, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; Isis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa^{T}M. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms,” such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zamestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bel-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosage or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastinc fumarate, promcthazinc, loratidinc, dcsloratidinc, diphcnhydraminc and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention were prepared by methods substantially similar to those described herein in the Examples and methods known to one skilled in the art.

LCMS-MS analytical experiments were conducted using a Shimadzu LCMS-2020 machine with solvents, gradients and stationary phases listed in the table below.

| | | Stationary | Method | Mobile | Mobile |
|---|---|---|---|---|---|
| Code | Gradient | Phase | Description | Phase A | Phase B |
| LCMS- | 5% B to 70% B over | HALO C18 | column: | Water + | ACN + |
| A | 1.7 min, 70% B to 95 | | 30*3.0 mm, | 0.05% TFA | 0.05% |
| | % B over 0.3 min, | | 2.7 μm | | TFA |
| | then a 0.60 min hold | | | | |
| | at 95% B; Flow: 1.5 | | | | |
| | mL/min; | | | | |
| LCMS- | 5% B to 65% B over | HALO C18 | column: | Water + | ACN + |
| B | 1.7 min, 65% B to 95 | | 30*3.0 mm, | 0.05% TFA | 0.05% |
| | % B over 0.3 min, | | 2.7 μm | | TFA |
| | then a 0.60 min hold | | | | |
| | at 95% B; Flow: 1.5 | | | | |
| | mL/min; | | | | |
| LCMS- | 5% B to 75% B over | HALO C18 | column: | Water + | ACN + |
| C | 1.7 min, 75% B to 95 | | 30*3.0 mm, | 0.05% TFA | 0.05% |
| | % B over 0.3 min, | | 2.7 μm | | TFA |
| | then a 0.60 min hold | | | | |
| | at 95% B; Flow: 1.5 | | | | |
| | mL/min; | | | | |
| LCMS- | 20% B to 70% B | HALO C18 | column: | Water + | ACN + |
| D | over 1.9 min, 70% B | | 30*3.0 mm, | 0.05% TFA | 0.05% |
| | to 100% B over 0.1 | | 2.7 μm | | TFA |
| | min, then a 0.60 min | | | | |
| | hold at 100% B; | | | | |
| | Flow: 1.5 mL/min; | | | | |
| LCMS- | 5% B to 100% B | HALO C18 | column: | Water + | ACN + |
| E | over 1.3 min, then a | | 30*3.0 mm, | 0.05% TFA | 0.05% |
| | 0.50 min hold at 100 | | 2.7 μm | | TFA |
| | % B; Flow: 1.5 | | | | |
| | mL/min; | | | | |
| LCMS- | 5% B to 55% B over | HALO C18 | column: | Water + | ACN + |
| F | 1.7 min, 55% B to 95 | | 30*3.0 mm, | 0.05% TFA | 0.05% |
| | % B over 0.3 min, | | 2.7 μm | | TFA |
| | then a 0.60 min hold | | | | |
| | at 95% B; Flow: 1.5 | | | | |
| | mL/min; | | | | |
| LCMS- | 5% B to 60% B over | HALO C18 | column: | Water + | ACN + |
| G | 1.8 min, 60% B to | | 30*3.0 mm, | 0.05% TFA | 0.05% |
| | 100% B over 0.15 | | 2.7 μm | | TFA |
| | min, then a 0.70 min | | | | |
| | hold at 100% B; | | | | |
| | Flow: 1.5 mL/min; | | | | |
| LCMS- | 5% B to 70% B over | HALO C18 | column: | Water + | ACN + |
| H | 1.9 min, 70% B to | | 30*3.0 mm, | 0.05% TFA | 0.05% |
| | 100% B over 0.15 | | 2.7 μm | | TFA |
| | min, then a 0.55 min | | | | |
| | hold at 100% B; | | | | |
| | Flow: 1.5 mL/min; | | | | |
| LCMS- | 30% B to 70% B | HALO C18 | column: | Water + | ACN + |
| I | over 1.75 min, 70 | | 30*3.0 mm, | 0.05% TFA | 0.05% |
| | % B to 100% B over | | 2.7 μm | | TFA |
| | 0.25 min, then a 0.65 | | | | |
| | min hold at 100% B; | | | | |
| | Flow: 1.5 mL/min; | | | | |
| LCMS- | 5% B to 60% B over | HALO C18 | column: | Water + | ACN + |
| J | 2.1 min, 60% B to | | 30*3.0 mm, | 0.05% TFA | 0.05% |
| | 100% B over 0.3 | | 2.7 μm | | TFA |
| | min, then a 0.4 min | | | | |
| | hold at 100% B; | | | | |
| | Flow: 1.2 mL/min; | | | | |
| LCMS- | 5% B to 55% B over | HALO C18 | column: | Water + | ACN + |
| K | 2.1 min, 55% B to | | 30*3.0 mm, | 0.05% TFA | 0.05% |
| | 100% B over 0.3 | | 2.7 μm | | TFA |
| | min, then a 0.4 min | | | | |
| | hold at 100% B; | | | | |
| | Flow: 1.5 mL/min; | | | | |
| LCMS- | 5% B to 100% B | HALO C18 | column: | Water + | ACN + |
| L | over 1.3 min, then a | | 30*3.0 mm, | 0.05% TFA | 0.05% |
| | 0.50 min hold at 100 | | 2.0 μm | | TFA |
| | % B; Flow: 1.2 | | | | |
| | mL/min; | | | | |
| LCMS- | 30% B to 70% B | HALO C18 | column: | Water + | ACN + |
| M | over 2 min, 70% B to | | 30*3.0 mm, | 0.05% TFA | 0.05% |
| | 100% B over 0.4 | | 2.0 μm | | TFA |
| | min, then a 0.4 min | | | | |
| | hold at 100% B; | | | | |
| | Flow: 1.2 mL/min; | | | | |
| LCMS- | 20% B to 70% B | HALO C18 | column: | Water + | ACN + |
| N | over 2.1 min, 70% B | | 30*3.0 mm, | 0.05% TFA | 0.05% |
| | to 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 20% B to 60% B | HALO C18 | column: | Water + | ACN + |
| O | over 2.1 min, 60% B | | 30*3.0 mm, | 0.05% TFA | 0.05% |
| | to 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 5% B to 100% B | HALO | column: | Water + | ACN + |
| P | over 1.3 min, then a | AQ-C18 | 30*3.0 mm, | 0.05% TFA | 0.05% |
| | 0.50 min hold at 100 | | 2.0 μm | | TFA |
| | % B; Flow: 1.2 | | | | |
| | mL/min; | | | | |
| LCMS- | 5% B to 60% B over | HALO | column: | Water + | ACN + |
| Q | 2.1 min, 60% B to | AQ-C18 | 30*3.0 mm, | 0.05% TFA | 0.05% |
| | 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 5% B to 70% B over | HALO | column: | Water + | ACN + |
| R | 2.1 min, 70% B to | AQ-C18 | 30*3.0 mm, | 0.05% TFA | 0.05% |
| | 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 20% B to 60% B | HALO | column: | Water + | ACN + |
| S | over 2.1 min, 60% B | AQ-C18 | 30*3.0 mm, | 0.05% TFA | 0.05% |
| | to 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 5% B to 65% B over | HALO | column: | Water + | ACN + |
| T | 2.1 min, 65% B to | AQ-C18 | 30*3.0 mm, | 0.05% TFA | 0.05% |
| | 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 20% B to 70% B | HALO | column: | Water + | ACN + |
| U | over 2.1 min, 70% B | AQ-C18 | 30*3.0 mm, | 0.05% TFA | 0.05% |
| | to 100% B over 0.3 | | 2.0 μm | | TFA |
| | min, then a 0.4 min | | | | |
| | hold at 100% B; | | | | |
| | Flow: 1.2 mL/min; | | | | |
| LCMS- | 5% B to 65% B over | HALOWA | column: | Water + | ACN + |
| V | 2.1 min, 65% B to | C18 | 30*3.0 mm, | 0.05% TFA | 0.05% |
| | 100% B over 0.3 | | 1.9 μm | | TFA |
| | min, then a 0.4 min | | | | |
| | hold at 100% B; | | | | |
| | Flow: 1.2 mL/min; | | | | |
| LCMS- | 5% B to 100% B | HALOWA | column: | Water + | ACN + |
| W | over 1.3 min, then a | C18 | 30*3.0 mm, | 0.05% TFA | 0.05% |
| | 0.50 min hold at 100 | | 1.9 μm | | TFA |
| | % B; Flow: 1.2 | | | | |
| | mL/min; | | | | |
| LCMS- | 5% B to 55% B over | HALOWA | column: | Water + | ACN + |
| X | 2.1 min, 55% B to | C18 | 30*3.0 mm, | 0.05% TFA | 0.05% |
| | 100% B over 0.3 | | 1.9 μm | | TFA |
| | min, then a 0.4 min | | | | |
| | hold at 100% B; | | | | |
| | Flow: 1.2 mL/min; | | | | |
| LCMS- | 5% B to 60% B over | HALOWA | column: | Water + | ACN + |
| Y | 2.1 min, 60% B to | C18 | 30*3.0 mm, | 0.05% TFA | 0.05% |
| | 100% B over 0.3 | | 1.9 μm | | TFA |
| | min, thon a 0.4 min | | | | |
| | hold at 100% B; | | | | |
| | Flow: 1.2 mL/min; | | | | |
| LCMS- | 5% B to 70% B over | HALOWA | column: | Water + | ACN + |
| Z | 2.1 min, 70% B to | C18 | 30*3.0 mm, | 0.05% TFA | 0.05% |
| | 100% B over 0.3 | | 1.9 μm | | TFA |
| | min, then a 0.4 min | | | | |
| | hold at 100% B; | | | | |
| | Flow: 1.2 mL/min; | | | | |
| LCMS- | 20% B to 60% B over | ACE Excel | column: | Water + | ACN + |
| AA | 1.75 min, 60% B to | 2 C18 | 50*3.0 mm, | 0.05% TFA | 0.05% |
| | 100% B over 0.25 | | 2.0 μm | | TFA |
| | min, then a 0.65 min | | | | |
| | hold at 100% B; | | | | |
| | Flow: 1.2 mL/min; | | | | |
| LCMS- | 20% B to 75% B over | ACE Excel | column: | Water + | ACN + |
| AB | 1.9 min, 75% B to | 2 C18 | 50*3.0 mm, | 0.05% TFA | 0.05% |
| | 100% B over 0.1 | | 2.0 μm | | TFA |
| | min, then a 0.6 min | | | | |
| | hold at 100% B; | | | | |
| | Flow: 1.2 mL/min; | | | | |
| LCMS- | 5% B to 100% B | ACE Excel | column: | Water + | ACN + |
| AC | over 1.2 min, then a | 2 C18 | 50*3.0 mm, | 0.05% TFA | 0.05% |
| | 0.60 min hold at 100 | | 2.0 μm | | TFA |
| | % B; Flow: 1.2 | | | | |
| | mL/min; | | | | |
| LCMS- | 5% B to 55% B over | ACE Excel | column: | Water + | ACN + |
| AD | 1.7 min, 55% B to | 2 C18 | 50*3.0 mm, | 0.05% TFA | 0.05% |
| | 100% B over 0.3 | | 2.0 μm | | TFA |
| | min, then a 0.6 min | | | | |
| | hold at 100% B; | | | | |
| | Flow: 1.2 mL/min; | | | | |
| LCMS- | 5% B to 60% B over | ACE Excel | column: | Water + | ACN + |
| AE | 1.8 min, 60% B to | 2 C18 | 50*3.0 mm, | 0.05% TFA | 0.05% |
| | 100% B over 0.15 | | 2.0 μm | | TFA |
| | min, then a 0.7 min | | | | |
| | hold at 100% B; | | | | |
| | Flow: 1.2 mL/min; | | | | |
| LCMS- | 5% B to 65% B over | ACE Excel | column: | Water + | ACN + |
| AF | 1.7 min, 65% B to | 2 C18 | 50*3.0 mm, | 0.05% TFA | 0.05% |
| | 100% B over 0.3 | | 2.0 μm | | TFA |
| | min, then a 0.6 min | | | | |
| | hold at 100% B; | | | | |
| | Flow: 1.2 mL/min; | | | | |
| LCMS- | 5% B to 70% B over | ACE Excel | column: | Water + | ACN + |
| AG | 1.7 min, 70% B to | 2 C18 | 50*3.0 mm, | 0.05% TFA | 0.05% |
| | 100% B over 0.3 | | 2.0 μm | | TFA |
| | min, then a 0.6 min | | | | |
| | hold at 100% B; | | | | |
| | Flow: 1.2 mL/min; | | | | |
| LCMS- | 20% B to 50% B | Ascentis | column: | Water + | ACN + |
| AH | over 1.9 min, 50% B | Express | 50*3.0 mm, | 0.05% TFA | 0.05% |
| | to 100% B over 0.1 | C18 | 2.7 μm | | TFA |
| | min, then a 0.7 min | | | | |
| | hold at 100% B; | | | | |
| | Flow: 1.5 mL/min; | | | | |
| LCMS- | 5% B to 100% B | XSelect | column: | Water + | ACN + |
| AI | over 1.3 min, then a | HSS T3 | 30*2.1 mm, | 0.05% TFA | 0.05% |
| | 0.50 min hold at 100% | | 2.5 μm | | TFA |
| | B; Flow: 1.2 | | | | |
| | mL/min; | | | | |
| LCMS- | 20% B to 70% B | XSelect | column: | Water + | ACN + |
| AJ | over 2.1 min, 70% B | HSS T3 | 30*2.1 mm, | 0.05% TFA | 0.05% |
| | to 100% B over 0.3 | | 2.5 μm | | TFA |
| | min, then a 0.4 min | | | | |
| | hold at 100% B; | | | | |
| | Flow: 1.2 mL/min; | | | | |
| LCMS- | 5% B to 60% B over | XSelect | column: | Water + | ACN + |
| AK | 2.1 min, 60% B to | HSS T3 | 30*2.1 mm, | 0.05% TFA | 0.05% |
| | 100% B over 0.3 | | 2.5 μm | | TFA |
| | min, then a 0.4 min | | | | |
| | hold at 100% B; | | | | |
| | Flow: 1.2 mL/min; | | | | |
| LCMS- | 5% B to 100% B | XSelect | column: | 0.1% | ACN |
| AL | over 1.3 min, then a | HSS T3 | 100*4.6 mm, | H₃PO₄ + | |
| | 0.50 min hold at 100 | | 2.5 μm | H₂O | |
| | % B; Flow: 1.2 | | | | |
| | mL/min; | | | | |
| LCMS- | 5% B to 100% B | Luna Omega | column: | water + | ACN + |
| AM | over 1.2 min, then a | PS C18 | 50*2.1 mm | 0.1% FA | 0.1% FA |
| | 0.60 min hold at 100% | | | | |
| | B; Flow: 1.5 | | | | |
| | mL/min; | | | | |
| LCMS- | 5% B to 100% B | Luna | column: | Water + | ACN + |
| AN | over 1.2 min, then a | Omega PS | 50*3.0 mm, | 0.09% FA | 0.1% FA |
| | 0.60 min hold at 100% | C18 | 3.0 μm | | |
| | B; Flow: 1.5 | | | | |
| | mL/min; | | | | |
| LCMS- | 20% B to 50% B | Luna | column: | Water + | ACN + |
| AO | over 1.9 min, 60% B | Omega PS | 50*3.0 mm, | 0.09% FA | 0.1% FA |
| | to 100% B over 0.1 | C18 | 3.0 μm | | |
| | min, then a 0.7 min | | | | |
| | hold at 100% B; | | | | |
| | Flow: 1.5 mL/min; | | | | |
| LCMS- | 10% B to 45% B | Kinetex | column: | Water + 5 | ACN |
| AP | over 1.80 min, 45% | EVO C18 | 30*3.0 mm, | mM | |
| | B to 95% B over | | 2.6 μm | NH₄HCO₃ | |
| | 0.45 min, then a 0.55 | | | | |
| | min hold at 95% B; | | | | |
| | Flow: 1.5 mL/min; | | | | |
| LCMS- | 10% B to 50% B | Kinetex | column: | Water + | ACN |
| AQ | over 1.9 min, 50% B | EVO C18 | 30*3.0 mm, | 5 mM | |
| | to 95% B over 0.5 | | 2.6 μm | NH₄HCO₃ | |
| | min, then a 0.4 min | | | | |
| | hold at 95% B; Flow: | | | | |
| | 1.5 mL/min; | | | | |
| LCMS- | 10% B to 50% B | Kinetex | column: | Water + | ACN |
| AR | over 1.9 min, 50% B | EVO C18 | 100*4.6 mm, | 5 mM | |
| | to 95% B over 0.5 | | 2.6 μm | NH₄HCO₃ | |
| | min, then a 0.4 min | | | | |
| | hold at 95% B; Flow: | | | | |
| | 1.5 mL/min; | | | | |
| LCMS- | 10% B to 55% B | Kinetex | column: | Water + | ACN |
| AS | over 1.85 min, 55% | EVO C18 | 100*4.6 mm, | 5 mM | |
| | B to 95% B over | | 2.6 μm | NH₄HCO₃ | |
| | 0.45 min, then a 0.5 | | | | |
| | min hold at 95% B; | | | | |
| | Flow: 1.5 mL/min; | | | | |
| LCMS- | 10% B to 60% B | Kinetex | column: | Water + | ACN |
| AT | over 1.85 min, 60% | EVO C18 | 30*3.0 mm, | 5 mM | |
| | B to 95% B over | | 2.6 μm | NH₄HCO₃ | |
| | 0.45 min, then a 0.5 | | | | |
| | min hold at 95% B; | | | | |
| | Flow: 1.5 mL/min; | | | | |
| LCMS- | 10% B to 95% B | Kinetex | column: | Water + | ACN |
| AU | over 1.2 min, then a | EVO C18 | 30*3.0 mm, | 5 mM | |
| | 0.58 min hold at | | 2.6 μm | NH₄HCO₃ | |
| | 95% B; Flow: 1.5 | | | | |
| | mL/min; | | | | |
| LCMS- | 10% B to 95% B | Kinetex | column: | Water + | ACN |
| AV | over 1.2 min, then a | EVO C18 | 100*4.6 mm, | 5 mM | |
| | 0.58 min hold at | | 2.6 μm | NH₄HCO₃ | |
| | 95% B; Flow: 1.5 | | | | |
| | mL/min; | | | | |
| LCMS- | 10% B to 50% B | Kinetex | column: | Water + | ACN |
| AW | over 1.9 min, 50% B | EVO C18 - | 30*3.0 mm, | 6.5 mM | |
| | to 95% B over 0.1 | 100A | 2.6 μm | NH₄HCO₃ | |
| | min, then a 0.7 min | | | | |
| | hold at 95% B; Flow: | | | | |
| | 1.2 mL/min; | | | | |
| LCMS- | 10% B to 95% B over | L-column3 | column: | Water + | ACN |
| AX | 1.2 min, then a 0.58 | C18 | 30*3.0 mm, | 5 mM | |
| | min hold at 95% B; | | 2.0 μm | NH₄HCO₃ | |
| | Flow: 1.5 mL/min; | | | | |
| LCMS- | 10% B to 95% B | L- | column: | Water + | ACN |
| AY | over 1.2 min, then a | column3 | 30*3.0 mm, | 5 mM | |
| | 0.58 min hold at | C18 | 2.7 μm | NH₄HCO₃ | |
| | 95% B; Flow: 1.5 | | | | |
| | mL/min; | | | | |
| LCMS- | 10% B to 95% B | Poroshell | column: | Water + | ACN |
| AZ | over 1.2 min, then a | HPH-C18 | 50*3.0 mm, | 6.5 mM | |
| | 0.6 min hold at | | 2.7 μm | NH₄HCO₃ | |
| | 95% B; Flow: 1.2 | | | | |
| | mL/min; | | | | |
| LCMS- | 10% B to 95% B | Shim Pack | column: | Water + | ACN |
| BA | over 1.2 min, then a | Scepter | 33*3.0 mm, | 6.5 mM | |
| | 0.6 min hold at | C18 | 3.0 μm | NH₄HCO₃ | |
| | 95% B; Flow: 1.2 | | | | |
| | mL/min; | | | | |
| LCMS- | 10% B to 95% B | Shim-pack | column: | Water + | ACN |
| BB | over 1.2 min, then a | Scepter | 33*3.0 mm, | 5 mM | |
| | 0.58 min hold at | C18 | 3.0 μm | NH₄HCO₃ | |
| | 95% B; Flow: 1.5 | | | | |
| | mL/min; | | | | |
| LCMS- | 10% B to 50% B | Shim- | column: | Water + | ACN |
| BC | over 1.85 min, 50% | pack | 33*3.0 mm, | 5 mM | |
| | B to 95% B over | ScepterC18 | 3.0 μm | NH₄HCO₃ | |
| | 0.45 min, then a 0.5 | | | | |
| | min hold at 95% B; | | | | |
| | Flow: 1.5 mL/min; | | | | |
| LCMS- | 10% B to 45% B | Shim- | column: | Water + | ACN |
| BD | over 1.85 min, 45% | pack | 33*3.0 mm, | 5 mM | |
| | B to 95% B over | ScepterC18 | 3.0 μm | NH₄HCO₃ | |
| | 0.45 min, then a 0.55 | | | | |
| | min hold at 95% B; | | | | |
| | Flow: 1.5 mL/min; | | | | |
| LCMS- | 10% B to 55% B | Shim- | column: | Water + | ACN |
| BE | over 1.85 min, 55% | pack | 33*3.0 mm, | 5 mM | |
| | B to 95% B over | ScepterC18 | 3.0 μm | NH₄HCO₃3 | |
| | 0.45 min, then a 0.50 | | | | |
| | min hold at 95% B; | | | | |
| | Flow: 1.5 mL/min; | | | | |
| LCMS- | 10% B to 60% B | Shim- | column: | Water + | ACN |
| BF | over 1.85 min, 60% | pack | 33*3.0 mm, | 5 mM | |
| | B to 95% B over | SccpterC18 | 3.0 μm | NH₄HCO₃ | |
| | 0.45 min, then a 0.50 | | | | |
| | min hold at 95% B; | | | | |
| | Flow: 1.5 mL/min; | | | | |
| LCMS- | 10% B to 65% B | Shim- | column: | Water + | ACN |
| BG | over 1.9 min, 65% B | pack | 33*3.0 mm, | 5 mM | |
| | to 95% B over 0.4 | ScepterC18 | 3.0 μm | NH₄HCO₃ | |
| | min, then a 0.50 min | | | | |
| | hold at 95% B; Flow: | | | | |
| | 1.5 mL/min; | | | | |
| LCMS- | 10% B to 70% B | Shim-pack | column: | Water + | ACN |
| BH | over 2.1 min, 70% B | Scepter | 33*3.0 mm, | 5 mM | |
| | to 95% B over 0.2 | C18 | 3.0 μm | NH₄HCO₃ | |
| | min, then a 0.50 min | | | | |
| | hold at 95% B; Flow: | | | | |
| | 1.5 mL/min; | | | | |
| LCMS- | 30% B to 60% B | Shim- | column: | Water + | ACN |
| BI | over 1.85 min, 60 | pack | 33*3.0 mm, | 5 mM | |
| | % B to 95% B over | ScepterC18 | 3.0 μm | NH₄HCO₃ | |
| | 0.45 min, then a 0.50 | | | | |
| | min hold at 95% B; | | | | |
| | Flow: 1.5 mL/min; | | | | |

### Example 1: Preparation of Intermediate a (Int. A) and Compounds of the Disclosure

### Synthesis of Int. A.6

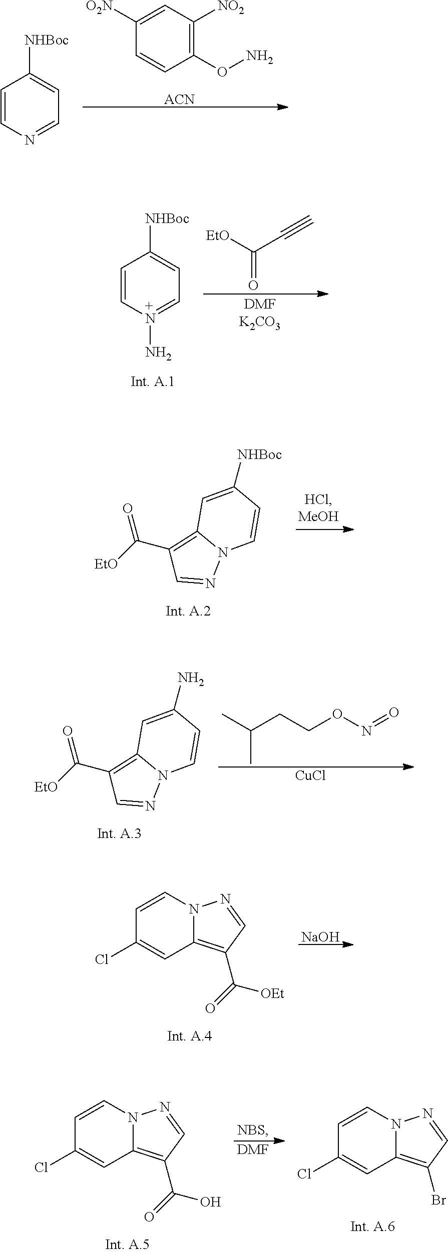

Synthesis of Int. A.2. To a stirred solution of tert-butyl N-(pyridin-4-yl)carbamate (20 g, 103 mmol, 1 eq) in acetonitrile (520 mL) was added O-(2,4-dinitrophenyl)hydroxylamine (24.7 g, 123.6 mmol, 1.2 eq). The resulting mixture was stirred for 2 h at 70° C. The mixture was allowed to cool down to room temperature. The resulting mixture was concentrated under reduced pressure to obtain crude Int. A.1 (22 g, MS (ES): m/z 210 [M]⁺), as a brown yellow solid. Potassium carbonate (29 g) was added to a mixture of Int. A.1 (22 g, crude) and ethyl propiolate (10.6 mL) in N, N-dimethylformamide (320 mL). The resulting mixture was stirred for 16 h at room temperature, then was diluted water and the products were extracted with ethyl acetate. The combined organic layer was washed with brine, dried over sodium sulfate and concentrated under reduced pressure. The residue was purified by flash column chromatography on silica gel (elated with 20% ethyl acetate in petroleum ether) to afford ethyl 5-((tert-butoxycarbonyl)amino)pyrazolo[1,5-a]pyridine-3-carboxylate (Int. A.2, 8 g) as a light yellow solid. MS (ES): m/z 306 [M+H]⁺.

Synthesis of Int. A.3. To a stirred solution of Int. A.2 (8 g, 26.1 mmol) in methanol (50 mL) was added HCl in 1,4-dioxane (4M, 100 mL) dropwise at room temperature. The resulting mixture was stirred at room temperature for 3 h. The reaction mixture was filtered and the solids were collected, then dried under vacuum to afford ethyl 5-aminopyrazolo[1,5-a]pyridine-3-carboxylate hydrochloride (Int. A.3, 5.75 g, 91%) as a white solid. MS (ES): n/z 206 [M+H]⁺.

Synthesis of Int. A.4. A mixture of Int. A.3 (1.03 g, 4.26 mmol, 1 eq) and cuprous chloride (633 mg, 6.39 mmol, 1.5 eq) in acetonitrile (10 mL) was stirred for 15 min at room temperature. To the above mixture was added isoamyl nitrite (997 mg, 8.52 mmol, 2 eq). The resulting mixture was stirred for additional 1 h at 70° C. The mixture was allowed to cool down to room temperature. The resulting mixture was filtered, the filter cake was washed with ethyl acetate and water. The filtrate was extracted with ethyl acetate. The combined organic layer was washed with brine, dried over sodium sulfate and concentrated under reduced pressure. The residue was purified by reverse phase flash chromatography (C18 Column; Mobile Phase, water (0.1% FA) and ACN (10% ACN up to 40% in 20 min); UV detection at 254/220 nm). The product-containing fractions were combined and concentrated under reduced pressure to obtain ethyl 5-chloropyrazolo[1,5-a]pyridine-3-carboxylate (Int. A.4, 530 mg, 55%) as a brown yellow solid. MS (ES): m/z 225 [M+H]⁺.

Synthesis of Int. A.5. To a stirred mixture of Int. A.4 (400 mg, 1.78 mmol, 1 eq) in methyl alcohol (4 mL) and water (1 mL) was added sodium hydroxide (142 mg, 3.56 mmol, 2 eq). The resulting mixture was stirred for 1 h at 60° C. The mixture was allowed to cool down to room temperature and diluted with water. The pH value of the solution was adjusted to 3 with 1N aqueous hydrochloric acid. The solids were collected by filtration to obtain the crude product 5-chloropyrazolo[1,5-a]pyridine-3-carboxylic acid (Int. A.5, 300 mg, 86%) as a white solid. MS (ES): m/z 197 [M+H]⁺.

Synthesis of Int. A.6. To a stirred mixture of Int. A.5 (829 mg, 4.22 mmol, 1.0 eq) in N,N-dimethylformamide (15 mL) was added N-bromosuccinimide (NBS) (901 mg, 5.06 mmol, 1.2 eq). The resulting mixture was stirred for 1 h at room temperature. The mixture was diluted with water and extracted with ethyl acetate. The combined organic layer was washed with brine, dried over sodium sulfate and concentrated under reduced pressure. The residue was purified by Prep-TLC (petroleum ether/ethyl acetate=3/1) to afford 3-bromo-5-chloropyrazolo[1,5-a]pyridine (Int. A.6, 440 mg, 45%) as a yellow solid. MS (ES): m/z 231/233 [M+H]⁺.

### Synthesis of Int. A

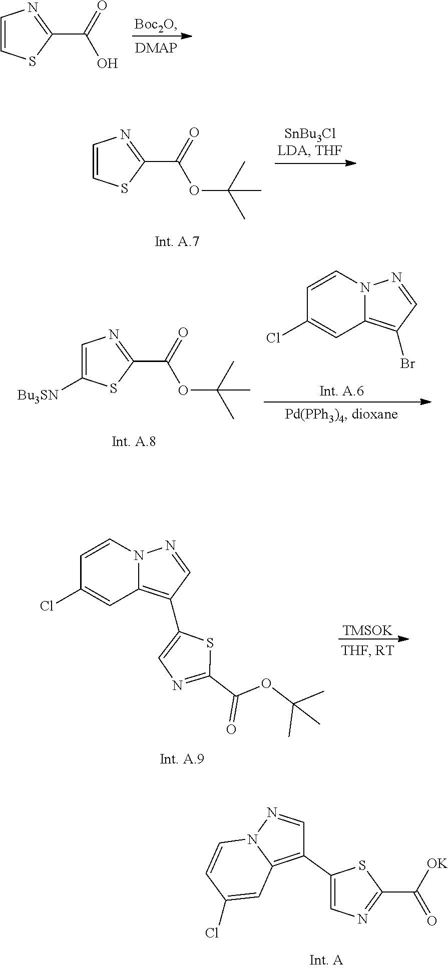

Synthesis of Int. A.7. To a solution of thiazole-2-carboxylic acid (50 g, 387 mmol, 1 eq) and di-tert-butyl pyrocarbonate (168.7 g, 774 mmol, 2 eq) in dichloromethane (800 mL) was added triethylamine (117.2 g, 1161 mmol, 3 eq) and DMAP (47.6 g, 387 mmol, 1 eq). The resulting solution was stirred for 16 h at room temperature. The mixture was diluted with water and extracted with dichloromethane. The combined organic layer was washed with brine, dried over sodium sulfate and concentrated under reduced pressure. The residue was purified by flash column chromatography on silica gel (eluted with 5% ethyl acetate in petroleum ether) to afford tert-butyl thiazole-2-carboxylate (Int. A.7, 59 g, 82%) as a yellow oil. MS (ES): m/z 186 [M+H]⁺.

Synthesis of Int. A.8. A stirred mixture of Int. A.7 (5 g, 27 mmol, 1 eq) in tetrahydrofuran (400 mL) was degassed three times with nitrogen and cooled to −80° C. Lithium diisopropylamide (1N, 40.5 mL, 40.5 mmol, 1.5 eq) was added and the resulting mixture was stirred for 0.5 h at −80° C. under nitrogen atmosphere. To the above mixture was added tributylchlorostannane (8.79 g, 27 mmol, 1 eq) in tetrahydrofuran (50 mL) at −80° C. under nitrogen atmosphere. The resulting mixture was additional for 1 h at −80° C. under nitrogen atmosphere. The reaction was quenched with saturated aqueous ammonium chloride solution at 0° C. The resulting mixture was extracted with ethyl acetate, the combined organic layer was washed with brine, dried over sodium sulfate and concentrated under reduced pressure. The residue was purified by flash column chromatography on silica gel (eluted with 2% ethyl acetate in petroleum ether) to afford tert-butyl 5-(tributylstannyl)thiazole-2-carboxylate (Int. A.8, 2.54 g, 20%) as a yellow oil. MS (ES): m/z 474 [M+H]⁺.

Synthesis of Int. A.9. To a solution of Int. A.6 (6 g, 21.6 mmol, 1 eq.) and Int. A.8 (10.2 g, 21.6 mmol, 1 eq) in dioxane (500 mL) was added Pd(PPh₃)₄ (242 mg, 0.21 mmol, 0.01 eq). The resulting solution was degassed three times with nitrogen and stirred for 48 h at 100° C. The mixture was concentrated under reduced pressure. The residue was purified by flash column chromatography on silica gel (eluted with 20% ethyl acetate in petroleum ether) to afford tert-butyl 5-(5-chloropyrazolo[1,5-a]pyridin-3-yl)thiazole-2-carboxylate (Int. A.9, 2.32 g, 31%) as a yellow solid. MS (ES): m/z 336 [M+H]⁺.

Synthesis of Int. A. To a stirred solution of Int. A.9 (1 g, 2.9 mmol, 1 eq) in tetrahydrofuran (20 mL) was added potassium trimethylsilanolate (742 mg, 5.8 mmol, 2 eq). The resulting mixture was stirred for 1 h at room temperature. The resulting mixture was concentrated under reduced pressure to obtain the crude product potassium 5-(5-chloropyrazolo[1,5-a]pyridin-3-yl)thiazole-2-carboxylate (Int. A, 1.2 g), which was used in subsequent steps without further purification. MS (ES): m/z 280 [M+H]⁺.

### Synthesis of (S)-5-(5-chloropyrazolo[1,5-a]pyridin-3-yl)-N-(1-(4-(cyclopropanesulfonamido)pyridin-2-yl)-3-methoxypropyl)thiazole-2-carboxamide, I-1 and (R)-5-(5-chloropyrazolo[1,5-a]pyridin-3-yl)-N-(1-(4-(cyclopropanesulfonamido)pyridin-2-yl)-3-methoxypropyl)thiazole-2-carboxamide, I-2

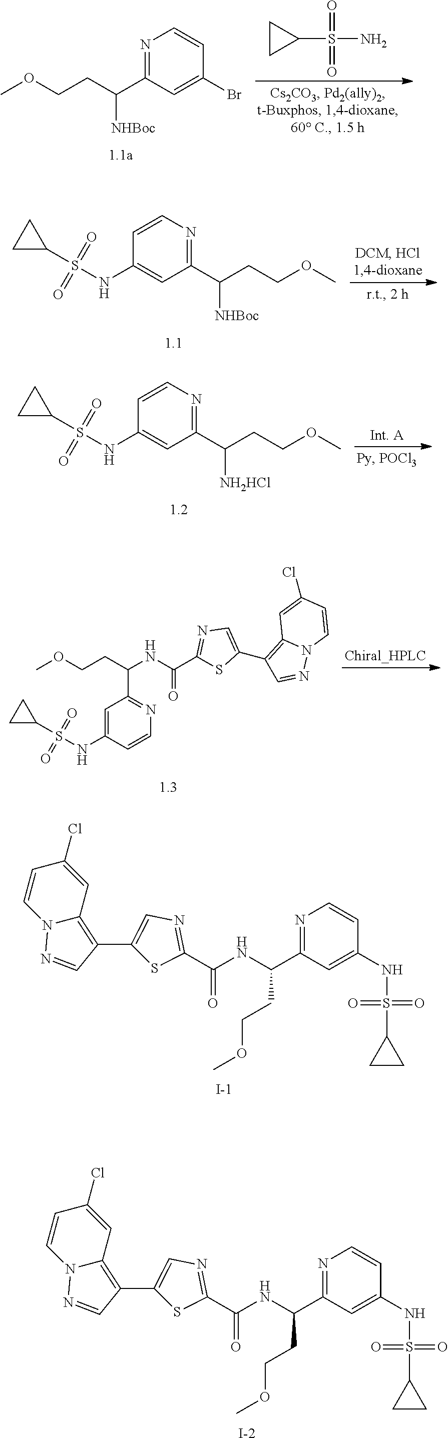

Synthesis of 1.1. To a stirred mixture of 1.1a (The synthesis of this intermediate is found in US patent application U.S. Ser. No. 17/452,011, filed on Oct. 22, 2021, the disclosure of which is hereby incorporated by reference) (300 mg, 0.87 mmol, 1 eq) and cyclopropanesulfonamide (210 mg, 1.74 mmol, 2 eq) in 1,4-dioxane (10 mL) were added cesium carbonate (845 mg, 2.61 mmol, 3 eq), t-BuXPhos (112 mg, 0.26 mmol, 0.3 eq) and Pd₂(ally)₂Cl₂ (51 mg, 0.13 mmol, 0.15 eq). The resulting solution was degassed three times with nitrogen and stirred for 1.5 h at 60° C. The mixture was allowed to cool down to room temperature. The resulting mixture was concentrated under reduced pressure and purified by reverse flash chromatography (C18 Column; Mobile Phase, water (0.1% NH₄HCO₃) and ACN (5% ACN up to 100% in 20 min); UV detection at 254/220 nm). The resulting mixture was concentrated under reduced pressure to obtain tert-butyl (1-(4-(cyclopropanesulfonamido)pyridin-2-yl)-3-methoxypropyl)carbamate (1.1, 170 mg, 50%) as a yellow solid. MS (ES): m/z 386 [M+H]⁺.

Synthesis of 1.2. To a stirred mixture of 1.1 (200 mg, 0.51 mmol, 1 eq) in dichloromethane (5 mL) was added hydrochloric acid in 1,4-dioxane (4N, 5 mL) at room temperature. The resulting mixture was stirred for 1.5 h at room temperature. The resulting mixture was concentrated under reduced pressure to afford the crude product N-(2-(1-amino-3-methoxypropyl)pyridin-4-yl)cyclopropanesulfonamide hydrochloride (1.2, 150 mg), which was used in the next step directly without further purification, MS (ES): m/z 286 [M+H]⁺.

Synthesis of 1.3. To a stirred mixture of 1.2 (150 mg, 0.47 mmol, 1.0 eq.) and Int. A (180 mg, 0.64 mmol, 1.4 eq.) in pyridine (Py) (7 mL) was added phosphoryl trichloride (0.1 mL, 1.08 mmol, 2.3 eq.) at 0° C. The resulting mixture was stirred for 1 h at room temperature. The residue was concentrated under reduced pressure and purified by reverse flash chromatography (C18 Column; Mobile Phase, water (0.1% FA) and ACN (5% ACN up to 100% in 20 min); UV detection at 254/220 nm). The resulting mixture was concentrated under reduced pressure. The crude product was purified by Prep-HPLC (Xbridge Prep C18 OBD Column; Mobile Phase, water (10 mmol/L NH₄HCO₃) and ACN (35% ACN up to 50% in 5.5 min); UV detection at 254/220 nm). The product-containing fractions were combined, evaporated partially in vacuum and lyophilized overnight to afford 5-(5-chloropyrazolo[1,5-a]pyridin-3-yl)-N-(1-(4-(cyclopropanesulfonamido)pyridin-2-yl)-3-methoxypropyl) thiazole-2-carboxamide (1.3, 40 mg, 16%) as a white solid. MS (ES): m/z 547 [M+H]⁺.

Synthesis of I-1 and I-2. 1.3 (40 mg, 0.07 mmol) was purified by Chiral-Prep-HPLC (CHIRALPAK IG, 2*25 cm, 5 μm; Mobile Phase A: Hex:DCM=3:1 (0.5% 2M NH₃-MeOH), Mobile Phase B: EtOH; Wavelength: 220/254 nm). The product-containing fractions were combined, evaporated partially in vacuum and lyophilized overnight to afford (S)-5-(5-chloropyrazolo[1,5-a]pyridin-3-yl)-N-(1-(4-(cyclopropanesulfonamido)pyridin-2-yl)-3-methoxypropyl)thiazole-2-carboxamide (1^{st} eluting peak, I-1, 15 mg, 38%) as a white solid and (R)-5-(5-chloropyrazolo[1,5-a]pyridin-3-yl)-N-(1-(4-(cyclopropanesulfonamido)pyridin-2-yl)-3-methoxypropyl)thiazole-2-carboxamide, (2^{nd} eluting peak, I-2, 14 mg, 34%) as a white solid.

I-1: MS (ES): m/z 547 [M+H]⁺; ¹H NMR (400 MHz, Methanol-d₄) δ 8.62 (dd, J=7.4, 0.8 Hz, 1H), 8.38 (s, 1H), 8.29 (d, J=5.5 Hz, 1H), 8.22 (s, 1H), 7.96 (dd, J=2.3, 0.8 Hz, 1H), 7.27 (d, J=2.3 Hz, 1H), 7.12 (dd, J=5.9, 2.3 Hz, 1H), 7.02 (dd, J=7.4, 2.2 Hz, 1H), 5.25 (dd, J=7.8, 5.7 Hz, 1H), 3.49-3.45 (m, 2H), 3.36 (s, 3H), 2.72-2.68 (m, 1H), 2.26-2.21 (m, 2H), 1.14-1.12 (m, 2H), 1.06-0.92 (m, 2H).

I-2: MS (ES): m/z 547 [M+H]⁺; ¹H NMR (400 MHz, Methanol-d₄) δ 8.62 (dd, J=7.4, 0.8 Hz, 1H), 8.38 (s, 1H), 8.28 (d, J=5.9 Hz, 1H), 8.21 (s, 1H), 7.96 (dd, J=2.3, 0.8 Hz, 1H), 7.27 (d, J=2.3 Hz, 1H), 7.12 (dd, J=5.9, 2.3 Hz, 1H), 7.02 (dd, J=7.4, 2.2 Hz, 1H), 5.25 (dd, J=7.8, 5.7 Hz, 1H), 3.49-3.45 (m, 2H), 3.36 (s, 3H), 2.72-2.68 (m, 1H), 2.26-2.21 (m, 2H), 1.14-1.10 (m, 2H), 1.01-0.95 (m, 2H).

### Example 2: Synthesis of (R)-5-(5-chloropyrazolo[1,5-a]pyridin-3-yl)-N-(1-(4-(cyclopropanesulfonamido)pyridin-2-yl)-2-methoxyethyl)thiazole-2-carboxamide, I-3 and (S)-5-(5-chloropyrazolo[1,5-a]pyridin-3-yl)-N-(1-(4-(cyclopropanesulfonamido)pyridin-2-yl)-2-methoxyethyl)thiazole-2-carboxamide, I-4

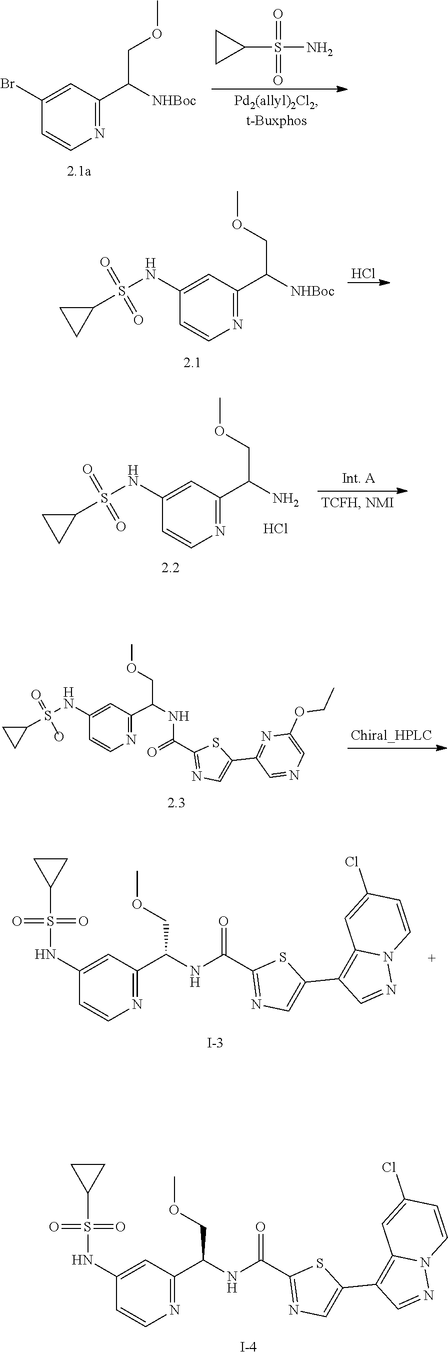

Synthesis of 2.1. To a stirred mixture of 2.1a (The synthesis of this intermediate is found in US patent application U.S. Ser. No. 17/452,011, filed on Oct. 22, 2021, the disclosure of which is hereby incorporated by reference) (750 mg, 2.27 mmol, 1 eq) and cyclopropanesulfonamide (548 mg, 4.54 mmol, 2 eq) in 1,4-dioxane (10 mL) were added cesium carbonate (2.2 g, 6.81 mmol, 3 eq), t-BuXPhos (292 mg, 0.98 mmol, 0.3 eq) and Pd₂(ally)₂Cl₂ (133 mg, 0.34 mmol, 0.15 eq). The resulting solution was degassed three times with nitrogen and stirred for 1.5 h at 60° C. The mixture was allowed to cool down to room temperature. The resulting mixture was concentrated under reduced pressure and purified by reverse flash chromatography (C18 Column; Mobile Phase, water (0.1% NH₄HCO₃) and ACN (5% ACN up to 100% in 20 min); UV detection at 254/220 nm). The resulting mixture was concentrated under reduced pressure to obtain tert-butyl (1-(4-(cyclopropanesulfonamido)pyridin-2-yl)-2-methoxyethyl)carbamate (2.1, 680 mg, 80%) as a yellow solid. MS (ES): m/z 372 [M+H]⁺.

Synthesis of 2.2. To a stirred mixture of 2.1 (680 mg, 1.83 mmol, 1 eq) in dichloromethane (10 mL) was added hydrochloric acid in 1,4-dioxane (4N, 10 mL) at room temperature. The resulting mixture was stirred for 1.5 h at room temperature. The resulting mixture was concentrated under reduced pressure to afford the crude product N-(2-(1-amino-2-methoxyethyl)pyridin-4-yl)cyclopropanesulfonamide hydrochloride (2.2, 560 mg), which was used in the next step directly without further purification. MS (ES): m/z 272 [M+H]⁺.

Synthesis of 2.3. To a stirred mixture of 2.2 (500 mg, 1.62 mmol, 1.0 eq.) and Int. A (300 mg, 1.07 eq, 0.66 eq.) in acetonitrile (10 mL) was added chloro-N,N,N′,N′-tetramethylformamidinium hexafluorophosphate (TCFH) (2.2 g, 16.2 mmol, 10 eq.) and 1-methylimidazole (NMI) (1.3 g, 15.8 mmol, 9.8 eq.) at 0° C. The resulting mixture was stirred for 1 h at room temperature. The residue was concentrated under reduced pressure and purified by reverse flash chromatography (C18 Column; Mobile Phase, water (0.1% FA) and ACN (5% ACN up to 100% in 40 min); UV detection at 254/220 nm). The product-containing fractions were combined, evaporated partially in vacuum and lyophilized overnight to afford N-(1-(4-(cyclopropanesulfonamido)pyridin-2-yl)-2-methoxyethyl)-5-(6-ethoxypyrazin-2-yl)thiazole-2-carboxamide (2.3, 130 mg, 16%) as a white solid. MS (ES): m/z 505 [M+H]⁺.

Synthesis of I-3 and I-4. 2.3 (130 mg, 0.25 mmol) was purified by Chiral-Prep-HPLC (CHIRALPAK IA, 2*25 cm, 20 μm; Mobile Phase A: EtOH-HPLC, Mobile Phase B: Hex:DCM=3:1 (0.1% formic acid (FA)); Wavelength: 220/254 nm). The product-containing fractions were combined, evaporated partially in vacuum and lyophilized overnight to afford (R)-5-(5-chloropyrazolo[1,5-a]pyridin-3-yl)-N-(1-(4-(cyclopropanesulfonamido)pyridin-2-yl)-2-methoxyethyl)thiazole-2-carboxamide (1^{st} eluting peak, I-3, 28 mg, 21%) as a white solid and (S)-5-(5-chloropyrazolo[1,5-a]pyridin-3-yl)-N-(1-(4-(cyclopropanesulfonamido)pyridin-2-yl)-2-methoxyethyl)thiazole-2-carboxamide (2^{nd} eluting peak, 1-4, 21 mg, 16%) as a white solid.

I-3: MS (ES): m/z 505 [M+H]⁺; ¹H NMR (300 MHz, Methanol-d₄) δ 8.63 (dd, J=7.4, 0.8 Hz, 1H), 8.39 (s, 1H), 8.31 (s, 1H), 8.23 (s, 1H), 7.97 (dd, J=2.3, 0.8 Hz, 1H), 7.29 (d, J=2.2 Hz, 1H), 7.16 (dd, J=5.9, 2.3 Hz, 1H), 7.03 (dd, J=7.4, 2.2 Hz, 1H), 5.28 (t, J=5.6 Hz, 1H), 3.90-3.81 (m, 2H), 3.39 (s, 3H), 2.73-2.68 (m, 1H), 1.14-1.11 (m, 2H), 1.01-0.97 (m, 2H).

I-4: MS (ES): m/z 505 [M+H]⁺; ¹H NMR (400 MHz, Methanol-d₄) δ 8.62 (dd, J=7.4, 0.8 Hz, 1H), 8.39 (s, 1H), 8.31 (s, 1H), 8.23 (s, 1H), 7.97 (dd, J=2.3, 0.8 Hz, 1H), 7.29 (d, J=2.2 Hz, 1H), 7.16 (dd, J=5.9, 2.3 Hz, 1H), 7.03 (dd, J=7.4, 2.2 Hz, 1H), 5.28 (t, J=5.6 Hz, 1H), 3.89-3.80 (m, 2H), 3.39 (s, 3H), 2.73-2.68 (m, 1H), 1.16-1.11 (m, 2H), 1.03-0.98 (m, 2H).

### Example 3: Synthesis of N-((6-(cyclopropanesulfonamido)pyrimidin-4-yl)methyl)-5-(6-ethoxypyrazin-2-yl)thiazole-2-carboxamide I-5

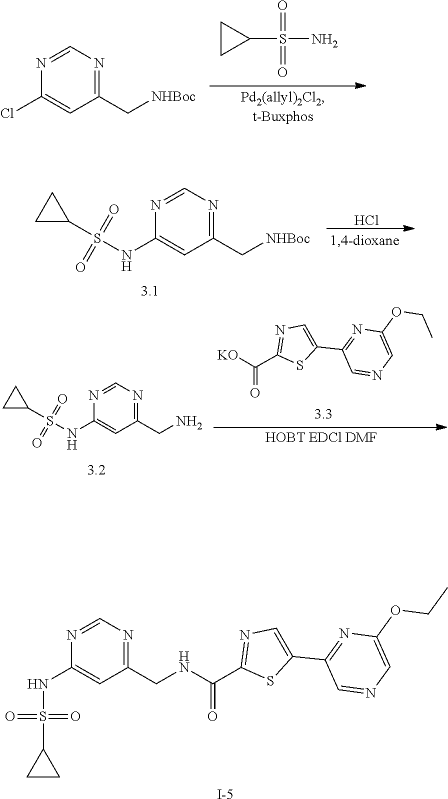

Synthesis of 3.1. To a stirred mixture of tert-butyl ((6-chloropyrimidin-4-yl)methyl)carbamate (200 mg, 0.82 mmol, 1 eq) and cyclopropanesulfonamide (198 mg, 1.64 mmol, 2 eq) in 1,4-dioxane (8 mL) were added cesium carbonate (797 mg, 2.46 mmol, 3 eq), t-BuXPhos (106 mg, 0.24 mmol, 0.3 eq) and Pd₂(ally)₂Cl₂ (48 mg, 0.12 mmol, 0.15 eq). The resulting solution was degassed three times with nitrogen and stirred for 1.5 h at 60° C. The mixture was allowed to cool down to room temperature. The resulting mixture was concentrated under reduced pressure and purified by reverse flash chromatography (C18 Column; Mobile Phase, water (0.10% NH₄HCO₃) and ACN (5% ACN up to 100% in 20 min); UV detection at 254/220 nm). The resulting mixture was concentrated under reduced pressure to obtain tert-butyl ((6-(cyclopropanesulfonamido)pyrimidin-4-yl)methyl)carbamate (3.1, 190 mg, 70%) as a brown solid. MS (ES): m/z 329 [M+H]⁺.

Synthesis of 3.2. To a stirred mixture of 3.1 (150 mg, 0.45 mmol, 1 eq) in dichloromethane (5 mL) was added hydrochloric acid in 1,4-dioxane (4N, 5 mL) at room temperature. The resulting mixture was stirred for 1.5 h at room temperature. The resulting mixture was concentrated under reduced pressure to obtain the crude product N-(6-(aminomethyl)pyrimidin-4-yl)cyclopropanesulfonamide hydrochloride (3.2, 100 mg), which was used in the next step directly without further purification, MS (ES): m/z 229 [M+H]⁺.

Synthesis of I-5. To a stirred mixture of 3.2 (100 mg) and 3.3 (The synthesis of this intermediate is found in US patent application U.S. Ser. No. 17/452,011, filed on Oct. 22, 2021, the disclosure of which is hereby incorporated by reference) (150 mg) in N,N-dimethylformamide (5 mL) was added 1-hydroxybenzotriazole (135 mg, 1 mmol) and 1-ethyl-3-(3-dimethylaminopropyl)carbodiimide hydrochloride (281 mg, 1.46 mmol). The resulting mixture was stirred for 1 h at room temperature. The mixture was diluted with water and extracted with ethyl acetate. The residue was concentrated under reduced pressure and purified by reverse flash chromatography (C18 Column; Mobile Phase, water (0.1% FA) and ACN (5% ACN up to 100% in 20 min); UV detection at 254/220 nm). The resulting mixture was concentrated under reduced pressure. The crude product was purified by Prep-HPLC (XBridge Prep C18 OBD Column; Mobile Phase, water (10 mmol/L NH₄HCO₃) and ACN (18% ACN up to 28% in 9 min); UV detection at 254/220 nm). The product-containing fractions were combined, evaporated partially in vacuum and lyophilized overnight to afford N-((6-(cyclopropanesulfonamido)pyrimidin-4-yl)methyl)-5-(6-ethoxypyrazin-2-yl)thiazole-2-carboxamide (I-5, 22 mg, 11% over 2 steps) as a white solid. MS (ES): m/z 462 [M+H]⁺; ¹H NMR (400 MHz, DMSO-d₆) δ 9.47 (d, J=5.7 Hz, 1H), 8.93 (s, 1H), 8.82 (s, 1H), 8.58 (s, 1H), 8.28 (s, 1H), 6.69 (s, 1H), 4.60-4.37 (m, 4H), 2.99-2.78 (m, 1H), 1.40 (t, J=7.0 Hz, 3H), 0.93-0.84 (m, 4H).

### Example 4: Synthesis of (S)—N-(1-(4-(cyclopropanesulfonamido)pyridin-2-yl)-3-(piperidin-1-yl)propyl)-4-(6-ethoxypyrazin-2-yl)-2,3-difluorobenzamide, I-6 and (R)—N-(1-(4-(cyclopropanesulfonamido)pyridin-2-yl)-3-(piperidin-1-yl)propyl)-4-(6-ethoxypyrazin-2-yl)-2,3-difluorobenzamide, I-7

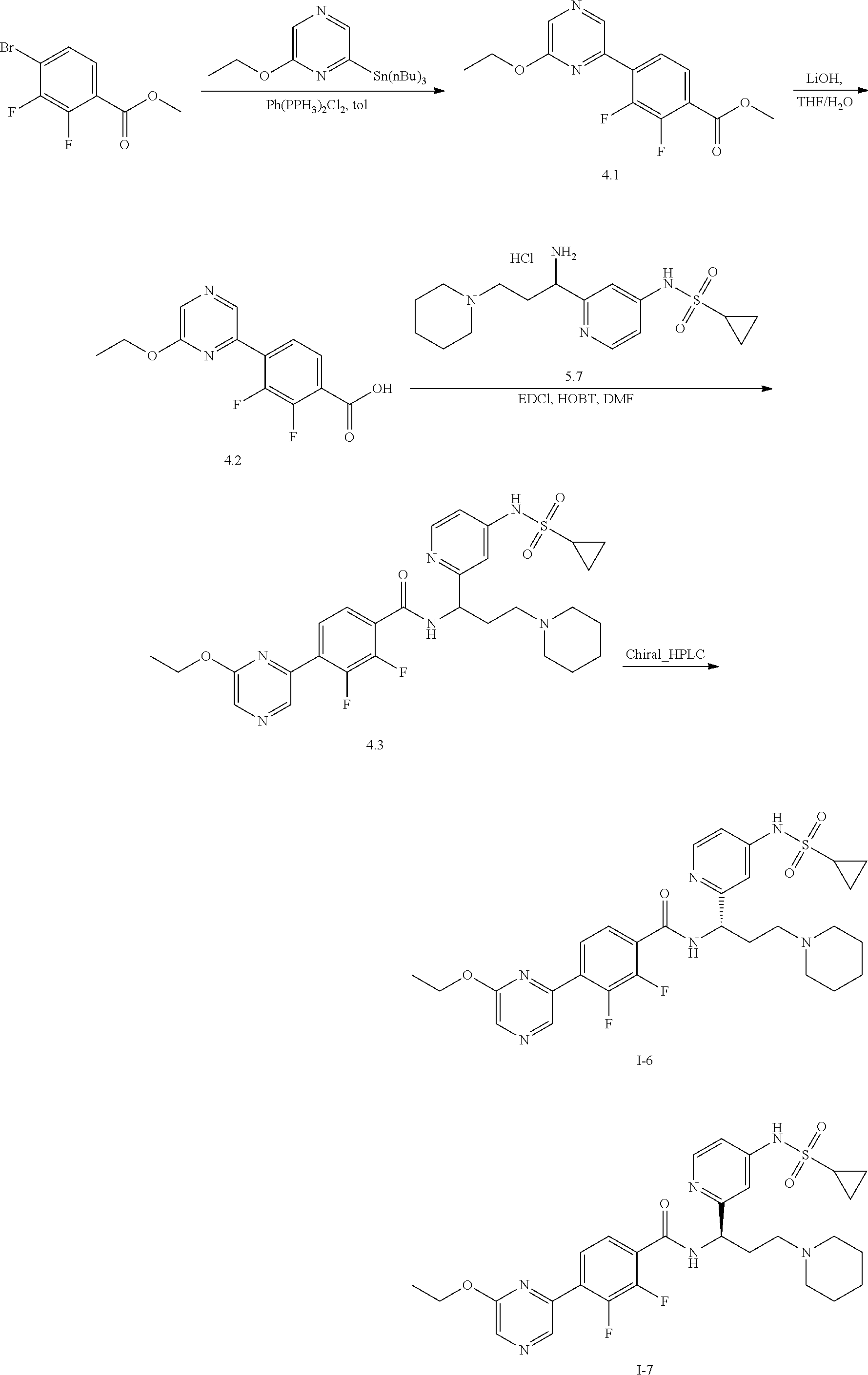

Synthesis of 4.1. To a solution of methyl 4-bromo-2,3-difluorobenzoate (1 g, 4 mmol, 1 eq) and 2-ethoxy-6-(tributylstannyl)pyrazine (1.65 g, 4 mmol, 1 eq) in toluene (15 mL) was added Pd(PPh₃)₂Cl₂ (280 mg, 0.4 mmol, 0.1 eq). The resulting solution was degassed three times with nitrogen and stirred for 16 h at 100° C. The mixture was cooled to room temperature and concentrated under reduced pressure. The residue was purified by flash column chromatography on silica gel (eluted with 20% ethyl acetate in petroleum ether) to afford methyl 4-(6-ethoxypyrazin-2-yl)-2,3-difluorobenzoate (4.1, 1 g, 87%) as a yellow solid. MS (ES): m/z 295 [M+H]⁺.

Synthesis of 4.2. To a stirred solution of 4.1 (500 mg, 1.69 mmol, 1 eq) in tetrahydrofuran (5 mL) was added lithium hydroxide (122 mg, 5.08 mmol, 3 eq) in water (1 mL). The resulting solution was stirred for 3 h at room temperature. The residue was diluted with water. The pH value of the solution was adjusted to 3 with 1N aqueous hydrochloric acid. The resulting mixture was extracted with ethyl acetate. The combined organic layers were washed with brine, dried over anhydrous sodium sulfate and concentrated under reduced pressure to afford the crude product 4-(6-ethoxypyrazin-2-yl)-2,3-difluorobenzoic acid (4.2, 450 mg), which was used in the next step directly without further purification. MS (ES): m/z 281 [M+H]⁺.

Synthesis of 4.3. To a solution of 4.2 (410 mg) and 5.7 (as prepared infra) (600 mg) in N,N-dimethylformamide (5 mL) was added 1-hydroxybenzotriazole (384 mg, 2.84 mmol) and 1-ethyl-3-(3-dimethylaminopropyl)carbodiimide hydrochloride (816 mg, 4.24 mmol). The resulting solution was stirred for 2 h at room temperature. The mixture was diluted with water and extracted with ethyl acetate. The combined organic layer was washed with brine, dried over sodium sulfate and concentrated under reduced pressure. The crude product purified by Prep-HPLC with the following conditions (Column: XBridge Prep OBD C18 Column, 30*150, 5 μm; Mobile Phase, water (10 mmol/L NH₄HCO₃) and ACN (24% ACN up to 35% in 10 min); UV detection at 254/220 nm). The product-containing fractions were combined, evaporated partially in vacuum and lyophilized overnight to afford N-(1-(4-(cyclopropanesulfonamido)pyridin-2-yl)-3-(piperidin-1-yl)propyl)-4-(6-ethoxypyrazin-2-yl)-2,3-difluorobenzamide (4.3, 160 mg) as a white solid. MS (ES): m/z 601 [M+H]⁺.

Synthesis of I-6 and I-7. 4.3 (160 mg, 0.26 mmol) was purified by Chiral-Prep-HPLC (CHIRALPAK IG, 2*25 cm, 5 μm; Mobile Phase A: Hex:DCM=3:1 (0.5% 2M NH₃-MeOH)-HPLC, Mobile Phase B: EtOH-HPLC; Wavelength: 220/254 nm). The product-containing fractions were combined, evaporated partially in vacuum and lyophilized overnight to afford (S)—N-(1-(4-(cyclopropanesulfonamido)pyridin-2-yl)-3-(piperidin-1-yl)propyl)-4-(6-ethoxypyrazin-2-yl)-2,3-difluorobenzamide (1^{st} eluting peak, I-6, 39 mg, 24%) as a white solid and (R)—N-(1-(4-(cyclopropanesulfonamido)pyridin-2-yl)-3-(piperidin-1-yl)propyl)-4-(6-ethoxypyrazin-2-yl)-2,3-difluorobenzamide, (2^{nd} eluting peak, I-7, 37 mg, 23%) as a white solid.

I-6: MS (ES): m/z 601 [M+H]⁺; ¹H NMR (400 MHz, Methanol-d₄) δ 8.63 (d, J=2.3 Hz, 1H), 8.25-8.22 (m, 2H), 7.96-7.95 (m, 1H), 7.67-7.66 (m, 1H), 7.31 (d, J=2.2 Hz, 1H), 7.06 (dd, J=5.9, 2.2 Hz, 1H), 5.19-5.15 (m, 1H), 4.52 (q, J=7.0 Hz, 2H), 2.71-2.52 (m, 7H), 2.35-2.22 (m, 1H), 2.15-2.09 (m, 1H), 1.70-1.58 (m, 4H), 1.52-1.47 (m, 2H), 1.45 (t, J=7.1 Hz, 3H), 1.13-1.11 (m, 2H), 0.99-0.96 (m, 2H).

I-7: MS (ES): m/z 601 [M+H]⁺; ¹H NMR (400 MHz, Methanol-d₄) δ 8.63 (d, J 2.3 Hz, 1H), 8.25-8.22 (m, 2H), 7.96-7.95 (m, 1H), 7.66-7.65 (m, 1H), 7.31 (d, J=2.2 Hz, 1H), 7.06 (dd, J=5.9, 2.2 Hz, 1H), 5.19-5.17 (m, 1H), 4.52 (q, J=7.0 Hz, 2H), 2.70-2.57 (m, 7H), 2.35-2.22 (m, 1H), 2.15-2.09 (m, 1H), 1.67-1.58 (m, 4H), 1.52-1.47 (m, 2H), 1.45 (t, J=7.1 Hz, 3H), 1.13-1.11 (m, 2H), 0.99-0.96 (m, 2H).

### Example 5: Preparation of Intermediate B (Int. B) and Compounds of the Disclosure

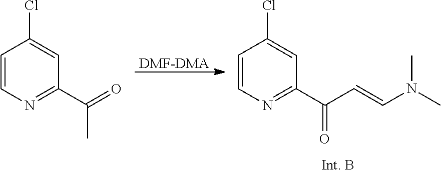

Synthesis of Int. B: A solution of 1-(4-chloropyridin-2-yl)ethanone (1 g, 6.43 mmol, 1 equiv) and DMA-DMF (0.22 g, 9.64 mmol, 1.5 equiv) in N,N-dimethylformamide (10 mL) was stirred for 2 h at 80° C. The reaction was diluted with water (30 mL) and extracted with ethyl acetate (3×25 mL). The combined organic layers were concentrated under reduced pressure to afford (E)-1-(4-chloropyridin-2-yl)-3-(dimethylamino)prop-2-en-1-one (Int. B, 1.2 g, 90%) as a colorless oil, which was used in the next step directly without further purification. MS (ES): m/z 211 [M+H]⁺.

### Synthesis of 5-(5-chloropyrazolo[1,5-a]pyridin-3-yl)-N-(1-(4-(cyclopropanesulfonamido)pyridin-2-yl)-3-(piperidin-1-yl)propyl)thiazole-2-carboxamide, 1-8

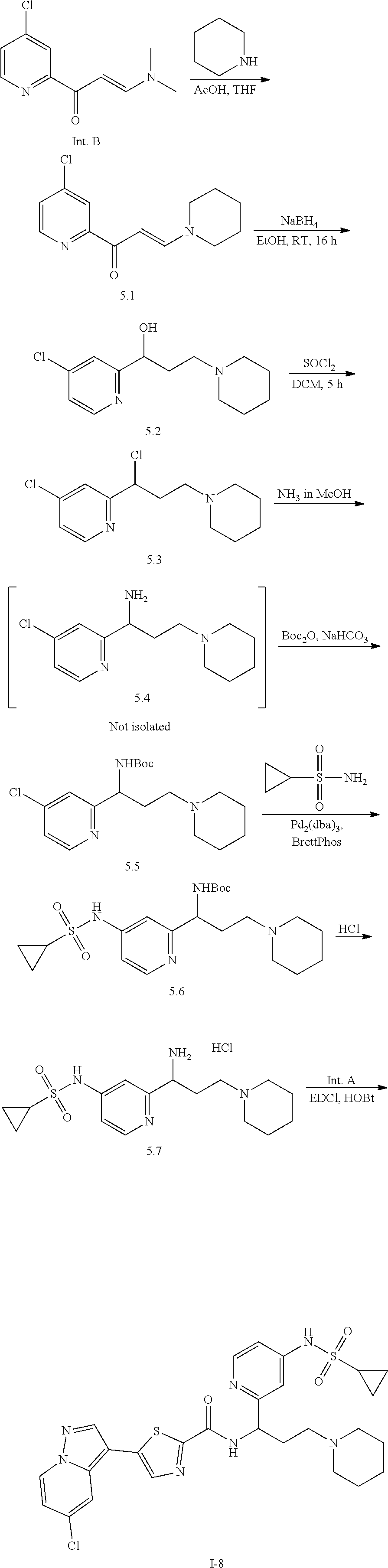

Synthesis of 5.1: To a stirred mixture of Int. B (3 g, 14.28 mmol, 1 equiv) and piperidine (3.64 g, 42.84 mmol, 3 equiv) in THF (30 mL) was added acetic acid (10 mL) dropwise at room temperature. The resulting mixture was stirred for 2 h at 60° C. The reaction mixture was concentrated under reduced pressure. The residue was neutralized to pH 10 with saturated aqueous sodium bicarbonate and extracted with ethyl acetate (3×30 mL). The combined organic layers were concentrated under reduced pressure. The residue was purified by flash column chromatography on C18 silica (eluted with 50% acetonitrile in water) to afford (E)-1-(4-chloropyridin-2-yl)-3-(piperidin-1-yl)prop-2-en-1-one (5.1, 2.5 g, 70%) as a yellow solid. MS (ES): m/z 251/253 [M+H]⁺.

Synthesis of 5.2. To a stirred solution of 5.1 (2.5 g, 10 mmol, 1 equiv) in ethanol (20 mL) was added sodium borohydride (3.78 g, 100 mmol, 10 equiv) in portions at 0° C. The resulting mixture was stirred for 16 h at room temperature. Methanol (5 mL) was added at 0° C., the reaction mixture was diluted with water (30 mL) and the products were extracted with dichloromethane (4×30 mL). The combined organic layers were concentrated under reduced pressure. The residue was purified by flash column chromatography on C18 silica (eluted with 43% acetonitrile in water) to afford 1-(4-chloropyridin-2-yl)-3-(piperidin-1-yl)propan-1-ol (5.2, 2.0 g, 78%) as an yellow oil. MS (ES): m/z 255/257 [M+H]⁺.

Synthesis of 5.3. To a stirred solution of 5.2 (2.0 g, 7.87 mmol, 1 equiv) in dichloromethane (10 mL) was added thionyl chloride (1.89 mL, 15.74 mmol, 2.2 equiv) dropwise at 0° C. The resulting mixture was stirred for 5 h at room temperature. The reaction was concentrated under reduced pressure to afford 4-chloro-2-(1-chloro-3-(piperidin-1-yl)propyl)pyridine (5.3, 2.2 g, crude) as a colorless oil, which was used in the next step directly without further purification. MS (ES): m/z 273/275 [M+H]⁺.

Synthesis of 5.5. A mixture of 5.3 (2.2 g, 8.05 mmol, 1 equiv) and sodium iodide (2.42 g, 16.1 mmol, 2 equiv) in ammonium methanol solution (2M, 25 mL) was stirred for 16 h at 40° C. The solid was removed by filtration and washed with methanol (10 mL). The filtrate was concentrated under reduced pressure. The residue (5.4) was dissolved with methanol (20 mL), followed by the addition of di-tert-butyl pyrocarbonate (3.51 g, 16.1 mmol, 2 equiv) and saturated sodium bicarbonate aqueous solution (20 mL). The resulting mixture was stirred for 4 h at room temperature. The reaction was extracted with dichloromethane (4×10 mL). The combined organic layers were concentrated under reduced pressure. The residue was purified by flash column chromatography on C18 silica (eluted with 73% acetonitrile in water) to afford tert-butyl (1-(4-chloropyridin-2-yl)-3-(piperidin-1-yl)propyl)carbamate (5.5, 800 mg, 29% from 5.2) as a white solid. MS (ES): m/z 354/356 [M+H]⁺.

Synthesis of 5.6. To a stirred mixture of 5.5 (800 mg, 2.26 mmol, 1 equiv), cyclopropanesulfonamide (548 mg, 4.53 mmol, 2 equiv) and cesium carbonate (2.2 g, 6.79 mmol, 3 equiv) in 1,4-dioxane (20 mL) were added Pd₂(dba)₃ (227 mg, 0.22 mmol, 0.1 equiv) and BrettPhos (241 mg, 0.45 mmol, 0.2 equiv) in portions at room temperature. The resulting solution was degassed three times with nitrogen and stirred for 2 h at 100° C. The mixture was cooled to room temperature and concentrated under reduced pressure. The residue was purified by flash column chromatography on C18 silica (eluted with 38% acetonitrile in water) to afford tert-butyl (1-(4-(cyclopropanesulfonamido)pyridin-2-yl)-3-(piperidin-1-yl)propyl)carbamate (5.6, 420 mg, 42%) as a yellow solid. MS (ES): m/z 439 [M+H]⁺.

Synthesis of 5.7. To a mixture of 5.6 (150 mg, 0.34 mmol, 1 equiv) in dichloromethane (4 mL) was added HCl in 1,4-dioxane (4M, 4 mL) dropwise at room temperature. The resulting mixture was stirred at room temperature for 1 h. The reaction mixture was concentrated under reduced pressure to afford N-(2-(1-amino-3-(piperidin-1-yl)propyl)pyridin-4-yl)cyclopropanesulfonamide hydrochloride (5.7, 140 mg) as a yellow solid, which was used in the next step directly without further purification. MS (ES): m/z 339 [M+H]⁺.

Synthesis of I-8. To a solution of 5.7 (100 mg) and Int. A (90 mg) in N,N-dimethylformamide (5 mL) was added 1-hydroxybenzotriazole (80 mg, 0.6 mmol) and 1-ethyl-3-(3-dimethylaminopropyl)carbodiimide hydrochloride (170 mg, 0.88 mmol). The resulting solution was stirred for 2 h at room temperature. The residue was purified by flash column chromatography on C18 silica (eluted with 44% acetonitrile in water) to afford the crude product. The crude product was further purified by Prep-HPLC (XBridge Prep OBD C18 Column, 30*150, 5 μm; Mobile Phase, water (10 mmol/L NH₄HCO₃) and ACN (28% ACN up to 32% in 15 min); UV detection at 254/220 nm). The product-containing fractions were combined, evaporated partially in vacuum and then lyophilized overnight to afford 5-(5-chloropyrazolo[1,5-a]pyridin-3-yl)-N-(1-(4-(cyclopropanesulfonamido) pyridin-2-yl)-3-(piperidin-1-yl)propyl)thiazole-2-carboxamide (I-8, 18 mg) as a white solid. MS (ES): m/z 600 [M+H]⁺; ¹H NMR (300 MHz, Methanol-d₄) δ 8.59 (d, J=7.4 Hz, 1H), 8.34 (s, 1H), 8.22-8.17 (m, 2H), 7.89 (d, J=1.5 Hz, 1H), 7.22 (d, J=2.1 Hz, 1H), 7.06 (dd, J=5.9, 2.2 Hz, 1H), 7.02 (dd, J=5.9, 2.2 Hz, 1H), 5.18-5.14 (m, 1H), 2.66-2.52 (m, 7H), 2.32-2.22 (m, 1H), 2.16-2.08 (m, 1H), 1.76-1.70 (m, 4H), 1.54-1.46 (m, 2H), 1.09-1.07 (m, 2H), 0.95-0.89 (m, 2H).

### Example 6: Synthesis of (S)-5-(5-chloropyrazolo[1,5-a]pyridin-3-yl)-N-(1-(4-(cyclopropanesulfonamido)pyridin-2-yl)-3-(piperidin-1-yl)propyl)thiazole-2-carboxamide, I-9 and (R)-5-(5-chloropyrazolo[1,5-a]pyridin-3-yl)-N-(1-(4-(cyclopropanesulfonamido)pyridin-2-yl)-3-(piperidin-1-yl)propyl)thiazole-2-carboxamide, I-10

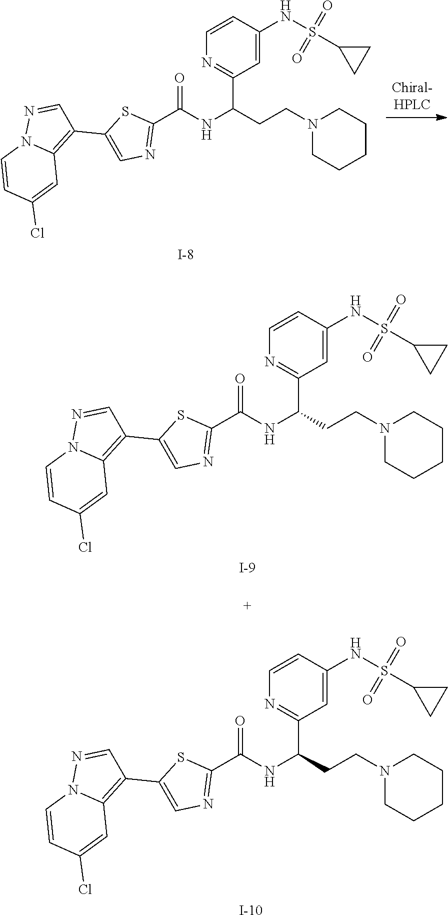

Synthesis of I-9 and I-10. I-8 (16 mg), as prepared supra, was purified by chiral HPLC (Column: CHIRALPAK IG, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 30% B to 30% B in 24 min; Wavelength: 220/254 nm) to afford (S)-5-(5-chloropyrazolo[1,5-a]pyridin-3-yl)-N-(1-(4-(cyclopropanesulfonamido) pyridine-2-yl)-3-(piperidin-1-yl)propyl)thiazole-2-carboxamide, (I-9, first eluting peak, 3.4 mg) and (R)-5-(5-chloropyrazolo[1,5-a]pyridin-3-yl)-N-(1-(4-(cyclopropanesulfonamido)pyridin-2-yl)-3-(piperidin-1-yl)propyl)thiazole-2-carboxamide, (I-10, second eluting peak, 4.2 mg) both as white solids.

I-9. MS (ES): m/z 600 [M+H]⁺; ¹H NMR (300 MHz, Methanol-d4) δ 8.60 (d, J=7.4 Hz, 1H), 8.37 (s, 1H), 8.24-8.18 (m, 2H), 7.93 (d, J=2.2 Hz, 1H), 7.24 (d, J=2.2 Hz, 1H), 7.06 (dd, J=5.9, 2.2 Hz, 1H), 7.02 (dd, J=5.9, 2.2 Hz, 1H), 5.19-5.15 (m, 1H), 2.67-2.55 (m, 7H), 2.32-2.22 (m, 1H), 2.16-2.08 (m, 1H), 1.78-1.68 (m, 4H), 1.58-1.46 (m, 2H), 1.10-1.08 (m, 2H), 0.96-0.91 (m, 2H).

I-10. MS (ES): m/z 600 [M+H]⁺; ¹H NMR (300 MHz, Methanol-d₄) δ 8.60 (d, J=7.4 Hz, 1H), 8.37 (s, 1H), 8.23-8.18 (m, 2H), 7.94 (d, J=2.2 Hz, 1H), 7.23 (d, J=2.2 Hz, 1H), 7.06 (dd, J=5.9, 2.2 Hz, 1H), 7.02 (dd, J=5.9, 2.2 Hz, 1H), 5.18-5.14 (m, 1H), 2.67-2.53 (m, 7H), 2.32-2.22 (m, 1H), 2.16-2.08 (m, 1H), 1.78-1.71 (m, 4H), 1.58-1.46 (m, 2H), 1.10-1.08 (m, 2H), 0.95-0.90 (m, 2H).

### Example 7: Synthesis of N-[(1R)-1-(4-cyclopropanesulfonamido-3-fluoropyridin-2-yl)-2-methoxyethyl]-5-(6-ethoxypyrazin-2-yl)-1,3-thiazole-2-carboxamide, I-11 and N-[(1S)-1-(4-cyclopropanesulfonamido-3-fluoropyridin-2-yl)-2-methoxyethyl]-5-(6-ethoxypyrazin-2-yl)-1,3-thiazole-2-carboxamide, I-12

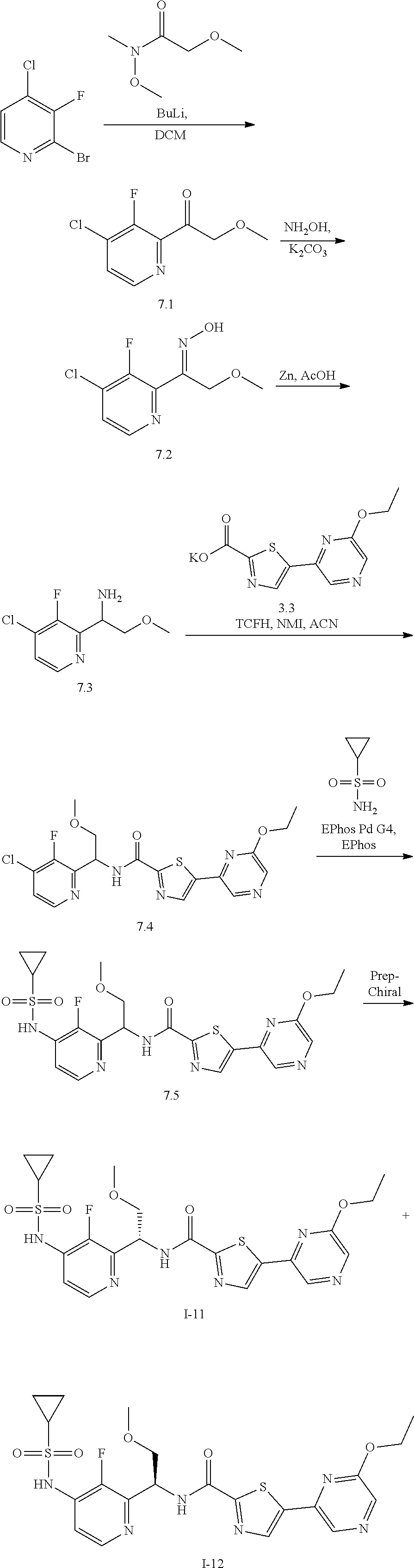

Synthesis of 7.1. To a solution of 2-bromo-4-chloro-3-fluoropyridine (4 g, 19.1 mmol, 1.5 eq) in DCM (15 mL) was added n-butyllithium solution (2.5 M in THF, 7.6 mL, 19.1 mmol, 1.5 eq) dropwise at −78° C. under N₂ atmosphere. The resulting mixture was stirred at −78° C. for 30 mins. Then a solution of N-2-dimethoxy-N-methylacetamide (1.7 g, 12.8 mmol, 1.0 eq) in DCM (5 mL) was added dropwise at −78° C. under nitrogen atmosphere. The resulting mixture was stirred at −78° C. for 1.5 h. The reaction was quenched with aqueous saturated ammonium chloride (20 mL) and the aqueous layer was extracted with ethyl acetate (3×15 mL). The combined organic layers were concentrated under vacuum The residue was purified by flash column chromatography on silica gel (eluted with 25% ethyl acetate in petro ether) to afford 1-(4-chloro-3-fluoropyridin-2-yl)-2-methoxyethanone (7.1, 700 mg, 27%) as a light yellow solid. MS (ES): m/z 204 [M+H]⁺.

Synthesis of 7.2. A solution of 7.1 (700 mg, 3.4 mmol, 1.0 eq), hydroxylamine hydrochloride (358 mg, 5.2 mmol, 1.5 eq) and K₂CO₃ (950 mg, 6.8 mmol, 2.0 eq) in MeOH (15 mL) was stirred for 2 h at room temperature. The reaction was diluted with methanol (30 mL) and filtered through Celite. The filtrate was concentrated under reduced pressure to afford (Z)—N-[1-(4-chloro-3-fluoropyridin-2-yl)-2-methoxyethylidene]hydroxylamine (7.2, 700 mg) as a yellow oil, which was used directly in the next step without further purification. MS (ES): m/z 219 [M+H]⁺.

Synthesis of 7.3. A mixture of 7.2 (700 mg) and zinc powder (1.0 g, 16.0 mmol) in MeOH (10 mL) and AcOH (1 mL) was stirred for 2 h at room temperature. The reaction was diluted with methanol (30 mL) and filtered through Celite. The filtrate was concentrated under reduced pressure to afford 1-(4-chloro-3-fluoropyridin-2-yl)-2-methoxyethanamine (7.3, 400 mg) as a light yellow oil, which was used directly in the next step without further purification. MS (ES): m/z 205 [M+H]⁺.

Synthesis of 7.4. A mixture of 7.3 (400 mg), 3.3 (491 mg), NMI (481 mg, 5.8 mmol) and TCFH (1.6 g, 5.8 mmol) in acetonitrile (10 mL) was stirred overnight at room temperature. The reaction was diluted with water (30 mL) and the organic products were extracted with ethyl acetate (3×15 mL). The combined organic layers were concentrated under reduced pressure. The residue was purified by flash column chromatography on silica gel (eluted with 50% ethyl acetate in petro ether) to afford N-[(1-(4-chloro-3-fluoropyridin-2-yl)-2-methoxyethyl]-5-(6-ethoxypyrazin-2-yl)-1,3-thiazole-2-carboxamide (7.4, 300 mg, 20% over 3 steps) as a light yellow oil. MS (ES): m/z 438 [M+H]⁺.

Synthesis of 7.5. A mixture of 7.4 (300 mg, 0.69 mmol, 1.0 eq), cyclopropanesulfonamide (249. mg, 2.05 mmol, 3.0 eq), EPhos Pd G4 (63 mg, 0.069 mmol, 0.1 eq), EPhos (73 mg, 0.137 mmol, 0.2 eq) and Cs₂CO₃ (670 mg, 2.01 mmol, 3 eq) in 1,4-dioxane (6 mL) was stirred for 3 h at 90° C. under nitrogen atmosphere. The reaction was diluted with methanol (30 mL) and filtered through Celite. The filtrate was concentrated under reduced pressure. The residue was purified by Prep-HPLC (XBridge Prep OBD C18 Column, 30*150 mm, 5 m; Mobile Phase A: water (0.10% FA), Mobile Phase B: ACN; Flow rate: 60 mL/min; Gradient: 33% B to 63% B in 10 min; Wavelength: 254 nm) to afford N-[1-(4-cyclopropanesulfonamido-3-fluoropyridin-2-yl)-2-methoxyethyl]-5-(6-ethoxypyrazin-2-yl)-1,3-thiazole-2-carboxamide (7.5, 40 mg, 11%) as a white solid. MS (ES): m/z 523 [M+H]⁺.

Synthesis of I-11 and I-12. 7.5 (40 mg) was purified by Prep-SFC (Column: Lux 5 μm Cellulose-4, 3*25 cm, 10 m; Mobile Phase A: CO₂, Mobile Phase B: MeOH:ACN=1:1 (0.1% 2M NH₃-MeOH); Flow rate: 100 mL/min; Gradient: isocratic 65% B; Wavelength: 220 nm) to afford N-[(1R)-1-(4-cyclopropanesulfonamido-3-fluoropyridin-2-yl)-2-methoxyethyl]-5-(6-ethoxypyrazin-2-yl)-1,3-thiazole-2-carboxamide, I-11, first eluting peak, 11 mg) and N-[(1S)-1-(4-cyclopropanesulfonamido-3-fluoropyridin-2-yl)-2-methoxyethyl]-5-(6-ethoxypyrazin-2-yl)-1,3-thiazole-2-carboxamide, I-12, second eluting peak, 8 mg) both as a white solid.

I-11. MS (ES): m/z 523 [M+H]⁺; ¹H NMR (300 MHz, Methanol-d₄) δ 8.69 (s, 1H), 8.57 (s, 1H), 8.21 (d, J=5.7 Hz, 1H), 8.14 (s, 1H), 7.61 (dd, J=6.6, 5.7 Hz, 1H), 5.72-5.68 (m, 1H), 4.47 (q, J=6.9 Hz, 2H), 3.87-3.75 (m, 2H), 3.37 (s, 3H), 2.83-2.74 (m, 1H), 1.45 (t, J=6.9 Hz, 3H), 1.21-1.15 (m, 2H), 1.06-1.01 (m, 2H).

I-12. MS (ES): m/z 523 [M+H]⁺; ¹H NMR (400 MHz, Methanol-d₄) δ 8.69 (s, 1H), 8.57 (s, 1H), 8.14 (s, 1H), 8.06 (d, J=5.6 Hz, 1H), 7.52-7.49 (m, 1H), 5.71-5.69 (m, 1H), 4.47 (q, J=7.2 Hz, 2H), 3.83-3.74 (m, 2H), 3.35 (s, 3H), 2.72-2.66 (m, 1H), 1.45 (t, J=7.2 Hz, 3H), 1.13-1.11 (m, 2H), 0.96-0.93 (m, 2H).

### General Method 1

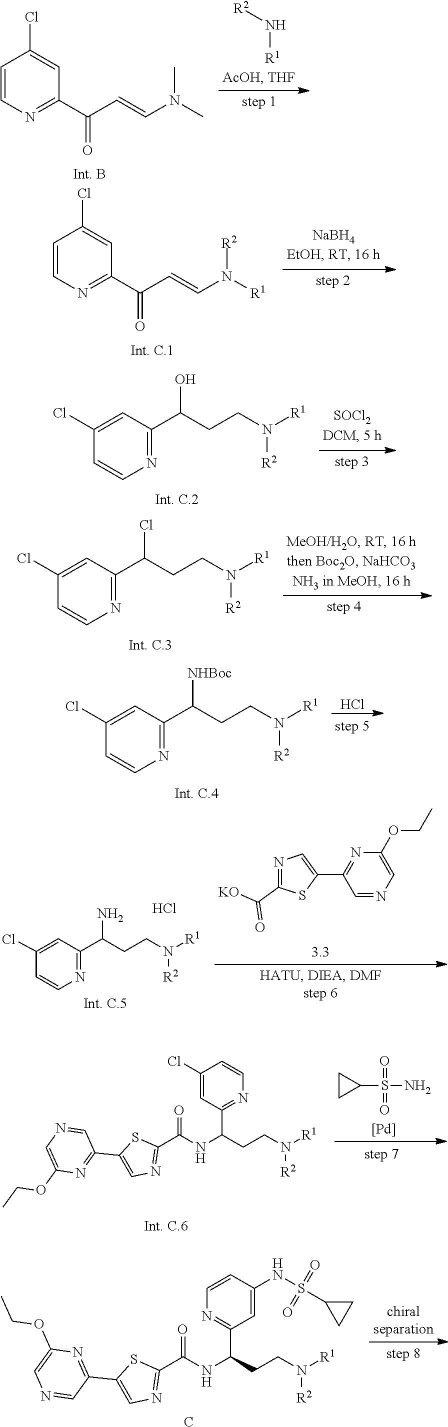

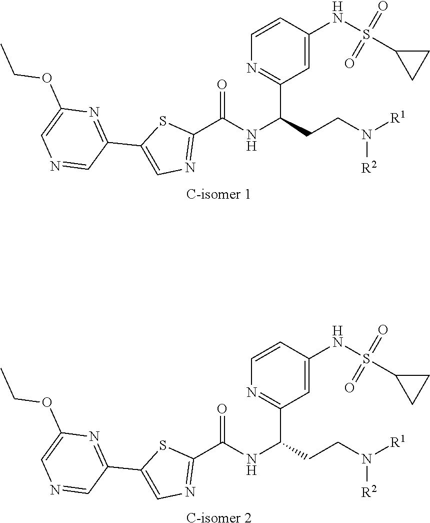

### Example 8: Synthesis of (S)—N-(3-(cyclohexyl(methyl)amino)-1-(4-(cyclopropanesulfonamido)pyridin-2-yl)propyl)-5-(6-ethoxypyrazin-2-yl)thiazole-2-carboxamide, 1-13 and (R)—N-(3-(cyclohexyl(methyl)amino)-1-(4-(cyclopropanesulfonamido)pyridin-2-yl)propyl)-5-(6-ethoxypyrazin-2-yl)thiazole-2-carboxamide, I-14

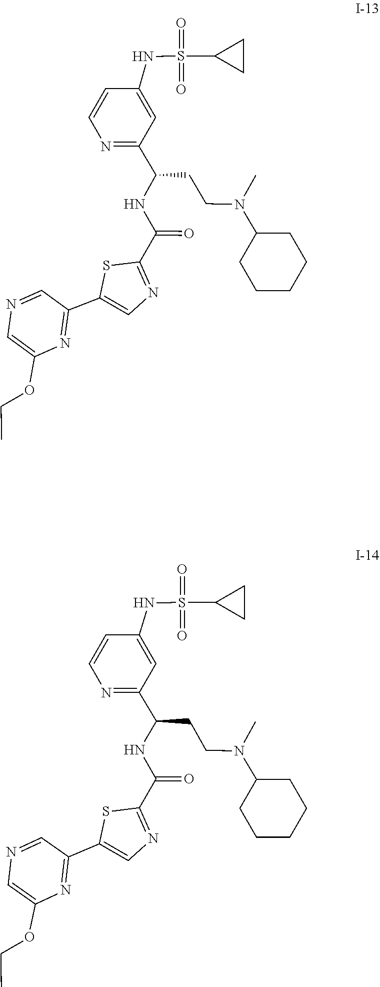 [SMILES: CCOc1cncc(-c2cnc(C(=O)N[C@@H](CCN(C)C3CCCCC3)c3cc(NS(=O)(=O)C4CC4)ccn3)s2)n1 ; CCOc1cncc(-c2cnc(C(=O)N[C@H](CCN(C)C3CCCCC3)c3cc(NS(=O)(=O)C4CC4)ccn3)s2)n1]

### General Method 1, Step 1: Synthesis of 8.1

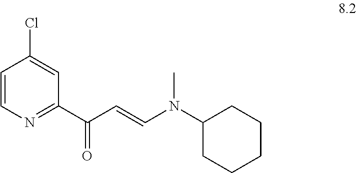 [SMILES: CN(/C=C/C(=O)c1cc(Cl)ccn1)C1CCCCC1]

To a stirred mixture of Int. B (2 g, 9.52 mmol, 1 equiv) and N-methylcyclohexanamine (3.23 g, 28.56 mmol, 3 equiv) in THF (45 mL) was added acetic acid (15 mL) dropwise at room temperature. The resulting mixture was stirred for 2 h at 60° C. The reaction mixture was concentrated under reduced pressure. The residue was neutralized to pH 10 with saturated aqueous sodium bicarbonate and extracted with ethyl acetate (3×30 mL). The combined organic layers were concentrated under reduced pressure. The residue was purified by flash column chromatography on C18 silica (eluted with 63% acetonitrile in water) to afford (E)-1-(4-chloropyridin-2-yl)-3-(cyclohexyl(methyl)amino)prop-2-en-1-one (8.1, 1.32 g, 50%) as a light brown solid. MS (ES): m/z 279/281 [M+H]⁺.

### General Method 1, Step 2: Synthesis of 8.2

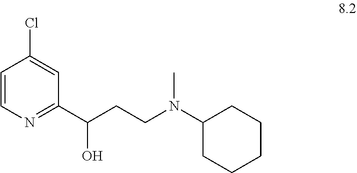 [SMILES: CN(CCC(O)c1cc(Cl)ccn1)C1CCCCC1]

To a stirred solution of 8.1 (1.32 g, 4.73 mmol, 1 equiv) in ethanol (20 mL) was added sodium borohydride (1.78 g, 47.3 mmol, 10 equiv) in portions at 0° C. The resulting mixture was stirred for 16 h at room temperature. The reaction was quenched with methanol (10 mL) at 0° C., diluted with water (30 mL) and extracted with dichloromethane (4×30 mL). The combined organic layers were concentrated under reduced pressure. The residue was purified by flash column chromatography on C18 silica (eluted with 46% acetonitrile in water) to afford 1-(4-chloropyridin-2-yl)-3-(cyclohexyl(methyl)amino)propan-1-ol (8.2, 0.88 g, 66%) as an yellow oil. MS (ES): m/z 283/285 [M+H]⁺.

### General Method 1, Step 3: Synthesis of 8.3

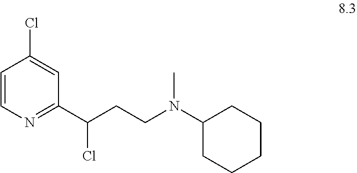 [SMILES: CN(CCC(Cl)c1cc(Cl)ccn1)C1CCCCC1]

To a stirred solution of 8.2 (850 mg, 3 mmol, 1 equiv) in dichloromethane (4 mL) was added thionyl chloride (0.72 mL, 6.6 mmol, 2.2 equiv) dropwise at 0° C. The resulting mixture was stirred for 1 h at room temperature. The reaction was concentrated under reduced pressure to afford N-(3-chloro-3-(4-chloropyridin-2-yl)propyl)-N-methylcyclohexanamine (8.3, 900 mg, crude) as a colorless oil, which was used in the next step directly without further purification. MS (ES): m/z 301/303 [M+H]⁺.

### General Method 1, Step 4: Synthesis of 8.4

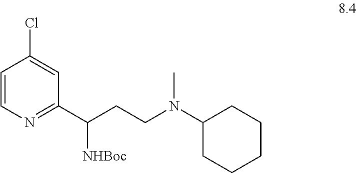 [SMILES: CN(CCC(NC(=O)OC(C)(C)C)c1cc(Cl)ccn1)C1CCCCC1]

A mixture of 8.3 (900 mg) and sodium iodide (900 mg, 6 mmol) in ammonia in methanol solution (2M, 10 mL) was stirred for 16 h at 40° C. The solid was removed by filtration and washed with methanol (5 mL). The filtrate was concentrated and the residue was dissolved with methanol (10 mL), followed by the addition of di-tert-butyl pyrocarbonate (1.31 g, 6 mmol) and saturated sodium bicarbonate aqueous solution (10 mL). The resulting mixture was stirred for 4 h at room temperature. The reaction was extracted with dichloromethane (4×10 mL). The combined organic layers were concentrated under reduced pressure. The residue was purified by flash column chromatography on C18 silica (eluted with 72% acetonitrile in water) to afford tert-butyl (1-(4-chloropyridin-2-yl)-3-(cyclohexyl(methyl)amino)propyl)carbamate (8.4, 520 mg, 45% over 2 steps) as a yellow solid. MS (ES): m/z 382/384 [M+H]⁺.

### General Method 1, Step 5: Synthesis of 8.5

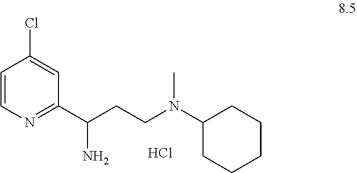 [SMILES: CN(CCC(N)c1cc(Cl)ccn1)C1CCCCC1 ; Cl]

To a mixture of 8.4 (500 mg, 1.31 mmol, 1 equiv) in dichloromethane (5 mL) was added HCl in 1,4-dioxane (4M, 5 mL) dropwise at room temperature. The resulting mixture was stirred at room temperature for 30 min. The reaction was concentrated under reduced pressure to afford 1-(4-chloropyridin-2-yl)-N3-cyclohexyl-N3-methylpropane-1,3-diamine hydrochloride (8.5, 400 mg) as a yellow solid, which was used in the next step directly without further purification. MS (ES): m/z 282/284 [M+H]⁺.

### General Method 1, Step 6: Synthesis of 8.6

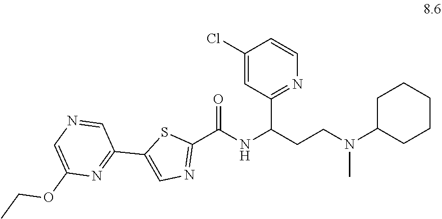 [SMILES: CCOc1cncc(-c2cnc(C(=O)NC(CCN(C)C3CCCCC3)c3cc(Cl)ccn3)s2)n1]

To a stirred mixture of 8.5 (380 mg) and 3.3 (350 mg) in N,N-dimethylformamide (5 mL) were added 1-hydroxybenzotriazole (359 mg, 2.69 mmol) and 1-ethyl-3-(3-dimethylaminopropyl)carbodiimide hydrochloride (650 mg, 3.37 mmol). The resulting mixture was stirred for 2 h at room temperature. The mixture was diluted with water (20 mL) and the product was extracted with ethyl acetate (3×15 mL). The combined organic layers were concentrated under reduced pressure. The residue was purified by flash column chromatography on silica gel (eluted with 10% methanol in dichloromethane) to afford N-(1-(4-chloropyridin-2-yl)-3-(cyclohexyl(methyl)amino)propyl)-5-(6-ethoxypyrazin-2-yl)thiazole-2-carboxamide (8.6, 350 mg) as a white solid. MS (ES): m/z 515/517 [M+H]⁺.

### General Method 1, Step 7: Synthesis of 8.7

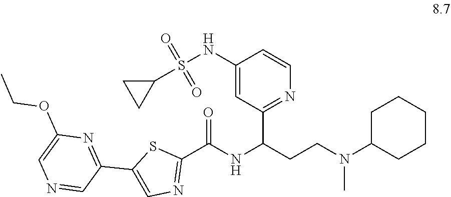 [SMILES: CCOc1cncc(-c2cnc(C(=O)NC(CCN(C)C3CCCCC3)c3cc(NS(=O)(=O)C4CC4)ccn3)s2)n1]

To a stirred mixture of 8.6 (330 mg, 0.64 mmol, 1 equiv), cyclopropanesulfonamide (156 mg, 1.28 mmol, 2 equiv) and cesium carbonate (626 mg, 1.92 mmol, 3 equiv) in 1,4-dioxane (10 mL) were added EPhos Pd G4 (35 mg, 0.06 mmol, 0.1 equiv) and EPhos (29 mg, 0.06 mmol, 0.1 equiv) in portions at room temperature. The resulting mixture was stirred for 1 h at 100° C. under nitrogen atmosphere. The solid was removed by filtering through Celite and washed with methanol (20 mL). The combined organic layers were concentrated under reduced pressure. The residue was purified by Prep-HPLC (Sunfire prep C18 column, 30*150 mm, 5 m; Mobile Phase: water (0.1% FA) and ACN (15% to 40% in 7 min); Wavelength: 254/220 nm) to afford N-(3-(cyclohexyl(methyl)amino)-1-(4-(cyclopropanesulfonamido)pyridin-2-yl)propyl)-5-(6-ethoxypyrazin-2-yl)thiazole-2-carboxamide (8.7, 140 mg, 36%) as a white solid. MS (ES): m/z 600 [M+H]⁺.

General Method 1, Step 8: synthesis of I-13 and I-14. 8.7 (140 mg) was purified by chiral HPLC (Column: CHIRALPAK IG, 2*25 cm, 5 m; Mobile Phase A: Hex:DCM=3:1 (0.5% 2M NH₃-MeOH)-HPLC, Mobile Phase B: EtOH-HPLC; Flow rate: 20 mL/min; Gradient: 20% B to 20% B in 15 min; Wavelength: 220/254 nm) to afford (S)—N-(3-(cyclohexyl(methyl)amino)-1-(4-(cyclopropanesulfonamido)pyridin-2-yl)propyl)-5-(6-ethoxypyrazin-2-yl)thiazole-2-carboxamide (I-13, first eluting peak, 24 mg, 17%) and (R)—N-(3-(cyclohexyl(methyl)amino)-1-(4-(cyclopropanesulfonamido)pyridin-2-yl)propyl)-5-(6-ethoxypyrazin-2-yl)thiazole-2-carboxamide (I-14, second eluting peak, 25 mg, 17%) both as white solid.

I-13. MS (ES): m/z 600 [M+H]⁺; ¹H NMR (300 MHz, DMSO-d₆) δ 9.88 (d, J=7.7 Hz, 1H), 8.93 (s, 1H), 8.81 (s, 1H), 8.28 (s, 2H), 7.10 (dd, J=5.8, 2.2 Hz, 1H), 7.01 (dd, J=5.8, 2.2 Hz, 1H), 5.10-5.06 (m, 1H), 4.42 (q, J=7.0 Hz, 2H), 2.78-2.66 (m, 1H), 2.49-2.34 (m, 3H), 2.25 (s, 3H), 2.14-2.01 (m, 2H), 1.80-1.62 (m, 4H), 1.58-1.52 (m, 1H), 1.39 (t, J=7.0 Hz, 3H), 1.23-1.01 (m, 4H), 0.99-0.88 (m, 5H).

I-14. MS (ES): m/z 600[M+H]⁺; ¹H NMR (300 MHz, DMSO-d₆) δ 9.87 (d, J=7.7 Hz, 1H), 8.93 (s, 1H), 8.81 (s, 1H), 8.29 (s, 2H), 7.10 (dd, J=5.8, 2.2 Hz, 1H), 7.01 (dd, J=5.8, 2.2 Hz, 1H), 5.10-5.07 (m, 1H), 4.42 (q, J=7.0 Hz, 2H), 2.78-2.66 (m, 1H), 2.49-2.34 (m, 3H), 2.27 (s, 3H), 2.14-1.98 (m, 2H), 1.80-1.62 (m, 4H), 1.58-1.52 (m, 1H), 1.39 (t, J=7.0 Hz, 3H), 1.23-1.09 (m, 4H), 1.05-0.88 (m, 5H).

### Example 9: Synthesis of (S)—N-(1-(4-(cyclopropanesulfonamido)pyridin-2-yl)-3-(4-fluoropiperidin-1-yl)propyl)-5-(6-ethoxypyrazin-2-yl)thiazole-2-carboxamide, 1-15 and (R)—N-(1-(4-(cyclopropanesulfonamido)pyridin-2-yl)-3-(4-fluoropiperidin-1-yl)propyl)-5-(6-ethoxypyrazin-2-yl)thiazole-2-carboxamide, I-16

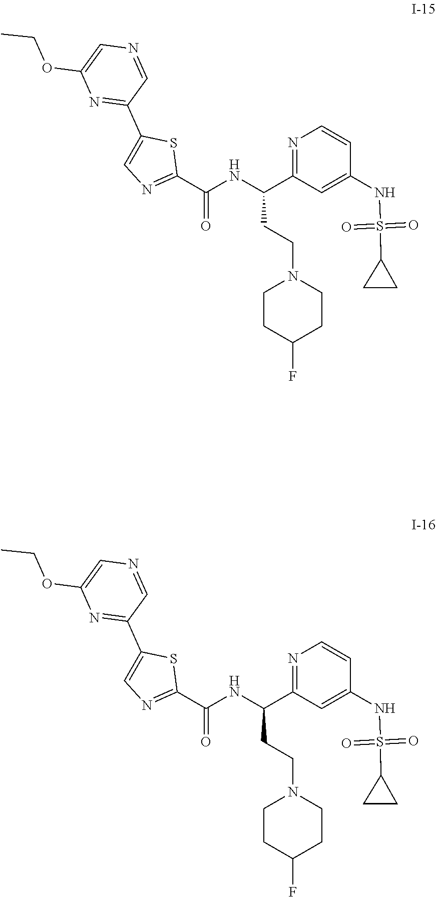 [SMILES: CCOc1cncc(-c2cnc(C(=O)N[C@@H](CCN3CCC(F)CC3)c3cc(NS(=O)(=O)C4CC4)ccn3)s2)n1 ; CCOc1cncc(-c2cnc(C(=O)N[C@H](CCN3CCC(F)CC3)c3cc(NS(=O)(=O)C4CC4)ccn3)s2)n1]

Synthesis of I-15 and I-16. Starting from commercially available 4-fluoropiperidine, I-15 and I-16 was prepared following General Method 1. Chiral purification in Step 8 with the following method: chiral HPLC, Column: CHIRALPAK IG, 2*25 cm, 5 m; Mobile Phase A: Hex:DCM=3:1 (0.5% 2M NH₃-MeOH)-HPLC, Mobile Phase B: EtOH-HPLC; Flow rate: 20 mL/min; Gradient: 20% B to 20% B in 15 min; Wavelength: 220/254 nm.

I-15. First eluting peak, MS (ES): m/z 590 [M+H]⁻; ¹H NMR (300 MHz, DMSO-d₆) δ 10.52 (s, 1H), 10.03 (d, J=7.5 Hz, 1H), 8.94 (s, 1H), 8.80 (s, 1H), 8.32-8.28 (m, 2H), 7.16-7.02 (m, 2H), 5.14-5.06 (m, 1H), 4.84-4.68 (m, 1H), 4.42 (q, J=7.0 Hz, 2H), 2.75-2.61 (m, 3H), 2.41-1.78 (m, 10H), 1.39 (t, J=7.0 Hz, 3H), 1.01-0.79 (m, 4H).

I-16. Second eluting peak, MS (ES): m/z 590 [M+H]⁺; ¹H NMR (300 MHz, DMSO-d₆) δ 10.52 (s, 1H), 10.03 (d, J=7.5 Hz, 1H), 8.94 (s, 1H), 8.80 (s, 1H), 8.38-8.28 (m, 2H), 7.16-6.98 (m, 2H), 5.14-5.04 (m, 1H), 4.84-4.62 (m, 1H), 4.42 (q, J=7.0 Hz, 2H), 2.75-2.61 (m, 2H), 2.41-1.78 (m, 1H), 1.39 (t, J=7.0 Hz, 3H), 1.01-0.82 (m, 4H).

### Example 10: Synthesis of (R)—N-(1-(4-(cyclopropanesulfonamido)pyridin-2-yl)-3-(5-azaspiro[2.5]octan-5-yl)propyl)-5-(6-ethoxypyrazin-2-yl)thiazole-2-carboxamide, I-17 and (S)—N-(1-(4-(cyclopropanesulfonamido)pyridin-2-yl)-3-(5-azaspiro[2.5]octan-5-yl)propyl)-5-(6-ethoxypyrazin-2-yl)thiazole-2-carboxamide, I-18

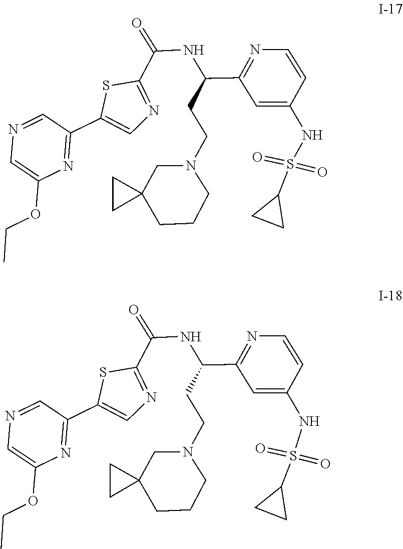 [SMILES: CCOc1cncc(-c2cnc(C(=O)N[C@H](CCN3CCCC4(CC4)C3)c3cc(NS(=O)(=O)C4CC4)ccn3)s2)n1 ; CCOc1cncc(-c2cnc(C(=O)N[C@@H](CCN3CCCC4(CC4)C3)c3cc(NS(=O)(=O)C4CC4)ccn3)s2)n1]

Synthesis of I-17 and I-18. Starting from commercially available 5-azaspiro[2.5]octane, I-17 and I-18 was prepared following General Method 1. Chiral purification in Step 8 with the following method: chiral HPLC, Column: CHIRALPAK IG, 2*25 cm, 5 m; Mobile Phase A: Hex:DCM=3:1 (0.5% 2M NH₃-MeOH)-HPLC, Mobile Phase B: EtOH-HPLC; Flow rate: 20 mL/min; Gradient: 20% B to 20% B in 16 min; Wavelength: 220/254 nm.

I-18. First eluting peak, MS (ES): m/z 598 [M+H]⁺; ¹H NMR (400 MHz, DMSO-d₆) δ 9.94 (d, J=7.5 Hz, 1H), 8.94 (s, 1H), 8.83 (s, 1H), 8.28 (s, 2H), 7.12-6.95 (m, 2H), 5.14-5.06 (m, 1H), 4.43 (q, J=7.1 Hz, 2H), 2.75-2.58 (m, 2H), 2.41-2.22 (m, 4H), 2.20-1.94 (m, 3H), 1.82-1.62 (m, 2H), 1.39 (t, J=7.0 Hz, 3H), 1.32-1.22 (m, 2H), 0.95-0.82 (m, 4H), 0.41-0.25 (m, 4H).

I-17. Second eluting peak, MS (ES): m/z 598 [M+H]⁺; ¹H NMR (400 MHz, DMSO-d₆) δ 9.94 (d, J=7.5 Hz, 1H), 8.94 (s, 1H), 8.83 (s, 1H), 8.28 (s, 2H), 7.12-6.95 (m, 2H), 5.14-5.06 (m, 1H), 4.43 (q, J=7.1 Hz, 2H), 2.75-2.61 (m, 2H), 2.45-2.22 (m, 4H), 2.20-1.92 (m, 3H), 1.82-1.66 (m, 2H), 1.39 (t, J=7.0 Hz, 3H), 1.32-1.22 (m, 2H), 0.95-0.82 (m, 4H), 0.41-0.25 (m, 4H).

### Example 11: Synthesis of (R)—N-((4-(cyclopropanesulfonamido) pyridin-2-yl)(tetrahydro-2H-pyran-4-yl)methyl)-5-(6-ethoxypyrazin-2-yl)thiazole-2-carboxamide, I-19 and (S)—N-((4-(cyclopropanesulfonamido) pyridin-2-yl)(tetrahydro-2H-pyran-4-yl)methyl)-5-(6-ethoxypyrazin-2-yl)thiazole-2-carboxamide, I-20

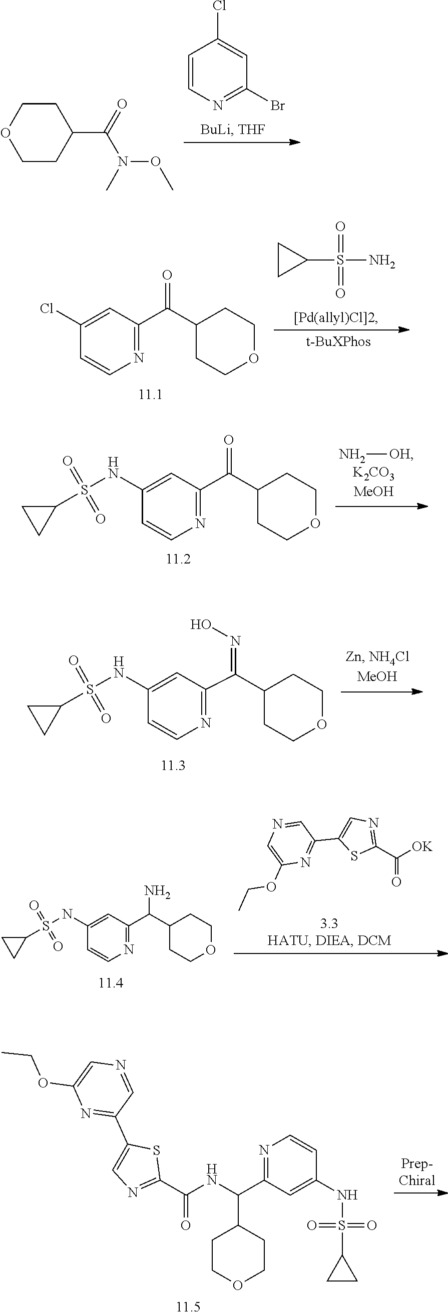

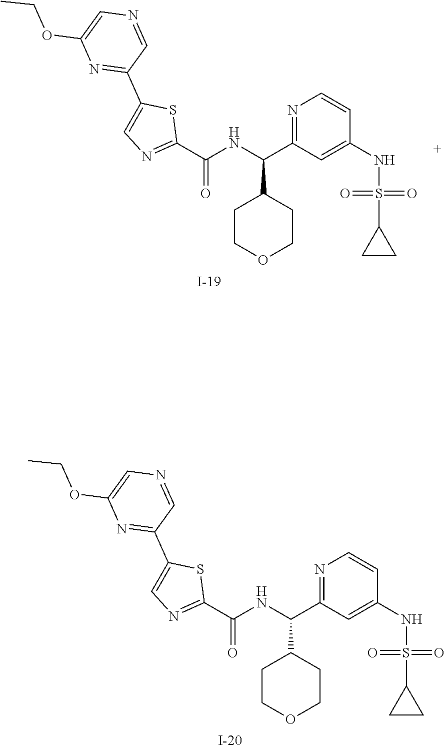

Synthesis of 11.1. To a solution of 2-bromo-4-chloropyridine (5 g, 25.9 mmol, 1.5 eq) in THE (10 mL) was added n-butyllithium solution (2.5 M in THF/hexane, 3.5 mL, 8.7 mmol, 0.5 eq) dropwise at −78° C. under N₂ atmosphere. The resulting mixture was stirred at −78° C. for 30 mins. Then a solution of N-methoxy-N-methyloxane-4-carboxamide (3 g, 17.3 mmol, 1.0 eq) in THE (3 mL) was added dropwise at −78° C. The resulting mixture was allowed to warm to room temperature and stirred for 2 h. The reaction was quenched with aqueous saturated ammonium chloride (20 mL) and the product was extracted with ethyl acetate (3×15 mL). The combined organic layers were concentrated under vacuum. The residue was purified by flash column chromatography on silica gel (eluted with 18% ethyl acetate in petro ether) to afford 4-chloro-2-(oxane-4-carbonyl) pyridine (11.1, 1.5 g, 38%) as a yellow solid. MS (ES): m/z 226/228 [M+H]⁺.

Synthesis of 11.2. To a stirred solution of 11.1 (1 g, 4.4 mmol, 1.0 eq) and cyclopropanesulfonamide (1.1 g, 8.8 mmol, 2.0 eq) in 1,4-dioxane (10 mL) were added t-BuXPhos (190 mg, 0.4 mmol, 0.1 eq), allylpalladium chloride dimer (80 mg, 0.2 mmol, 0.05 eq) and cesium carbonate (2.8 g, 8.8 mmol, 2.0 eq). The resulting mixture was stirred at 60° C. for 2 h under nitrogen atmosphere. The reaction was diluted with methanol (30 mL) and filtered through Celite. The filtrate was concentrated under reduced pressure. The residue was purified by flash column chromatography on silica gel (eluted with 65% ethyl acetate in petro ether) to afford N-(2-(tetrahydro-2H-pyran-4-carbonyl)pyridin-4-yl)cyclopropanesulfonamide (11.2, 650 mg, 48%) as a yellow solid. MS (ES): m/z 311 [M+H]⁺.

Synthesis of 11.3. To a stirred solution of 11.2 (650 mg, 2.1 mmol, 1.0 eq) and hydroxylamine hydrochloride (728 mg, 10.5 mmol, 5.0 eq) in methanol (3 mL) was added potassium carbonate (1.4 g, 10.5 mmol, 5.0 eq). The resulting mixture was stirred for 2 h at room temperature. The reaction was diluted with methanol (20 mL) and filtered through Celite. The filtrate was concentrated under reduced pressure to afford (Z)—N-(2-((hydroxyimino)(tetrahydro-2H-pyran-4-yl)methyl)pyridin-4-yl)cyclopropanesulfonamide (11.3, 400 mg) as a yellow oil, which was used directly in the next step without further purification. MS (ES): m/z 326 [M+H]⁺.

Synthesis of 11.4. To a stirred solution of 11.3 (400 mg) in methanol (3 mL) were added zinc powder (402 mg, 6.1 mmol) and ammonium chloride (329 mg, 6.1 mmol). The resulting mixture was stirred for 4 h at 60° C. The reaction was diluted with methanol (20 mL) and filtered through Celite. The filtrate was concentrated under reduced pressure. The residue was purified by flash column chromatography on C18 silica (eluted with 25% acetonitrile in water) to afford N-(2-(amino(tetrahydro-2H-pyran-4-yl)methyl)pyridin-4-yl)cyclopropanesulfonamide (11.4, 300 mg, 46% over 2 steps) as a yellow solid. MS (ES): m/z 312 [M+H]⁺.

Synthesis of 11.5. To a stirred solution of 11.4 (300 mg, 0.96 mmol, 1.0 eq) and 3.3¹ (323 mg) in DMF (3 mL) were added HATU (977 mg, 2.6 mmol, 2.5 eq) and DIEA (664 mg, 5.1 mmol, 5.3 eq). The resulting mixture was stirred for 2 h at room temperature. The reaction was diluted with water (15 mL) and the product was extracted with ethyl acetate (3×15 mL). The combined organic layers were concentrated under reduced pressure. The residue was purified by prep-HPLC (XBridge Prep OBD C18 Column, 30*150 mm, 5 m; Mobile Phase A: water (10 mM NH₄HCO₃), Mobile Phase B: ACN; Flow rate: 60 mL/min; Gradient: 25% B to 45% B in 10 min; Wavelength: 254/220 nm) to afford N-[(4-cyclopropanesulfonamidopyridin-2-yl) (oxan-4-yl)methyl]-5-(6-ethoxypyrazin-2-yl)-1,3-thiazole-2-carboxamide (11.5, 120 mg, 23%) as a yellow oil. MS (ES): m/z 545 [M+H]⁺.

Synthesis of I-19 and I-20. 11.5 (120 mg) was purified by chiral-HPLC (Column: CHIRAL ART Amylose-SA, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 20% B to 20% B in 10 min; Wavelength: 220/254 nm) to afford (R)—N-((4-(cyclopropanesulfonamido) pyridin-2-yl)(tetrahydro-2H-pyran-4-yl)methyl)-5-(6-ethoxypyrazin-2-yl)thiazole-2-carboxamide, (I-19, first eluting peak, 27 mg) and (S)—N-((4-(cyclopropanesulfonamido) pyridin-2-yl)(tetrahydro-2H-pyran-4-yl)methyl)-5-(6-ethoxypyrazin-2-yl)thiazole-2-carboxamide, (I-20, second eluting peak, 35 mg) both as white solid.

I-19. MS (ES): m/z 545 [M+H]⁺; ¹H NMR (300 MHz, Methanol-d₄) δ 8.69 (s, 1H), 8.58 (s, 1H), 8.31 (d, J=6.0 Hz, 1H), 8.14 (s, 1H), 7.22 (d, J=2.1 Hz, 1H), 7.13 (dd, J=6.0, 2.1 Hz, 1H), 4.90-4.86 (m, 1H), 4.47 (q, J=7.2 Hz, 2H), 4.01-3.87 (m, 2H), 3.46-3.38 (m, 2H), 2.76-2.68 (m, 1H), 2.30-2.19 (m, 1H), 1.85-1.80 (m, 1H), 1.55-1.50 (m, 1H), 1.44 (t, J=7.2 Hz, 3H), 1.39-1.24 (m, 2H), 1.18-1.10 (m, 2H), 1.04-0.96 (m, 2H).

I-20. MS (ES): m/z 545 [M+H]⁺; ¹H NMR (400 MHz, Methanol-d₄) δ 8.68 (s, 1H), 8.57 (s, 1H), 8.34 (d, J=5.6 Hz, 1H), 8.14 (s, 1H), 7.24 (d, J=2.4 Hz, 1H), 7.15 (dd, J=5.6, 2.4 Hz, 1H), 4.90-4.87 (m, 1H), 4.47 (q, J=7.2 Hz, 2H), 4.00-3.89 (m, 2H), 3.46-3.33 (m, 2H), 2.77-2.70 (m, 1H), 2.29-2.20 (m, 1H), 1.85-1.81 (m, 1H), 1.53-1.47 (m, 1H), 1.44 (t, J=7.2 Hz, 3H), 1.41-1.24 (m, 2H), 1.16-1.13 (m, 2H), 1.03-0.99 (m, 2H).

### Example 12: 5-{5-chloropyrazolo[1,5-a]pyridin-3-yl}-N—[(S)-(4-cyclopropanesulfonamidopyridin-2-yl)((3R)-oxolan-3-yl)methyl]-1,3-thiazole-2-carboxamide, I-22, 5-{5-chloropyrazolo[1,5-a]pyridin-3-yl}-N—[(R)-(4-cyclopropanesulfonamidopyridin-2-yl)((3R)-oxolan-3-yl)methyl]-1,3-thiazole-2-carboxamide, I-23, 5-{5-chloropyrazolo[1,5-a]pyridin-3-yl}-N—[(S)-(4-cyclopropanesulfonamidopyridin-2-yl)((3R)-oxolan-3-yl)methyl]-1,3-thiazole-2-carboxamide, I-24 and 5-{5-chloropyrazolo[1,5-a]pyridin-3-yl}-N—[(R)-(4-cyclopropanesulfonamidopyridin-2-yl)((3R)-oxolan-3-yl)methyl]-1,3-thiazole-2-carboxamide, I-25

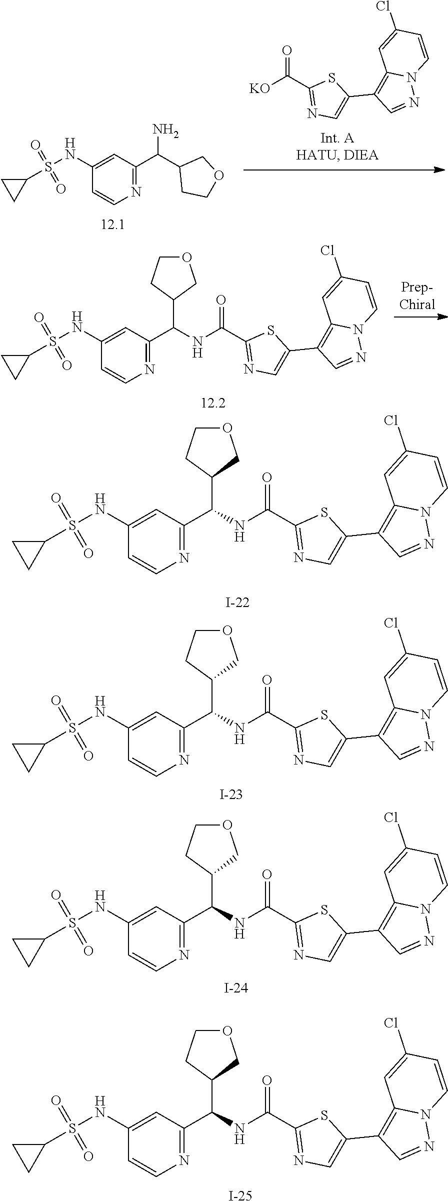

Synthesis of 12.1. 12.1 was synthesized from N-methoxy-N-methyltetrahydrofuran-3-carboxamide in a manner similar to that in the synthesis of 11.4. MS (ES): m/z 298/300 [M+H]⁺.

Synthesis of 12.2. To a stirred solution of 12.1 (680 mg, 2.3 mmol, 1.0 eq) and Int. A (640 mg) in acetonitrile (10 mL) were added TCFH (3.2 g, 11.5 mmol, 5.0 eq) and NMI (1.9 g, 22.8 mmol, 10 eq) in portions at room temperature. The resulting mixture was stirred overnight at room temperature. The reaction was diluted with water (30 mL) and extracted with ethyl acetate (3×15 mL). The combined organic layers were concentrated under reduced pressure. The residue was purified by prep-HPLC (XBridge Prep OBD C18 Column, 30*150 mm, 5 m; Mobile Phase A: water (10 mML NH₄HCO₃+0.1% NH₃—H₂O), Mobile Phase B: ACN; Flow rate: 60 mL/min; Gradient: 25% B to 35% B in 8 min; Wavelength: 254/220 nm) to afford 5-{5-chloropyrazolo[1,5-a]pyridin-3-yl}-N-[(4-cyclopropanesulfonamidopyridin-2-yl)(oxolan-3-yl)methyl]-1,3-thiazole-2-carboxamide (12.2, 250 mg, 19%) as a yellow solid. MS (ES): m/z 559/561 [M+H]⁺.

Synthesis of I-22, I-23, I-24, and I-25. 12.2 (250 mg) was purified by Chiral-HPLC (Column: CHIRAL ART Cellulose-SB, 2*25 cm, 5 m; Mobile Phase A: MtBE (0.1% FA)-HPLC, Mobile Phase B: IPA-HPLC; Flow rate: 20 mL/min; Gradient: 50% B to 50% B in 17 min; Wavelength: 220/254 nm) to afford a mixture of 2 isomers (first eluting peak, 50 mg) and another mixture of 2 isomers (second eluting peak, 60 mg) both as yellow solid. The first eluting mixture (50 mg) was purified by Chiral-HPLC (Column: CHIRALPAK IG, 2*25 cm, 5 m; Mobile Phase A: MtBE (0.10% FA)-HPLC, Mobile Phase B: EtOH-HPLC; Flow rate: 15 mL/min; Gradient: 50% B to 50% B in 18 min; Wavelength: 220/254 nm) to afford 5-{5-chloropyrazolo[1,5-a]pyridin-3-yl}-N—[(S)-(4-cyclopropanesulfonamidopyridin-2-yl)((3R)-oxolan-3-yl)methyl]-1,3-thiazole-2-carboxamide, I-22, first eluting peak, 25 mg) and 5-{5-chloropyrazolo[1,5-a]pyridin-3-yl}-N—[(R)-(4-cyclopropanesulfonamidopyridin-2-yl)((3R)-oxolan-3-yl)methyl]-1,3-thiazole-2-carboxamide, I-23, second eluting peak, 15 mg) both as light yellow solid. The second eluting mixture (60 mg) was purified by Chiral-HPLC (Column: CHIRAL ART Amylose-SA, 2*25 cm, 5 μm; Mobile Phase A: MtBE (0.1% FA)-HPLC, Mobile Phase B: EtOH-HPLC; Flow rate: 20 mL/min; Gradient: 50% B to 50% B in 14 min; Wavelength: 220/254 nm) to afford 5-{5-chloropyrazolo[1,5-a]pyridin-3-yl}-N—[(S)-(4-cyclopropanesulfonamidopyridin-2-yl)((3R)-oxolan-3-yl)methyl]-1,3-thiazole-2-carboxamide, I-24, first eluting peak, 20 mg) and 5-{5-chloropyrazolo[1,5-a]pyridin-3-yl}-N—[(R)-(4-cyclopropanesulfonamidopyridin-2-yl)((3R)-oxolan-3-yl)methyl]-1,3-thiazole-2-carboxamide, I-25, second eluting peak, 19 mg) both as light yellow solids.

I-22. MS (ES): m/z 559/561 [M+H]⁺; ¹H NMR (400 MHz, DMSO-d₆) δ 10.57 (s, 1H), 9.22 (s, 1H), 8.86 (d, J=7.2 Hz, 1H), 8.59 (s, 1H), 8.41 (s, 1H), 8.37 (s, 1H), 8.12 (s, 1H), 7.26 (s, 1H), 7.14-7.06 (m, 2H), 4.98 (t, J=9.2 Hz, 1H), 3.81-3.75 (m, 1H), 3.66-3.61 (m, 1H), 3.56-3.52 (m, 1H), 3.46-3.42 (m, 1H), 3.01-2.83 (m, 2H), 2.06-1.99 (m, 1H), 1.80-1.71 (m, 1H), 1.04-0.96 (m, 4H).

I-23. MS (ES): m/z 559/561 [M+H]⁺; ¹H NMR (400 MHz, DMSO-d₆) δ 10.57 (s, 1H), 9.21 (s, 1H), 8.87 (d, J=7.2 Hz, 1H), 8.60 (s, 1H), 8.41 (s, 1H), 8.37 (s, 1H), 8.12 (d, J=2.0 Hz, 1H), 7.26 (s, 1H), 7.13 (dd, J=7.2, 2.0 Hz, 1H), 7.06 (s, 1H), 4.98 (t, J=9.6 Hz, 1H), 3.80-3.75 (m, 1H), 3.66-3.60 (m, 1H), 3.56-3.51 (m, 1H), 3.46-3.42 (m, 1H), 3.01-2.84 (m, 2H), 2.05-1.99 (m, 1H), 1.80-1.71 (m, 1H), 1.04-0.96 (m, 4H).

I-24. MS (ES): m/z 559/561 [M+H]⁺; ¹H NMR (400 MHz, DMSO-d₆) δ 10.87 (s, 1H), 9.20 (d, J=8.4 Hz, 1H), 8.87 (d, J=7.2 Hz, 1H), 8.59 (s, 1H), 8.41 (s, 1H), 8.37 (d, J=5.6 Hz, 1H), 8.12 (d, J=2.0 Hz, 1H), 7.24 (s, 1H), 7.14-7.11 (m, 1H), 7.04-7.03 (m, 1H), 4.94 (t, J=9.2 Hz, 1H), 3.82-3.76 (m, 2H), 3.64-3.58 (m, 2H), 3.00-2.91 (m, 1H), 2.79-2.76 (m, 1H), 1.77-1.69 (m, 1H), 1.66-1.57 (m, 1H), 1.05-0.93 (m, 4H).

I-25. MS (ES): m/z 559/561 [M+H]⁺; ¹H NMR (400 MHz, DMSO-d₆) δ 10.87 (s, 1H), 9.22 (d, J=8.8 Hz, 1H), 8.86 (d, J=7.2 Hz, 1H), 8.59 (s, 1H), 8.41 (s, 1H), 8.32 (d, J=6.0 Hz, 1H), 8.12 (s, 1H), 7.26 (s, 1H), 7.14-7.11 (m, 1H), 7.06-7.04 (m, 1H), 4.95 (t, J=9.2 Hz, 1H), 3.82-3.76 (m, 2H), 3.65-3.59 (m, 2H), 3.01-2.91 (m, 1H), 2.82-2.76 (m, 1H), 1.77-1.69 (m, 1H), 1.66-1.57 (m, 1H), 1.05-0.93 (m, 4H).

### Example 13: N-[(1S)-1-(4-cyclopropanesulfonamidopyridin-2-yl)-2-[(2S)-1-isopropylpiperidin-2-yl]ethyl]-5-(6-ethoxypyrazin-2-yl)-1,3-thiazole-2-carboxamide, I-26, N-[(1R)-1-(4-cyclopropanesulfonamidopyridin-2-yl)-2-[(2R)-1-isopropylpiperidin-2-yl]ethyl]-5-(6-ethoxypyrazin-2-yl)-1,3-thiazole-2-carboxamide, I-27, N-[(1S)-1-(4-cyclopropanesulfonamidopyridin-2-yl)-2-[(2S)-1-isopropylpiperidin-2-yl]ethyl]-5-(6-ethoxypyrazin-2-yl)-1,3-thiazole-2-carboxamide, 1-28 and N-[(1R)-1-(4-cyclopropanesulfonamidopyridin-2-yl)-2-[(2R)-1-isopropylpiperidin-2-yl]ethyl]-5-(6-ethoxypyrazin-2-yl)-1,3-thiazole-2-carboxamide, I-29

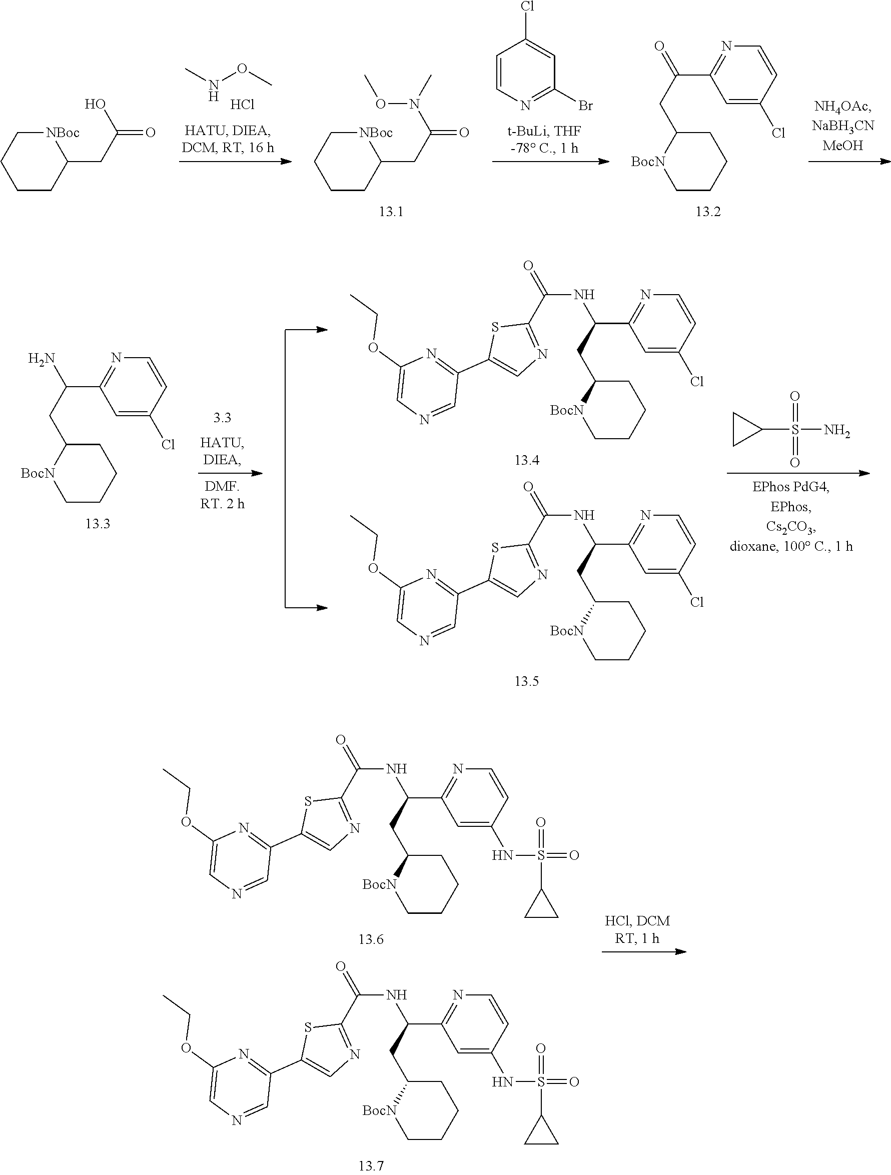

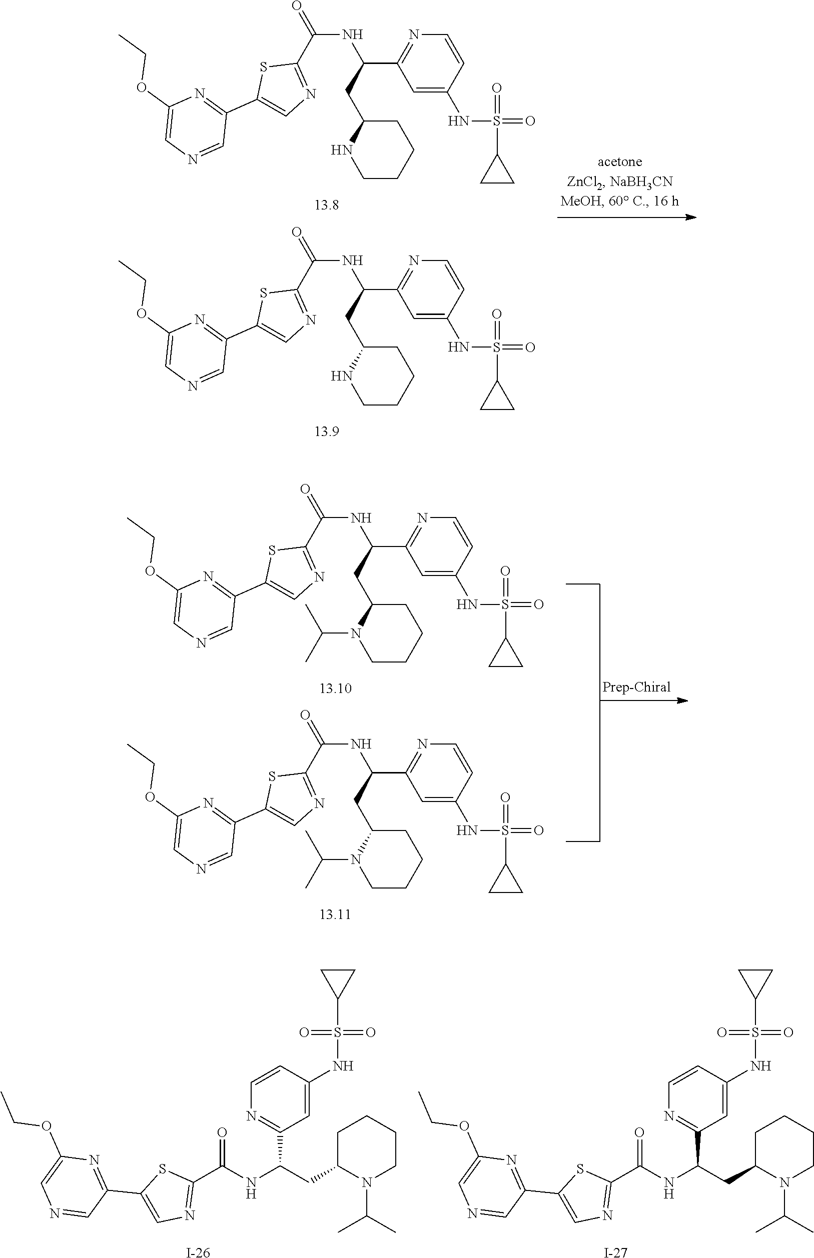

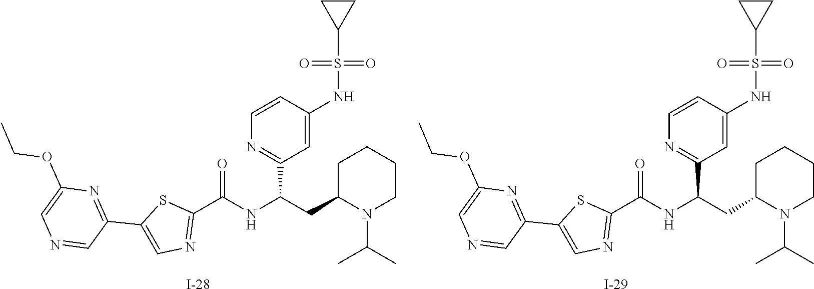

Synthesis of 13.1. To a solution of [1-(tert-butoxycarbonyl)piperidin-2-yl]acetic acid (10 g, 41.1 mmol, 1 eq) and N,O-dimethylhydroxylaminehydrochloride (5.2 g 53.4 mmol, 1.3 eq) in DMF (100 mL) was added HATU (20.3 g, 53.4 mmol, 1.3 eq) and DIEA (15.9 g, 123 mmol, 3 eq) in portions. The resulting mixture was stirred at room temperature for 2 h. The reaction was diluted with water (400 mL) and extracted with ethyl acetate (3×150 mL). The combined organic layers were concentrated under reduced pressure. The residue was purified by flash column chromatography on C18 silica (eluted with 40% acetonitrile in water) to afford tert-butyl 2-1{[methoxy(methyl)carbamoyl]methyl}piperidine-1-carboxylate (13.1, 5 g, 42%) as a light yellow oil. MS (ES): m/z 287 [M+H]⁺.

Synthesis of 13.2. To a stirred mixture of 2-bromo-4-chloropyridine (10.1 g, 52.4 mmol, 1.5 eq) in tetrahydrofuran (200 mL) was added n-BuLi (2.5M in THF, 26 mL, 52.4 mmol, 1.5 eq) dropwise at −78° C. under nitrogen atmosphere. The resulting mixture was stirred at −78° C. for 30 mins. Then a solution of 13.1 (10 g, 34.9 mmol, 1 eq) in tetrahydrofuran (40 mL) was added dropwise over 1 h at −78° C. under nitrogen atmosphere. The resulting mixture was allowed to warm to room temperature and stirred for 2 h. The reaction was quenched with aqueous saturated ammonium chloride (300 mL) and extracted with ethyl acetate (3×150 mL). The combined organic layers were concentrated under vacuum. The residue was purified by flash column chromatography on C18 silica (eluted with 30% acetonitrile in water) to afford tert-butyl 2-[2-(4-chloropyridin-2-yl)-2-oxoethyl]piperidine-1-carboxylate (13.2, 2.0 g, 17%) as a yellow oil. MS (ES): m/z 339/341 [M+H]⁺.

Synthesis of 13.3. To a stirred solution of 13.2 (1.1 g, 3.1 mmol, 1.0 eq) in methanol (30 mL) was added ammonium acetate (2.4 g, 31 mmol, 10 eq) in portions at 0° C. The resulting mixture was stirred for 7 h at room temperature. Sodium cyanoborohydride (390 mg, 6.2 mmol, 2.0 eq) was added in portions over 10 min at 0° C. The resulting mixture was stirred for additional 10 h at room temperature. The reaction was diluted with water (100 mL) and the product was extracted with ethyl acetate (3×30 mL). The combined organic layers were concentrated under reduced pressure. The residue was purified by flash column chromatography on C18 silica (eluted with 50% acetonitrile in water) to afford tert-butyl 2-[2-amino-2-(4-chloropyridin-2-yl)ethyl]piperidine-1-carboxylate (13.3, 600 mg, 57%) as a white solid. MS (ES): m/z 340/342 [M+H]⁺.

Synthesis of 13.4 and 13.5. To a stirred mixture of 13.3 (890 mg, 2.6 mmol, 1.0 eq), 3.3¹ (658 mg) and DIEA (1.0 g, 7.8 mmol, 3.0 eq) in N,N-dimethylformamide (15 mL) was added HATU (1.2 g, 3.1 mmol, 1.2 eq) in portions at room temperature. The resulting mixture was stirred for 1 h at room temperature. The reaction was diluted with water (50 mL) and extracted with ethyl acetate (3×20 mL). The combined organic layers were concentrated under reduced pressure. The residue was purified by flash column chromatography on silica gel (eluted with 50% ethyl acetate in dichloromethane) to afford tert-butyl (2S)-2-[(2S)-2-(4-chloropyridin-2-yl)-2-{[5-(6-ethoxypyrazin-2-yl)-1,3-thiazol-2-yl]formamido}ethyl]piperidine-1-carboxylate and tert-butyl (2R)-2-[(2R)-2-(4-chloropyridin-2-yl)-2-{[5-(6-ethoxypyrazin-2-yl)-1,3-thiazol-2-yl]formamido}ethyl]piperidine-1-carboxylate (13.4, first eluting peak, 370 mg, 25%) and tert-butyl (2R)-2-[(2S)-2-(4-chloropyridin-2-yl)-2-{[5-(6-ethoxypyrazin-2-yl)-1,3-thiazol-2-yl]formamido}ethyl]piperidine-1-carboxylate, tert-butyl (2S)-2-[(2R)-2-(4-chloropyridin-2-yl)-2-{[5-(6-ethoxypyrazin-2-yl)-1,3-thiazol-2-yl]formamido}ethyl]piperidine-1-carboxylate (13.5, second eluting peak, 400 mg, 27%) both as a white oil. MS (ES): m/z 573/575 [M+H]⁺ for both solids.

Synthesis of 13.6. To a stirred mixture of 13.4 (370 mg, 0.64 mmol, 1.0 eq), cyclopropanesulfonamide (156 mg, 1.3 mmol, 2.0 eq) and cesium carbonate (631 mg, 1.9 mmol, 3.0 eq) in dioxane (5 mL) were added EPhos Pd G4 (59 mg, 0.06 mmol, 0.1 eq) and EPhos (35 mg, 0.06 mmol, 0.1 eq) in portions at room temperature under nitrogen atmosphere. The resulting mixture was stirred for 1 h at 100° C. under nitrogen atmosphere. The reaction was diluted with methanol (30 mL) and filtered through Celite. The filtrate was concentrated under reduced pressure. The residue was purified by flash column chromatography on silica gel (eluted with 20% ethyl acetate in dichloromethane) to afford tert-butyl (2S)-2-[(2S)-2-(4-cyclopropanesulfonamidopyridin-2-yl)-2-{[5-(6-ethoxypyrazin-2-yl)-1,3-thiazol-2-yl]formamido}ethyl]piperidine-1-carboxylate, tert-butyl (2R)-2-[(2R)-2-(4-cyclopropanesulfonamidopyridin-2-yl)-2-{[5-(6-ethoxypyrazin-2-yl)-1,3-thiazol-2-yl]formamido}ethyl]piperidine-1-carboxylate (13.6, 308 mg, 73%) as a yellow oil. MS (ES): m/z 658 [M+H]⁺.

Synthesis of 13.7. Starting from 13.5 (600 mg, 1.04 mmol), in the similar manner to the synthesis of 13.6, tert-butyl (2R)-2-[(2S)-2-(4-cyclopropanesulfonamidopyridin-2-yl)-2-{[5-(6-ethoxypyrazin-2-yl)-1,3-thiazol-2-yl]formamido}ethyl]piperidine-1-carboxylate, tert-butyl (2S)-2-[(2R)-2-(4-cyclopropanesulfonamidopyridin-2-yl)-2-{[5-(6-ethoxypyrazin-2-yl)-1,3-thiazol-2-yl]formamido}ethyl]piperidine-1-carboxylate (13.7, 577 mg, 84%) was obtained as a yellow oil. MS (ES): m/z 658 [M+H]⁺.

Synthesis of 13.8. A mixture of 13.6 (308 mg, 0.46 mmol, 1.0 eq) and HCl (4M in 1,4-dioxane, 5 mL) in dichloromethane (5 mL) was stirred for 1 h at room temperature. The reaction was concentrated to afford N—((S)-1-(4-(cyclopropanesulfonamido)pyridin-2-yl)-2-((S)-piperidin-2-yl)ethyl)-5-(6-ethoxypyrazin-2-yl)thiazole-2-carboxamide, N—((R)-1-(4-(cyclopropanesulfonamido)pyridin-2-yl)-2-((R)-piperidin-2-yl)ethyl)-5-(6-ethoxypyrazin-2-yl)thiazole-2-carboxamide, (13.8, 254 mg) as a yellow solid, which was used in the next step directly without further purification. MS (ES): m/z 558 [M+H]⁺.

Synthesis of 13.9. Starting from 13.7 (577 mg, 0.87 mmol), in the similar manner to the synthesis of 13.8, N—((R)-1-(4-(cyclopropanesulfonamido)pyridin-2-yl)-2-((S)-piperidin-2-yl)ethyl)-5-(6-ethoxypyrazin-2-yl)thiazole-2-carboxamide, N—((S)-1-(4-(cyclopropanesulfonamido)pyridin-2-yl)-2-((R)-piperidin-2-yl)ethyl)-5-(6-ethoxypyrazin-2-yl)thiazole-2-carboxamide, (13.9, 489 mg) was obtained as a yellow solid, which was used in the next step directly without further purification. MS (ES): m/z 558 [M+H]⁺.

Synthesis of 13.10. A mixture of sodium cyanoborohydride (140 mg, 2.3 mmol) and ZnCl₂ (1.9 M in THF, 1.4 mL, 2.7 mmol) in methanol (5 mL) was stirred for 10 min at room temperature. Then a solution of 13.8 (254 mg) in acetone (3 mL) was added dropwise at room temperature. The resulting mixture was stirred for additional 16 h at 60° C. The reaction was diluted with methanol (20 mL) and filtered through Celite. The filtrate was concentrated under reduced pressure. The residue was purified by Prep-HPLC (Sunfire prep C18 column, 30*150 mm, m; Mobile Phase: water (0.1% FA) and ACN (15% up to 33% in 9 min); UV detection at 254/220 nm) to afford N—((S)-1-(4-(cyclopropanesulfonamido)pyridin-2-yl)-2-((S)-1-isopropylpiperidin-2-yl)ethyl)-5-(6-ethoxypyrazin-2-yl)thiazole-2-carboxamide, N—((R)-1-(4-(cyclopropanesulfonamido)pyridin-2-yl)-2-((R)-1-isopropylpiperidin-2-yl)ethyl)-5-(6-ethoxypyrazin-2-yl)thiazole-2-carboxamide, (13.10, 78 mg, 28% over 2 steps) as a yellow oil. MS (ES): m/z 600 [M+H]⁺.

Synthesis of 13.11. Starting from 13.9 (577 mg), in the similar manner to the synthesis of 13.10, N—((R)-1-(4-(cyclopropanesulfonamido)pyridin-2-yl)-2-((S)-1-isopropylpiperidin-2-yl)ethyl)-5-(6-ethoxypyrazin-2-yl)thiazole-2-carboxamide, N—((S)-1-(4-(cyclopropanesulfonamido)pyridin-2-yl)-2-((R)-1-isopropylpiperidin-2-yl)ethyl)-5-(6-ethoxypyrazin-2-yl)thiazole-2-carboxamide (13.11, 80 mg) was obtained as a yellow oil. MS (ES): m/z 600 [M+H]⁺.

Synthesis of I-26, I-27, I-28 and I-29. 13.10 (70 mg) was purified by Prep-Chrial-HPLC (Column: CHIRALPAK IE, 2*25 cm, 5 μm; Mobile Phase A: Hex:DCM=3:1 (0.5% 2M NH₃-MeOH)—-HPLC, Mobile Phase B: EtOH-HPLC; Flow rate: 20 mL/min; Gradient: 30% B to 30% B in 28 min; Wavelength: 220/254 nm) to afford N-[(1S)-1-(4-cyclopropanesulfonamidopyridin-2-yl)-2-[(2S)-1-isopropylpiperidin-2-yl]ethyl]-5-(6-ethoxypyrazin-2-yl)-1,3-thiazole-2-carboxamide (I-26, first eluting peak, 13 mg) and N-[(1R)-1-(4-cyclopropanesulfonamidopyridin-2-yl)-2-[(2R)-1-isopropylpiperidin-2-yl]ethyl]-5-(6-ethoxypyrazin-2-yl)-1,3-thiazole-2-carboxamide, (I-27, second eluting peak, 17 mg) both as a white solids. 13.11 (80 mg) was purified by Prep-Chrial-HPLC (Column: CHIRALPAK IE-3, 4.6*50 mm 3 μm; Mobile Phase A: Hex:DCM=3:1)(0.1% DEA):EtOH=70:30; Flow rate: 1 mL/min; 254/210 nm) to afford N-[(1S)-1-(4-cyclopropanesulfonamidopyridin-2-yl)-2-[(2R)-1-isopropylpiperidin-2-yl]ethyl]-5-(6-ethoxypyrazin-2-yl)-1,3-thiazole-2-carboxamide, (I-28, first eluting peak, 18.6 mg) and N-[(1R)-1-(4-cyclopropanesulfonamidopyridin-2-yl)-2-[(2S)-1-isopropylpiperidin-2-yl]ethyl]-5-(6-ethoxypyrazine-2-yl)-1,3-thiazole-2-carboxamide, (I-29, second eluting peak, 18.0 mg) both as a white solids.

I-26. MS (ES): m/z 600 [M+H]⁺. ¹H NMR (400 MHz, DMSO-d6) δ 9.77 (d, J=7.6 Hz, 1H), 8.92 (s, 1H), 8.80 (s, 1H), 8.29-8.26 (m, 2H), 7.11 (s, 1H), 7.02-7.00 (m, 1H), 5.07 (q, J=7.6 Hz, 1H), 4.42 (q, J=7.2 Hz, 2H), 3.24-3.19 (m, 1H), 2.87-2.82 (m, 1H), 2.72-2.68 (m, 2H), 2.45-2.40 (m, 1H), 1.98-1.91 (m, 1H), 1.78-1.74 (m, 1H), 1.62-1.58 (m, 2H), 1.39 (t, J=7.2 Hz, 3H), 1.38-1.23 (m, 4H), 1.03-0.90 (m, 10H).

I-27. MS (ES): m/z 600 [M+H]⁺. ¹H NMR (400 MHz, DMSO-d6) δ 9.77 (s, 1H), 8.93 (s, 1H), 8.80 (s, 1H), 8.28 (s, 2H), 7.11 (s, 1H), 7.02-7.00 (m, 1H), 5.08-5.05 (m, 1H), 4.42 (q, J=7.2 Hz, 2H), 2.87-2.82 (m, 1H), 2.72-2.68 (m, 2H), 2.45-2.40 (m, 1H), 2.34-2.32 (m, 1H), 1.98-1.91 (m, 1H), 1.78-1.74 (m, 1H), 1.62-1.58 (m, 2H), 1.39 (t, J=7.2 Hz, 3H), 1.37-1.24 (m, 4H), 1.03-0.90 (m, 10H).

I-28. MS (ES): m/z 600 [M+H]⁺. ¹H NMR (400 MHz, DMSO-d6) δ 10.5 (s, 1H), 9.45 (d, J=8.0 Hz, 1H), 8.93 (s, 1H), 8.82 (s, 1H), 8.29 (s, 1H), 8.23 (s, 1H), 7.11 (s, 1H), 6.97 (d, J=8.0 Hz, 1H), 5.09-5.03 (m, 1H), 4.42 (q, J=7.2 Hz, 2H), 2.79-2.72 (m, 3H), 2.39-2.32 (m, 2H), 1.95-1.91 (m, 1H), 1.78-1.74 (m, 1H), 1.62-1.53 (m, 2H), 1.39 (t, J=7.2 Hz, 3H), 1.38-1.23 (m, 4H), 1.03-0.88 (m, 10H).

I-29. MS (ES): m/z 600 [M+H]⁺. ¹H NMR (400 MHz, DMSO-d6) δ 10.5 (s, 1H), 9.46 (s, 1H), 8.93 (s, 1H), 8.82 (s, 1H), 8.29 (s, 1H), 8.24 (s, 1H), 7.11 (s, 1H), 6.9-6.95 (m, 1H), 5.09-5.03 (m, 1H), 4.42 (q, J=7.2 Hz, 2H), 2.79-2.72 (m, 3H), 2.39-2.32 (m, 2H), 1.95-1.91 (m, 1H), 1.78-1.74 (m, 1H), 1.62-1.53 (m, 2H), 1.39 (t, J=7.2 Hz, 3H), 1.38-1.23 (m, 4H), 1.03-0.86 (m, 10H).

### Example 14: Preparation of Intermediate D (Int. D) and Compounds of the Disclosure

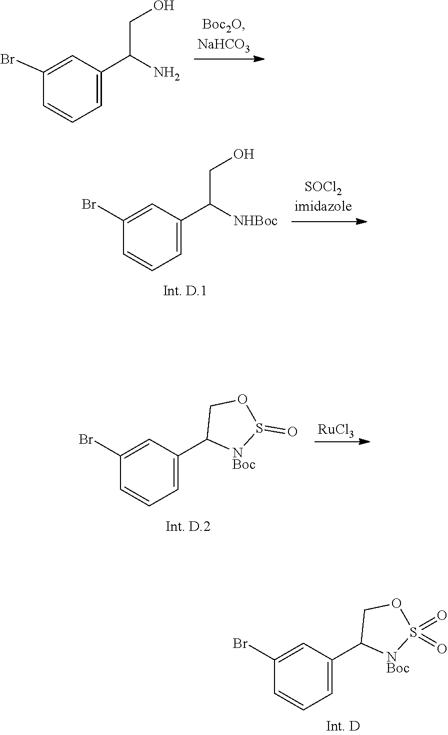

Synthesis of Int. D.1. To a solution of 2-amino-2-(3-bromophenyl)ethanol (10 g, 46.2 mmol, 1.0 eq.) and Boc₂O (15.1 g, 69.3 mmol, 1.5 eq.) in THF (60 mL) and water (60 mL) was added NaHCO₃ (3.9 g, 46.2 mmol, 1.0 eq.). The resulting mixture was stirred at room temperature for 1 h. The organic products were extracted with EtOAc (40 mL×3). The combined organic layers were concentrated under reduced pressure. The residue was purified by flash column chromatography on silica gel (eluted with 32% ethyl acetate in petro ether) to afford tert-butyl N-[1-(3-bromophenyl)-2-hydroxyethyl]carbamate (Int. D.1, 12.6 g, 86%) as a white solid. MS (ES): m/z 316/318 [M+H]⁺.

Synthesis of Int. D.2. A solution of imidazole (6.72 g, 98.7 mmol, 6.0 eq.) and SOCl₂ (1M in DCM, 30 mL, 29.6 mmol, 1.8 eq.) in DCM (50 mL) was stirred for 1 h at 0° C. under nitrogen atmosphere. Then a solution of Int D.1 (5.2 g, 16.4 mmol, 1.0 eq.) in DCM (50 mL) was added dropwise over 30 min at 0° C. The resulting mixture was stirred for 1 h at 0° C. under nitrogen atmosphere. The reaction was quenched with water (100 mL) at 0° C. The separated aqueous layer was extracted with DCM (40 mL×3). The combined organic layers were concentrated under reduced pressure. The residue was purified by flash column chromatography on silica gel (eluted with 50% ethyl acetate in petro ether) to afford tert-butyl 4-(3-bromophenyl)-1,2,3-oxathiazolidine-3-carboxylate 2-oxide (Int. D.2, 4.87 g, 82%) as a light red oil. MS (ES): m/z 362/364 [M+H]⁺.

Synthesis of Int. D. To a stirred solution of Int. D.2 (5.57 g, 15.4 mmol, 1 eq.) in ACN (70 mL) was added a solution of RuCl₃·H₂O (0.35 g, 1.54 mmol, 0.1 eq.) and NaIO₄ (3.62 g, 16.9 mmol, 1.1 eq.) in water (35 mL) dropwise at 0° C. The resulting mixture was stirred for 1 h at room temperature. The reaction was concentrated under reduced pressure. The residue was purified by flash column chromatography on silica gel (eluted with 100% ethyl acetate) to afford tert-butyl 4-(3-bromophenyl)-1,2,3-oxathiazolidine-3-carboxylate 2,2-dioxide (Int. D, 5.23 g, 90%) as a white solid. MS (ES): m/z 378/380 [M+H]⁺.

### General Method 2

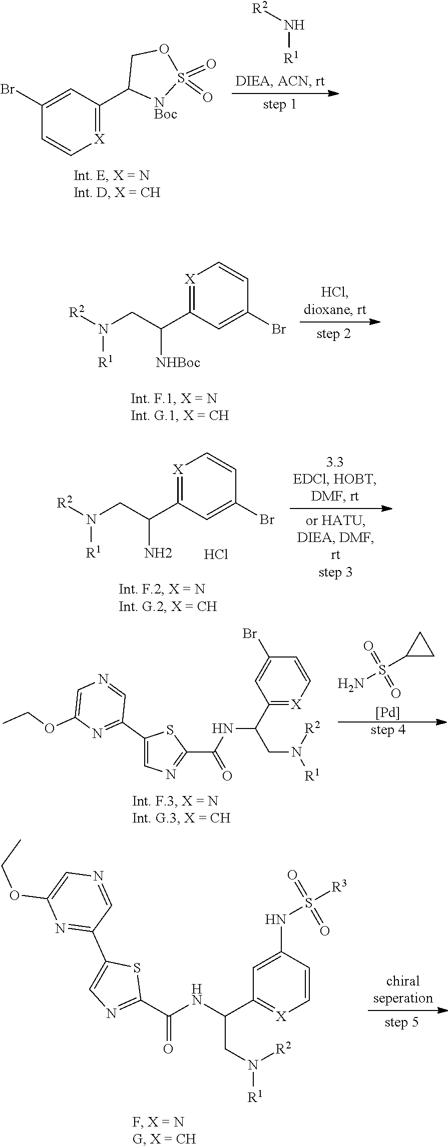

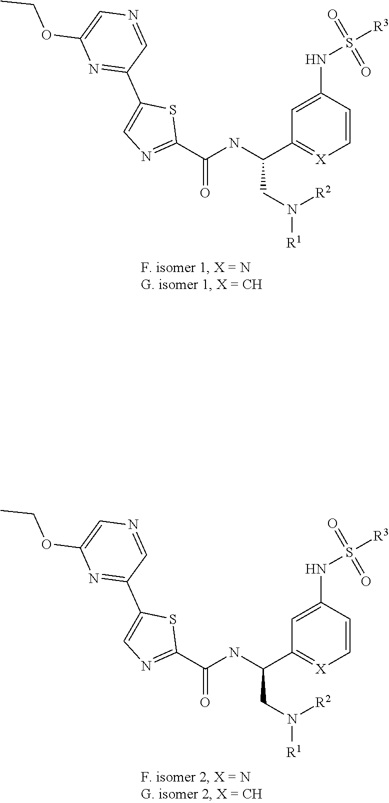

### Synthesis of N—((S)-1-(4-(cyclopropanesulfonamido) pyridin-2-yl)-2-((3aR,6aS)-tetrahydro-1H-furo[3,4-c]pyrrol-5(3H)-yl)ethyl)-5-(6-ethoxypyrazin-2-yl)thiazole-2-carboxamide, I-30 and N—((R)-1-(4-(cyclopropanesulfonamido) pyridin-2-yl)-2-((3aR,6aS)-tetrahydro-1H-furo[3,4-c]pyrrol-5(3H)-yl) ethyl)-5-(6-ethoxypyrazin-2-yl)thiazole-2-carboxamide, I-31

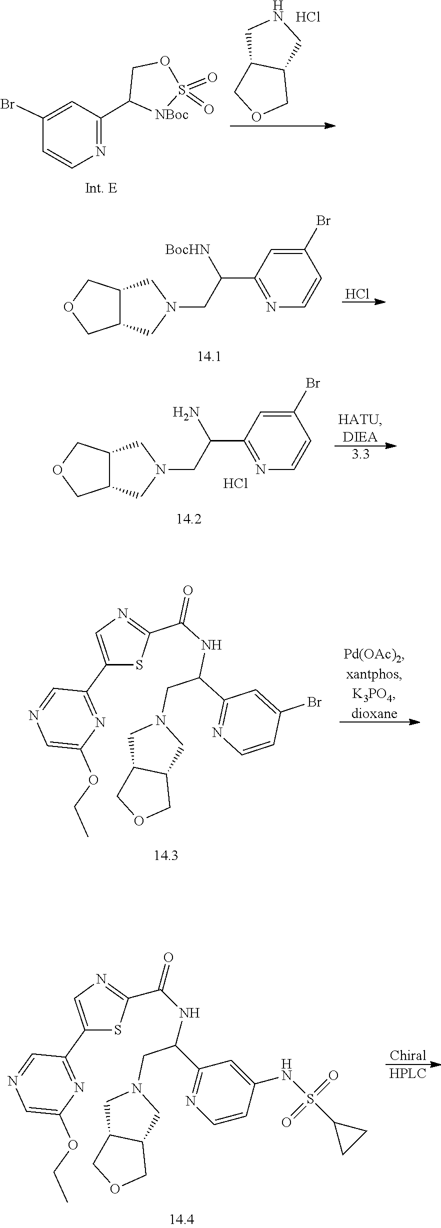

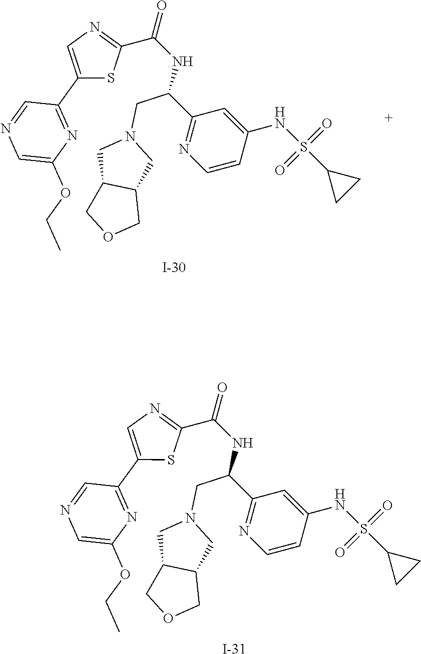

General Method 2, Step 1, synthesis of 14.1. To a stirred solution of Int. E (The synthesis of this intermediate is found in US patent application U.S. Ser. No. 17/452,011, filed on Oct. 22, 2021, the disclosure of which is hereby incorporated by reference) (500 mg, 1.3 mmol, 1.0 eq) and (3aR,6aS)-hexahydro-1H-furo[3,4-c]pyrrole (298 mg, 2.6 mmol, 2.0 eq) in acetonitrile (5 mL) was added DIEA (682 mg, 5.2 mmol, 4.0 eq) dropwise. The resulting mixture was stirred overnight at room temperature. The reaction mixture was concentrated under reduced pressure. The residue was purified by flash column chromatography on C18 silica (eluted with 50% acetonitrile in water) to afford tert-butyl N-{2-[(3aR,6aS)-hexahydrofuro[3,4-c]pyrrol-5-yl]-1-(4-bromopyridin-2-yl) ethyl}carbamate (14.1, 400 mg, 75%) as a white solid. LCMS (ESI, m/z): 412/414 [M+H]⁺.

General Method 2, Step 2, synthesis of 14.2. To a stirred solution of 14.1 (400 mg, 0.97 mmol, 1.0 eq) in DCM (2 mL) was added HCl (4M in 1,4-dioxane, 2 mL, 8.0 mmol, 8.2 eq). The resulting mixture was stirred for 1 h at room temperature. The reaction mixture was concentrated under reduced pressure to afford 2-[(3aR,6aS)-hexahydrofuro[3,4-c]pyrrol-5-yl]-1-(4-bromopyridin-2-yl) ethanamine hydrochloride (14.2, 280 mg) as a white solid, which was used in the next step without further purification. MS (ES): m/z 312/214 [M+H]⁺

General Method 2, Step 3, synthesis of 14.3. To a stirred solution of 14.2 (280 mg) and 3.3¹ (225 mg) in DCM (3 mL) were added HATU (512 mg, 1.34 mmol) and DIEA (463 mg, 3.59 mmol). The resulting mixture was stirred overnight at room temperature. The reaction was diluted with water (20 mL) and extracted with dichloromethane (3×10 mL). The combined organic layers were concentrated under reduced pressure. The residue was purified by flash column chromatography on C18 silica (eluted with 63% acetonitrile in water) to afford N-{2-[(3aR,6aS)-hexahydrofuro[3,4-c]pyrrol-5-yl]-1-(4-bromopyridin-2-yl) ethyl}-5-(6-ethoxypyrazin-2-yl)-1,3-thiazole-2-carboxamide (14.3, 250 mg, 47% over 2 steps) as a yellow oil. MS (ES): m/z 545/547 [M+H]⁺.

General Method 2, Step 4, synthesis of 14.4. To a stirred solution of 14.3 (250 mg, 0.46 mmol, 1.0 eq) and cyclopropanesulfonamide (111 mg, 0.92 mmol, 2.0 eq) in 1,4-dioxane (3 mL) were added XantPhos (53 mg, 0.09 mmol, 0.2 eq), Pd(OAc)₂ (11 mg, 0.05 mmol, 0.10 eq) and K₃PO₄ (195 mg, 0.92 mmol, 2.0 eq). The resulting mixture was stirred for 2 h at 90° C. under nitrogen atmosphere. The reaction was diluted with methanol (30 mL) and filtered through Celite. The filtrate was concentrated under reduced pressure. The residue was purified by flash column chromatography on C18 silica (eluted with 66% acetonitrile in water) to afford N-{2-[(3aR,6aS)-hexahydrofuro[3,4-c]pyrrol-5-yl]-1-(4-cyclopropanesulfonamidopyridin-2-yl)ethyl}-5-(6-ethoxypyrazin-2-yl)-1,3-thiazole-2-carboxamide (14.4, 120 mg, 45%) as a yellow oil. MS (ES): m/z 586 [M+H]⁺.

General Method 2, Step 5, synthesis of I-30 and I-31. 14.4 (120 mg) was separated by chiral-HPLC (Column: CHIRALPAK ID, 2*25 cm, 5 μm; Mobile Phase A: Hex:DCM=3:1 (0.5% 2M NH₃-MeOH)—-HPLC, Mobile Phase B: EtOH-HPLC; Flow rate: 20 mL/min; Gradient: 20% B to 20% B in 14 min; Wavelength: 220/254 nm) to afford N—((S)-1-(4-(cyclopropanesulfonamido) pyridin-2-yl)-2-((3aR,6aS)-tetrahydro-1H-furo[3,4-c]pyrrol-5(3H)-yl)ethyl)-5-(6-ethoxypyrazin-2-yl)thiazole-2-carboxamide, I-30, first eluting peak, 34 mg) and N—((R)-1-(4-(cyclopropanesulfonamido) pyridin-2-yl)-2-((3aR,6aS)-tetrahydro-1H-furo[3,4-c]pyrrol-5(3H)-yl) ethyl)-5-(6-ethoxypyrazin-2-yl)thiazole-2-carboxamide, I-31, first eluting peak, 30 mg) both as off-white solid.

I-30. MS (ES): m/z 586 [M+H]⁺; ¹H NMR (300 MHz, Methanol-d₄) δ 8.70 (s, 1H), 8.59 (s, 1H), 8.28 (d, J=5.7 Hz, 1H), 8.15 (s, 1H), 7.28 (d, J=2.1 Hz, 1H), 7.13 (dd, J=5.7, 2.1 Hz, 1H), 5.20-5.15 (m, 1H), 4.48 (q, J=7.2 Hz, 2H), 3.86-3.79 (m, 2H), 3.53-3.45 (m, 2H), 3.07-3.00 (m, 1H), 2.95-2.87 (m, 1H), 2.79-2.66 (m, 5H), 2.58-2.51 (m, 2H), 1.45 (t, J=7.2 Hz, 3H), 1.14-1.09 (m, 2H), 1.01-0.96 (m, 2H).

I-31. MS (ES): m/z 586 [M+H]*; ¹H NMR (300 MHz, Methanol-d₄) δ 8.70 (s, 1H), 8.59 (s, 1H), 8.28 (d, J=6.0 Hz, 1H), 8.15 (s, 1H), 7.28 (d, J=2.1 Hz, 1H), 7.14 (dd, J=6.0, 2.1 Hz, 1H), 5.20-5.16 (m, 1H), 4.48 (q, J=7.2 Hz, 2H), 3.85-3.79 (m, 2H), 3.54-3.45 (m, 2H), 3.08-3.01 (m, 1H), 2.95-2.89 (m, 1H), 2.79-2.67 (m, 5H), 2.59-2.52 (m, 2H), 1.45 (t, J=7.2 Hz, 3H), 1.16-1.09 (m, 2H), 1.01-0.94 (m, 2H).

### Example 15: Further Compounds Synthesized According to General Methods

The synthesized compounds in the following Table 2 were prepared according to General Method 2.

TABLE 2
Compounds prepared according to General Method 2

| | | Int | Amine | | |
|---|---|---|---|---|---|
| Com- | | used | reagent | | |
| pound | | in | used in | Sulfonamide | Chiral chromatography |
| No. | Name/Structure | step 1 | Step 1 | used in Step 4 | method |
| I-32 | 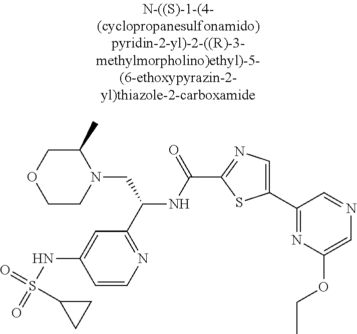 [SMILES: CCOc1cncc(-c2cnc(C(=O)N[C@@H](CN3CCOC[C@H]3C)c3cc(NS(=O)(=O)C4CC4)ccn3)s2)n1] | Int. E | (3R)-3- methyl morpholine | Cyclopropane- sulfonamide | chiral HPLC (Column: CHIRALPAK IG, 2*25 cm, 5 μm; Mobile Phase A: Hex:DCM = 3:1 (0.5% 2M NH₃—MeOH)--HPLC, Mobile Phase B: IPA--HPLC; Flow rate: 20 mL/min; Gradient: 30% B to 30% B in 16 min; Wave Length: 220/254 nm) First eluting peak |
| [symbol] | 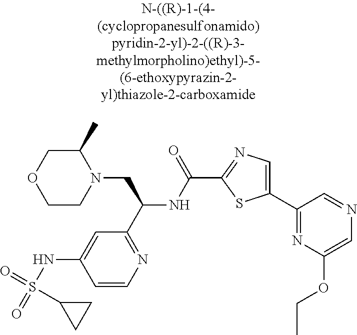 [SMILES: CCOc1cncc(-c2cnc(C(=O)N[C@H](CN3CCOC[C@H]3C)c3cc(NS(=O)(=O)C4CC4)ccn3)s2)n1] | Int. E | (3R)-3- methyl morpholine | Cyclopropane- sulfonamide | Same as I-32 Second eluting peak |
| I-34 | 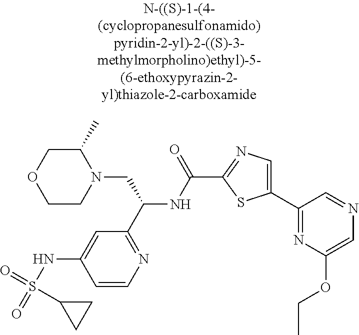 [SMILES: CCOc1cncc(-c2cnc(C(=O)N[C@@H](CN3CCOC[C@@H]3C)c3cc(NS(=O)(=O)C4CC4)ccn3)s2)n1] | Int. E | (3S)-3- methyl morpholine | Cyclopropane- sulfonamide | chiral HPLC (Column: CHIRALPAK IG, 2*25 cm, 5 μm; Mobile Phase A: Hex:DCM = 3:1 (0.5% 2M NH₃—MeOH)--HPLC, Mobile Phase B: EtOH--HPLC; Flow rate: 20 mL/min; Gradient: 20% B to 20% B in 9 min; Wave Length: 220/254 nm) First eluting peak |
| I-35 | 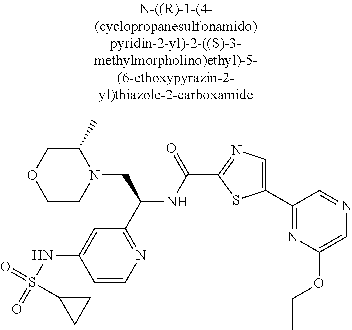 [SMILES: CCOc1cncc(-c2cnc(C(=O)N[C@H](CN3CCOC[C@@H]3C)c3cc(NS(=O)(=O)C4CC4)ccn3)s2)n1] | Int. E | (3S)-3- methyl morpholine | Cyclopropane- sulfonamide | Same as I-34 Second eluting peak |
| I-36 | 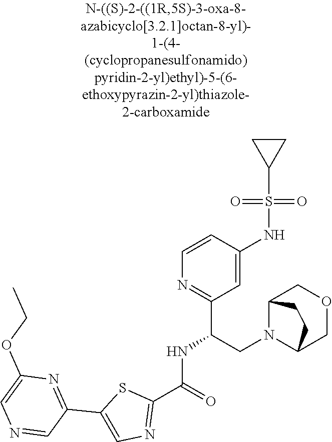 [SMILES: CCOc1cncc(-c2cnc(C(=O)N[C@@H](CN3[C@@H]4CC[C@H]3COC4)c3cc(NS(=O)(=O)C4CC4)ccn3)s2)n1] | Int. E | (1R,5S)- 3-oxa-8- azabicyclo [3.2.1] octan-8- ium chloride | Cyclopropane- sulfonamide | chiral HPLC (Column: CHIRALPAK IG, 2*25 cm, 5 μm; Mobile Phase A: Hex:DCM = 3:1 (0.5% 2M NH₃—MeOH)--HPLC, Mobile Phase B: EtOH--HPLC; Flow rate: 18 mL/min; Gradient: 50% B to 50% B in 20 min; Wave Length: 220/254 nm) First eluting peak |
| I-37 | 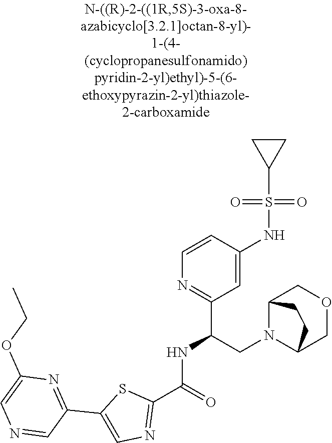 [SMILES: CCOc1cncc(-c2cnc(C(=O)N[C@H](CN3[C@@H]4CC[C@H]3COC4)c3cc(NS(=O)(=O)C4CC4)ccn3)s2)n1] | Int. E | (1R,5S)- 3-oxa- 8- azabicyclo [3.2.1] octan-8- ium chloride | Cyclopropane- sulfonamide | Same as I-36 Second eluting peak |
| [symbol] | 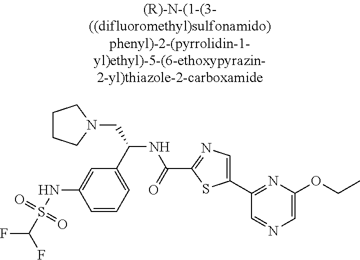 [SMILES: CCOc1cncc(-c2cnc(C(=O)N[C@@H](CN3CCCC3)c3cccc(NS(=O)(=O)C(F)F)c3)s2)n1] | Int. D | pyrrolidine | Difluoromeathane sulfonamide | chiral HPLC (Column: CHIRALPAK IE, 2*25 cm, 5 μm; Mobile Phase A: Hex:DCM = 3:1 (0.1% FA)-- HPLC, Mobile Phase B: EtOH--HPLC; Flow rate: 17 mL/min; Gradient: 50% B to 50% B in 30 min; Wave Length: 220/254 nm) First eluting peak |
| I-39 | 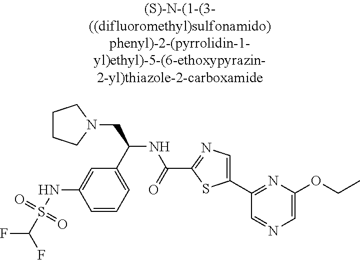 [SMILES: CCOc1cncc(-c2cnc(C(=O)N[C@H](CN3CCCC3)c3cccc(NS(=O)(=O)C(F)F)c3)s2)n1] | Int. D | pyrrolidine | Difluoromeathane sulfonamide | Same as I-38 Second eluting peak |
| I-40 | 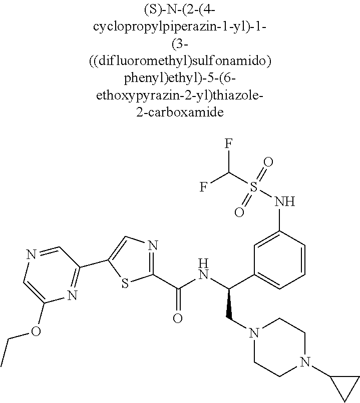 [SMILES: CCOc1cncc(-c2cnc(C(=O)N[C@H](CN3CCN(C4CC4)CC3)c3cccc(NS(=O)(=O)C(F)F)c3)s2)n1] | Int. D | 1- cyclopropyl- piperazine | Difluoromeathane sulfonamide | chiral HPLC (Column: CHIRALPAK IG, 2*25 cm, 5 μm; Mobile Phase A: Hex:DCM = 3:1 (0.1% FA)-- HPLC, Mobile Phase B: EtOH--HPLC; Flow rate: 20 mL/min; Gradient: 30% B to 30% B in 13 min; Wave Length: 220/254 nm) First eluting peak |
| I-41 | 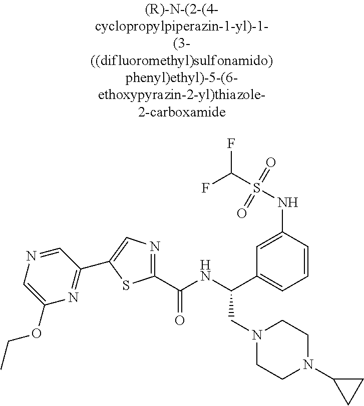 [SMILES: CCOc1cncc(-c2cnc(C(=O)N[C@@H](CN3CCN(C4CC4)CC3)c3cccc(NS(=O)(=O)C(F)F)c3)s2)n1] | Int. D | 1- cyclopropyl- piperazine | Difluoromeathane sulfonamide | Same as I-40 Second eluting peak |

| | MS (ES) | |
|---|---|---|
| | m/z | |
| Compound No. | [M + H]⁺ | ¹H NMR |
| I-32 | 574 | (300 MHz, DMSO-d₆) δ 10.55 (s, 1H), |
| | | 9.08 (s, 1H), 8.92 (s, 1H), 8.82 (s, 1H), |
| | | 8.36 (s, 1H), 8.29 (s, 1H), 7.26 (s, 1H), |
| | | 7.06 (s, 1H), 5.16-5.08 (m, 1H), 4.43 (q, |
| | | J = 7.2 Hz, 2H), 3.65-3.41 (m, 3H), 3.13- |
| | | 3.00 (m, 2H), 2.78-2.72 (m, 3H), 2.47- |
| | | 2.31 (m, 2H), 1.39 (t, J = 7.2 Hz, 3H), |
| | | 1.05-0.96 (m, 4H), 0.79 (d, J = 6.0 Hz, |
| | | 3H). |
| [symbol] | 574 | (300 MHz, DMSO-d₆) δ 10.55 (s, 1H), |
| | | 9.07 (s, 1H), 8.93 (s, 1H), 8.82 (s, 1H), |
| | | 8.37 (s, 1H), 8.29 (s, 1H), 7.27 (s, 1H), |
| | | 7.07 (s, 1H), 5.18-5.11 (m, 1H), 4.43 (q, |
| | | J = 7.2 Hz, 2H), 3.66-3.55 (m, 2H), 3.44- |
| | | 3.37 (m, 1H), 3.25-3.03 (m, 2H), 2.81- |
| | | 2.63 (m, 3H), 2.45-2.32 (m, 2H), 1.39 (t, |
| | | J = 7.2 Hz, 3H), 1.01-0.96 (m, 7H). |
| I-34 | 574 | (400 MHz, DMSO-d₆) δ 10.55 (s, 1H), |
| | | 9.06 (s, 1H), 8.93 (s, 1H), 8.82 (s, 1H), |
| | | 8.34 (s, 1H), 8.31 (s, 1H), 7.25 (s, 1H), |
| | | 7.06 (s, 1H), 5.17-5.11 (m, 1H), 4.43 (q, |
| | | J = 7.2 Hz, 2H), 3.64-3.55 (m, 1H), 3.42- |
| | | 3.36 (m, 1H), 3.23-3.18 (m, 1H), 3.08- |
| | | 3.04 (m, 1H), 2.78-2.64 (m, 3H), 2.45- |
| | | 2.36 (m, 2H), 1.39 (t, J = 7.2 Hz, 3H), |
| | | 1.01-0.96 (m, 7H). |
| I-35 | 574 | (400 MHz, DMSO-d₆) δ 10.54 (s, 1H), |
| | | 9.07 (s, 1H), 8.93 (s, 1H), 8.82 (s, 1H), |
| | | 8.37 (s, 1H), 8.29 (s, 1H), 7.26 (s, 1H), |
| | | 7.06 (s, 1H), 5.15-5.09 (m, 1H), 4.43 (q, |
| | | J = 7.2 Hz, 2H), 3.62-3.43 (m, 3H), 3.15- |
| | | 3.00 (m, 2H), 2.78-2.72 (m, 3H), 2.47- |
| | | 2.32 (m, 2H), 1.39 (t, J = 7.2 Hz, 3H), |
| | | 1.05-0.96 (m, 4H), 0.79 (d, J = 6.0 Hz, |
| | | 3H). |
| I-36 | 586 | (300 MHz, Methanol-d₄) δ 8.71 (s, 1H), |
| | | 8.61 (s, 1H), 8.29 (d, J = 6.0 Hz, 1H), 8.15 |
| | | (s, 1H), 7.33 (d, J = 2.4 Hz, 1H), 7.14 (dd, |
| | | J = 6.0, 2.4 Hz, 1H), 5.08 (t, J = 6.6 Hz, |
| | | 1H), 4.47 (q, J = 7.2 Hz, 2H), 3.70-3.64 |
| | | (m, 2H), 3.50-3.43 (m, 2H), 3.08-3.05 |
| | | (m, 1H), 2.92-2.88 (m, 1H), 2.86-2.80 |
| | | (m, 2H), 2.78-2.67 (m, 1H), 1.96-1.83 |
| | | (m, 4H), 1.45 (t, J = 7.2 Hz, 3H), 1.17- |
| | | 1.07 (m, 2H), 1.02-0.95 (m, 2H). |
| I-37 | 586 | (300 MHz, Methanol-d₄) δ 8.69 (s, 1H), |
| | | 8.58 (s, 1H), 8.29 (d, J = 6.0 Hz, 1H), 8.13 |
| | | (s, 1H), 7.33 (d, J = 2.1 Hz, 1H), 7.14 (dd, |
| | | J = 6.0, 2.1 Hz, 1H), 5.08 (t, J = 6.6 Hz, |
| | | 1H), 4.47 (q, J = 7.2 Hz, 2H), 3.70-3.62 |
| | | (m, 2H), 3.49-3.43 (m, 2H), 3.08-3.05 |
| | | (m, 1H), 2.94-2.89 (m, 1H), 2.86-2.82 |
| | | (m, 2H), 2.75-2.70 (m, 1H), 1.98-1.79 |
| | | (m, 4H), 1.44 (t, J = 7.2 Hz, 3H), 1.21- |
| | | 1.10 (m, 2H), 1.02-0.93 (m, 2H). |
| [symbol] | 553 | (400 MHz, DMSO-d₆) δ 8.69 (s, 1H), |
| | | 8.58 (s, 1H), 8.15 (s, 1H), 7.33-7.30 (m, |
| | | 2H), 7.23-7.18 (m, 2H), 6.54 (t, J = 53.6 |
| | | Hz, 1H), 5.39-5.35 (m, 1H), 4.47 (q, J = |
| | | 7.2 Hz, 2H), 3.50-3.44 (m, 1H), 3.17-3.10 |
| | | (m, 1H), 3.05-2.95 (m, 4H), 1.97-1.91 |
| | | (m, 4H), 1.44 (t, J = 7.2 Hz, 3H). |
| I-39 | 553 | (300 MHz, DMSO-d₆) δ 8.69 (s, 1H), |
| | | 8.59 (s, 1H), 8.15 (s, 1H), 7.38-7.34 (m, |
| | | 2H), 7.29-7.21 (m, 2H), 6.60 (t, J = 53.4 |
| | | Hz, 1H), 5.51-5.46 (m, 1H), 4.47 (q, J = |
| | | 7.2 Hz, 2H), 3.71-3.63 (m, 1H), 3.39-3.33 |
| | | (m, 1H), 3.29-3.16 (m, 4H), 2.03-1.98 |
| | | (m, 4H), 1.44 (t, J = 7.2 Hz, 3H). |
| I-40 | 608 | (400 MHz, Methanol-d₄) δ 8.70 (s, 1H), |
| | | 8.60 (s, 1H), 8.15 (s, 1H), 7.35-7.31 (m, |
| | | 2H), 7.28-7.18 (m, 2H), 6.64 (t, J = 53.2 |
| | | Hz, 1H), 5.24-5.21 (m, 1H), 4.47 (q, J = |
| | | 7.2 Hz, 2H), 3.00-2.94 (m, 1H), 2.80-2.68 |
| | | (m, 7H), 2.65-2.54 (m, 2H), 1.84-1.81 |
| | | (m, 1H), 1.44 (t, J = 7.2 Hz, 3H), 0.56- |
| | | 0.44 (m, 4H). |
| I-41 | 608 | (400 MHz, Methanol-d₄) δ 8.70 (s, 1H), |
| | | 8.60 (s, 1H), 8.15 (s, 1H), 7.35-7.31 (m, |
| | | 2H), 7.28-7.18 (m, 2H), 6.64 (t, J = 53.2 |
| | | Hz, 1H), 5.24-5.21 (m, 1H), 4.47 (q, J = |
| | | 7.2 Hz, 2H), 2.99-2.94 (m, 1H), 2.80-2.68 |
| | | (m, 7H), 2.65-2.54 (m, 2H), 1.84-1.81 |
| | | (m, 1H), 1.44 (t, J = 7.2 Hz, 3H), 0.57- |
| | | 0.42 (m, 4H). |

[symbol]

indicates data missing or illegible when filed

### Synthesis of (R)—N-(3-(4-(cyclopropanesulfonamido)pyridin-2-yl)tetrahydrofuran-3-yl)-5-(6-ethoxypyrazin-2-yl)thiazole-2-carboxamide, isomer 1 I-71 and (S)—N-(3-(4-(cyclopropanesulfonamido)pyridin-2-yl)tetrahydrofuran-3-yl)-5-(6-ethoxypyrazin-2-yl)thiazole-2-carboxamide, isomer 2 I-72. Arbitrary Stereochemical Assignment

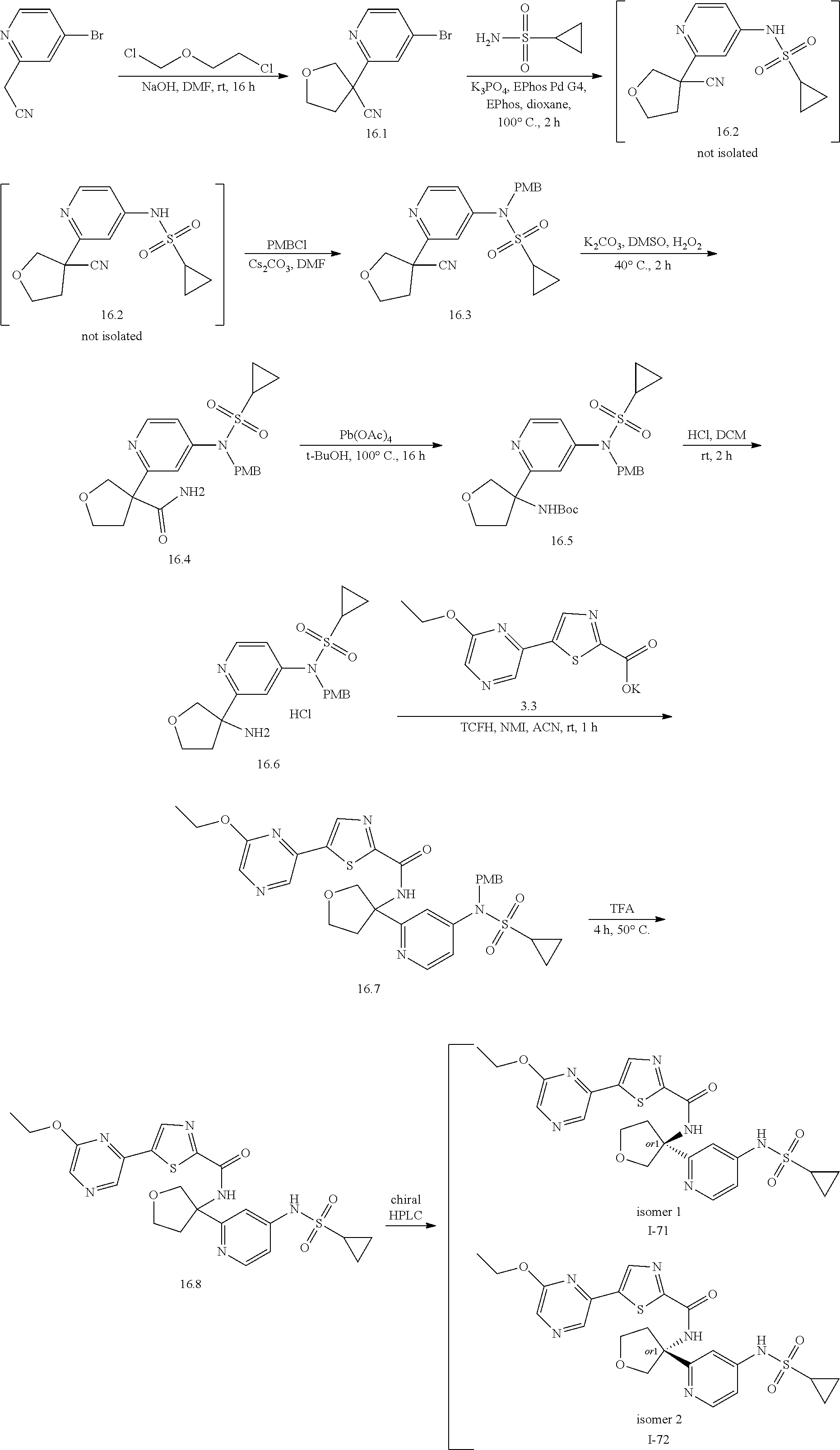

Synthesis of 16.1. To a stirred solution of 2-(4-bromopyridin-2-yl) acetonitrile (5 g, 25.5 mmol, 1 eq) and sodium hydrate (3.06 g, 76.5 mmol, 3 eq) in N, N-dimethylformamide (65 mL) was added 1-chloro-2-(chloromethoxy)ethane (3.91 g, 30.6 mmol, 1.2 eq) in portions at room temperature. The resulting mixture was stirred for 16 h at room temperature. The mixture was diluted with water and extracted with ethyl acetate. The combined organic layer was washed with brine, dried over sodium sulfate and concentrated under reduced pressure. The residue was purified by silica gel column chromatography eluting with 25% ethyl acetate in petroleum ether to obtain 3-(4-bromopyridin-2-yl)tetrahydrofuran-3-carbonitrile (16.1, 1.02 g, 16%) as a white solid. MS (ES): m/z 253/255 [M+H]⁺.

Synthesis of 16.3. To a solution of 16.1 (1.02 g, 4 mmol, 1 eq) and cyclopropanesulfonamide (968 mg, 8 mmol, 2 eq) in dioxane (10 mL) was added EPhos (214 mg, 0.4 mmol, 0.1 eq), EPhos Pd G4 (367 mg, 0.4 mmol, 0.1 eq) and potassium phosphate (1.69 g, 8 mmol, 2.0 eq) in portions at room temperature under nitrogen atmosphere. The resulting mixture was degassed three times with nitrogen and vacuum, the stirred for 2.5 h at 100° C. under nitrogen atmosphere. The mixture was cooled to room temperature, diluted with N, N-dimethylformamide (10 mL), and cesium carbonate (2.6 g, 8 mmol, 2 eq) and PMBCl (936 mg, 6 mmol, 1.5 eq) were added. The resulting mixture was stirred for 2 days at 80° C. The mixture was cooled to room temperature, diluted with water and extracted with ethyl acetate. The organic layer was washed with brine, dried over sodium sulfate and concentrated under reduced pressure. The residue was purified by reverse flash chromatography (column: C18 silica gel; mobile phase, ACN in Water (0.1% FA), 10% to 100% gradient in 20 min; detector, UV 254 nm) to obtain N-(2-(3-cyanotetrahydrofuran-3-yl)pyridin-4-yl)-N-(4-methoxybenzyl)cyclopropanesulfonamide (16.3, 730 mg, 43%) as a yellow solid. MS (ES): m/z 414 [M+H]f.

Synthesis of 16.4. To a stirred solution of 16.3 (730 mg, 1.76 mmol, 1 eq) in dimethysulfoxide (10 mL) were added potassium carbonate (96 mg, 0.7 mmol, 0.4 eq) and hydrogen peroxide (30% w/w in water, 0.6 mL) dropwise at 0° C. under nitrogen atmosphere. The resulting mixture was stirred for 2 h at 40° C. under nitrogen atmosphere. The mixture was cooled to room temperature, diluted with water and extracted with ethyl acetate. The combined organic layer was washed with brine, dried over sodium sulfate and concentrated under reduced pressure. The residue was purified by silica gel column chromatography eluting with 2% methanol in dichloromethane to obtain 3-(4-(N-(4-methoxybenzyl)cyclopropanesulfonamido)pyridin-2-yl)tetrahydrofuran-3-carboxamide (16.4, 730 mg, 95%) as a white solid. MS (ES): m/z 432 [M+H]⁻.

Synthesis of 16.5. To a stirred solution of 16.4 (730 mg, 1.69 mmol, 1 eq) in tert-butyl alcohol (10 mL) were added lead (IV) acetate (3 g, 6.76 mmol, 4 eq) in portions at room temperature under nitrogen atmosphere. The resulting mixture was stirred for 16 h at 100° C. under nitrogen atmosphere. The mixture was cooled to room temperature, diluted with water and extracted with ethyl acetate. The organic layer was washed with brine, dried over sodium sulfate and concentrated under reduced pressure to afford crude tert-butyl (3-(4-(N-(4-methoxybenzyl)cyclopropanesulfonamido)pyridin-2-yl)tetrahydrofuran-3-yl)carbamate (16.5, 500 mg, crude) as a yellow oil, which was used directly in the next step without further purification. MS (ES): m/z 504 [M+H]⁺.

Synthesis of 16.6. To a stirred solution of 16.5 (500 mg, crude) in dichloromethane (3 mL) were added hydrochloric acid (4M in 1,4-dioxane, 3 mL) dropwise at room temperature. The resulting mixture was stirred for 2 h at room temperature. The reaction mixture was concentrated under reduced pressure to afford N-(2-(3-aminotetrahydrofuran-3-yl)pyridin-4-yl)-N-(4-methoxybenzyl)cyclopropane sulfonamide hydrochloride (16.6, 400 mg, crude) as a white solid. MS (ES): m/z 404 [M+H]⁺.

Synthesis of 16.7. To a stirred solution of 16.6 (400 mg, crude) and 3.3 (235 mg) in acetonitrile (5 mL) were added N, N, N′, N′-tetramethyl chloro forma midinium-hexafluorophosphate (300 mg) and N-methyl imidazole (219 mg) in portions at room temperature. The resulting mixture was stirred for 1 h at room temperature. The residue was purified by reverse phase flash chromatography (column, C18 silica gel; mobile phase, ACN in Water (0.1% FA), 10% to 50% gradient in 10 min; detector, UV 254 nm). The desired fractions were combined and concentrated under vacuum to obtain 5-(6-ethoxypyrazin-2-yl)-N-(3-(4-(N-(4-methoxybenzyl)cyclopropanesulfonamido) pyridine-2-yl)tetrahydrofuran-3-yl)thiazole-2-carboxamide (16.7, 140 mg, 13% over 3 steps) as a white solid. MS (ES): m/z 637 [M+H]f.

Synthesis of 16.8. To a stirred solution of 16.7 (140 mg, 0.22 mmol, 1 eq) was added trifluoroacetic acid (2 mL) in portions at room temperature. The resulting mixture was stirred for 4 h at 50° C. under nitrogen atmosphere. The residue was purified by reverse phase flash chromatography (column, C18 silica gel; mobile phase, ACN in Water (0.1% FA), 10% to 50% gradient in 10 min; detector, UV 254 nm). The desired fractions were combined and concentrated under vacuum to obtain N-(3-(4-(cyclopropanesulfonamido)pyridin-2-yl)tetrahydrofuran-3-yl)-5-(6-ethoxypyrazin-2-yl)thiazole-2-carboxamide (16.8, 54 mg, 45%) as a white solid. MS (ES): m/z 517 [M+H]V.

Synthesis of I-71 and I-72. 16.8 (54 mg) was purified by chiral HPLC (Column: CHIRAL ART Cellulose-SC, 2*25 cm, 5 μm; Mobile Phase A: Hex:DCM=3:1 (0.5% 2M NH₃-MeOH)—-HPLC, Mobile Phase B: EtOH-HPLC; Flow rate: 20 mL/min; Gradient: 50% B to 50% B in 10 min; Wave Length: 220/254 nm) to afford (R)—N-(3-(4-(cyclopropanesulfonamido)pyridin-2-yl)tetrahydrofuran-3-yl)-5-(6-ethoxypyrazin-2-yl)thiazole-2-carboxamide (I-71, first eluting peak, 21 mg, 38%) and (S)—N-(3-(4-(cyclopropanesulfonamido)pyridin-2-yl)tetrahydrofuran-3-yl)-5-(6-ethoxypyrazin-2-yl)thiazole-2-carboxamide (I-72, second eluting peak, 27 mg, 49%) both as white solids.

I-71. MS (ES): m/z 517 [M+H]⁺; Rt=1.687 (LCMS-Q); ¹H NMR (300 MHz, DMSO-d₆) δ 10.49 (s, 1H), 9.54 (s, 1H), 8.95 (s, 1H), 8.84 (s, 1H), 8.37 (d, J=5.6 Hz, 1H), 8.29 (s, 1H), 7.27 (s, 1H), 7.05 (d, J=5.4 Hz, 1H), 4.46-4.30 (m, 3H), 4.12 (d, J=9.0 Hz, 1H), 3.98 (q, J=7.3 Hz, 2H), 2.82-2.73 (m, 2H), 2.60-2.55 (m, 1H), 1.39 (t, J=7.0 Hz, 3H), 1.02-0.90 (m, 4H).

I-72. MS (ES): m/z 517 [M+H]⁺; Rt=1.687 (LCMS-Q); ¹H NMR (300 MHz, DMSO-d₆) δ 10.50 (s, 1H), 9.53 (s, 1H), 8.95 (s, 1H), 8.84 (s, 1H), 8.36 (d, J=5.6 Hz, 1H), 8.29 (s, 1H), 7.27 (s, 1H), 7.05 (dd, J=5.6, 2.1 Hz, 1H), 4.46-4.30 (m, 3H), 4.12 (d, J=9.0 Hz, 1H), 3.98 (q, J=7.3 Hz, 2H), 2.82-2.73 (m, 2H), 2.60-2.55 (m, 1H), 1.39 (t, J=7.0 Hz, 3H), 1.02-0.90 (m, 4H).

### Synthesis of (S)—N-((6-(cyclopropanesulfonamido)pyrimidin-4-yl)(tetrahydro-2H-pyran-4-yl)methyl)-5-(6-ethoxypyrazin-2-yl)thiazole-2-carboxamide, isomer 1 I-254 and (R)—N-((6-(cyclopropanesulfonamido)pyrimidin-4-yl)(tetrahydro-2H-pyran-4-yl)methyl)-5-(6-ethoxypyrazin-2-yl)thiazole-2-carboxamide, isomer 2 I-255. Arbitrary stereochemical assignment

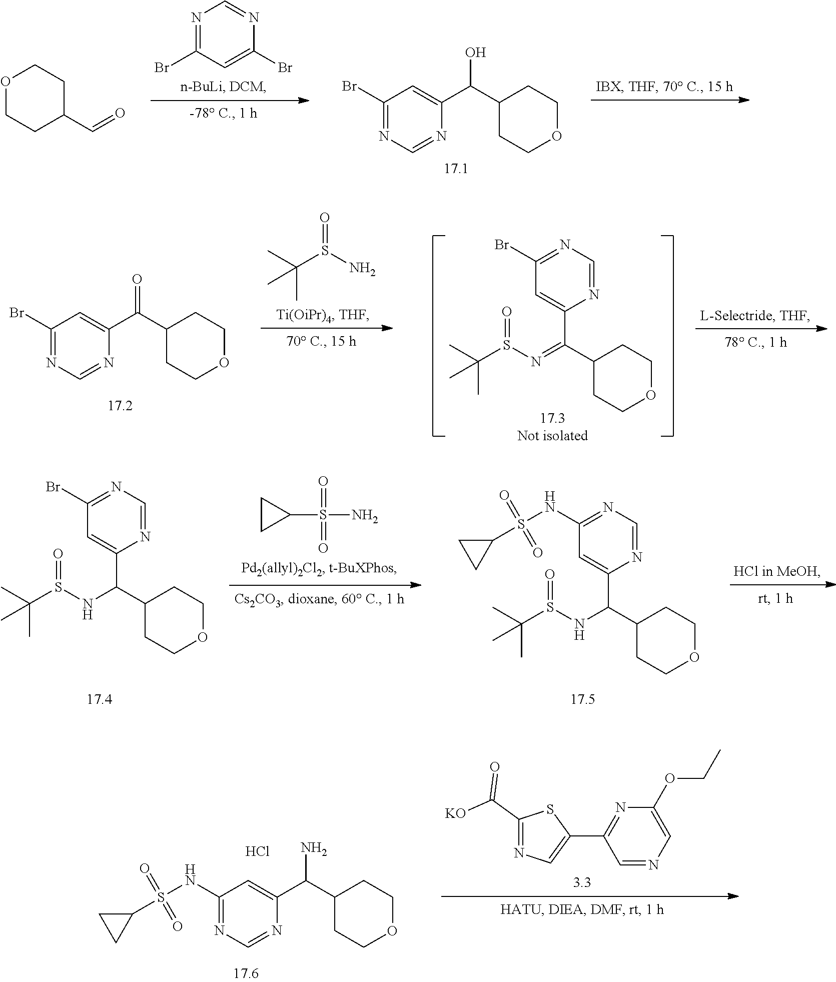

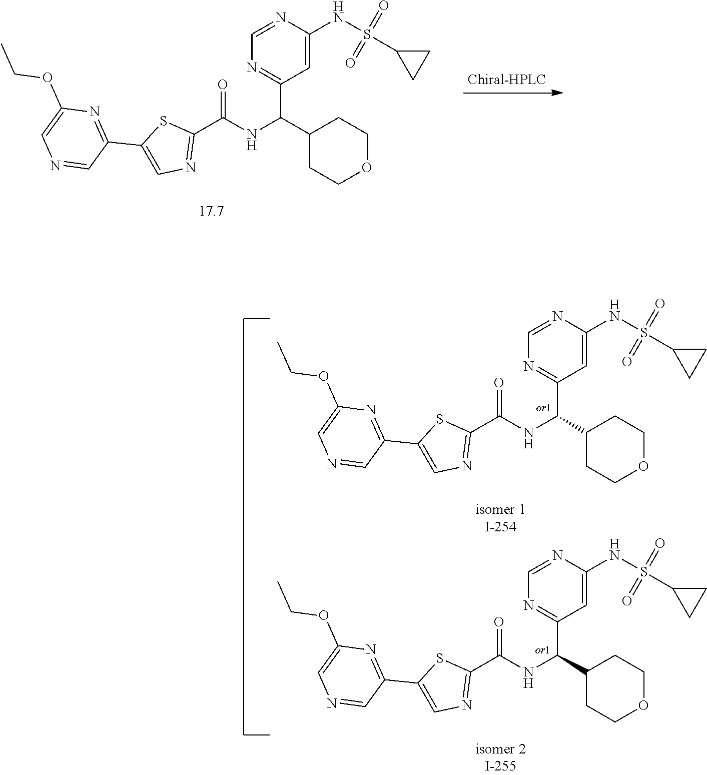 [SMILES: CCOc1cncc(-c2cnc(C(=O)NC(c3cc(NS(=O)(=O)C4CC4)ncn3)C3CCOCC3)s2)n1 ; "Chiral-HPLC" ; CCOc1cncc(-c2cnc(C(=O)N[C@H](c3cc(NS(=O)(=O)C4CC4)ncn3)C3CCOCC3)s2)n1 ; CCOc1cncc(-c2cnc(C(=O)N[C@@H](c3cc(NS(=O)(=O)C4CC4)ncn3)C3CCOCC3)s2)n1]

Synthesis of 17.1. To a stirred mixture of 4,6-dibromopyrimidine (4.2 g, 17.8 mmol, 1 eq) in dichloromethane (40 mL) was added n-butyllithium (2.5 M in THF, 8.5 mL, 21.3 mmol, 1.2 eq) dropwise at −78° C. under nitrogen atmosphere. The mixture was stirred for 1 h at −78° C. A solution of oxane-4-carbaldehyde (2.02 g, 17.8 mmol, 1 eq) in dichloromethane (2 mL) was added dropwise at −78° C. under nitrogen atmosphere. The resulting mixture was allowed to warm to room temperature and stirred for 1 h. The reaction was quenched with aqueous saturated ammonium chloride and extracted with dichloromethane. After filtration, the filtrate was concentrated under reduced pressure. The residue was purified by silica gel column chromatography (compound was eluted with 35% ethyl acetate in petroleum ether) to afford (6-bromopyrimidin-4-yl) (oxan-4-yl) methanol (17.1, 1.86 g, 38%) as a yellow oil. MS (ES): m/z 273/275 [M+H]⁻.

Synthesis of 17.2. To a stirred mixture of 17.1 (1.86 g, 6.83 mmol, 1 eq) in tetrahydrofuran (40 mL) was added IBX (7.64 g, 27.32 mmol, 4 eq) at room temperature. The mixture was stirred for 15 h at 70° C. The mixture was allowed to cool down to room temperature. The resulting mixture was filtered; the filter cake was washed with dichloromethane. The filtrate was diluted water and extracted with dichloromethane. The combined organic layers were washed with brine, dried over anhydrous sodium sulfate. After filtration, the filtrate was concentrated under reduced pressure. The residue was purified by silica gel column chromatography (compound was eluted with 20% ethyl acetate in petroleum ether) to obtain 4-bromo-6-(oxane-4-carbonyl) pyrimidine (17.2, 1 g, 54%) as a white solid. MS (ES, m/z): 271/273 [M+H]⁺.

Synthesis of 17.4. To a stirred solution of 17.2 (1 g, 3.7 mmol, 1 eq) and tert-butanesulfinamide (671 mg, 5.55 mmol, 1.5 eq) in tetrahydrofuran (20 mL) was added titanium tetraisopropanolate (2.1 g, 7.4 mmol, 2 eq) in portions at room temperature. The resulting mixture stirred for 15 h at 70° C. under nitrogen atmosphere. The mixture was cooled to −78° C. and L-Selectride (5.3 mL) was added at −78° C. under nitrogen atmosphere. The mixture was stirred for 1 h at −78° C. The reaction was quenched with aqueous saturated ammonium chloride and extracted with dichloromethane. After filtration, the filtrate was concentrated under reduced pressure. The residue was purified by silica gel column chromatography (compound was eluted with 50% ethyl acetate in petroleum ether) to afford N-[(6-bromopyrimidin-4-yl) (oxan-4-yl) methyl]-2-methylpropane-2-sulfinamide (17.4, 900 mg, 64%) as a brown yellow solid. MS (ES, m/z): 376/378 [M+H]⁺.

Synthesis of 17.5. To a stirred mixture of 17.4 (900 mg, 2.4 mmol, 1 eq) and cyclopropanesulfonamide (580 mg, 4.8 mmol, 2 eq) in dioxane (15 mL) was added Pd₂(allyl)₂Cl₂ (86 mg, 0.24 mmol, 0.1 eq), t-BuXPhos (203 mg, 0.48 mmol, 0.2 eq) and Cs₂CO₃ (2.34 g, 7.2 mmol, 3 eq) at room temperature under nitrogen atmosphere. The mixture was stirred for 1 h at 60° C. The mixture was allowed to cool down to room temperature. The resulting mixture was concentrated under reduced pressure. The residue was purified by reverse flash chromatography (Column C18; mobile phase, Water (0.1% NH₄HCO₃) and ACN (0% ACN up to 33% in 20 min); Detector, UV 254/220 nm). The reaction was concentrated under reduced pressure to afford N-(6-(((tert-butylsulfinyl)amino)(tetrahydro-2H-pyran-4-yl)methyl)pyrimidin-4-yl)cyclopropanesulfonamide (17.5, 800 mg, 76%) as a brown yellow solid. MS (ES, m/z): 417 [M+H]⁺.

Synthesis of 17.6. To a stirred solution of 17.5 (200 mg, 0.48 mmol, 1 eq) in dichloromethane (3 mL) was added hydrochloric acid (4M in 1,4-dioxane, 3 mL) dropwise at room temperature. The resulting mixture was stirred for 2 h at room temperature. The reaction mixture was concentrated under reduced pressure to afford N-(6-(amino(tetrahydro-2H-pyran-4-yl)methyl)pyrimidin-4-yl)cyclopropane sulfonamide, hydrochloride (17.6, 200 mg, crude) as a brown yellow solid. MS (ES): m/z 313 [M+H]⁺.

Synthesis of 17.7. To a stirred mixture of 17.6 (200 mg, crude) and 3.3 (145 mg) in N, N-dimethylformamide (5 mL) was added HATU (326 mg, 0.86 mmol) and DIEA (296 mg, 2.3 mmol) at room temperature. The mixture was stirred for 1 h at room temperature. The residue was purified by reverse flash chromatography (Column C18; mobile phase, Water (0.10% NH₄HCO₃) and ACN (0% ACN up to 35% in 20 min); Detector, UV 254/220 nm). The reaction was concentrated under reduced pressure to afford N-((6-(cyclopropanesulfonamido)pyrimidin-4-yl)(tetrahydro-2H-pyran-4-yl)methyl)-5-(6-ethoxypyrazin-2-yl)thiazole-2-carboxamide (17.7, 120 mg, 46% over 2 steps) as a brown yellow solid. MS (ES, m/z): 546 [M+H]⁺.

Synthesis of I-254 and I-255. 17.7 (120 mg) was purified by chiral HPLC (Column: CHIRALPAK IG, 2*25 cm, 5 μm; Mobile Phase A: Hex: DCM=1:1 (0.1% FA)-HPLC, Mobile Phase B: EtOH-HPLC; Flow rate: 20 mL/min; Gradient: 50% B to 50% B in 12 min; Wave Length: 220/254 nm; RT1(min): 6.17; RT2(min): 9.73). The product-containing fractions were combined, evaporated partially in vacuum then lyophilized overnight to afford (S)—N-((6-(cyclopropanesulfonamido)pyrimidin-4-yl)(tetrahydro-2H-pyran-4-yl)methyl)-5-(6-ethoxypyrazin-2-yl)thiazole-2-carboxamide (I-254, first eluting peak, 25 mg, 20%) and (R)—N-((6-(cyclopropanesulfonamido)pyrimidin-4-yl)(tetrahydro-2H-pyran-4-yl)methyl)-5-(6-ethoxypyrazin-2-yl)thiazole-2-carboxamide (I-255, second eluting peak, 27 mg, 22%) both as white solids.

I-254. MS (ES): m/z 546 [M+H]⁺; Rt=1.088 (LCMS-AI); ¹H NMR (300 MHz, Methanol-d₄) δ 8.75 (d, J=1.2 Hz, 1H), 8.68 (s, 1H), 8.57 (s, 1H), 8.14 (s, 1H), 7.12 (d, J=1.2 Hz, 1H), 4.90-4.87 (m, 1H), 4.47 (q, J=7.1 Hz, 2H), 4.02-3.82 (m, 2H), 3.46-3.34 (m, 2H), 3.04-2.98 (m, 1H), 2.32-2.22 (m, 1H), 1.80-1.72 (m, 1H), 1.56-1.30 (m, 6H), 1.28-1.18 (m, 2H), 1.05-0.96 (m, 2H).

I-255. MS (ES): m/z 546 [M+H]⁺; Rt=1.095 (LCMS-AI); ¹H NMR (300 MHz, Methanol-d₄) δ 8.75 (d, J=1.2 Hz, 1H), 8.69 (s, 1H), 8.58 (s, 1H), 8.14 (s, 1H), 7.12 (d, J=1.2 Hz, 1H), 4.90-4.87 (m, 1H), 4.47 (q, J=7.1 Hz, 2H), 4.03-3.86 (m, 2H), 3.46-3.34 (m, 2H), 3.04-2.98 (m, 1H), 2.32-2.20 (m, 1H), 1.80-1.72 (m, 1H), 1.56-1.30 (m, 6H), 1.28-1.18 (m, 2H), 1.07-0.99 (m, 2H).

TABLE 3
Additional Compounds

| Com- | | MS (ES): | LCMS Rt |
|---|---|---|---|
| pound | Name/Structure | m/z | (mins) |
| No. | Stereochemistry alpha to central amide arbitrary assigned | [M + H]+ | Method |
| I-42 | (R)-N-(1-(4-(cyclopropanesulfonamido)pyridin-2-yl)-3- | 560 | 1.044 |
| | (dimethylamino)-2,2-dimethylpropyl)-5-(6-ethoxypyrazin-2- | | LCMS-BB |
| | yl)thiazole-2-carboxamide | | |
| | 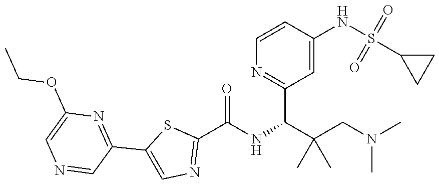 [SMILES: CCOc1cncc(-c2cnc(C(=O)N[C@H](c3cc(NS(=O)(=O)C4CC4)ccn3)C(C)(C)CN(C)C)s2)n1] | | |
| I-43 | (R)-N-(1-(4-(cyclopropanesulfonamido)pyridin-2-yl)-3- | 560 | 1.041 |
| | (dimethylamino)-2,2-dimethylpropyl)-5-(6-ethoxypyrazin-2- | | LCMS-BB |
| | yl)thiazole-2-carboxamide | | |
| | 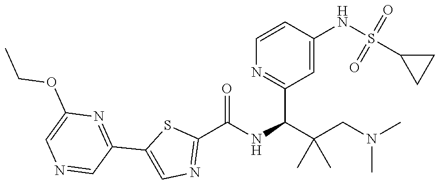 [SMILES: CCOc1cncc(-c2cnc(C(=O)N[C@@H](c3cc(NS(=O)(=O)C4CC4)ccn3)C(C)(C)CN(C)C)s2)n1] | | |
| I-44 | N-((1S,2S)-1-(4-(cyclopropanesulfonamido)pyridin-2-yl)-2- | 519 | 1.397 |
| | methoxypropyl)-5-(6-ethoxypyrazin-2-yl)thiazole-2- | | LCMS-C |
| | carboxamide | | |
| | 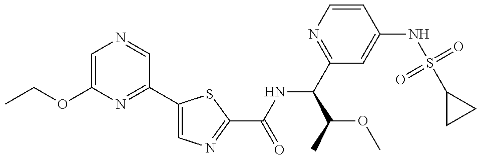 [SMILES: CCOc1cncc(-c2cnc(C(=O)N[C@@H](c3cc(NS(=O)(=O)C4CC4)ccn3)[C@H](C)OC)s2)n1] | | |
| I-45 | N-((1R,2R)-1-(4-(cyclopropanesulfonamido)pyridin-2-yl)-2- | 519 | 1.662 |
| | methoxypropyl)-5-(6-ethoxypyrazin-2-yl)thiazole-2- | | LCMS-V |
| | carboxamide | | |
| | 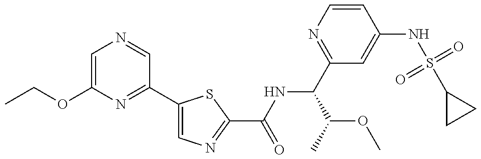 [SMILES: CCOc1cncc(-c2cnc(C(=O)N[C@H](c3cc(NS(=O)(=O)C4CC4)ccn3)[C@@H](C)OC)s2)n1] | | |
| I-46 | N-((1S,2R)-1-(4-(cyclopropanesulfonamido)pyridin-2-yl)-2- | 519 | 1.635 |
| | methoxypropyl)-5-(6-ethoxypyrazin-2-yl)thiazole-2- | | LCMS-Z |
| | carboxamide | | |
| | 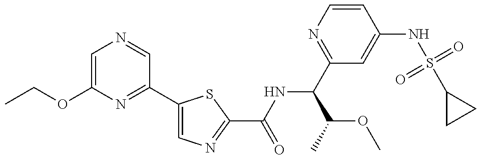 [SMILES: CCOc1cncc(-c2cnc(C(=O)N[C@@H](c3cc(NS(=O)(=O)C4CC4)ccn3)[C@@H](C)OC)s2)n1] | | |
| I-47 | N-((1S,2S)-1-(4-(cyclopropanesulfonamido)pyridin-2-yl)-2- | 519 | 1.637 |
| | methoxypropyl)-5-(6-ethoxypyrazin-2-yl)thiazole-2- | | LCMS-Z |
| | carboxamide | | |
| | 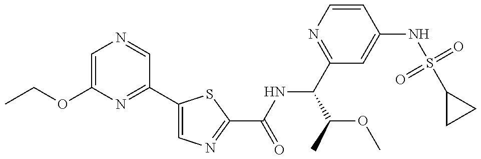 [SMILES: CCOc1cncc(-c2cnc(C(=O)N[C@H](c3cc(NS(=O)(=O)C4CC4)ccn3)[C@H](C)OC)s2)n1] | | |
| I-48 | N-(1-(4-(cyclopropanesulfonamido)pyridin-2-yl)-3- | 560 | 3.175 |
| | (dimethylamino)-3-methylbutyl)-5-(6-ethoxypyrazin-2- | | LCMS-AL |
| | yl)thiazole-2-carboxamide | | |
| | 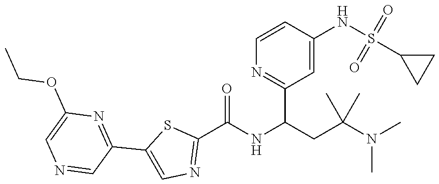 [SMILES: CCOc1cncc(-c2cnc(C(=O)NC(CC(C)(C)N(C)C)c3cc(NS(=O)(=O)C4CC4)ccn3)s2)n1] | | |
| I-49 | (R)-N-(1-(4-(cyclopropanesulfonamido)pyridin-2-yl)-3- | 560 | 1.202 |
| | (dimethylamino)-3-methylbutyl)-5-(6-ethoxypyrazin-2- | | LCMS-B |
| | yl)thiazole-2-carboxamide | | |
| | 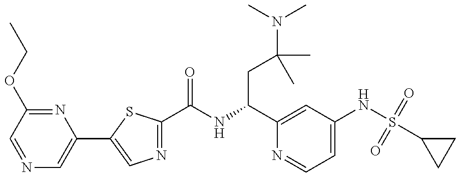 [SMILES: CCOc1cncc(-c2cnc(C(=O)N[C@H](CC(C)(C)N(C)C)c3cc(NS(=O)(=O)C4CC4)ccn3)s2)n1] | | |
| I-50 | (S)-N-(1-(4-(cyclopropanesulfonamido)pyridin-2-yl)-3- | 560 | 1.199 |
| | (dimethylamino)-3-methylbutyl)-5-(6-ethoxypyrazin-2- | | LCMS-B |
| | yl)thiazole-2-carboxamide | | |
| | 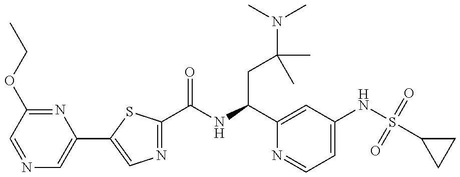 [SMILES: CCOc1cncc(-c2cnc(C(=O)N[C@@H](CC(C)(C)N(C)C)c3cc(NS(=O)(=O)C4CC4)ccn3)s2)n1] | | |
| I-51 | N-((1R,2R)-1-(4-(cyclopropanesulfonamido)pyridin-2-yl)-2- | 586 | 0.964 |
| | methyl-3-(piperidin-1-yl)propyl)-5-(6-ethoxypyrazin-2- | | LCMS-AN |
| | yl)thiazole-2-carboxamide | | |
| | 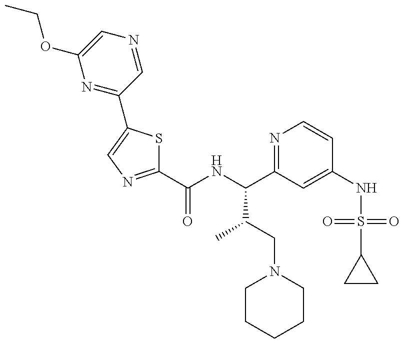 [SMILES: CCOc1cncc(-c2cnc(C(=O)N[C@H](c3cc(NS(=O)(=O)C4CC4)ccn3)[C@@H](C)CN3CCCCC3)s2)n1] | | |
| I-52 | N-((1R,2S)-1-(4-(cyclopropanesulfonamido)pyridin-2-yl)-2- | 586 | 0.985 |
| | methyl-3-(piperidin-1-yl)propyl)-5-(6-ethoxypyrazin-2- | | LCMS-AN |
| | yl)thiazole-2-carboxamide | | |
| | 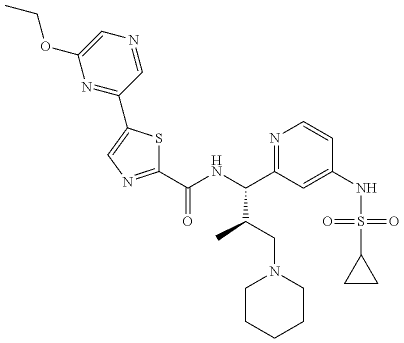 [SMILES: CCOc1cncc(-c2cnc(C(=O)N[C@H](c3cc(NS(=O)(=O)C4CC4)ccn3)[C@H](C)CN3CCCCC3)s2)n1] | | |
| I-53 | N-((1S,2R)-1-(4-(cyclopropanesulfonamido)pyridin-2-yl)-2- | 586 | 0.99 |
| | methyl-3-(piperidin-1-yl)propyl)-5-(6-ethoxypyrazin-2- | | LCMS-AN |
| | yl)thiazole-2-carboxamide | | |
| | 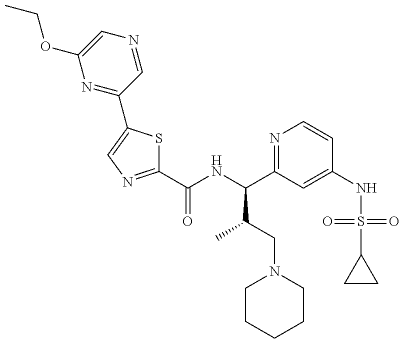 [SMILES: CCOc1cncc(-c2cnc(C(=O)N[C@@H](c3cc(NS(=O)(=O)C4CC4)ccn3)[C@@H](C)CN3CCCCC3)s2)n1] | | |
| I-54 | N-((1S,2S)-1-(4-(cyclopropanesulfonamido)pyridin-2-yl)-2- | 586 | 0.968 |
| | methyl-3-(piperidin-1-yl)propyl)-5-(6-ethoxypyrazin-2- | | LCMS-AN |
| | yl)thiazole-2-carboxamide | | |
| | 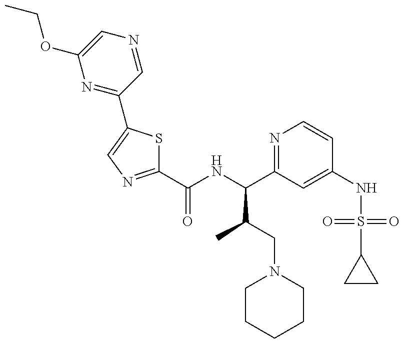 [SMILES: CCOc1cncc(-c2cnc(C(=O)N[C@@H](c3cc(NS(=O)(=O)C4CC4)ccn3)[C@H](C)CN3CCCCC3)s2)n1] | | |
| I-55 | 5-(5-chloropyrazolo[1,5-a]pyridin-3-yl)-N-((1R,2S)-1-(4- | 614/616 | 1.564 |
| | (cyclopropanesulfonamido)pyridin-2-yl)-2-methyl-3-(piperidin- | | LCMS-AD |
| | 1-yl)propyl)thiazole-2-carboxamide | | |
| | 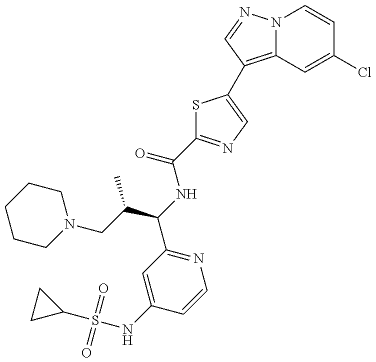 [SMILES: C[C@@H](CN1CCCCC1)[C@@H](NC(=O)c1ncc(-c2cnn3ccc(Cl)cc23)s1)c1cc(NS(=O)(=O)C2CC2)ccn1] | | |
| I-56 | 5-(5-chloropyrazolo[1,5-a]pyridin-3-yl)-N-((1R,2R)-1-(4- | 614/616 | 1.471 |
| | (cyclopropanesulfonamido)pyridin-2-yl)-2-methyl-3-(piperidin- | | LCMS-AE |
| | 1-yl)propyl)thiazole-2-carboxamide | | |
| | 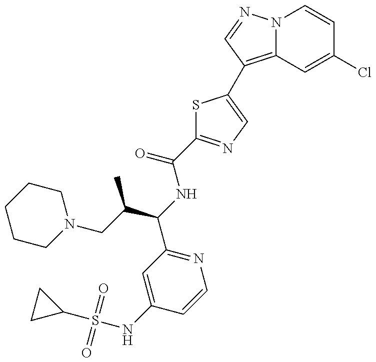 [SMILES: C[C@H](CN1CCCCC1)[C@@H](NC(=O)c1ncc(-c2cnn3ccc(Cl)cc23)s1)c1cc(NS(=O)(=O)C2CC2)ccn1] | | |
| I-57 | 5-(5-chloropyrazolo[1,5-a]pyridin-3-yl)-N-((1S,2R)-1-(4- | 614/616 | 1.462 |
| | (cyclopropanesulfonamido)pyridin-2-yl)-2-methyl-3-(piperidin- | | LCMS-AE |
| | 1-yl)propyl)thiazole-2-carboxamide | | |
| | 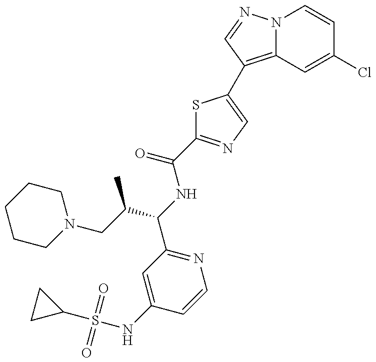 [SMILES: C[C@H](CN1CCCCC1)[C@H](NC(=O)c1ncc(-c2cnn3ccc(Cl)cc23)s1)c1cc(NS(=O)(=O)C2CC2)ccn1] | | |
| I-58 | 5-(5-chloropyrazolo[1,5-a]pyridin-3-yl)-N-((1S,2S)-1-(4- | 614/616 | 1.53 |
| | (cyclopropanesulfonamido)pyridin-2-yl)-2-methyl-3-(piperidin- | | LCMS-AE |
| | 1-yl)propyl)thiazole-2-carboxamide | | |
| | 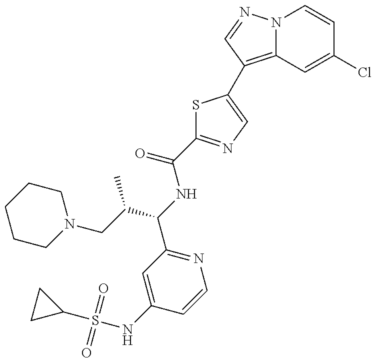 [SMILES: C[C@@H](CN1CCCCC1)[C@H](NC(=O)c1ncc(-c2cnn3ccc(Cl)cc23)s1)c1cc(NS(=O)(=O)C2CC2)ccn1] | | |
| I-59 | N-((R)-(4-(cyclopropanesulfonamido)pyridin-2-yl)((R)- | 531 | 1.747 |
| | tetrahydrofuran-2-yl)methyl)-5-(6-ethoxypyrazin-2-yl)thiazole- | | LCMS-Q |
| | 2-carboxamide | | |
| | 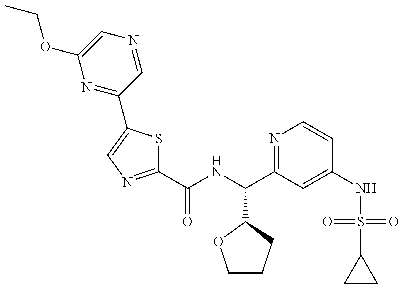 [SMILES: CCOc1cncc(-c2cnc(C(=O)N[C@H](c3cc(NS(=O)(=O)C4CC4)ccn3)[C@H]3CCCO3)s2)n1] | | |
| I-60 | N-((R)-(4-(cyclopropanesulfonamido)pyridin-2-yl)((S)- | 531 | 1.627 |
| | tetrahydrofuran-2-yl)methyl)-5-(6-ethoxypyrazin-2-yl)thiazole- | | LCMS-R |
| | 2-carboxamide | | |
| | 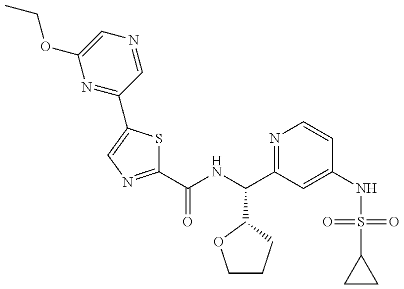 [SMILES: CCOc1cncc(-c2cnc(C(=O)N[C@H](c3cc(NS(=O)(=O)C4CC4)ccn3)[C@@H]3CCCO3)s2)n1] | | |
| I-61 | N-((S)-(4-(cyclopropanesulfonamido)pyridin-2-yl)((S)- | 531 | 1.761 |
| | tetrahydrofuran-2-yl)methyl)-5-(6-ethoxypyrazin-2-yl)thiazole- | | LCMS-D |
| | 2-carboxamide | | |
| | 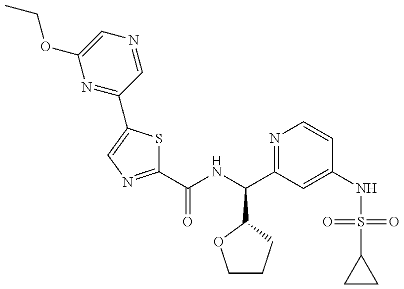 [SMILES: CCOc1cncc(-c2cnc(C(=O)N[C@@H](c3cc(NS(=O)(=O)C4CC4)ccn3)[C@@H]3CCCO3)s2)n1] | | |
| I-62 | N-((S)-(4-(cyclopropanesulfonamido)pyridin-2-yl)((R)- | 531 | 0.927 |
| | tetrahydrofuran-2-yl)methyl)-5-(6-ethoxypyrazin-2-yl)thiazole- | | LCMS-AV |
| | 2-carboxamide | | |
| | 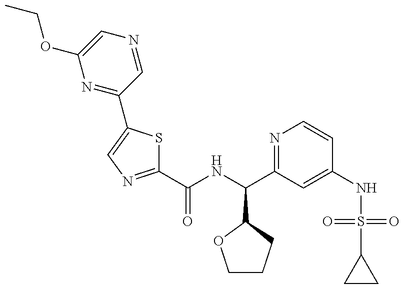 [SMILES: CCOc1cncc(-c2cnc(C(=O)N[C@@H](c3cc(NS(=O)(=O)C4CC4)ccn3)[C@H]3CCCO3)s2)n1] | | |
| I-63 | N-((S)-(4-(cyclopropanesulfonamido)pyridin-2-yl)((R)- | 545 | 1.185 |
| | tetrahydrofuran-3-yl)methyl)-5-(6-ethoxypyrazin-2-yl)-N- | | LCMS-AN |
| | methylthiazole-2-carboxamide | | |
| | 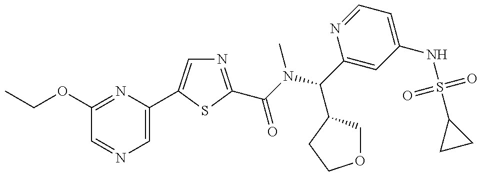 [SMILES: CCOc1cncc(-c2cnc(C(=O)N(C)[C@H](c3cc(NS(=O)(=O)C4CC4)ccn3)[C@H]3CCOC3)s2)n1] | | |
| I-64 | N-((S)-(4-(cyclopropanesulfonamido)pyridin-2-yl)((S)- | 545 | 1.499 |
| | tetrahydrofuran-3-yl)methyl)-5-(6-ethoxypyrazin-2-yl)-N- | | LCMS-AH |
| | methylthiazole-2-carboxamide | | |
| | 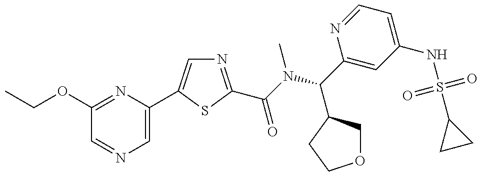 [SMILES: CCOc1cncc(-c2cnc(C(=O)N(C)[C@H](c3cc(NS(=O)(=O)C4CC4)ccn3)[C@@H]3CCOC3)s2)n1] | | |
| I-65 | N-((R)-(4-(cyclopropanesulfonamido)pyridin-2-yl)((R)- | 545 | 1.509 |
| | tetrahydrofuran-3-yl)methyl)-5-(6-ethoxypyrazin-2-yl)-N- | | LCMS-AH |
| | methylthiazole-2-carboxamide | | |
| | 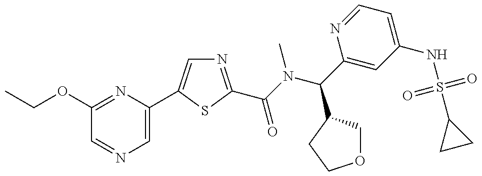 [SMILES: CCOc1cncc(-c2cnc(C(=O)N(C)[C@@H](c3cc(NS(=O)(=O)C4CC4)ccn3)[C@H]3CCOC3)s2)n1] | | |
| I-66 | N-((R)-(4-(cyclopropanesulfonamido)pyridin-2-yl)((S)- | 545 | 1.182 |
| | tetrahydrofuran-3-yl)methyl)-5-(6-ethoxypyrazin-2-yl)-N- | | LCMS-AN |
| | methylthiazole-2-carboxamide | | |
| | 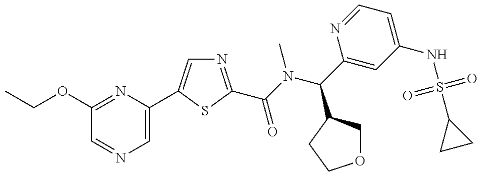 [SMILES: CCOc1cncc(-c2cnc(C(=O)N(C)[C@@H](c3cc(NS(=O)(=O)C4CC4)ccn3)[C@@H]3CCOC3)s2)n1] | | |
| I-67 | N-((R)-(4-(cyclopropanesulfonamido)pyridin-2-yl)((R)- | 525 | 1.002 |
| | tetrahydrofuran-2-yl)methyl)-5-(6-ethoxypyrazin-2- | | LCMS-W |
| | yl)picolinamide | | |
| | 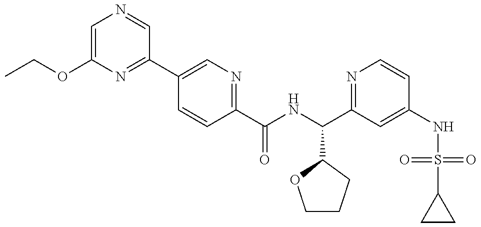 [SMILES: CCOc1cncc(-c2ccc(C(=O)N[C@H](c3cc(NS(=O)(=O)C4CC4)ccn3)[C@H]3CCCO3)nc2)n1] | | |
| I-68 | N-((R)-(4-(cyclopropanesulfonamido)pyridin-2-yl)((S)- | 525 | 1.008 |
| | tetrahydrofuran-2-yl)methyl)-5-(6-ethoxypyrazin-2- | | LCMS-W |
| | yl)picolinamide | | |
| | 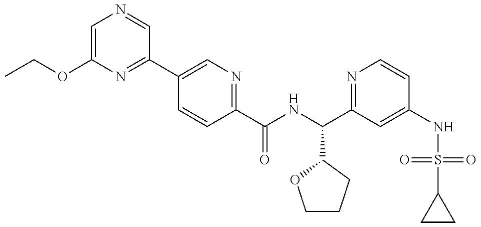 [SMILES: CCOc1cncc(-c2ccc(C(=O)N[C@H](c3cc(NS(=O)(=O)C4CC4)ccn3)[C@@H]3CCCO3)nc2)n1] | | |
| I-69 | N-((S)-(4-(cyclopropanesulfonamido)pyridin-2-yl)((S)- | 525 | 1.008 |
| | tetrahydrofuran-2-yl)methyl)-5-(6-ethoxypyrazin-2- | | LCMS-W |
| | yl)picolinamide | | |
| | 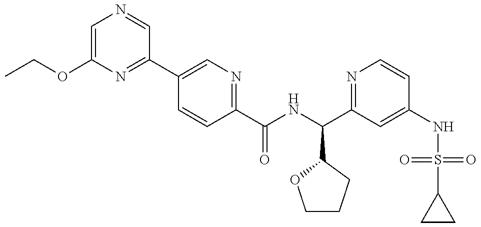 [SMILES: CCOc1cncc(-c2ccc(C(=O)N[C@@H](c3cc(NS(=O)(=O)C4CC4)ccn3)[C@@H]3CCCO3)nc2)n1] | | |
| I-70 | N-((S)-(4-(cyclopropanesulfonamido)pyridin-2-yl)((R)- | 525 | 1.015 |
| | tetrahydrofuran-2-yl)methyl)-5-(6-ethoxypyrazin-2- | | LCMS-W |
| | yl)picolinamide | | |
| | 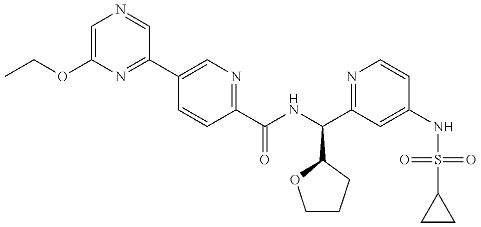 [SMILES: CCOc1cncc(-c2ccc(C(=O)N[C@@H](c3cc(NS(=O)(=O)C4CC4)ccn3)[C@H]3CCCO3)nc2)n1] | | |
| I-71 | (R)-N-(3-(4-(cyclopropanesulfonamido)pyridin-2- | 517 | 1.687 |
| | yl)tetrahydrofuran-3-yl)-5-(6-ethoxypyrazin-2-yl)thiazole-2- | | LCMS-Q |
| | carboxamide | | |
| | 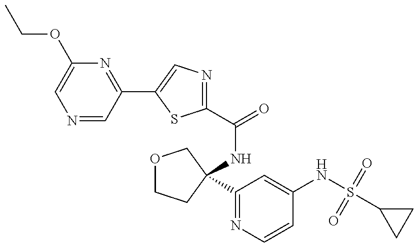 [SMILES: CCOc1cncc(-c2cnc(C(=O)N[C@@]3(c4cc(NS(=O)(=O)C5CC5)ccn4)CCOC3)s2)n1] | | |
| I-72 | (S)-N-(3-(4-(cyclopropanesulfonamido)pyridin-2- | 517 | 1.687 |
| | yl)tetrahydrofuran-3-yl)-5-(6-ethoxypyrazin-2-yl)thiazole-2- | | LCMS-Q |
| | carboxamide | | |
| | 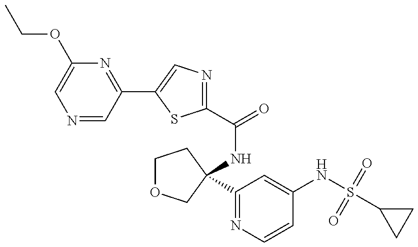 [SMILES: CCOc1cncc(-c2cnc(C(=O)N[C@]3(c4cc(NS(=O)(=O)C5CC5)ccn4)CCOC3)s2)n1] | | |
| I-73 | N-((R)-(4-(cyclopropanesulfonamido)pyridin-2-yl)((S)-1,4- | 547 | 0.873 |
| | dioxan-2-yl)methyl)-5-(6-ethoxypyrazin-2-yl)thiazole-2- | | LCMS-AV |
| | carboxamide | | |
| | 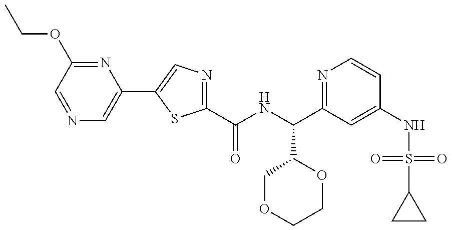 [SMILES: CCOc1cncc(-c2cnc(C(=O)N[C@H](c3cc(NS(=O)(=O)C4CC4)ccn3)[C@H]3COCCO3)s2)n1] | | |
| I-74 | N-((R)-(4-(cyclopropanesulfonamido)pyridin-2-yl)((R)-1,4- | 547 | 0.86 |
| | dioxan-2-yl)methyl)-5-(6-ethoxypyrazin-2-yl)thiazole-2- | | LCMS-AU |
| | carboxamide | | |
| | 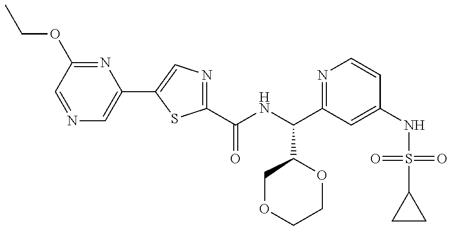 [SMILES: CCOc1cncc(-c2cnc(C(=O)N[C@H](c3cc(NS(=O)(=O)C4CC4)ccn3)[C@@H]3COCCO3)s2)n1] | | |
| I-75 | N-((S)-(4-(cyclopropanesulfonamido)pyridin-2-yl)((S)-1,4- | 547 | 1.485 |
| | dioxan-2-yl)methyl)-5-(6-ethoxypyrazin-2-yl)thiazole-2- | | LCMS-BF |
| | carboxamide | | |
| | 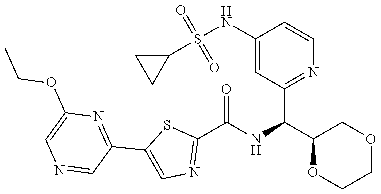 [SMILES: CCOc1cncc(-c2cnc(C(=O)N[C@@H](c3cc(NS(=O)(=O)C4CC4)ccn3)[C@H]3COCCO3)s2)n1] | | |
| I-76 | N-((S)-(4-(cyclopropanesulfonamido)pyridin-2-yl)((R)-1,4- | 547 | 1.477 |
| | dioxan-2-yl)methyl)-5-(6-ethoxypyrazin-2-yl)thiazole-2- | | LCMS-BF |
| | carboxamide | | |
| | 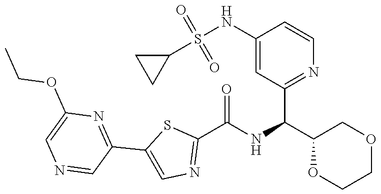 [SMILES: CCOc1cncc(-c2cnc(C(=O)N[C@@H](c3cc(NS(=O)(=O)C4CC4)ccn3)[C@@H]3COCCO3)s2)n1] | | |
| I-77 | 5-(5-chloropyrazolo[1,5-a]pyridin-3-yl)-N-((R)-(4- | 575/577 | 0.856 |
| | (cyclopropanesulfonamido)pyridin-2-yl)((S)-1,4-dioxan-2- | | LCMS-BA |
| | yl)methyl)thiazole-2-carboxamide | | |
| | 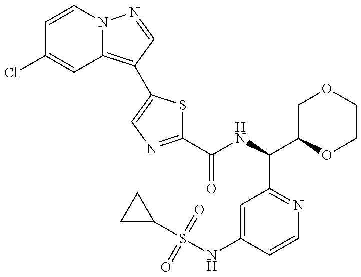 [SMILES: O=C(N[C@H](c1cc(NS(=O)(=O)C2CC2)ccn1)[C@H]1COCCO1)c1ncc(-c2cnn3ccc(Cl)cc23)s1] | | |
| I-78 | 5-(5-chloropyrazolo[1,5-a]pyridin-3-yl)-N-((R)-(4- | 575/577 | 1.594 |
| | (cyclopropanesulfonamido)pyridin-2-yl)((R)-1,4-dioxan-2- | | LCMS-AO |
| | yl)methyl)thiazole-2-carboxamide | | |
| | 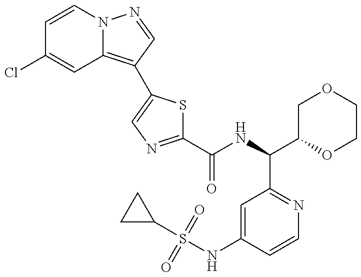 [SMILES: O=C(N[C@H](c1cc(NS(=O)(=O)C2CC2)ccn1)[C@@H]1COCCO1)c1ncc(-c2cnn3ccc(Cl)cc23)s1] | | |
| I-79 | 5-(5-chloropyrazolo[1,5-a]pyridin-3-yl)-N-((S)-(4- | 575/577 | 1.674 |
| | (cyclopropanesulfonamido)pyridin-2-yl)((R)-1,4-dioxan-2- | | LCMS-AO |
| | yl)methyl)thiazole-2-carboxamide | | |
| | 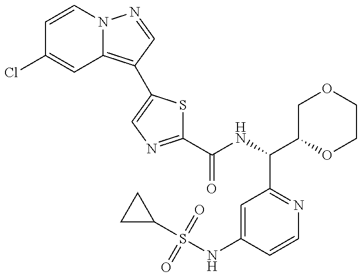 [SMILES: O=C(N[C@@H](c1cc(NS(=O)(=O)C2CC2)ccn1)[C@@H]1COCCO1)c1ncc(-c2cnn3ccc(Cl)cc23)s1] | | |
| I-80 | 5-(5-chloropyrazolo[1,5-a]pyridin-3-yl)-N-((S)-(4- | 575/577 | 1.673 |
| | (cyclopropanesulfonamido)pyridin-2-yl)((S)-1,4-dioxan-2- | | LCMS-AO |
| | yl)methyl)thiazole-2-carboxamide | | |
| | 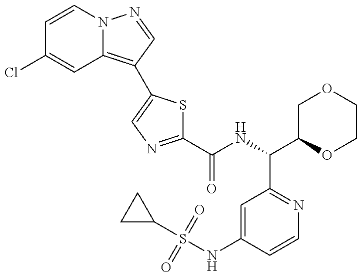 [SMILES: O=C(N[C@@H](c1cc(NS(=O)(=O)C2CC2)ccn1)[C@H]1COCCO1)c1ncc(-c2cnn3ccc(Cl)cc23)s1] | | |
| I-81 | N-((R)-((1R,2S,4S)-7-oxabicyclo[2.2.1]heptan-2-yl)(4- | 557 | 1.055 |
| | (cyclopropanesulfonamido)pyridin-2-yl)methyl)-5-(6- | | LCMS-P |
| | ethoxypyrazin-2-yl)thiazole-2-carboxamide | | |
| | 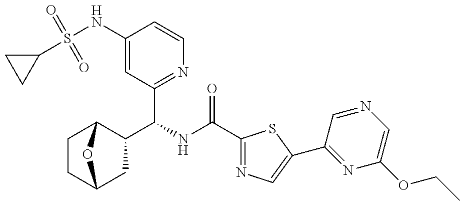 [SMILES: CCOc1cncc(-c2cnc(C(=O)N[C@@H](c3cc(NS(=O)(=O)C4CC4)ccn3)[C@@H]3C[C@@H]4CC[C@H]3O4)s2)n1] | | |
| I-82 | N-((S)-((1R,2S,4S)-7-oxabicyclo[2.2.1]heptan-2-yl)(4- | 557 | 1.055 |
| | (cyclopropanesulfonamido)pyridin-2-yl)methyl)-5-(6- | | LCMS-P |
| | ethoxypyrazin-2-yl)thiazole-2-carboxamide | | |
| | 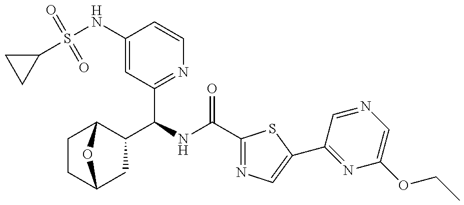 [SMILES: CCOc1cncc(-c2cnc(C(=O)N[C@H](c3cc(NS(=O)(=O)C4CC4)ccn3)[C@@H]3C[C@@H]4CC[C@H]3O4)s2)n1] | | |
| I-83 | N-((S)-((1S,2R,4R)-7-oxabicyclo[2.2.1]heptan-2-yl)(4- | 557 | 1.055 |
| | (cyclopropanesulfonamido)pyridin-2-yl)methyl)-5-(6- | | LCMS-P |
| | ethoxypyrazin-2-yl)thiazole-2-carboxamide | | |
| | 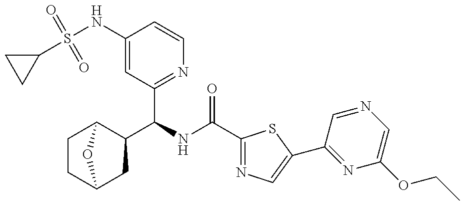 [SMILES: CCOc1cncc(-c2cnc(C(=O)N[C@H](c3cc(NS(=O)(=O)C4CC4)ccn3)[C@H]3C[C@H]4CC[C@@H]3O4)s2)n1] | | |
| I-84 | N-((R)-((1S,2R,4R)-7-oxabicyclo[2.2.1]heptan-2-yl)(4- | 557 | 1.062 |
| | (cyclopropanesulfonamido)pyridin-2-yl)methyl)-5-(6- | | LCMS-P |
| | ethoxypyrazin-2-yl)thiazole-2-carboxamide | | |
| | 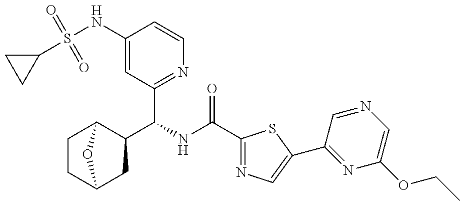 [SMILES: CCOc1cncc(-c2cnc(C(=O)N[C@@H](c3cc(NS(=O)(=O)C4CC4)ccn3)[C@H]3C[C@H]4CC[C@@H]3O4)s2)n1] | | |
| I-85 | (S)-5-(5-chloropyrazolo[1,5-a]pyridin-3-yl)-N-((4- | 573/575 | 1.577 |
| | (cyclopropanesulfonamido)pyridin-2-yl)(tetrahydro-2H-pyran- | | LCMS-AD |
| | 4-yl)methyl)thiazole-2-carboxamide | | |
| | 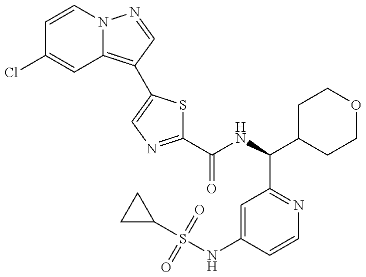 [SMILES: O=C(N[C@H](c1cc(NS(=O)(=O)C2CC2)ccn1)C1CCOCC1)c1ncc(-c2cnn3ccc(Cl)cc23)s1] | | |
| I-86 | (R)-5-(5-chloropyrazolo[1,5-a]pyridin-3-yl)-N-((4- | 573/575 | 0.877 |
| | (cyclopropanesulfonamido)pyridin-2-yl)(tetrahydro-2H-pyran- | | LCMS-AC |
| | 4-yl)methyl)thiazole-2-carboxamide | | |
| | 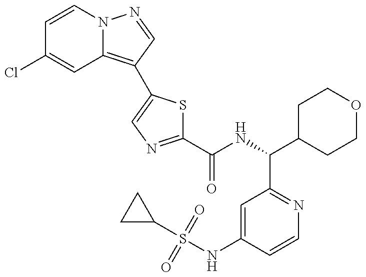 [SMILES: O=C(N[C@@H](c1cc(NS(=O)(=O)C2CC2)ccn1)C1CCOCC1)c1ncc(-c2cnn3ccc(Cl)cc23)s1] | | |
| I-87 | N-((1s,3s)-1-(4-(cyclopropanesulfonamido)pyridin-2-yl)-3- | 517 | 1.422 |
| | hydroxycyclobutyl)-5-(6-ethoxypyrazin-2-yl)thiazole-2- | | LCMS-F |
| | carboxamide | | |
| | 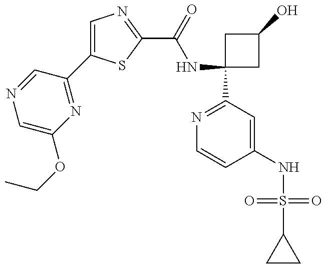 [SMILES: CCOc1cncc(-c2cnc(C(=O)N[C@]3(c4cc(NS(=O)(=O)C5CC5)ccn4)C[C@H](O)C3)s2)n1] | | |
| I-88 | N-((1r,3r)-1-(4-(cyclopropanesulfonamido)pyridin-2-yl)-3- | 517 | 1.404 |
| | hydroxycyclobutyl)-5-(6-ethoxypyrazin-2-yl)thiazole-2- | | LCMS-BC |
| | carboxamide | | |
| | 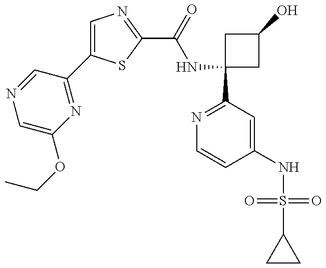 [SMILES: CCOc1cncc(-c2cnc(C(=O)N[C@]3(c4cc(NS(=O)(=O)C5CC5)ccn4)C[C@@H](O)C3)s2)n1] | | |
| I-89 | 5-(5-chloropyrazolo[1,5-a]pyridin-3-yl)-N-((1s,3s)-1-(4- | 545/547 | 1.438 |
| | (cyclopropanesulfonamido)pyridin-2-yl)-3- | | LCMS-AD |
| | hydroxycyclobutyl)thiazole-2-carboxamide | | |
| | 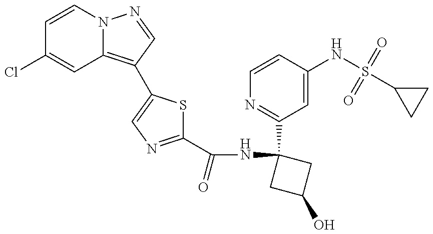 [SMILES: O=C(N[C@]1(c2cc(NS(=O)(=O)C3CC3)ccn2)C[C@H](O)C1)c1ncc(-c2cnn3ccc(Cl)cc23)s1] | | |
| I-90 | 5-(5-chloropyrazolo[1,5-a]pyridin-3-yl)-N-((1r,3r)-1-(4- | 545/547 | 1.4 |
| | (cyclopropanesulfonamido)pyridin-2-yl)-3- | | LCMS-AD |
| | hydroxycyclobutyl)thiazole-2-carboxamide | | |
| | 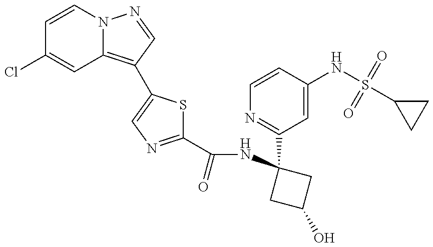 [SMILES: O=C(N[C@]1(c2cc(NS(=O)(=O)C3CC3)ccn2)C[C@@H](O)C1)c1ncc(-c2cnn3ccc(Cl)cc23)s1] | | |
| I-91 | (S)-N-(1-(4-(cyclopropanesulfonamido)pyridin-2-yl)-2- | 533 | 1.634 |
| | methoxy-2-methylpropyl)-5-(6-ethoxypyrazin-2-yl)thiazole-2- | | LCMS-BG |
| | carboxamide | | |
| | 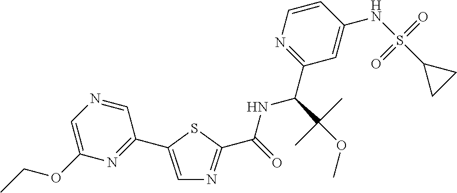 [SMILES: CCOc1cncc(-c2cnc(C(=O)N[C@@H](c3cc(NS(=O)(=O)C4CC4)ccn3)C(C)(C)OC)s2)n1] | | |
| I-92 | (R)-N-(1-(4-(cyclopropanesulfonamido)pyridin-2-yl)-2- | 533 | 1.641 |
| | methoxy-2-methylpropyl)-5-(6-ethoxypyrazin-2-yl)thiazole-2- | | LCMS-BG |
| | carboxamide | | |
| | 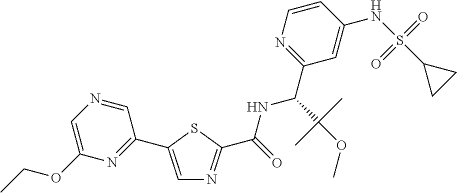 [SMILES: CCOc1cncc(-c2cnc(C(=O)N[C@H](c3cc(NS(=O)(=O)C4CC4)ccn3)C(C)(C)OC)s2)n1] | | |
| I-93 | N-((S)-((S)-1-cyclopentylpyrrolidin-3-yl)(4- | 598 | 1.66 |
| | (cyclopropanesulfonamido)pyridin-2-yl)methyl)-5-(6- | | LCMS-Y |
| | ethoxypyrazin-2-yl)thiazole-2-carboxamide | | |
| | 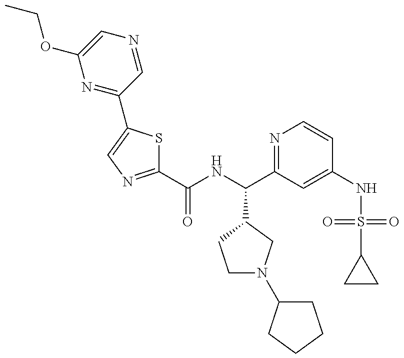 [SMILES: CCOc1cncc(-c2cnc(C(=O)N[C@H](c3cc(NS(=O)(=O)C4CC4)ccn3)[C@H]3CCN(C4CCCC4)C3)s2)n1] | | |
| I-94 | N-((S)-((R)-1-cyclopentylpyrrolidin-3-yl)(4- | 598 | 0.991 |
| | (cyclopropanesulfonamido)pyridin-2-yl)methyl)-5-(6- | | LCMS-W |
| | ethoxypyrazin-2-yl)thiazole-2-carboxamide | | |
| | 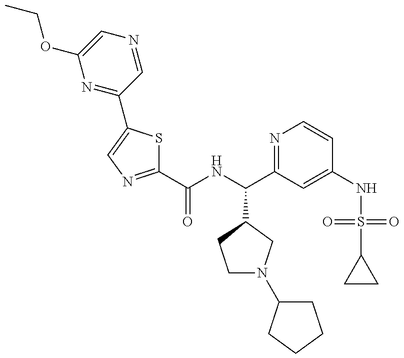 [SMILES: CCOc1cncc(-c2cnc(C(=O)N[C@H](c3cc(NS(=O)(=O)C4CC4)ccn3)[C@@H]3CCN(C4CCCC4)C3)s2)n1] | | |
| I-95 | N-((R)-((R)-1-cyclopentylpyrrolidin-3-yl)(4- | 598 | 0.987 |
| | (cyclopropanesulfonamido)pyridin-2-yl)methyl)-5-(6- | | LCMS-W |
| | ethoxypyrazin-2-yl)thiazole-2-carboxamide | | |
| | 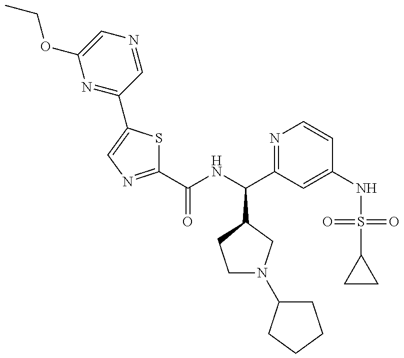 [SMILES: CCOc1cncc(-c2cnc(C(=O)N[C@@H](c3cc(NS(=O)(=O)C4CC4)ccn3)[C@@H]3CCN(C4CCCC4)C3)s2)n1] | | |
| I-96 | N-((R)-((S)-1-cyclopentylpyrrolidin-3-yl)(4- | 598 | 1.667 |
| | (cyclopropanesulfonamido)pyridin-2-yl)methyl)-5-(6- | | LCMS-Y |
| | ethoxypyrazin-2-yl)thiazole-2-carboxamide | | |
| | 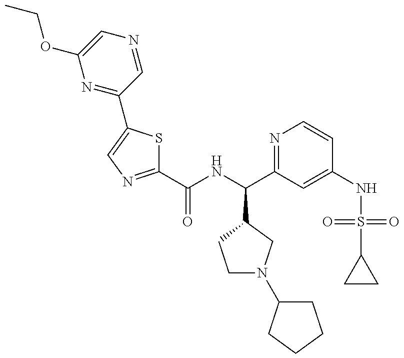 [SMILES: CCOc1cncc(-c2cnc(C(=O)N[C@@H](c3cc(NS(=O)(=O)C4CC4)ccn3)[C@H]3CCN(C4CCCC4)C3)s2)n1] | | |
| I-97 | N-((R)-(4-(cyclopropanesulfonamido)pyridin-2-yl)((R)-4- | 560 | 1.382 |
| | methylmorpholin-2-yl)methyl)-5-(6-ethoxypyrazin-2- | | LCMS-AD |
| | yl)thiazole-2-carboxamide | | |
| | 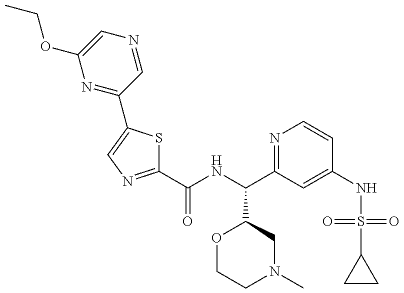 [SMILES: CCOc1cncc(-c2cnc(C(=O)N[C@H](c3cc(NS(=O)(=O)C4CC4)ccn3)[C@H]3CN(C)CCO3)s2)n1] | | |
| I-99 | N-((R)-(4-(cyclopropanesulfonamido)pyridin-2-yl)((S)-4- | 560 | 1.351 |
| | methylmorpholin-2-yl)methyl)-5-(6-ethoxypyrazin-2- | | LCMS-AD |
| | yl)thiazole-2-carboxamide | | |
| | 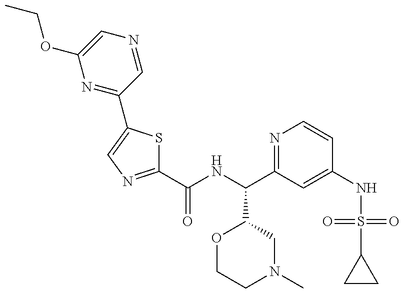 [SMILES: CCOc1cncc(-c2cnc(C(=O)N[C@H](c3cc(NS(=O)(=O)C4CC4)ccn3)[C@@H]3CN(C)CCO3)s2)n1] | | |
| I-100 | N-((S)-(4-(cyclopropanesulfonamido)pyridin-2-yl)((R)-4- | 560 | 0.909 |
| | methylmorpholin-2-yl)methyl)-5-(6-ethoxypyrazin-2- | | LCMS-AN |
| | yl)thiazole-2-carboxamide | | |
| | 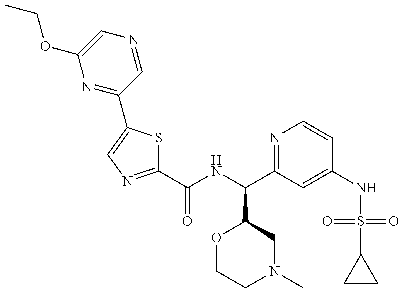 [SMILES: CCOc1cncc(-c2cnc(C(=O)N[C@@H](c3cc(NS(=O)(=O)C4CC4)ccn3)[C@H]3CN(C)CCO3)s2)n1] | | |
| I-101 | N-((S)-(4-(cyclopropanesulfonamido)pyridin-2-yl)((S)-4- | 560 | 0.904 |
| | methylmorpholin-2-yl)methyl)-5-(6-ethoxypyrazin-2- | | LCMS-AN |
| | yl)thiazole-2-carboxamide | | |
| | 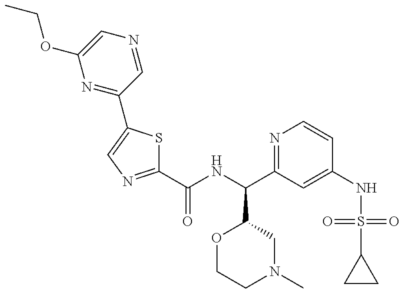 [SMILES: CCOc1cncc(-c2cnc(C(=O)N[C@@H](c3cc(NS(=O)(=O)C4CC4)ccn3)[C@@H]3CN(C)CCO3)s2)n1] | | |
| I-102 | N-((S)-1-(4-(cyclopropanesulfonamido)pyridin-2-yl)-2-((S)-4- | 574 | 1.368 |
| | methylmorpholin-3-yl)ethyl)-5-(6-ethoxypyrazin-2-yl)thiazole- | | LCMS-B |
| | 2-carboxamide | | |
| | 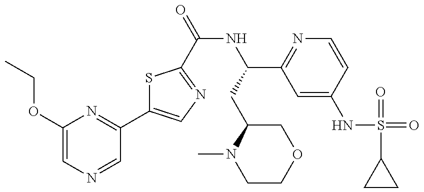 [SMILES: CCOc1cncc(-c2cnc(C(=O)N[C@@H](C[C@H]3COCCN3C)c3cc(NS(=O)(=O)C4CC4)ccn3)s2)n1] | | |
| I-103 | N-((R)-1-(4-(cyclopropanesulfonamido)pyridin-2-yl)-2-((R)-4- | 574 | 1.373 |
| | methylmorpholin-3-yl)ethyl)-5-(6-ethoxypyrazin-2-yl)thiazole- | | LCMS-B |
| | 2-carboxamide | | |
| | 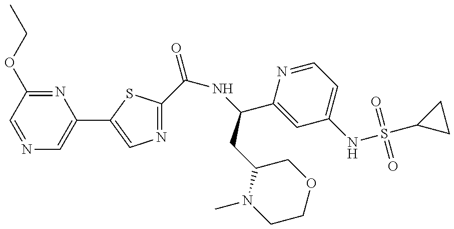 | | |
| I-104 | N-((R)-1-(4-(cyclopropanesulfonamido)pyridin-2-yl)-2-((S)-4- | 574 | 1.37 |
| | methylmorpholin-3-yl)ethyl)-5-(6-ethoxypyrazin-2-yl)thiazole- | | LCMS-B |
| | 2-carboxamide | | |
| | 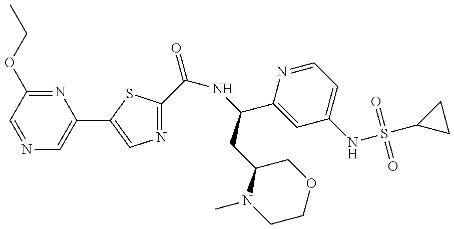 [SMILES: CCOc1cncc(-c2cnc(C(=O)N[C@H](C[C@H]3COCCN3C)c3cc(NS(=O)(=O)C4CC4)ccn3)s2)n1] | | |
| I-105 | N-((4-(cyclopropanesulfonamido)pyridin-2-yl)(4- | 560 | 0.935 |
| | methylmorpholin-2-yl)methyl)-5-(6-ethoxypyrazin-2- | | LCMS-P |
| | yl)thiazole-2-carboxamide | | |
| | 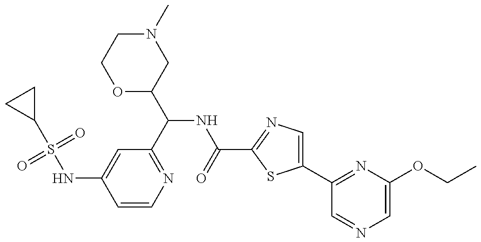 [SMILES: CCOc1cncc(-c2cnc(C(=O)NC(c3cc(NS(=O)(=O)C4CC4)ccn3)C3CN(C)CCO3)s2)n1] | | |
| I-106 | N-((4-(cyclopropanesulfonamido)pyridin-2-yl)(4- | 560 | 0.942 |
| | methylmorpholin-2-yl)methyl)-5-(6-ethoxypyrazin-2- | | LCMS-P |
| | yl)thiazole-2-carboxamide | | |
| | 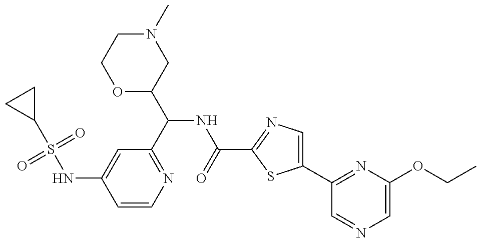 [SMILES: CCOc1cncc(-c2cnc(C(=O)NC(c3cc(NS(=O)(=O)C4CC4)ccn3)C3CN(C)CCO3)s2)n1] | | |
| I-107 | N-((S)-(4-(cyclopropanesulfonamido)pyridin-2-yl)((S)-1-(2- | 576 | 0.903 |
| | fluoroethyl)pyrrolidin-3-yl)methyl)-5-(6-ethoxypyrazin-2- | | LCMS-AU |
| | yl)thiazole-2-carboxamide | | |
| | 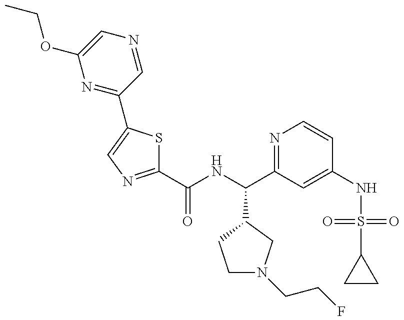 [SMILES: CCOc1cncc(-c2cnc(C(=O)N[C@H](c3cc(NS(=O)(=O)C4CC4)ccn3)[C@H]3CCN(CCF)C3)s2)n1] | | |
| I-108 | N-((S)-(4-(cyclopropanesulfonamido)pyridin-2-yl)((R)-1-(2- | 576 | 0.903 |
| | fluoroethyl)pyrrolidin-3-yl)methyl)-5-(6-ethoxypyrazin-2- | | LCMS-AU |
| | yl)thiazole-2-carboxamide | | |
| | 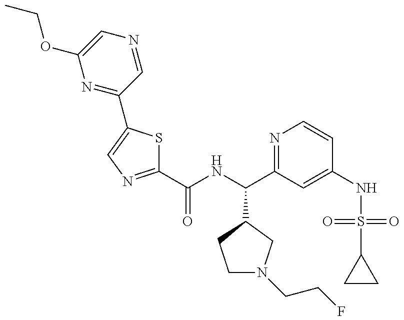 [SMILES: CCOc1cncc(-c2cnc(C(=O)N[C@H](c3cc(NS(=O)(=O)C4CC4)ccn3)[C@@H]3CCN(CCF)C3)s2)n1] | | |
| I-109 | N-((R)-(4-(cyclopropanesulfonamido)pyridin-2-yl)((R)-1-(2- | 576 | 0.917 |
| | fluoroethyl)pyrrolidin-3-yl)methyl)-5-(6-ethoxypyrazin-2- | | LCMS-AV |
| | yl)thiazole-2-carboxamide | | |
| | 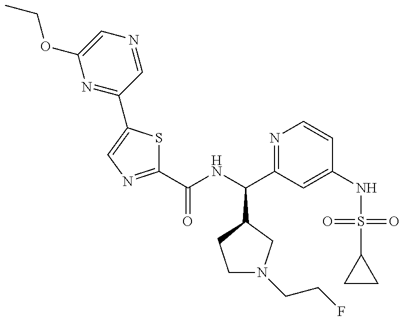 [SMILES: CCOc1cncc(-c2cnc(C(=O)N[C@@H](c3cc(NS(=O)(=O)C4CC4)ccn3)[C@@H]3CCN(CCF)C3)s2)n1] | | |
| I-110 | N-((R)-(4-(cyclopropanesulfonamido)pyridin-2-yl)((S)-1-(2- | 576 | 0.917 |
| | fluoroethyl)pyrrolidin-3-yl)methyl)-5-(6-ethoxypyrazin-2- | | LCMS-AV |
| | yl)thiazole-2-carboxamide | | |
| | 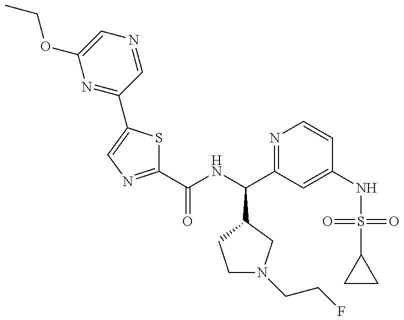 [SMILES: CCOc1cncc(-c2cnc(C(=O)N[C@@H](c3cc(NS(=O)(=O)C4CC4)ccn3)[C@H]3CCN(CCF)C3)s2)n1] | | |
| I-111 | N-((R)-(4-(cyclopropanesulfonamido)pyridin-2-yl)((S)-1- | 570 | 1.704 |
| | cyclopropylpyrrolidin-3-yl)methyl)-5-(6-ethoxypyrazin-2- | | LCMS-BH |
| | yl)thiazole-2-carboxamide | | |
| | 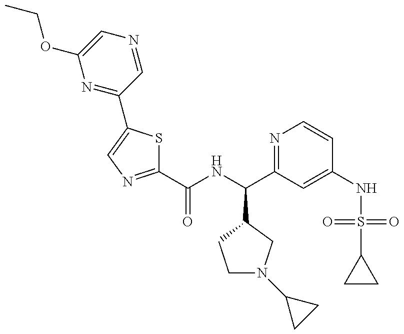 [SMILES: CCOc1cncc(-c2cnc(C(=O)N[C@@H](c3cc(NS(=O)(=O)C4CC4)ccn3)[C@H]3CCN(C4CC4)C3)s2)n1] | | |
| I-112 | N-((S)-(4-(cyclopropanesulfonamido)pyridin-2-yl)((S)-1- | 570 | 1.691 |
| | cyclopropylpyrrolidin-3-yl)methyl)-5-(6-ethoxypyrazin-2- | | LCMS-BH |
| | yl)thiazole-2-carboxamide | | |
| | 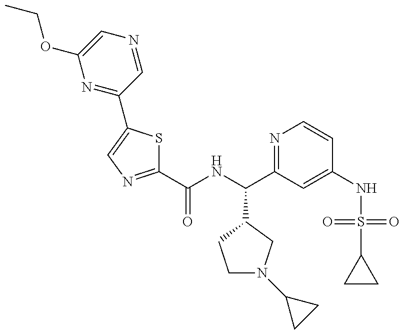 [SMILES: CCOc1cncc(-c2cnc(C(=O)N[C@H](c3cc(NS(=O)(=O)C4CC4)ccn3)[C@H]3CCN(C4CC4)C3)s2)n1] | | |
| I-113 | N-((S)-(4-(cyclopropanesulfonamido)pyridin-2-yl)((R)-1- | 570 | 1.796 |
| | cyclopropylpyrrolidin-3-yl)methyl)-5-(6-ethoxypyrazin-2- | | LCMS-BH |
| | yl)thiazole-2-carboxamide | | |
| | 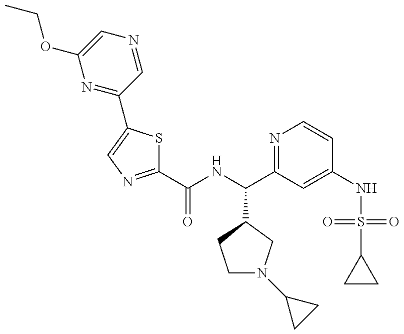 [SMILES: CCOc1cncc(-c2cnc(C(=O)N[C@H](c3cc(NS(=O)(=O)C4CC4)ccn3)[C@@H]3CCN(C4CC4)C3)s2)n1] | | |
| I-114 | N-((R)-(4-(cyclopropanesulfonamido)pyridin-2-yl)((R)-1- | 570 | 1.794 |
| | cyclopropylpyrrolidin-3-yl)methyl)-5-(6-ethoxypyrazin-2- | | LCMS-BH |
| | yl)thiazole-2-carboxamide | | |
| | 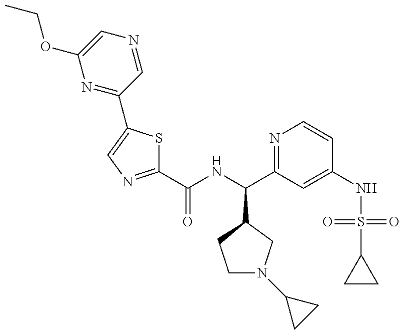 [SMILES: CCOc1cncc(-c2cnc(C(=O)N[C@@H](c3cc(NS(=O)(=O)C4CC4)ccn3)[C@@H]3CCN(C4CC4)C3)s2)n1] | | |
| I-115 | N-((S)-(4-(cyclopropanesulfonamido)pyridin-2-yl)((S)-1- | 544 | 0.908 |
| | methylpyrrolidin-2-yl)methyl)-5-(6-ethoxypyrazin-2- | | LCMS-AU |
| | yl)thiazole-2-carboxamide | | |
| | 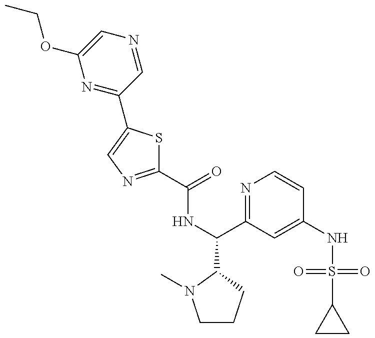 [SMILES: CCOc1cncc(-c2cnc(C(=O)N[C@H](c3cc(NS(=O)(=O)C4CC4)ccn3)[C@@H]3CCCN3C)s2)n1] | | |
| I-116 | N-((S)-(4-(cyclopropanesulfonamido)pyridin-2-yl)((R)-1- | 544 | 0.81 |
| | methylpyrrolidin-2-yl)methyl)-5-(6-ethoxypyrazin-2- | | LCMS-AW |
| | yl)thiazole-2-carboxamide | | |
| | 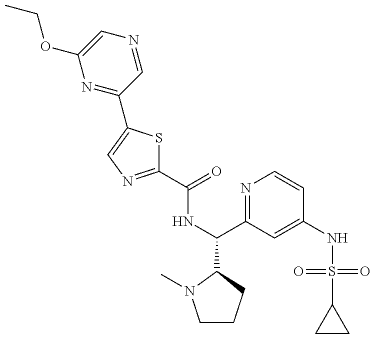 [SMILES: CCOc1cncc(-c2cnc(C(=O)N[C@H](c3cc(NS(=O)(=O)C4CC4)ccn3)[C@H]3CCCN3C)s2)n1] | | |
| I-117 | N-((R)-(4-(cyclopropanesulfonamido)pyridin-2-yl)((S)-1- | 544 | 0.903 |
| | methylpyrrolidin-2-yl)methyl)-5-(6-ethoxypyrazin-2- | | LCMS-AU |
| | yl)thiazole-2-carboxamide | | |
| | 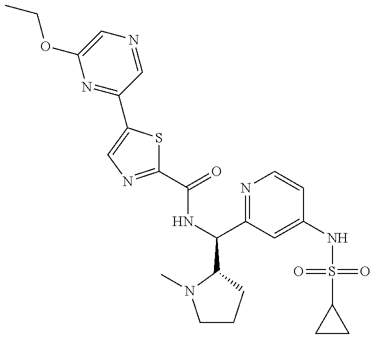 [SMILES: CCOc1cncc(-c2cnc(C(=O)N[C@@H](c3cc(NS(=O)(=O)C4CC4)ccn3)[C@@H]3CCCN3C)s2)n1] | | |
| I-118 | N-((R)-(4-(cyclopropanesulfonamido)pyridin-2-yl)((R)-1- | 544 | 1.543 |
| | methylpyrrolidin-2-yl)methyl)-5-(6-ethoxypyrazin-2- | | LCMS-AT |
| | yl)thiazole-2-carboxamide | | |
| | 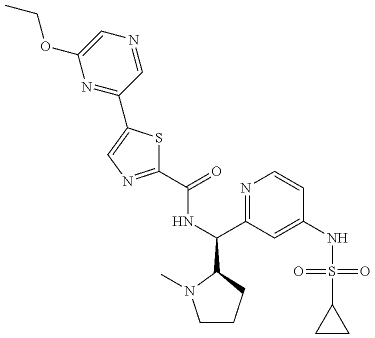 [SMILES: CCOc1cncc(-c2cnc(C(=O)N[C@@H](c3cc(NS(=O)(=O)C4CC4)ccn3)[C@H]3CCCN3C)s2)n1] | | |
| I-119 | N-((S)-(4-(cyclopropanesulfonamido)pyridin-2-yl)((R)-1- | 558 | 1.002 |
| | methyl-5-oxopyrrolidin-3-yl)methyl)-5-(6-ethoxypyrazin-2- | | LCMS-P |
| | yl)thiazole-2-carboxamide | | |
| | 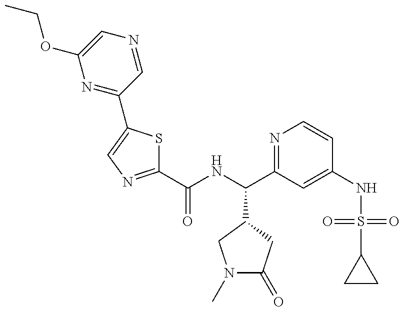 [SMILES: CCOc1cncc(-c2cnc(C(=O)N[C@H](c3cc(NS(=O)(=O)C4CC4)ccn3)[C@@H]3CC(=O)N(C)C3)s2)n1] | | |
| I-120 | N-((S)-(4-(cyclopropanesulfonamido)pyridin-2-yl)((S)-1- | 558 | 0.988 |
| | methyl-5-oxopyrrolidin-3-yl)methyl)-5-(6-ethoxypyrazin-2- | | LCMS-P |
| | yl)thiazole-2-carboxamide | | |
| | 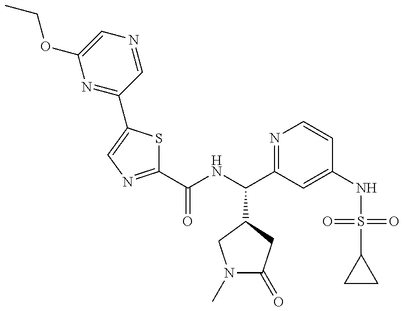 [SMILES: CCOc1cncc(-c2cnc(C(=O)N[C@H](c3cc(NS(=O)(=O)C4CC4)ccn3)[C@H]3CC(=O)N(C)C3)s2)n1] | | |
| I-121 | N-((R)-(4-(cyclopropanesulfonamido)pyridin-2-yl)((R)-1- | 558 | 1.633 |
| | methyl-5-oxopyrrolidin-3-yl)methyl)-5-(6-ethoxypyrazin-2- | | LCMS-Q |
| | yl)thiazole-2-carboxamide | | |
| | 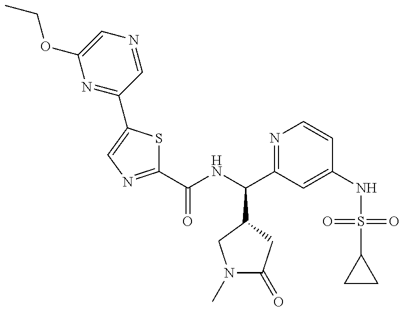 [SMILES: CCOc1cncc(-c2cnc(C(=O)N[C@@H](c3cc(NS(=O)(=O)C4CC4)ccn3)[C@@H]3CC(=O)N(C)C3)s2)n1] | | |
| I-122 | N-((R)-(4-(cyclopropanesulfonamido)pyridin-2-yl)((S)-1- | 558 | 1.135 |
| | methyl-5-oxopyrrolidin-3-yl)methyl)-5-(6-ethoxypyrazin-2- | | LCMS-S |
| | yl)thiazole-2-carboxamide | | |
| | 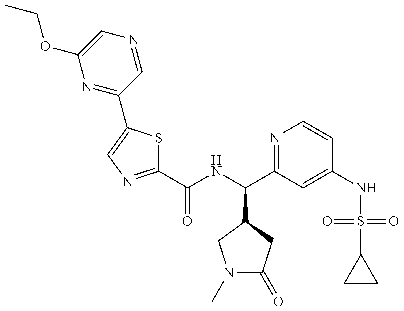 [SMILES: CCOc1cncc(-c2cnc(C(=O)N[C@@H](c3cc(NS(=O)(=O)C4CC4)ccn3)[C@H]3CC(=O)N(C)C3)s2)n1] | | |
| I-123 | N-((S)-2-((1R,4R)-2-oxa-5-azabicyclo[2.2.2]octan-5-yl)-1-(4- | 586 | 0.885 |
| | (cyclopropanesulfonamido)pyridin-2-yl)ethyl)-5-(6- | | LCMS-BB |
| | ethoxypyrazin-2-yl)thiazole-2-carboxamide | | |
| | 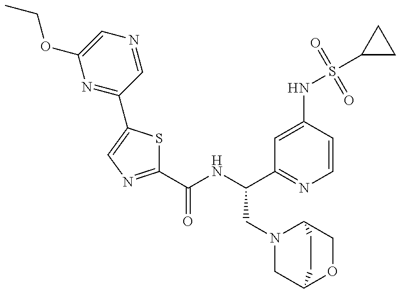 [SMILES: CCOc1cncc(-c2cnc(C(=O)N[C@@H](CN3C[C@H]4CC[C@@H]3CO4)c3cc(NS(=O)(=O)C4CC4)ccn3)s2)n1] | | |
| I-124 | N-((R)-2-((1R,4R)-2-oxa-5-azabicyclo[2.2.2]octan-5-yl)-1-(4- | 586 | 0.876 |
| | (cyclopropanesulfonamido)pyridin-2-yl)ethyl)-5-(6- | | LCMS-BB |
| | ethoxypyrazin-2-yl)thiazole-2-carboxamide | | |
| | 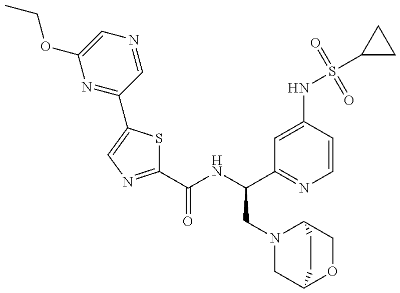 [SMILES: CCOc1cncc(-c2cnc(C(=O)N[C@H](CN3C[C@H]4CC[C@@H]3CO4)c3cc(NS(=O)(=O)C4CC4)ccn3)s2)n1] | | |
| I-125 | N-((S)-1-(4-(cyclopropanesulfonamido)pyridin-2-yl)-2-((R)-2- | 600 | 1.71 |
| | oxa-7-azaspiro[4.4]nonan-7-yl)ethyl)-5-(6-ethoxypyrazin-2- | | LCMS-X |
| | yl)thiazole-2-carboxamide | | |
| | 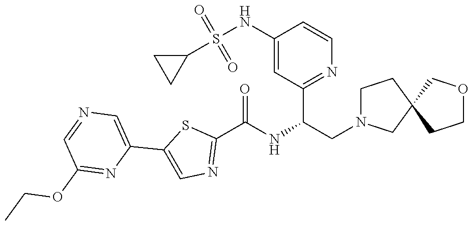 [SMILES: CCOc1cncc(-c2cnc(C(=O)N[C@@H](CN3CC[C@@]4(CCOC4)C3)c3cc(NS(=O)(=O)C4CC4)ccn3)s2)n1] | | |
| I-126 | N-((R)-1-(4-(cyclopropanesulfonamido)pyridin-2-yl)-2-((R)-2- | 600 | 1.54 |
| | oxa-7-azaspiro[4.4]nonan-7-yl)ethyl)-5-(6-ethoxypyrazin-2- | | LCMS-V |
| | yl)thiazole-2-carboxamide | | |
| | 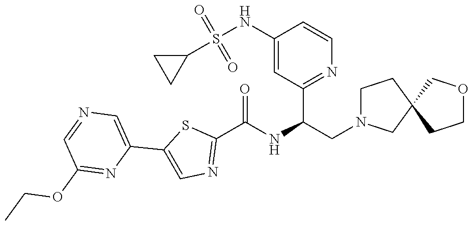 [SMILES: CCOc1cncc(-c2cnc(C(=O)N[C@H](CN3CC[C@@]4(CCOC4)C3)c3cc(NS(=O)(=O)C4CC4)ccn3)s2)n1] | | |
| I-127 | N-((S)-1-(4-(cyclopropanesulfonamido)pyridin-2-yl)-2-((S)-2- | 600 | 3.39 |
| | oxa-7-azaspiro[4.4]nonan-7-yl)ethyl)-5-(6-ethoxypyrazin-2- | | LCMS-AL |
| | yl)thiazole-2-carboxamide | | |
| | 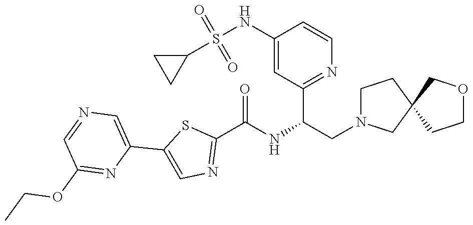 [SMILES: CCOc1cncc(-c2cnc(C(=O)N[C@@H](CN3CC[C@]4(CCOC4)C3)c3cc(NS(=O)(=O)C4CC4)ccn3)s2)n1] | | |
| I-128 | N-((R)-1-(4-(cyclopropanesulfonamido)pyridin-2-yl)-2-((S)-2- | 600 | 1.549 |
| | oxa-7-azaspiro[4.4]nonan-7-yl)ethyl)-5-(6-ethoxypyrazin-2- | | LCMS-V |
| | yl)thiazole-2-carboxamide | | |
| | 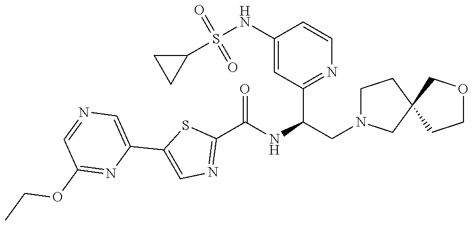 [SMILES: CCOc1cncc(-c2cnc(C(=O)N[C@H](CN3CC[C@]4(CCOC4)C3)c3cc(NS(=O)(=O)C4CC4)ccn3)s2)n1] | | |
| I-129 | N-((S)-1-(4-(cyclopropanesulfonamido)pyridin-2-yl)-2-((R)-3- | 574 | 1.342 |
| | hydroxy-3-methylpyrrolidin-1-yl)ethyl)-5-(6-ethoxypyrazin-2- | | LCMS-A |
| | yl)thiazole-2-carboxamide | | |
| | 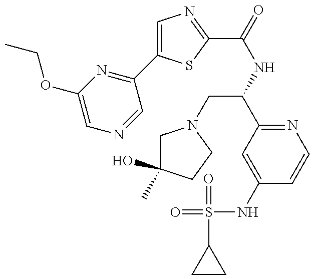 [SMILES: CCOc1cncc(-c2cnc(C(=O)N[C@@H](CN3CC[C@@](C)(O)C3)c3cc(NS(=O)(=O)C4CC4)ccn3)s2)n1] | | |
| I-130 | N-((S)-1-(4-(cyclopropanesulfonamido)pyridin-2-yl)-2-((S)-3- | 574 | 1.342 |
| | hydroxy-3-methylpyrrolidin-1-yl)ethyl)-5-(6-ethoxypyrazin-2- | | LCMS-A |
| | yl)thiazole-2-carboxamide | | |
| | 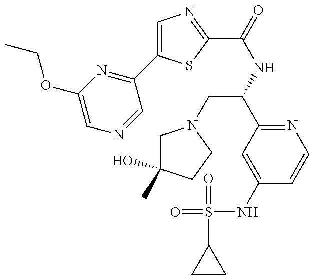 [SMILES: CCOc1cncc(-c2cnc(C(=O)N[C@@H](CN3CC[C@](C)(O)C3)c3cc(NS(=O)(=O)C4CC4)ccn3)s2)n1] | | |
| I-131 | N-((R)-1-(4-(cyclopropanesulfonamido)pyridin-2-yl)-2-((S)-3- | 574 | 1.348 |
| | hydroxy-3-methylpyrrolidin-1-yl)ethyl)-5-(6-ethoxypyrazin-2- | | LCMS-A |
| | yl)thiazole-2-carboxamide | | |
| | 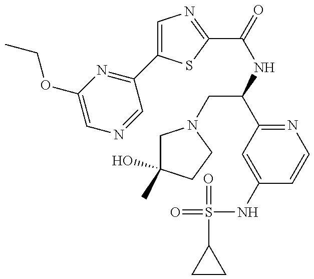 [SMILES: CCOc1cncc(-c2cnc(C(=O)N[C@H](CN3CC[C@](C)(O)C3)c3cc(NS(=O)(=O)C4CC4)ccn3)s2)n1] | | |
| I-132 | N-((R)-1-(4-(cyclopropanesulfonamido)pyridin-2-yl)-2-((R)-3- | 574 | 1.354 |
| | hydroxy-3-methylpyrrolidin-1-yl)ethyl)-5-(6-ethoxypyrazin-2- | | LCMS-A |
| | yl)thiazole-2-carboxamide | | |
| | 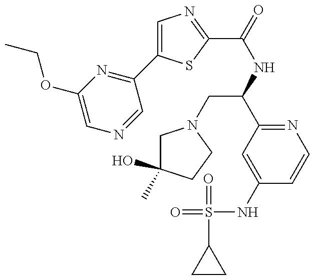 [SMILES: CCOc1cncc(-c2cnc(C(=O)N[C@H](CN3CC[C@@](C)(O)C3)c3cc(NS(=O)(=O)C4CC4)ccn3)s2)n1] | | |
| I-133 | (R)-N-(1-(4-(cyclopropanesulfonamido)pyridin-2-yl)-2-(1,4- | 574 | 1.53 |
| | oxazepan-4-yl)ethyl)-5-(6-ethoxypyrazin-2-yl)thiazole-2- | | LCMS-V |
| | carboxamide | | |
| | 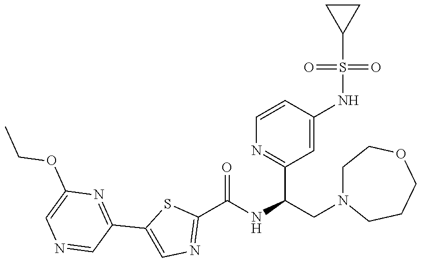 [SMILES: CCOc1cncc(-c2cnc(C(=O)N[C@H](CN3CCCOCC3)c3cc(NS(=O)(=O)C4CC4)ccn3)s2)n1] | | |
| I-134 | (S)-N-(1-(4-(cyclopropanesulfonamido)pyridin-2-yl)-2-(1,4- | 574 | 0.975 |
| | oxazepan-4-yl)ethyl)-5-(6-ethoxypyrazin-2-yl)thiazole-2- | | LCMS-W |
| | carboxamide | | |
| | 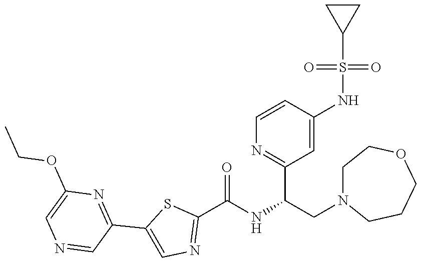 [SMILES: CCOc1cncc(-c2cnc(C(=O)N[C@@H](CN3CCCOCC3)c3cc(NS(=O)(=O)C4CC4)ccn3)s2)n1] | | |
| I-135 | N-((S)-2-((1S,4S)-2-oxa-5-azabicyclo[2.2.2]octan-5-yl)-1-(4- | 586 | 0.889 |
| | (cyclopropanesulfonamido)pyridin-2-yl)ethyl)-5-(6- | | LCMS-AY |
| | ethoxypyrazin-2-yl)thiazole-2-carboxamide | | |
| | 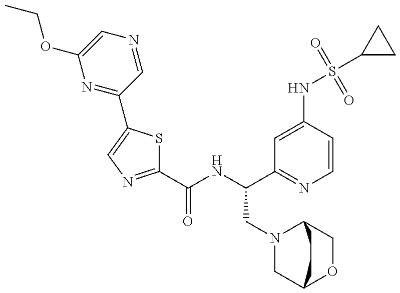 [SMILES: CCOc1cncc(-c2cnc(C(=O)N[C@@H](CN3C[C@@H]4CC[C@H]3CO4)c3cc(NS(=O)(=O)C4CC4)ccn3)s2)n1] | | |
| I-136 | N-((R)-2-((1S,4S)-2-oxa-5-azabicyclo[2.2.2]octan-5-yl)-1-(4- | 586 | 0.869 |
| | (cyclopropanesulfonamido)pyridin-2-yl)ethyl)-5-(6- | | LCMS-AY |
| | ethoxypyrazin-2-yl)thiazole-2-carboxamide | | |
| | 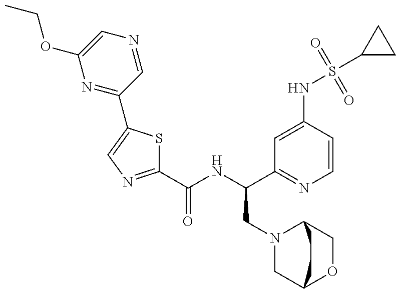 [SMILES: CCOc1cncc(-c2cnc(C(=O)N[C@H](CN3C[C@@H]4CC[C@H]3CO4)c3cc(NS(=O)(=O)C4CC4)ccn3)s2)n1] | | |
| I-137 | (R)-N-(1-(4-(cyclopropanesulfonamido)pyridin-2-yl)-2-(2- | 558 | 1.502 |
| | oxopyrrolidin-1-yl)ethyl)-5-(6-ethoxypyrazin-2-yl)thiazole-2- | | LCMS-BC |
| | carboxamide | | |
| | 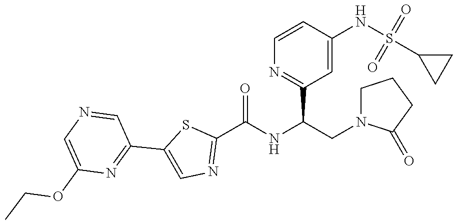 [SMILES: CCOc1cncc(-c2cnc(C(=O)N[C@H](CN3CCCC3=O)c3cc(NS(=O)(=O)C4CC4)ccn3)s2)n1] | | |
| I-138 | (S)-N-(1-(4-(cyclopropanesulfonamido)pyridin-2-yl)-2-(2- | 558 | 1.51 |
| | oxopyrrolidin-1-yl)ethyl)-5-(6-ethoxypyrazin-2-yl)thiazole-2- | | LCMS-BC |
| | carboxamide | | |
| | 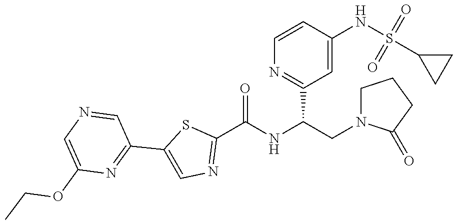 [SMILES: CCOc1cncc(-c2cnc(C(=O)N[C@@H](CN3CCCC3=O)c3cc(NS(=O)(=O)C4CC4)ccn3)s2)n1] | | |
| I-139 | (R)-N-(1-(4-(cyclopropanesulfonamido)pyridin-2-yl)-2-(2- | 568 | 1.375 |
| | oxopyridin-1(2H)-yl)ethyl)-5-(6-ethoxypyrazin-2-yl)thiazole-2- | | LCMS-A |
| | carboxamide | | |
| | 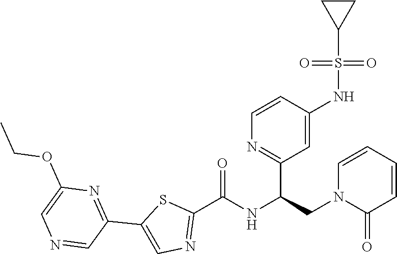 [SMILES: CCOc1cncc(-c2cnc(C(=O)N[C@H](Cn3ccccc3=O)c3cc(NS(=O)(=O)C4CC4)ccn3)s2)n1] | | |
| I-140 | (S)-N-(1-(4-(cyclopropanesulfonamido)pyridin-2-yl)-2-(2- | 568 | 1.355 |
| | oxopyridin-1(2H)-yl)ethyl)-5-(6-ethoxypyrazin-2-yl)thiazole-2- | | LCMS-A |
| | carboxamide | | |
| | 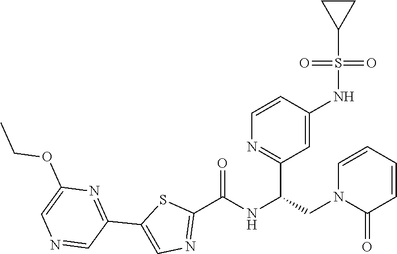 [SMILES: CCOc1cncc(-c2cnc(C(=O)N[C@@H](Cn3ccccc3=O)c3cc(NS(=O)(=O)C4CC4)ccn3)s2)n1] | | |
| I-141 | (S)-N-(1-(4-(cyclopropanesulfonamido)pyridin-2-yl)-2- | 574 | 0.999 |
| | morpholinoethyl)-5-(6-ethoxypyrazin-2-yl)-N-methylthiazole-2- | | LCMS-AN |
| | carboxamide | | |
| | 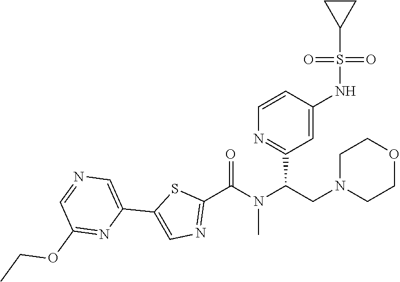 [SMILES: CCOc1cncc(-c2cnc(C(=O)N(C)[C@@H](CN3CCOCC3)c3cc(NS(=O)(=O)C4CC4)ccn3)s2)n1] | | |
| I-142 | (R)-N-(1-(4-(cyclopropanesulfonamido)pyridin-2-yl)-2- | 574 | 1.002 |
| | morpholinoethyl)-5-(6-ethoxypyrazin-2-yl)-N-methylthiazole-2- | | LCMS-AN |
| | carboxamide | | |
| | 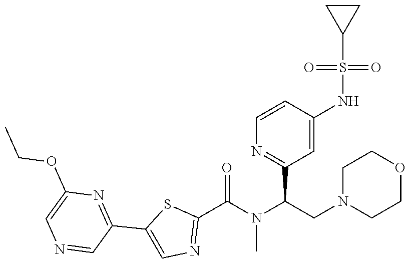 [SMILES: CCOc1cncc(-c2cnc(C(=O)N(C)[C@H](CN3CCOCC3)c3cc(NS(=O)(=O)C4CC4)ccn3)s2)n1] | | |
| I-143 | (S)-N-(1-(4-(cyclopropanesulfonamido)pyridin-2-yl)-2- | 571 | 0.875 |
| | morpholinoethyl)-4-(6-ethoxypyrazin-2-yl)-2-fluorobenzamide | | LCMS-BI |
| | 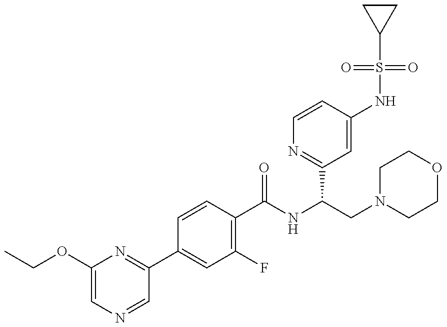 [SMILES: CCOc1cncc(-c2ccc(C(=O)N[C@@H](CN3CCOCC3)c3cc(NS(=O)(=O)C4CC4)ccn3)c(F)c2)n1] | | |
| I-144 | (R)-N-(1-(4-(cyclopropanesulfonamido)pyridin-2-yl)-2- | 571 | 0.848 |
| | morpholinoethyl)-4-(6-ethoxypyrazin-2-yl)-2-fluorobenzamide | | LCMS-BI |
| | 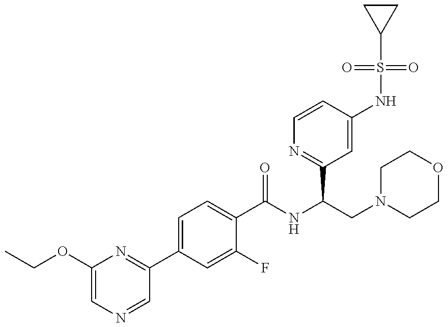 [SMILES: CCOc1cncc(-c2ccc(C(=O)N[C@H](CN3CCOCC3)c3cc(NS(=O)(=O)C4CC4)ccn3)c(F)c2)n1] | | |
| I-145 | (S)-5-(5-chloropyrazolo[1,5-a]pyridin-3-yl)-N-(1-(4- | 588/590 | 1.357 |
| | (cyclopropanesulfonamido)pyridin-2-yl)-2- | | LCMS-F |
| | morpholinoethyl)thiazole-2-carboxamide | | |
| | 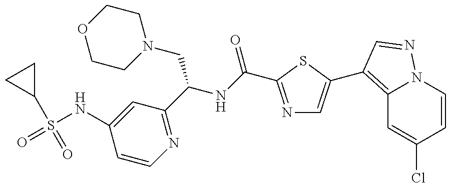 [SMILES: O=C(N[C@@H](CN1CCOCC1)c1cc(NS(=O)(=O)C2CC2)ccn1)c1ncc(-c2cnn3ccc(Cl)cc23)s1] | | |
| I-146 | (R)-5-(5-chloropyrazolo[1,5-a]pyridin-3-yl)-N-(1-(4- | 588/590 | 1.357 |
| | (cyclopropanesulfonamido)pyridin-2-yl)-2- | | LCMS-F |
| | morpholinoethyl)thiazole-2-carboxamide | | |
| | 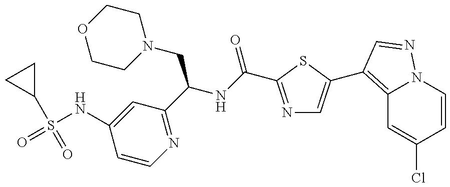 [SMILES: O=C(N[C@H](CN1CCOCC1)c1cc(NS(=O)(=O)C2CC2)ccn1)c1ncc(-c2cnn3ccc(Cl)cc23)s1] | | |
| I-147 | (S)-N-(1-(4-(cyclopropanesulfonamido)pyridin-2-yl)-2-(4- | 587 | 1.687 |
| | methyl-3-oxopiperazin-1-yl)ethyl)-5-(6-ethoxypyrazin-2- | | LCMS-X |
| | yl)thiazole-2-carboxamide | | |
| | 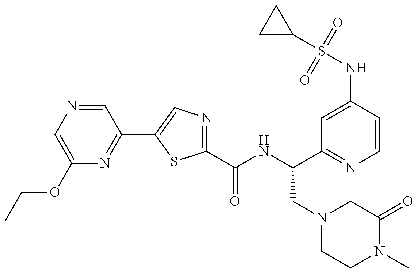 [SMILES: CCOc1cncc(-c2cnc(C(=O)N[C@@H](CN3CCN(C)C(=O)C3)c3cc(NS(=O)(=O)C4CC4)ccn3)s2)n1] | | |
| I-148 | (R)-N-(1-(4-(cyclopropanesulfonamido)pyridin-2-yl)-2-(4- | 587 | 1.527 |
| | methyl-3-oxopiperazin-1-yl)ethyl)-5-(6-ethoxypyrazin-2- | | LCMS-V |
| | yl)thiazole-2-carboxamide | | |
| | 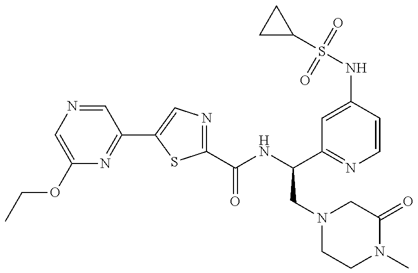 [SMILES: CCOc1cncc(-c2cnc(C(=O)N[C@H](CN3CCN(C)C(=O)C3)c3cc(NS(=O)(=O)C4CC4)ccn3)s2)n1] | | |
| I-149 | N-((S)-2-((1R,4S)-2-azabicyclo[2.2.1]heptan-2-yl)-1-(4- | 570 | 0.855 |
| | (cyclopropanesulfonamido)pyridin-2-yl)ethyl)-5-(6- | | LCMS-BB |
| | ethoxypyrazin-2-yl)thiazole-2-carboxamide | | |
| | 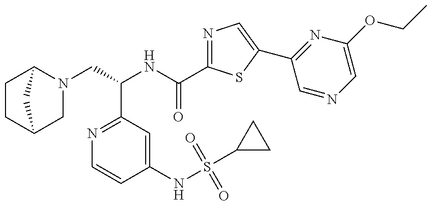 [SMILES: CCOc1cncc(-c2cnc(C(=O)N[C@@H](CN3C[C@H]4CC[C@@H]3C4)c3cc(NS(=O)(=O)C4CC4)ccn3)s2)n1] | | |
| I-150 | N-((R)-2-((1R,4S)-2-azabicyclo[2.2.1]heptan-2-yl)-1-(4- | 570 | 0.862 |
| | (cyclopropanesulfonamido)pyridin-2-yl)ethyl)-5-(6- | | LCMS-BB |
| | ethoxypyrazin-2-yl)thiazole-2-carboxamide | | |
| | 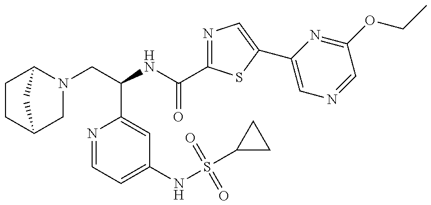 [SMILES: CCOc1cncc(-c2cnc(C(=O)N[C@H](CN3C[C@H]4CC[C@@H]3C4)c3cc(NS(=O)(=O)C4CC4)ccn3)s2)n1] | | |
| I-151 | N-((S)-2-((1R,5R)-3-azabicyclo[3.1.1]heptan-3-yl)-1-(4- | 570 | 1.593 |
| | (cyclopropanesulfonamido)pyridin-2-yl)ethyl)-5-(6- | | LCMS-R |
| | ethoxypyrazin-2-yl)thiazole-2-carboxamide | | |
| | 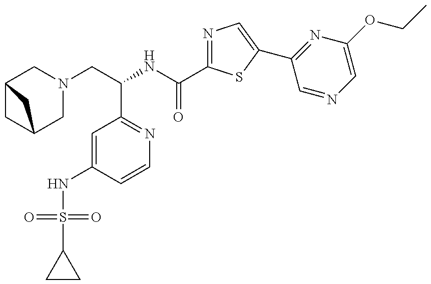 [SMILES: CCOc1cncc(-c2cnc(C(=O)N[C@@H](CN3C[C@H]4C[C@@H](C3)C4)c3cc(NS(=O)(=O)C4CC4)ccn3)s2)n1] | | |
| I-152 | N-((R)-2-((1R,5R)-3-azabicyclo[3.1.1]heptan-3-yl)-1-(4- | 570 | 1.135 |
| | (cyclopropanesulfonamido)pyridin-2-yl)ethyl)-5-(6- | | LCMS-BB |
| | ethoxypyrazin-2-yl)thiazole-2-carboxamide | | |
| | 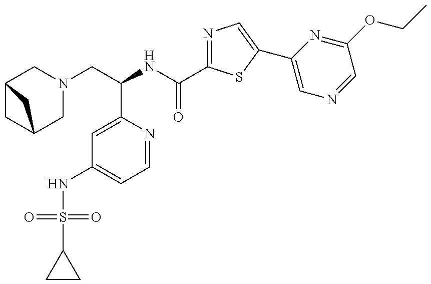 [SMILES: CCOc1cncc(-c2cnc(C(=O)N[C@H](CN3C[C@H]4C[C@@H](C3)C4)c3cc(NS(=O)(=O)C4CC4)ccn3)s2)n1] | | |
| I-153 | (S)-N-(1-(4-(cyclopropanesulfonamido)pyridin-2-yl)-2-(2- | 570 | 0.971 |
| | azaspiro[3.3]heptan-2-yl)ethyl)-5-(6-ethoxypyrazin-2- | | LCMS-AN |
| | yl)thiazole-2-carboxamide | | |
| | 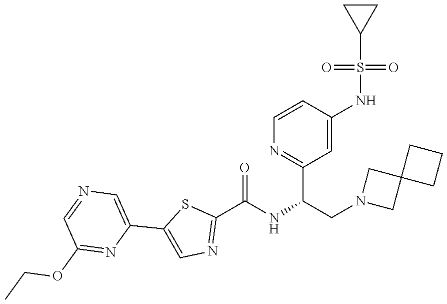 [SMILES: CCOc1cncc(-c2cnc(C(=O)N[C@@H](CN3CC4(CCC4)C3)c3cc(NS(=O)(=O)C4CC4)ccn3)s2)n1] | | |
| I-154 | (R)-N-(1-(4-(cyclopropanesulfonamido)pyridin-2-yl)-2-(2- | 570 | 0.971 |
| | azaspiro[3.3]heptan-2-yl)ethyl)-5-(6-ethoxypyrazin-2- | | LCMS-AN |
| | yl)thiazole-2-carboxamide | | |
| | 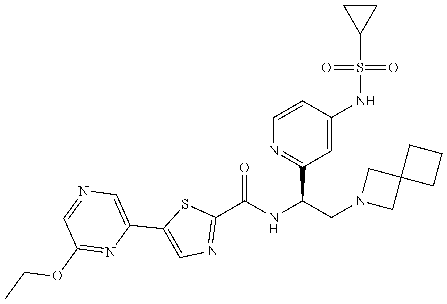 [SMILES: CCOc1cncc(-c2cnc(C(=O)N[C@H](CN3CC4(CCC4)C3)c3cc(NS(=O)(=O)C4CC4)ccn3)s2)n1] | | |
| I-155 | (S)-N-(1-(4-(cyclopropanesulfonamido)pyridin-2-yl)-2-(1H- | 542 | 0.873 |
| | 1,2,3-triazol-1-yl)ethyl)-5-(6-ethoxypyrazin-2-yl)thiazole-2- | | LCMS-AC |
| | carboxamide | | |
| | 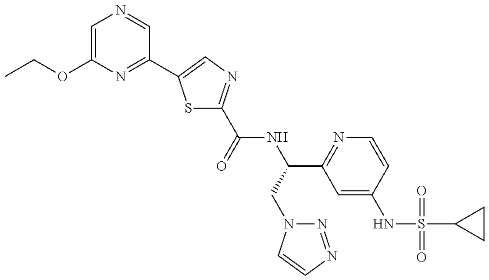 [SMILES: CCOc1cncc(-c2cnc(C(=O)N[C@@H](Cn3ccnn3)c3cc(NS(=O)(=O)C4CC4)ccn3)s2)n1] | | |
| I-156 | (R)-N-(1-(4-(cyclopropanesulfonamido)pyridin-2-yl)-2-(1H- | 542 | 0.88 |
| | 1,2,3-triazol-1-yl)ethyl)-5-(6-ethoxypyrazin-2-yl)thiazole-2- | | LCMS-AC |
| | carboxamide | | |
| | 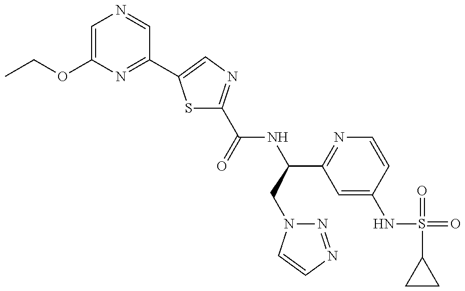 [SMILES: CCOc1cncc(-c2cnc(C(=O)N[C@H](Cn3ccnn3)c3cc(NS(=O)(=O)C4CC4)ccn3)s2)n1] | | |
| I-157 | (S)-N-(1-(4-(cyclopropanesulfonamido)pyridin-2-yl)-2-(2H- | 542 | 1.082 |
| | 1,2,3-triazol-2-yl)ethyl)-5-(6-ethoxypyrazin-2-yl)thiazole-2- | | LCMS-P |
| | carboxamide | | |
| | 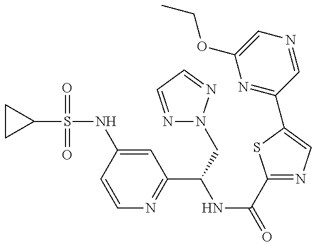 [SMILES: CCOc1cncc(-c2cnc(C(=O)N[C@@H](Cn3nccn3)c3cc(NS(=O)(=O)C4CC4)ccn3)s2)n1] | | |
| I-158 | (R)-N-(1-(4-(cyclopropanesulfonamido)pyridin-2-yl)-2-(2H- | 542 | 1.075 |
| | 1,2,3-triazol-2-yl)ethyl)-5-(6-ethoxypyrazin-2-yl)thiazole-2- | | LCMS-P |
| | carboxamide | | |
| | 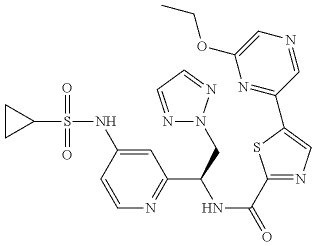 [SMILES: CCOc1cncc(-c2cnc(C(=O)N[C@H](Cn3nccn3)c3cc(NS(=O)(=O)C4CC4)ccn3)s2)n1] | | |
| I-159 | N-(2-((1R,4R)-2-oxa-5-azabicyclo[2.2.1]heptan-5-yl)-1-(4- | 600 | 0.858 |
| | (cyclopropanesulfonamido)pyridin-2-yl)ethyl)-5-(5- | | LCMS-E |
| | chloropyrazolo[1,5-a]pyridin-3-yl)thiazole-2-carboxamide | | |
| | 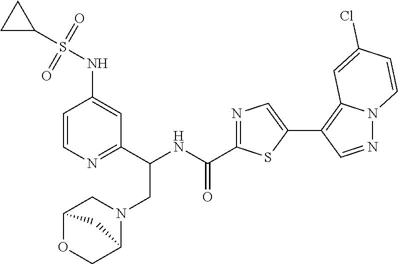 [SMILES: O=C(NC(CN1C[C@H]2C[C@@H]1CO2)c1cc(NS(=O)(=O)C2CC2)ccn1)c1ncc(-c2cnn3ccc(Cl)cc23)s1] | | |
| I-160 | (R)-N-(1-(4-(cyclopropanesulfonamido)pyridin-2-yl)-2- | 493 | 1.042 |
| | fluoroethyl)-5-(6-ethoxypyrazin-2-yl)thiazole-2-carboxamide | | LCMS-P |
| | 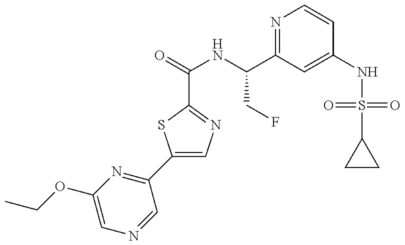 [SMILES: CCOc1cncc(-c2cnc(C(=O)N[C@@H](CF)c3cc(NS(=O)(=O)C4CC4)ccn3)s2)n1] | | |
| I-161 | (S)-N-(1-(4-(cyclopropanesulfonamido)pyridin-2-yl)-2- | 493 | 1.042 |
| | fluoroethyl)-5-(6-ethoxypyrazin-2-yl)thiazole-2-carboxamide | | LCMS-P |
| | 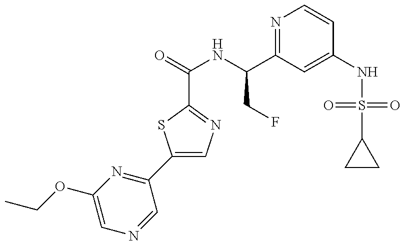 [SMILES: CCOc1cncc(-c2cnc(C(=O)N[C@H](CF)c3cc(NS(=O)(=O)C4CC4)ccn3)s2)n1] | | |
| I-162 | (S)-5-(5-chloropyrazolo[1,5-a]pyridin-3-yl)-N-(1-(4- | 572/574 | 1.521 |
| | (cyclopropanesulfonamido)pyridin-2-yl)-2-(pyrrolidin-1- | | LCMS-G |
| | yl)ethyl)thiazole-2-carboxamide | | |
| | 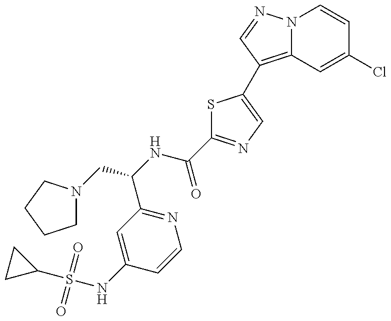 [SMILES: O=C(N[C@@H](CN1CCCC1)c1cc(NS(=O)(=O)C2CC2)ccn1)c1ncc(-c2cnn3ccc(Cl)cc23)s1] | | |
| I-163 | (R)-5-(5-chloropyrazolo[1,5-a]pyridin-3-yl)-N-(1-(4- | 572/574 | 1.519 |
| | (cyclopropanesulfonamido)pyridin-2-yl)-2-(pyrrolidin-1- | | LCMS-G |
| | yl)ethyl)thiazole-2-carboxamide | | |
| | 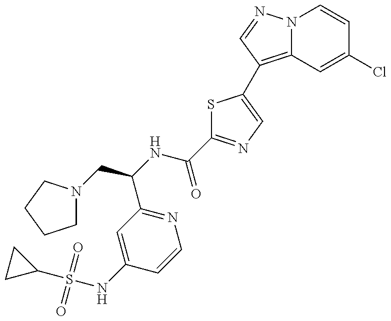 [SMILES: O=C(N[C@H](CN1CCCC1)c1cc(NS(=O)(=O)C2CC2)ccn1)c1ncc(-c2cnn3ccc(Cl)cc23)s1] | | |
| I-164 | (S)-5-(5-chloropyrazolo[1,5-a]pyridin-3-yl)-N-(1-(4- | 546/548 | 1.527 |
| | (cyclopropanesulfonamido)pyridin-2-yl)-2- | | LCMS-AK |
| | (dimethylamino)ethyl)thiazole-2-carboxamide, formic acid salt | | |
| | 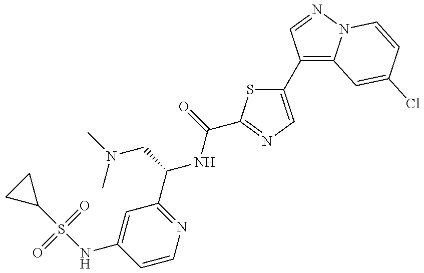 [SMILES: CN(C)C[C@H](NC(=O)c1ncc(-c2cnn3ccc(Cl)cc23)s1)c1cc(NS(=O)(=O)C2CC2)ccn1] | | |
| I-165 | (R)-5-(5-chloropyrazolo[1,5-a]pyridin-3-yl)-N-(1-(4- | 546/548 | 1.533 |
| | (cyclopropanesulfonamido)pyridin-2-yl)-2- | | LCMS-AK |
| | (dimethylamino)ethyl)thiazole-2-carboxamide, formic acid salt | | |
| | 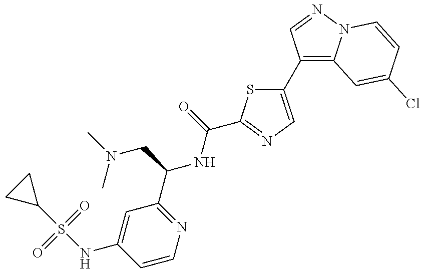 [SMILES: CN(C)C[C@@H](NC(=O)c1ncc(-c2cnn3ccc(Cl)cc23)s1)c1cc(NS(=O)(=O)C2CC2)ccn1] | | |
| I-166 | N-((1S,2S)-2-((1R,4R)-2-oxa-5-azabicyclo[2.2.1]heptan-5-yl)- | 586 | 0.873 |
| | 1-(4-(cyclopropanesulfonamido)pyridin-2-yl)propyl)-5-(6- | | LCMS-AV |
| | ethoxypyrazin-2-yl)thiazole-2-carboxamide | | |
| | 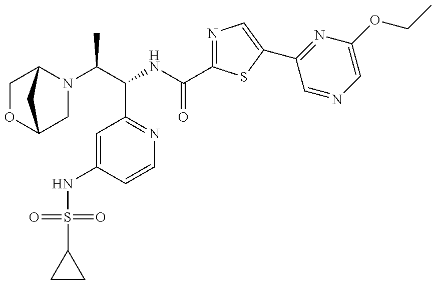 [SMILES: CCOc1cncc(-c2cnc(C(=O)N[C@H](c3cc(NS(=O)(=O)C4CC4)ccn3)[C@H](C)N3C[C@H]4C[C@@H]3CO4)s2)n1] | | |
| I-167 | N-((1S,2R)-2-((1R,4R)-2-oxa-5-azabicyclo[2.2.1]heptan-5-yl)- | 586 | 0.891 |
| | 1-(4-(cyclopropanesulfonamido)pyridin-2-yl)propyl)-5-(6- | | LCMS-E |
| | ethoxypyrazin-2-yl)thiazole-2-carboxamide | | |
| | 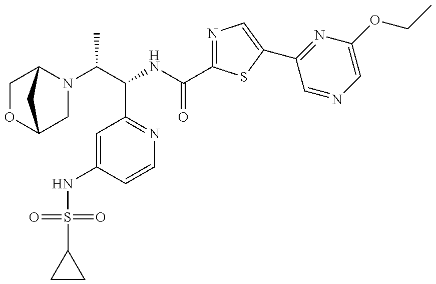 [SMILES: CCOc1cncc(-c2cnc(C(=O)N[C@H](c3cc(NS(=O)(=O)C4CC4)ccn3)[C@@H](C)N3C[C@H]4C[C@@H]3CO4)s2)n1] | | |
| I-168 | N-((1R,2S)-2-((1R,4R)-2-oxa-5-azabicyclo[2.2.1]heptan-5-yl)- | 586 | 0.899 |
| | 1-(4-(cyclopropanesulfonamido)pyridin-2-yl)propyl)-5-(6- | | LCMS-E |
| | ethoxypyrazin-2-yl)thiazole-2-carboxamide | | |
| | 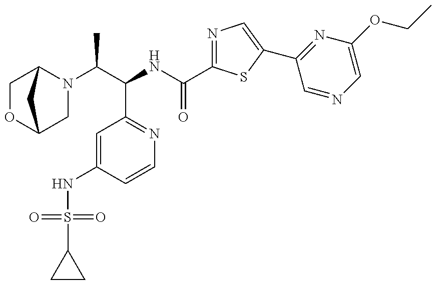 [SMILES: CCOc1cncc(-c2cnc(C(=O)N[C@@H](c3cc(NS(=O)(=O)C4CC4)ccn3)[C@H](C)N3C[C@H]4C[C@@H]3CO4)s2)n1] | | |
| I-169 | N-((1R,2R)-2-((1R,4R)-2-oxa-5-azabicyclo[2.2.1]heptan-5-yl)- | 586 | 0.967 |
| | 1-(4-(cyclopropanesulfonamido)pyridin-2-yl)propyl)-5-(6- | | LCMS-AU |
| | ethoxypyrazin-2-yl)thiazole-2-carboxamide | | |
| | 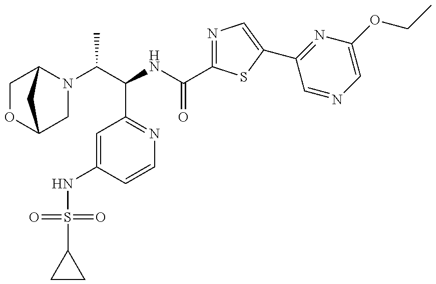 [SMILES: CCOc1cncc(-c2cnc(C(=O)N[C@@H](c3cc(NS(=O)(=O)C4CC4)ccn3)[C@@H](C)N3C[C@H]4C[C@@H]3CO4)s2)n1] | | |
| I-170 | N-((4-(cyclopropanesulfonamido)pyridin-2-yl)methyl)-5-(5- | 499 | 1.688 |
| | ethoxypyrazolo[1,5-a]pyridin-3-yl)thiazole-2-carboxamide | | LCMS-J |
| | 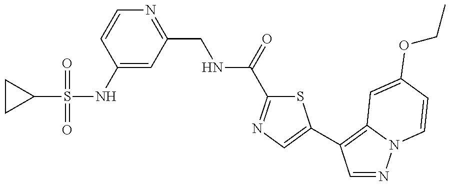 [SMILES: CCOc1ccn2ncc(-c3cnc(C(=O)NCc4cc(NS(=O)(=O)C5CC5)ccn4)s3)c2c1] | | |
| I-171 | 2-(4-(cyclopropanesulfonamido)pyridin-2-yl)-N-(5-(6- | 461 | 0.878 |
| | ethoxypyrazin-2-yl)thiazol-2-yl)acetamide | | LCMS-AC |
| | 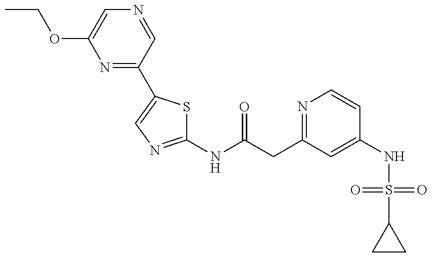 [SMILES: CCOc1cncc(-c2cnc(NC(=O)Cc3cc(NS(=O)(=O)C4CC4)ccn3)s2)n1] | | |
| I-172 | N-((4-(cyclopropanesulfonamido)pyridin-2-yl)methyl)-6-(6- | 455 | 1.275 |
| | ethoxypyrazin-2-yl)nicotinamide | | LCMS-BC |
| | 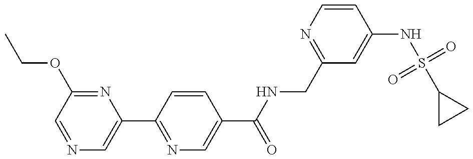 [SMILES: CCOc1cncc(-c2ccc(C(=O)NCc3cc(NS(=O)(=O)C4CC4)ccn3)cn2)n1] | | |
| I-173 | N-((4-(cyclopropanesulfonamido)pyridin-2-yl)methyl)-4-(6- | 472 | 0.863 |
| | ethoxypyrazin-2-yl)-3-fluorobenzamide | | LCMS-AU |
| | 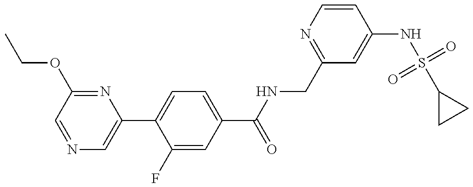 [SMILES: CCOc1cncc(-c2ccc(C(=O)NCc3cc(NS(=O)(=O)C4CC4)ccn3)cc2F)n1] | | |
| I-174 | N-((4-(cyclopropanesulfonamido)pyridin-2-yl)methyl)-4-(6- | 490 | 0.909 |
| | ethoxypyrazin-2-yl)-2,5-difluorobenzamide | | LCMS-E |
| | 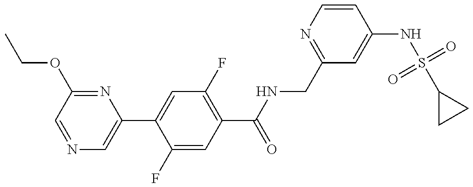 [SMILES: CCOc1cncc(-c2cc(F)c(C(=O)NCc3cc(NS(=O)(=O)C4CC4)ccn3)cc2F)n1] | | |
| I-175 | N-((4-(cyclopropanesulfonamido)pyridin-2-yl)methyl)-4-(6- | 490 | 0.873 |
| | ethoxypyrazin-2-yl)-2,3-difluorobenzamide | | LCMS-AU |
| | 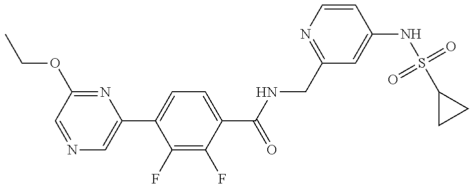 [SMILES: CCOc1cncc(-c2ccc(C(=O)NCc3cc(NS(=O)(=O)C4CC4)ccn3)c(F)c2F)n1] | | |
| I-176 | (R)-N-(1-(4-(cyclopropanesulfonamido)pyridin-2-yl)ethyl)-5- | 475 | 0.995 |
| | (6-ethoxypyrazin-2-yl)thiazole-2-carboxamide | | LCMS-P |
| | 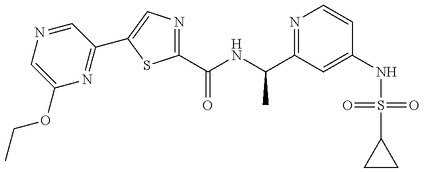 [SMILES: CCOc1cncc(-c2cnc(C(=O)N[C@H](C)c3cc(NS(=O)(=O)C4CC4)ccn3)s2)n1] | | |
| I-177 | (S)-N-(1-(4-(cyclopropanesulfonamido)pyridin-2-yl)ethyl)-5-(6- | 475 | 0.995 |
| | ethoxypyrazin-2-yl)thiazole-2-carboxamide | | LCMS-P |
| | 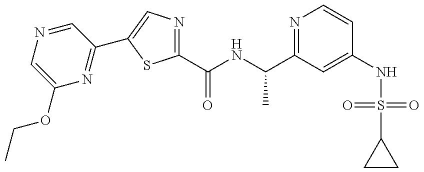 [SMILES: CCOc1cncc(-c2cnc(C(=O)N[C@@H](C)c3cc(NS(=O)(=O)C4CC4)ccn3)s2)n1] | | |
| I-178 | N-((2-(cyclopropanesulfonamido)pyrimidin-4-yl)methyl)-5-(6- | 462 | 1.115 |
| | ethoxypyrazin-2-yl)thiazole-2-carboxamide | | LCMS-P |
| | 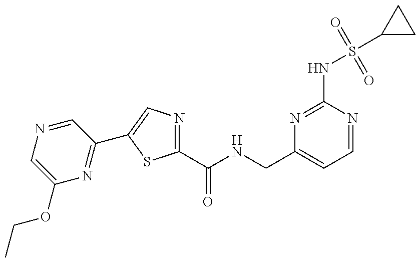 [SMILES: CCOc1cncc(-c2cnc(C(=O)NCc3ccnc(NS(=O)(=O)C4CC4)n3)s2)n1] | | |
| I-179 | N-(1-(4-(cyclopropanesulfonamido)pyridin-2-yl)cyclopropyl)- | 487 | 1.028 |
| | 5-(6-ethoxypyrazin-2-yl)thiazole-2-carboxamide | | LCMS-P |
| | 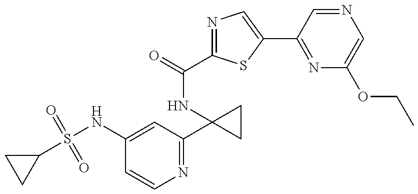 [SMILES: CCOc1cncc(-c2cnc(C(=O)NC3(c4cc(NS(=O)(=O)C5CC5)ccn4)CC3)s2)n1] | | |
| I-180 | N-((6-(cyclopropanesulfonamido)pyrazin-2-yl)methyl)-5-(6- | 462 | 1.368 |
| | ethoxypyrazin-2-yl)thiazole-2-carboxamide | | LCMS-U |
| | 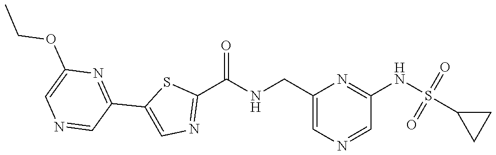 [SMILES: CCOc1cncc(-c2cnc(C(=O)NCc3cncc(NS(=O)(=O)C4CC4)n3)s2)n1] | | |
| I-181 | 4-(5-chloropyrazolo[1,5-a]pyridin-3-yl)-N-((4- | 500/502 | 1.593 |
| | (cyclopropanesulfonamido)pyridin-2-yl)methyl)-2- | | LCMS-O |
| | fluorobenzamide | | |
| | 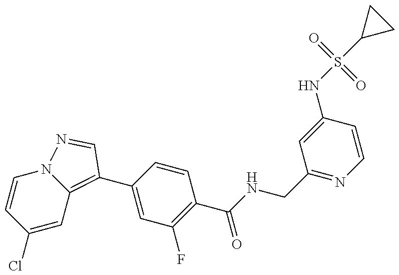 [SMILES: O=C(NCc1cc(NS(=O)(=O)C2CC2)ccn1)c1ccc(-c2cnn3ccc(Cl)cc23)cc1F] | | |
| I-182 | N-(3-(4-(cyclopropanesulfonamido)pyridin-2-yl)-1- | 530 | 1.52 |
| | methylpyrrolidin-3-yl)-5-(6-ethoxypyrazin-2-yl)thiazole-2- | | LCMS-O |
| | carboxamide, formic acid salt | | |
| | 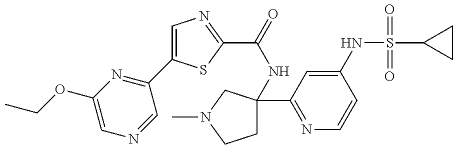 [SMILES: CCOc1cncc(-c2cnc(C(=O)NC3(c4cc(NS(=O)(=O)C5CC5)ccn4)CCN(C)C3)s2)n1] | | |
| I-183 | (S)-N-(1-(3-((difluoromethyl)sulfonamido)phenyl)-3-(piperidin- | 581 | 1.499 |
| | 1-yl)propyl)-5-(6-ethoxypyrazin-2-yl)thiazole-2-carboxamide | | LCMS-BF |
| | 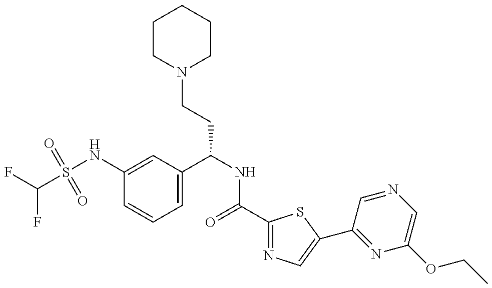 [SMILES: CCOc1cncc(-c2cnc(C(=O)N[C@@H](CCN3CCCCC3)c3cccc(NS(=O)(=O)C(F)F)c3)s2)n1] | | |
| I-184 | (R)-N-(1-(3-((difluoromethyl)sulfonamido)phenyl)-3- | 581 | 1.501 |
| | (piperidin-1-yl)propyl)-5-(6-ethoxypyrazin-2-yl)thiazole-2- | | LCMS-BF |
| | carboxamide | | |
| | 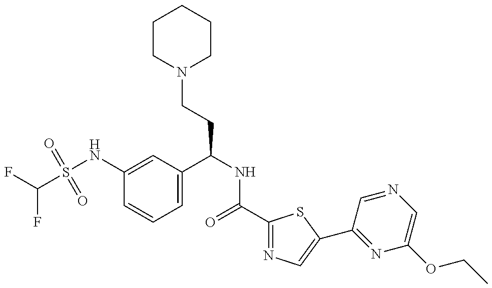 [SMILES: CCOc1cncc(-c2cnc(C(=O)N[C@H](CCN3CCCCC3)c3cccc(NS(=O)(=O)C(F)F)c3)s2)n1] | | |
| I-185 | (S)-N-(1-(4-(cyclopropanesulfonamido)pyridin-2-yl)-3- | 544 | 1.331 |
| | (cyclopropylamino)propyl)-5-(6-ethoxypyrazin-2-yl)thiazole-2- | | LCMS-B |
| | carboxamide | | |
| | 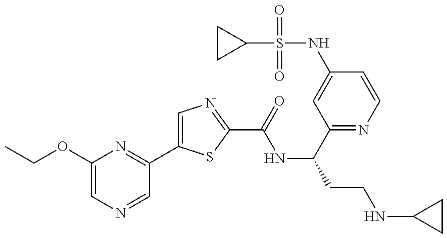 [SMILES: CCOc1cncc(-c2cnc(C(=O)N[C@@H](CCNC3CC3)c3cc(NS(=O)(=O)C4CC4)ccn3)s2)n1] | | |
| I-186 | (R)-N-(1-(4-(cyclopropanesulfonamido)pyridin-2-yl)-3- | 544 | 1.428 |
| | (cyclopropylamino)propyl)-5-(6-ethoxypyrazin-2-yl)thiazole-2- | | LCMS-AW |
| | carboxamide | | |
| | 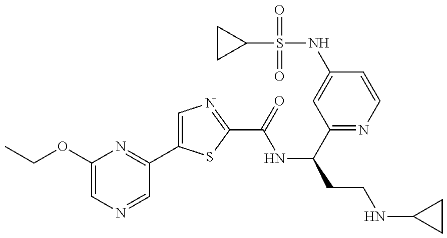 [SMILES: CCOc1cncc(-c2cnc(C(=O)N[C@H](CCNC3CC3)c3cc(NS(=O)(=O)C4CC4)ccn3)s2)n1] | | |
| I-187 | N-((S)-(3-((difluoromethyl)sulfonamido)phenyl)((R)- | 540 | 1.229 |
| | tetrahydrofuran-3-yl)methyl)-5-(6-ethoxypyrazin-2-yl)thiazole- | | LCMS-AN |
| | 2-carboxamide | | |
| | 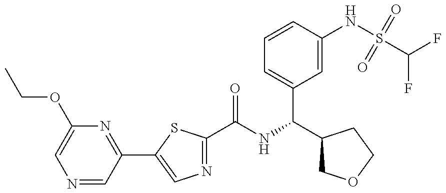 [SMILES: CCOc1cncc(-c2cnc(C(=O)N[C@H](c3cccc(NS(=O)(=O)C(F)F)c3)[C@H]3CCOC3)s2)n1] | | |
| I-188 | N-((S)-(3-((difluoromethyl)sulfonamido)phenyl)((S)- | 540 | 1.224 |
| | tetrahydrofuran-3-yl)methyl)-5-(6-ethoxypyrazin-2-yl)thiazole- | | LCMS-AN |
| | 2-carboxamide | | |
| | 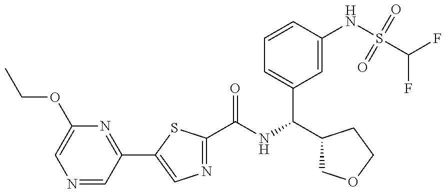 [SMILES: CCOc1cncc(-c2cnc(C(=O)N[C@H](c3cccc(NS(=O)(=O)C(F)F)c3)[C@@H]3CCOC3)s2)n1] | | |
| I-189 | N-((R)-(3-((difluoromethyl)sulfonamido)phenyl)((S)- | 540 | 1.233 |
| | tetrahydrofuran-3-yl)methyl)-5-(6-ethoxypyrazin-2-yl)thiazole- | | LCMS-AN |
| | 2-carboxamide | | |
| | 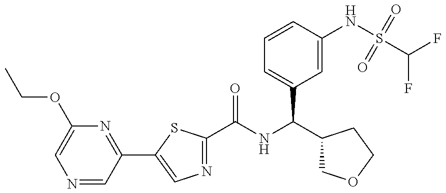 [SMILES: CCOc1cncc(-c2cnc(C(=O)N[C@@H](c3cccc(NS(=O)(=O)C(F)F)c3)[C@@H]3CCOC3)s2)n1] | | |
| I-190 | N-((R)-(3-((difluoromethyl)sulfonamido)phenyl)((R)- | 540 | 1.232 |
| | tetrahydrofuran-3-yl)methyl)-5-(6-ethoxypyrazin-2-yl)thiazole- | | LCMS-AN |
| | 2-carboxamide | | |
| | 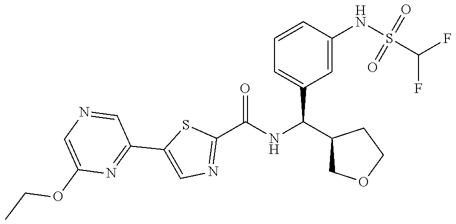 [SMILES: CCOc1cncc(-c2cnc(C(=O)N[C@@H](c3cccc(NS(=O)(=O)C(F)F)c3)[C@H]3CCOC3)s2)n1] | | |
| I-191 | N-((S)-1-(3-((difluoromethyl)sulfonamido)phenyl)-2-((R)-4- | 583 | 0.822 |
| | methylmorpholin-3-yl)ethyl)-5-(6-ethoxypyrazin-2-yl)thiazole- | | LCMS-AU |
| | 2-carboxamide | | |
| | 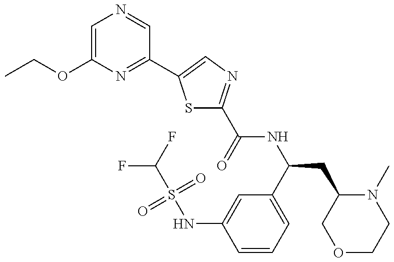 [SMILES: CCOc1cncc(-c2cnc(C(=O)N[C@@H](C[C@@H]3COCCN3C)c3cccc(NS(=O)(=O)C(F)F)c3)s2)n1] | | |
| I-192 | N-((S)-1-(3-((difluoromethyl)sulfonamido)phenyl)-2-((S)-4- | 583 | 0.822 |
| | methylmorpholin-3-yl)ethyl)-5-(6-ethoxypyrazin-2-yl)thiazole- | | LCMS-AU |
| | 2-carboxamide | | |
| | 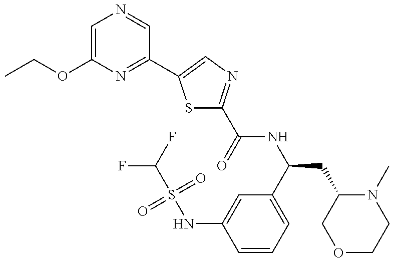 [SMILES: CCOc1cncc(-c2cnc(C(=O)N[C@@H](C[C@H]3COCCN3C)c3cccc(NS(=O)(=O)C(F)F)c3)s2)n1] | | |
| I-193 | N-((1R)-1-(3-((difluoromethyl)sulfonamido)phenyl)-2-(4- | 583 | 0.822 |
| | methylmorpholin-3-yl)ethyl)-5-(6-ethoxypyrazin-2-yl)thiazole- | | LCMS-AU |
| | 2-carboxamide | | |
| | 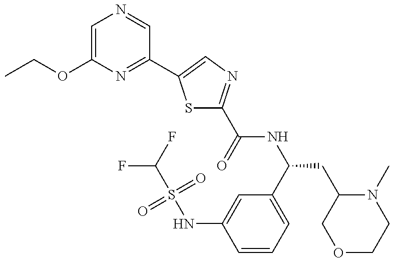 [SMILES: CCOc1cncc(-c2cnc(C(=O)N[C@H](CC3COCCN3C)c3cccc(NS(=O)(=O)C(F)F)c3)s2)n1] | | |
| I-194 | (S)-N-((3-((difluoromethyl)sulfonamido)phenyl)(tetrahydro-2H- | 554 | 1.612 |
| | pyran-4-yl)methyl)-5-(6-ethoxypyrazin-2-yl)thiazole-2- | | LCMS-BE |
| | carboxamide | | |
| | 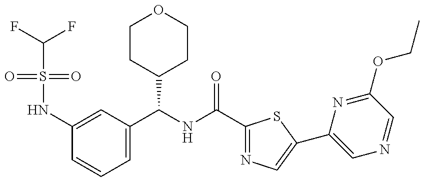 [SMILES: CCOc1cncc(-c2cnc(C(=O)N[C@H](c3cccc(NS(=O)(=O)C(F)F)c3)C3CCOCC3)s2)n1] | | |
| I-195 | (R)-N-((3-((difluoromethyl)sulfonamido)phenyl)(tetrahydro- | 554 | 1.611 |
| | 2H-pyran-4-yl)methyl)-5-(6-ethoxypyrazin-2-yl)thiazole-2- | | LCMS-BE |
| | carboxamide | | |
| | 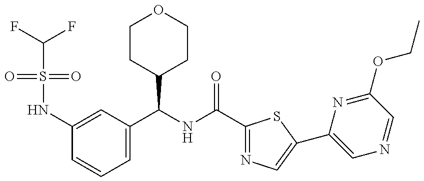 [SMILES: CCOc1cncc(-c2cnc(C(=O)N[C@@H](c3cccc(NS(=O)(=O)C(F)F)c3)C3CCOCC3)s2)n1] | | |
| I-196 | (S)-5-(5-chloropyrazolo[1,5-a]pyridin-3-yl)-N-((3- | 582/584 | 1.768 |
| | ((difluoromethyl)sulfonamido)phenyl)(tetrahydro-2H-pyran-4- | | LCMS-AJ |
| | yl)methyl)thiazole-2-carboxamide | | |
| | 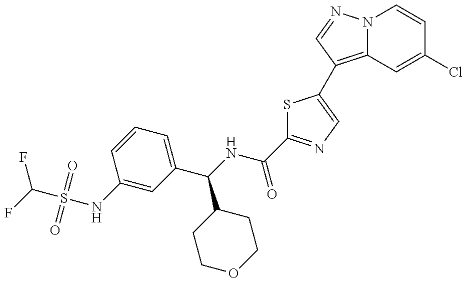 [SMILES: O=C(N[C@H](c1cccc(NS(=O)(=O)C(F)F)c1)C1CCOCC1)c1ncc(-c2cnn3ccc(Cl)cc23)s1] | | |
| I-197 | (R)-5-(5-chloropyrazolo[1,5-a]pyridin-3-yl)-N-((3- | 582/584 | 1.774 |
| | ((difluoromethyl)sulfonamido)phenyl)(tetrahydro-2H-pyran-4- | | LCMS-AJ |
| | yl)methyl)thiazole-2-carboxamide | | |
| | 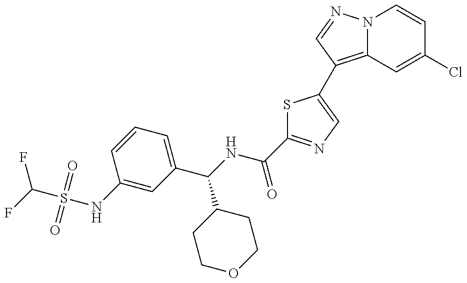 [SMILES: O=C(N[C@@H](c1cccc(NS(=O)(=O)C(F)F)c1)C1CCOCC1)c1ncc(-c2cnn3ccc(Cl)cc23)s1] | | |
| I-198 | N-((R)-(3-((difluoromethyl)sulfonamido)phenyl)((S)-1,4- | 556 | 1.242 |
| | dioxan-2-yl)methyl)-5-(6-ethoxypyrazin-2-yl)thiazole-2- | | LCMS-L |
| | carboxamide | | |
| | 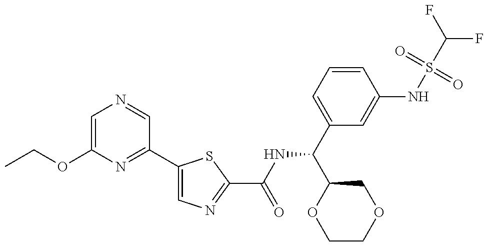 [SMILES: CCOc1cncc(-c2cnc(C(=O)N[C@H](c3cccc(NS(=O)(=O)C(F)F)c3)[C@H]3COCCO3)s2)n1] | | |
| I-199 | N-((R)-(3-((difluoromethyl)sulfonamido)phenyl)((R)-1,4- | 556 | 1.235 |
| | dioxan-2-yl)methyl)-5-(6-ethoxypyrazin-2-yl)thiazole-2- | | LCMS-L |
| | carboxamide | | |
| | 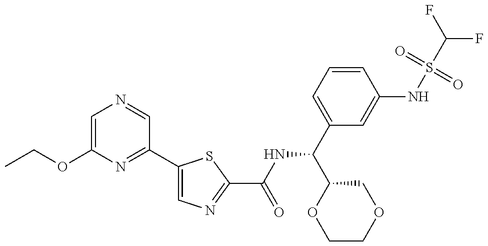 [SMILES: CCOc1cncc(-c2cnc(C(=O)N[C@H](c3cccc(NS(=O)(=O)C(F)F)c3)[C@@H]3COCCO3)s2)n1] | | |
| I-200 | N-((S)-(3-((difluoromethyl)sulfonamido)phenyl)((R)-1,4- | 556 | 1.235 |
| | dioxan-2-yl)methyl)-5-(6-ethoxypyrazin-2-yl)thiazole-2- | | LCMS-L |
| | carboxamide | | |
| | 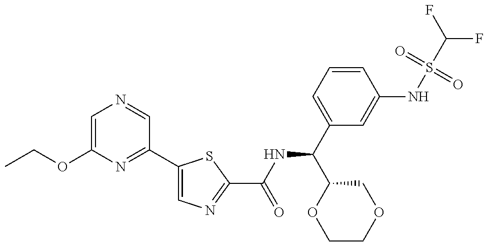 [SMILES: CCOc1cncc(-c2cnc(C(=O)N[C@@H](c3cccc(NS(=O)(=O)C(F)F)c3)[C@@H]3COCCO3)s2)n1] | | |
| I-201 | N-((S)-(3-((difluoromethyl)sulfonamido)phenyl)((S)-1,4- | 556 | 1.235 |
| | dioxan-2-yl)methyl)-5-(6-ethoxypyrazin-2-yl)thiazole-2- | | LCMS-L |
| | carboxamide | | |
| | 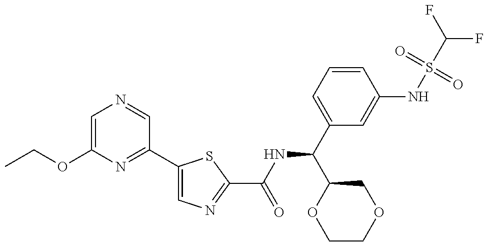 [SMILES: CCOc1cncc(-c2cnc(C(=O)N[C@@H](c3cccc(NS(=O)(=O)C(F)F)c3)[C@H]3COCCO3)s2)n1] | | |
| I-202 | N-((1s,3s)-1-(3-((difluoromethyl)sulfonamido)phenyl)-3- | 526 | 1.441 |
| | hydroxycyclobutyl)-5-(6-ethoxypyrazin-2-yl)thiazole-2- | | LCMS-AJ |
| | carboxamide | | |
| | 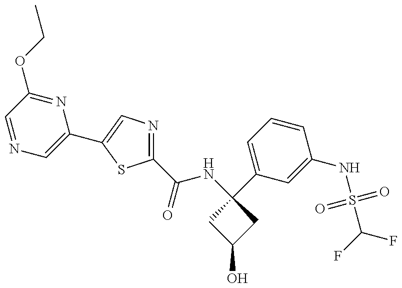 [SMILES: CCOc1cncc(-c2cnc(C(=O)N[C@]3(c4cccc(NS(=O)(=O)C(F)F)c4)C[C@H](O)C3)s2)n1] | | |
| I-203 | N-((1r,3r)-1-(3-((difluoromethyl)sulfonamido)phenyl)-3- | 526 | 1.98 |
| | hydroxycyclobutyl)-5-(6-ethoxypyrazin-2-yl)thiazole-2- | | LCMS-AK |
| | carboxamide | | |
| | 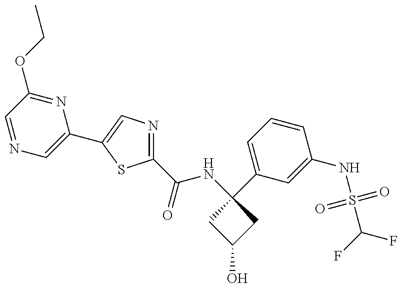 [SMILES: CCOc1cncc(-c2cnc(C(=O)N[C@]3(c4cccc(NS(=O)(=O)C(F)F)c4)C[C@@H](O)C3)s2)n1] | | |
| I-204 | N-((R)-2-((1R,4R)-2-oxa-5-azabicyclo[2.2.1]heptan-5-yl)-1-(3- | 581 | 1.5 |
| | ((difluoromethyl)sulfonamido)phenyl)ethyl)-5-(6- | | LCMS-AQ |
| | ethoxypyrazin-2-yl)thiazole-2-carboxamide | | |
| | 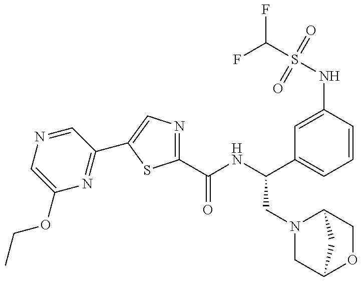 [SMILES: CCOc1cncc(-c2cnc(C(=O)N[C@@H](CN3C[C@H]4C[C@@H]3CO4)c3cccc(NS(=O)(=O)C(F)F)c3)s2)n1] | | |
| I-205 | N-((S)-2-((1R,4R)-2-oxa-5-azabicyclo[2.2.1]heptan-5-yl)-1-(3- | 581 | 1.5 |
| | ((difluoromethyl)sulfonamido)phenyl)ethyl)-5-(6- | | LCMS-AR |
| | ethoxypyrazin-2-yl)thiazole-2-carboxamide, formic acid salt | | |
| | 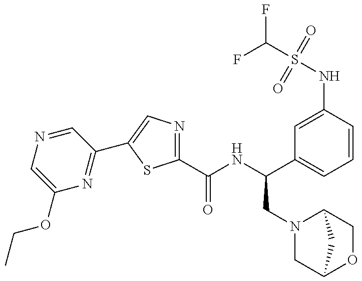 [SMILES: CCOc1cncc(-c2cnc(C(=O)N[C@H](CN3C[C@H]4C[C@@H]3CO4)c3cccc(NS(=O)(=O)C(F)F)c3)s2)n1] | | |
| I-206 | N-((R)-1-(3-((difluoromethyl)sulfonamido)phenyl)-2-((R)-3- | 583 | 0.775 |
| | hydroxy-3-methylpyrrolidin-1-yl)ethyl)-5-(6-ethoxypyrazin-2- | | LCMS-BB |
| | yl)thiazole-2-carboxamide | | |
| | 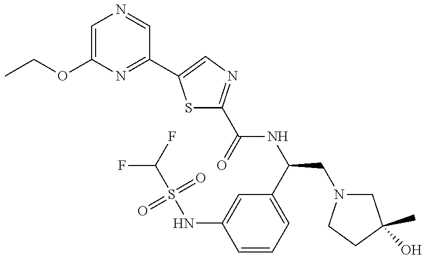 [SMILES: CCOc1cncc(-c2cnc(C(=O)N[C@@H](CN3CC[C@@](C)(O)C3)c3cccc(NS(=O)(=O)C(F)F)c3)s2)n1] | | |
| I-207 | N-((S)-1-(3-((difluoromethyl)sulfonamido)phenyl)-2-((R)-3- | 583 | 0.771 |
| | hydroxy-3-methylpyrrolidin-1-yl)ethyl)-5-(6-ethoxypyrazin-2- | | LCMS-BB |
| | yl)thiazole-2-carboxamide | | |
| | 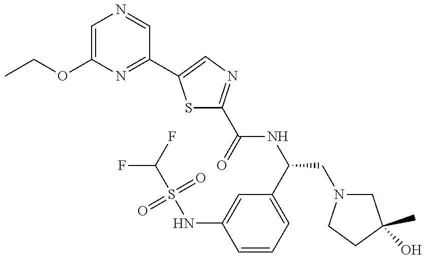 [SMILES: CCOc1cncc(-c2cnc(C(=O)N[C@H](CN3CC[C@@](C)(O)C3)c3cccc(NS(=O)(=O)C(F)F)c3)s2)n1] | | |
| I-208 | N-((S)-1-(3-((difluoromethyl)sulfonamido)phenyl)-2-((S)-3- | 583 | 1.541 |
| | hydroxy-3-methylpyrrolidin-1-yl)ethyl)-5-(6-ethoxypyrazin-2- | | LCMS-BC |
| | yl)thiazole-2-carboxamide, formic acid salt | | |
| | 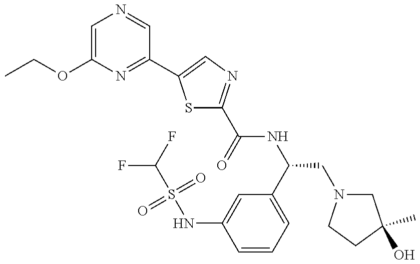 [SMILES: CCOc1cncc(-c2cnc(C(=O)N[C@H](CN3CC[C@](C)(O)C3)c3cccc(NS(=O)(=O)C(F)F)c3)s2)n1] | | |
| I-209 | N-((R)-1-(3-((difluoromethyl)sulfonamido)phenyl)-2-((S)-3- | 583 | 1.544 |
| | hydroxy-3-methylpyrrolidin-1-yl)ethyl)-5-(6-ethoxypyrazin-2- | | LCMS-BC |
| | yl)thiazole-2-carboxamide, formic acid salt | | |
| | 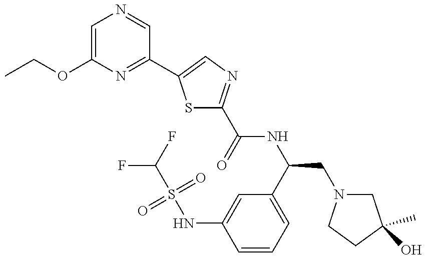 [SMILES: CCOc1cncc(-c2cnc(C(=O)N[C@@H](CN3CC[C@](C)(O)C3)c3cccc(NS(=O)(=O)C(F)F)c3)s2)n1] | | |
| I-210 | N-((R)-2-((1R,4S)-2-azabicyclo[2.2.1]heptan-2-yl)-1-(3- | 579 | 0.95 |
| | ((difluoromethyl)sulfonamido)phenyl)ethyl)-5-(6- | | LCMS-I |
| | ethoxypyrazin-2-yl)thiazole-2-carboxamide | | |
| | 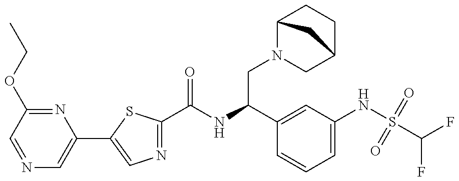 [SMILES: CCOc1cncc(-c2cnc(C(=O)N[C@@H](CN3C[C@H]4CC[C@@H]3C4)c3cccc(NS(=O)(=O)C(F)F)c3)s2)n1] | | |
| I-211 | N-((S)-2-((1R,4S)-2-azabicyclo[2.2.1]heptan-2-yl)-1-(3- | 579 | 1.579 |
| | ((difluoromethyl)sulfonamido)phenyl)ethyl)-5-(6- | | LCMS-BE |
| | ethoxypyrazin-2-yl)thiazole-2-carboxamide | | |
| | 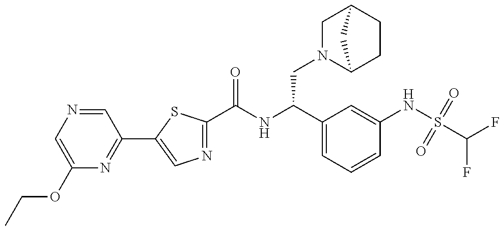 [SMILES: CCOc1cncc(-c2cnc(C(=O)N[C@H](CN3C[C@H]4CC[C@@H]3C4)c3cccc(NS(=O)(=O)C(F)F)c3)s2)n1] | | |
| I-212 | (R)-N-(1-(3-((difluoromethyl)sulfonamido)phenyl)-2- | 527 | 0.855 |
| | (dimethylamino)ethyl)-5-(6-ethoxypyrazin-2-yl)thiazole-2- | | LCMS-AU |
| | carboxamide | | |
| | 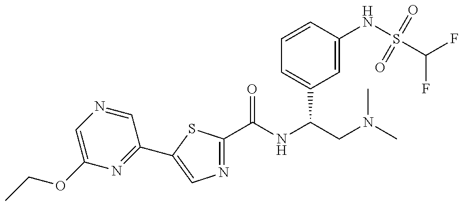 [SMILES: CCOc1cncc(-c2cnc(C(=O)N[C@@H](CN(C)C)c3cccc(NS(=O)(=O)C(F)F)c3)s2)n1] | | |
| I-213 | (S)-N-(1-(3-((difluoromethyl)sulfonamido)phenyl)-2- | 527 | 0.842 |
| | (dimethylamino)ethyl)-5-(6-ethoxypyrazin-2-yl)thiazole-2- | | LCMS-AU |
| | carboxamide | | |
| | 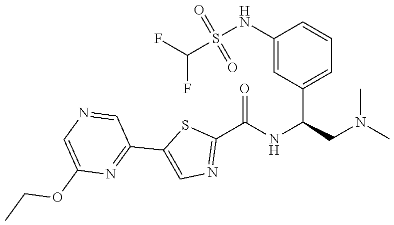 [SMILES: CCOc1cncc(-c2cnc(C(=O)N[C@H](CN(C)C)c3cccc(NS(=O)(=O)C(F)F)c3)s2)n1] | | |
| I-214 | (R)-N-(1-(3-((difluoromethyl)sulfonamido)phenyl)-2-(1H-1,2,3- | 551 | 1.197 |
| | triazol-1-yl)ethyl)-5-(6-ethoxypyrazin-2-yl)thiazole-2- | | LCMS-M |
| | carboxamide | | |
| | 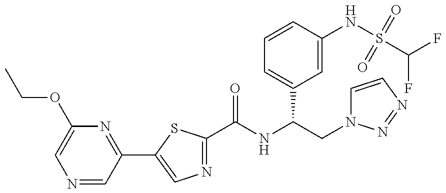 [SMILES: CCOc1cncc(-c2cnc(C(=O)N[C@@H](Cn3ccnn3)c3cccc(NS(=O)(=O)C(F)F)c3)s2)n1] | | |
| I-215 | (S)-N-(1-(3-((difluoromethyl)sulfonamido)phenyl)-2-(1H-1,2,3- | 551 | 1.188 |
| | triazol-1-yl)ethyl)-5-(6-ethoxypyrazin-2-yl)thiazole-2- | | LCMS-L |
| | carboxamide | | |
| | 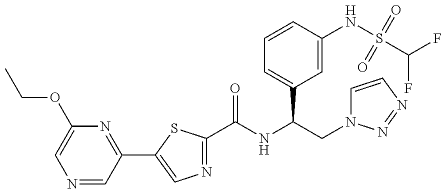 [SMILES: CCOc1cncc(-c2cnc(C(=O)N[C@H](Cn3ccnn3)c3cccc(NS(=O)(=O)C(F)F)c3)s2)n1] | | |
| I-216 | (S)-N-(1-(3-((difluoromethyl)sulfonamido)phenyl)-2-(2H-1,2,3- | 551 | 1.821 |
| | triazol-2-yl)ethyl)-5-(6-ethoxypyrazin-2-yl)thiazole-2- | | LCMS-BE |
| | carboxamide | | |
| | 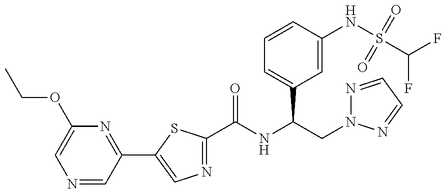 [SMILES: CCOc1cncc(-c2cnc(C(=O)N[C@H](Cn3nccn3)c3cccc(NS(=O)(=O)C(F)F)c3)s2)n1] | | |
| I-217 | (R)-N-(1-(3-((difluoromethyl)sulfonamido)phenyl)-2-(2H-1,2,3- | 551 | 1.47 |
| | triazol-2-yl)ethyl)-5-(6-ethoxypyrazin-2-yl)thiazole-2- | | LCMS-M |
| | carboxamide | | |
| | 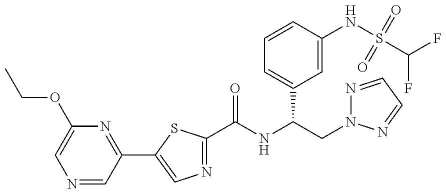 [SMILES: CCOc1cncc(-c2cnc(C(=O)N[C@@H](Cn3nccn3)c3cccc(NS(=O)(=O)C(F)F)c3)s2)n1] | | |
| I-218 | (S)-N-(1-(3-((difluoromethyl)sulfonamido)phenyl)-2-(piperidin- | 567 | 1.807 |
| | 1-yl)ethyl)-5-(6-ethoxypyrazin-2-yl)thiazole-2-carboxamide | | LCMS-AK |
| | 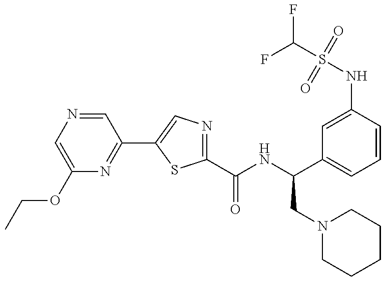 [SMILES: CCOc1cncc(-c2cnc(C(=O)N[C@H](CN3CCCCC3)c3cccc(NS(=O)(=O)C(F)F)c3)s2)n1] | | |
| I-219 | (R)-N-(1-(3-((difluoromethyl)sulfonamido)phenyl)-2- | 567 | 1.668 |
| | (piperidin-1-yl)ethyl)-5-(6-ethoxypyrazin-2-yl)thiazole-2- | | LCMS-BF |
| | carboxamide | | |
| | 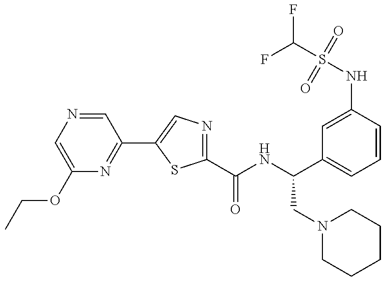 [SMILES: CCOc1cncc(-c2cnc(C(=O)N[C@@H](CN3CCCCC3)c3cccc(NS(=O)(=O)C(F)F)c3)s2)n1] | | |
| I-220 | (S)-5-(5-chloropyrazolo[1,5-a]pyridin-3-yl)-N-(1-(3- | 581/583 | 1.358 |
| | ((difluoromethyl)sulfonamido)phenyl)-2-(pyrrolidin-1- | | LCMS-AA |
| | yl)ethyl)thiazole-2-carboxamide | | |
| | 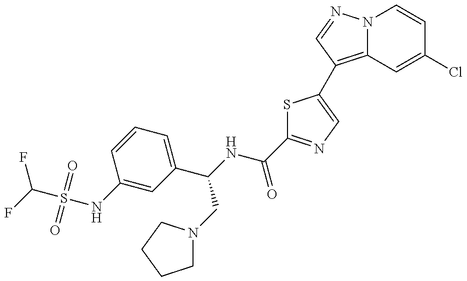 [SMILES: O=C(N[C@H](CN1CCCC1)c1cccc(NS(=O)(=O)C(F)F)c1)c1ncc(-c2cnn3ccc(Cl)cc23)s1] | | |
| I-221 | (R)-5-(5-chloropyrazolo[1,5-a]pyridin-3-yl)-N-(1-(3- | 581/583 | 1.648 |
| | ((difluoromethyl)sulfonamido)phenyl)-2-(pyrrolidin-1- | | LCMS-AE |
| | yl)ethyl)thiazole-2-carboxamide | | |
| | 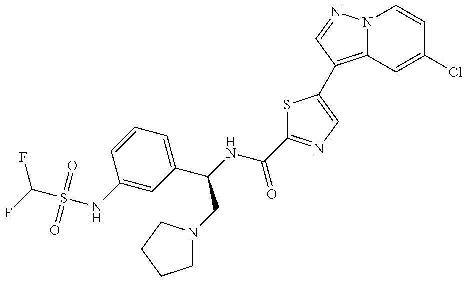 [SMILES: O=C(N[C@@H](CN1CCCC1)c1cccc(NS(=O)(=O)C(F)F)c1)c1ncc(-c2cnn3ccc(Cl)cc23)s1] | | |
| I-222 | N-((1S,2S)-1-(3-((difluoromethyl)sulfonamido)phenyl)-2- | 583 | 1.048 |
| | morpholinopropyl)-5-(6-ethoxypyrazin-2-yl)thiazole-2- | | LCMS-P |
| | carboxamide | | |
| | 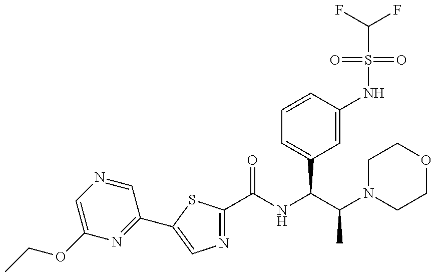 [SMILES: CCOc1cncc(-c2cnc(C(=O)N[C@@H](c3cccc(NS(=O)(=O)C(F)F)c3)[C@H](C)N3CCOCC3)s2)n1] | | |
| I-223 | N-((1S,2R)-1-(3-((difluoromethyl)sulfonamido)phenyl)-2- | 583 | 1.055 |
| | morpholinopropyl)-5-(6-ethoxypyrazin-2-yl)thiazole-2- | | LCMS-P |
| | carboxamide | | |
| | 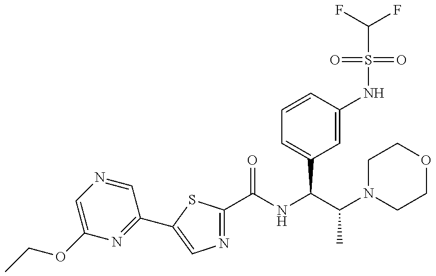 [SMILES: CCOc1cncc(-c2cnc(C(=O)N[C@@H](c3cccc(NS(=O)(=O)C(F)F)c3)[C@@H](C)N3CCOCC3)s2)n1] | | |
| I-224 | N-((1R,2R)-1-(3-((difluoromethyl)sulfonamido)phenyl)-2- | 583 | 1.062 |
| | morpholinopropyl)-5-(6-ethoxypyrazin-2-yl)thiazole-2- | | LCMS-P |
| | carboxamide | | |
| | 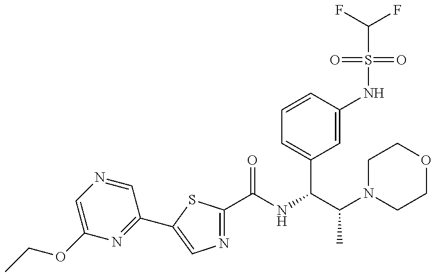 [SMILES: CCOc1cncc(-c2cnc(C(=O)N[C@H](c3cccc(NS(=O)(=O)C(F)F)c3)[C@@H](C)N3CCOCC3)s2)n1] | | |
| I-225 | N-((1R,2S)-1-(3-((difluoromethyl)sulfonamido)phenyl)-2- | 583 | 1.055 |
| | morpholinopropyl)-5-(6-ethoxypyrazin-2-yl)thiazole-2- | | LCMS-P |
| | carboxamide | | |
| | 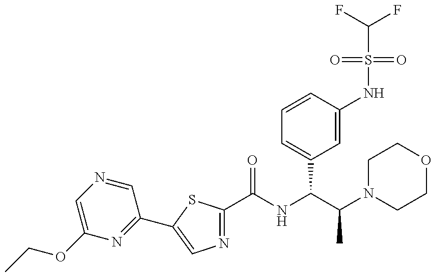 [SMILES: CCOc1cncc(-c2cnc(C(=O)N[C@H](c3cccc(NS(=O)(=O)C(F)F)c3)[C@H](C)N3CCOCC3)s2)n1] | | |
| I-226 | N-((1R,2R)-1-(4-(cyclopropanesulfonamido)pyridin-2-yl)-3- | 519 | 1.015 |
| | hydroxy-2-methylpropyl)-5-(6-ethoxypyrazin-2-yl)thiazole-2- | | LCMS-W |
| | carboxamide | | |
| | 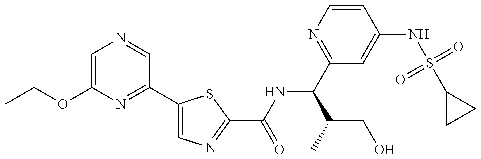 [SMILES: CCOc1cncc(-c2cnc(C(=O)N[C@@H](c3cc(NS(=O)(=O)C4CC4)ccn3)[C@@H](C)CO)s2)n1] | | |
| I-227 | N-((1R,2S)-1-(4-(cyclopropanesulfonamido)pyridin-2-yl)-3- | 519 | 1.015 |
| | hydroxy-2-methylpropyl)-5-(6-ethoxypyrazin-2-yl)thiazole-2- | | LCMS-W |
| | carboxamide | | |
| | 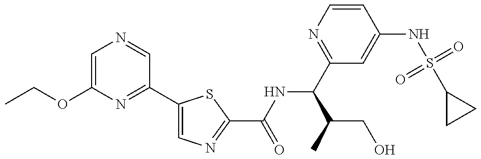 [SMILES: CCOc1cncc(-c2cnc(C(=O)N[C@@H](c3cc(NS(=O)(=O)C4CC4)ccn3)[C@H](C)CO)s2)n1] | | |
| I-228 | N-((1S,2R)-1-(4-(cyclopropanesulfonamido)pyridin-2-yl)-3- | 519 | 1.015 |
| | hydroxy-2-methylpropyl)-5-(6-ethoxypyrazin-2-yl)thiazole-2- | | LCMS-W |
| | carboxamide | | |
| | 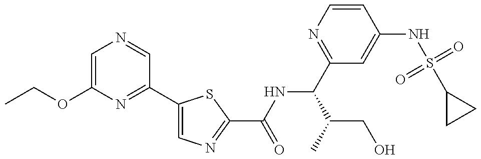 [SMILES: CCOc1cncc(-c2cnc(C(=O)N[C@H](c3cc(NS(=O)(=O)C4CC4)ccn3)[C@@H](C)CO)s2)n1] | | |
| I-229 | N-((1S,2S)-1-(4-(cyclopropanesulfonamido)pyridin-2-yl)-3- | 519 | 1.008 |
| | hydroxy-2-methylpropyl)-5-(6-ethoxypyrazin-2-yl)thiazole-2- | | LCMS-W |
| | carboxamide | | |
| | 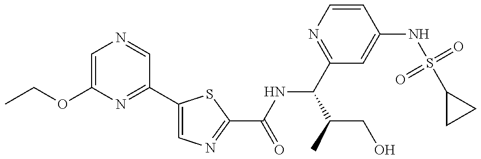 [SMILES: CCOc1cncc(-c2cnc(C(=O)N[C@H](c3cc(NS(=O)(=O)C4CC4)ccn3)[C@H](C)CO)s2)n1] | | |
| I-230 | 5-(5-chloropyrazolo[1,5-a]pyridin-3-yl)-N-((1R,2S)-1-(4- | 547/549 | 1.015 |
| | (cyclopropanesulfonamido)pyridin-2-yl)-3-hydroxy-2- | | LCMS-P |
| | methylpropyl)thiazole-2-carboxamide | | |
| | 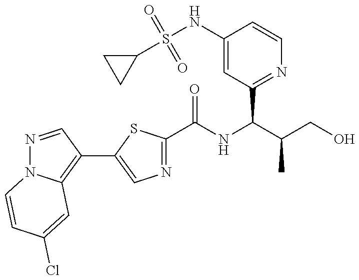 [SMILES: C[C@H](CO)[C@@H](NC(=O)c1ncc(-c2cnn3ccc(Cl)cc23)s1)c1cc(NS(=O)(=O)C2CC2)ccn1] | | |
| I-231 | 5-(5-chloropyrazolo[1,5-a]pyridin-3-yl)-N-((1R,2R)-1-(4- | 547/549 | 1.015 |
| | (cyclopropanesulfonamido)pyridin-2-yl)-3-hydroxy-2- | | LCMS-P |
| | methylpropyl)thiazole-2-carboxamide | | |
| | 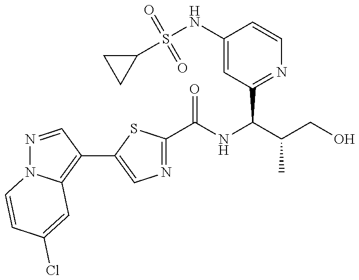 [SMILES: C[C@@H](CO)[C@@H](NC(=O)c1ncc(-c2cnn3ccc(Cl)cc23)s1)c1cc(NS(=O)(=O)C2CC2)ccn1] | | |
| I-232 | 5-(5-chloropyrazolo[1,5-a]pyridin-3-yl)-N-((1S,2R)-1-(4- | 547/549 | 1.673 |
| | (cyclopropanesulfonamido)pyridin-2-yl)-3-hydroxy-2- | | LCMS-Q |
| | methylpropyl)thiazole-2-carboxamide | | |
| | 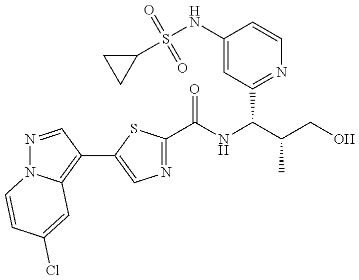 [SMILES: C[C@@H](CO)[C@H](NC(=O)c1ncc(-c2cnn3ccc(Cl)cc23)s1)c1cc(NS(=O)(=O)C2CC2)ccn1] | | |
| I-233 | 5-(5-chloropyrazolo[1,5-a]pyridin-3-yl)-N-((1S,2S)-1-(4- | 547/549 | 1.022 |
| | (cyclopropanesulfonamido)pyridin-2-yl)-3-hydroxy-2- | | LCMS-P |
| | methylpropyl)thiazole-2-carboxamide | | |
| | 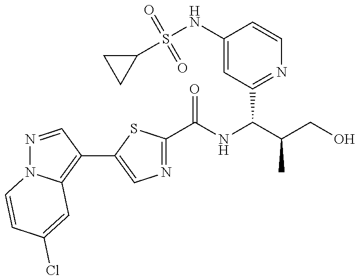 [SMILES: C[C@H](CO)[C@H](NC(=O)c1ncc(-c2cnn3ccc(Cl)cc23)s1)c1cc(NS(=O)(=O)C2CC2)ccn1] | | |
| I-234 | N-((1R,2S)-1-(4-(cyclopropanesulfonamido)pyridin-2-yl)-3- | 533 | 1.048 |
| | methoxy-2-methylpropyl)-5-(6-ethoxypyrazin-2-yl)thiazole-2- | | LCMS-L |
| | carboxamide | | |
| | 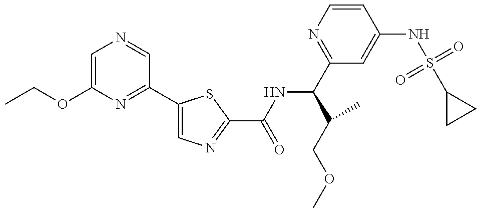 [SMILES: CCOc1cncc(-c2cnc(C(=O)N[C@@H](c3cc(NS(=O)(=O)C4CC4)ccn3)[C@H](C)COC)s2)n1] | | |
| I-235 | N-((1R,2R)-1-(4-(cyclopropanesulfonamido)pyridin-2-yl)-3- | 533 | 1.042 |
| | methoxy-2-methylpropyl)-5-(6-ethoxypyrazin-2-yl)thiazole-2- | | LCMS-L |
| | carboxamide | | |
| | 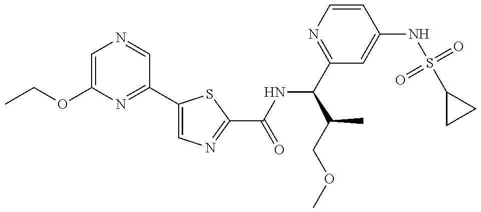 [SMILES: CCOc1cncc(-c2cnc(C(=O)N[C@@H](c3cc(NS(=O)(=O)C4CC4)ccn3)[C@@H](C)COC)s2)n1] | | |
| I-236 | N-((1S,2R)-1-(4-(cyclopropanesulfonamido)pyridin-2-yl)-3- | 533 | 1.66 |
| | methoxy-2-methylpropyl)-5-(6-ethoxypyrazin-2-yl)thiazole-2- | | LCMS-R |
| | carboxamide | | |
| | 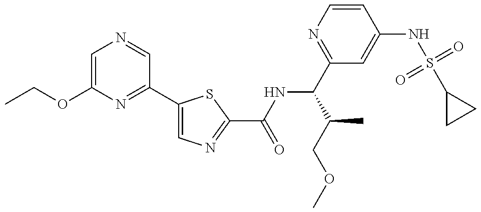 [SMILES: CCOc1cncc(-c2cnc(C(=O)N[C@H](c3cc(NS(=O)(=O)C4CC4)ccn3)[C@@H](C)COC)s2)n1] | | |
| I-237 | N-((1S,2S)-1-(4-(cyclopropanesulfonamido)pyridin-2-yl)-3- | 533 | 1.687 |
| | methoxy-2-methylpropyl)-5-(6-ethoxypyrazin-2-yl)thiazole-2- | | LCMS-R |
| | carboxamide | | |
| | 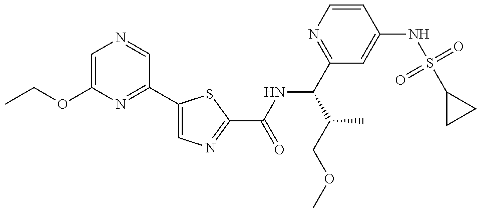 [SMILES: CCOc1cncc(-c2cnc(C(=O)N[C@H](c3cc(NS(=O)(=O)C4CC4)ccn3)[C@H](C)COC)s2)n1] | | |
| I-238 | N-((R)-1-(4-(cyclopropanesulfonamido)pyridin-2-yl)-2-(((S)- | 561 | 1.042 |
| | tetrahydrofuran-3-yl)oxy)ethyl)-5-(6-ethoxypyrazin-2- | | LCMS-AN |
| | yl)thiazole-2-carboxamide | | |
| | 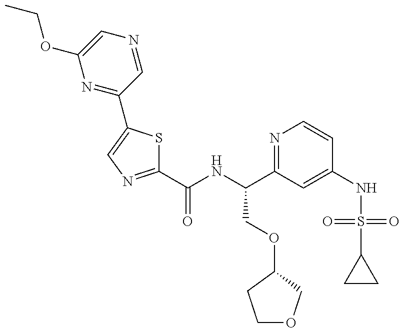 [SMILES: CCOc1cncc(-c2cnc(C(=O)N[C@@H](CO[C@H]3CCOC3)c3cc(NS(=O)(=O)C4CC4)ccn3)s2)n1] | | |
| I-239 | N-((R)-1-(4-(cyclopropanesulfonamido)pyridin-2-yl)-2-(((R)- | 561 | 1.051 |
| | tetrahydrofuran-3-yl)oxy)ethyl)-5-(6-ethoxypyrazin-2- | | LCMS-AN |
| | yl)thiazole-2-carboxamide | | |
| | 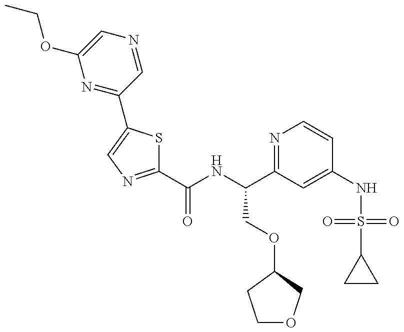 [SMILES: CCOc1cncc(-c2cnc(C(=O)N[C@@H](CO[C@@H]3CCOC3)c3cc(NS(=O)(=O)C4CC4)ccn3)s2)n1] | | |
| I-240 | N-((S)-1-(4-(cyclopropanesulfonamido)pyridin-2-yl)-2-(((R)- | 561 | 1.563 |
| | tetrahydrofuran-3-yl)oxy)ethyl)-5-(6-ethoxypyrazin-2- | | LCMS-AO |
| | yl)thiazole-2-carboxamide | | |
| | 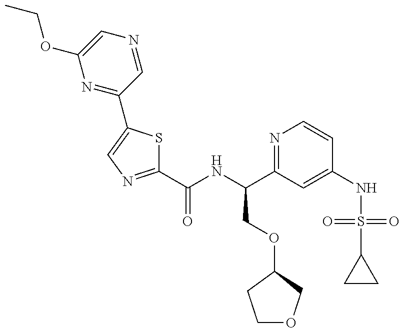 [SMILES: CCOc1cncc(-c2cnc(C(=O)N[C@H](CO[C@@H]3CCOC3)c3cc(NS(=O)(=O)C4CC4)ccn3)s2)n1] | | |
| I-241 | N-((S)-1-(4-(cyclopropanesulfonamido)pyridin-2-yl)-2-(((S)- | 561 | 1.056 |
| | tetrahydrofuran-3-yl)oxy)ethyl)-5-(6-ethoxypyrazin-2- | | LCMS-AN |
| | yl)thiazole-2-carboxamide | | |
| | 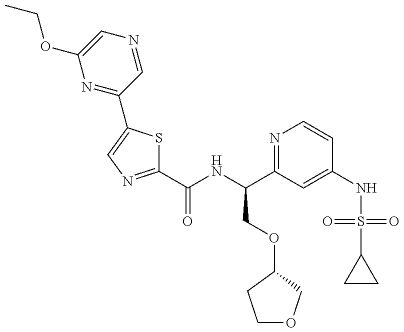 [SMILES: CCOc1cncc(-c2cnc(C(=O)N[C@H](CO[C@H]3CCOC3)c3cc(NS(=O)(=O)C4CC4)ccn3)s2)n1] | | |
| I-242 | N-((1S,2S)-1-(3-((difluoromethyl)sulfonamido)phenyl)-3- | 528 | 1.479 |
| | hydroxy-2-methylpropyl)-5-(6-ethoxypyrazin-2-yl)thiazole-2- | | LCMS-D |
| | carboxamide | | |
| | 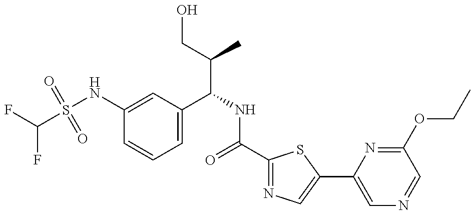 [SMILES: CCOc1cncc(-c2cnc(C(=O)N[C@H](c3cccc(NS(=O)(=O)C(F)F)c3)[C@H](C)CO)s2)n1] | | |
| I-243 | N-((1S,2R)-1-(3-((difluoromethyl)sulfonamido)phenyl)-3- | 528 | 1.475 |
| | hydroxy-2-methylpropyl)-5-(6-ethoxypyrazin-2-yl)thiazole-2- | | LCMS-D |
| | carboxamide | | |
| | 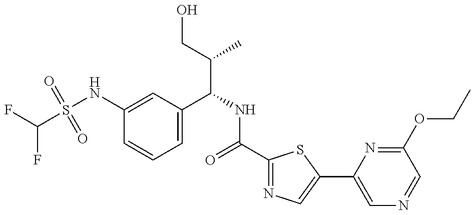 [SMILES: CCOc1cncc(-c2cnc(C(=O)N[C@H](c3cccc(NS(=O)(=O)C(F)F)c3)[C@@H](C)CO)s2)n1] | | |
| I-244 | N-((1R,2R)-1-(3-((difluoromethyl)sulfonamido)phenyl)-3- | 528 | 1.504 |
| | hydroxy-2-methylpropyl)-5-(6-ethoxypyrazin-2-yl)thiazole-2- | | LCMS-D |
| | carboxamide | | |
| | 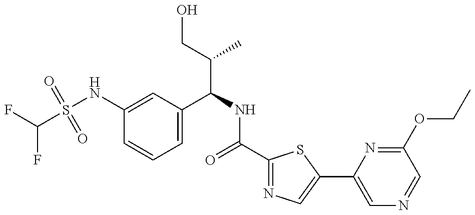 [SMILES: CCOc1cncc(-c2cnc(C(=O)N[C@@H](c3cccc(NS(=O)(=O)C(F)F)c3)[C@@H](C)CO)s2)n1] | | |
| I-245 | N-((1R,2S)-1-(3-((difluoromethyl)sulfonamido)phenyl)-3- | 528 | 1.721 |
| | hydroxy-2-methylpropyl)-5-(6-ethoxypyrazin-2-yl)thiazole-2- | | LCMS-H |
| | carboxamide | | |
| | 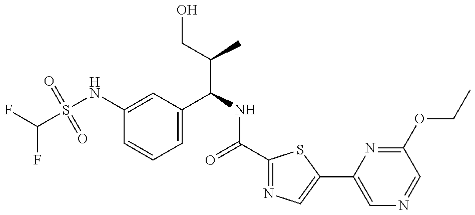 [SMILES: CCOc1cncc(-c2cnc(C(=O)N[C@@H](c3cccc(NS(=O)(=O)C(F)F)c3)[C@H](C)CO)s2)n1] | | |
| I-246 | (S)-N-(1-(6-(cyclopropanesulfonamido)pyrimidin-4-yl)-2- | 561 | 1.413 |
| | morpholinoethyl)-5-(6-ethoxypyrazin-2-yl)thiazole-2- | | LCMS-R |
| | carboxamide | | |
| | 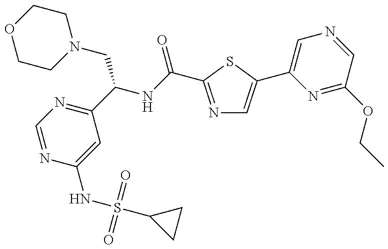 [SMILES: CCOc1cncc(-c2cnc(C(=O)N[C@@H](CN3CCOCC3)c3cc(NS(=O)(=O)C4CC4)ncn3)s2)n1] | | |
| I-247 | (R)-N-(1-(6-(cyclopropanesulfonamido)pyrimidin-4-yl)-2- | 561 | 1.328 |
| | morpholinoethyl)-5-(6-ethoxypyrazin-2-yl)thiazole-2- | | LCMS-G |
| | carboxamide | | |
| | 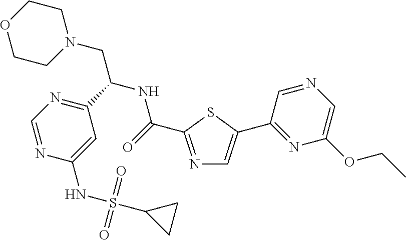 [SMILES: CCOc1cncc(-c2cnc(C(=O)N[C@@H](CN3CCOCC3)c3cc(NS(=O)(=O)C4CC4)ncn3)s2)n1] | | |
| I-248 | (S)-N-(1-(6-(cyclopropanesulfonamido)pyrimidin-4-yl)-2-(4- | 600 | 1.345 |
| | cyclopropylpiperazin-1-yl)ethyl)-5-(6-ethoxypyrazin-2- | | LCMS-AP |
| | yl)thiazole-2-carboxamide | | |
| | 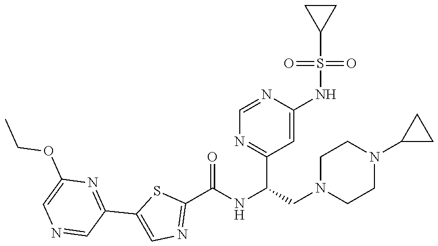 [SMILES: CCOc1cncc(-c2cnc(C(=O)N[C@@H](CN3CCN(C4CC4)CC3)c3cc(NS(=O)(=O)C4CC4)ncn3)s2)n1] | | |
| I-249 | (R)-N-(1-(6-(cyclopropanesulfonamido)pyrimidin-4-yl)-2-(4- | 600 | 1.349 |
| | cyclopropylpiperazin-1-yl)ethyl)-5-(6-ethoxypyrazin-2- | | LCMS-AP |
| | yl)thiazole-2-carboxamide | | |
| | 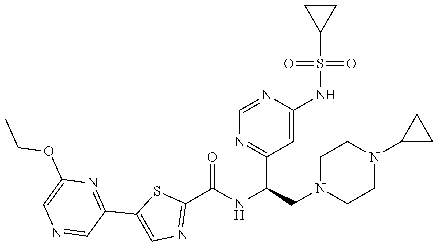 [SMILES: CCOc1cncc(-c2cnc(C(=O)N[C@H](CN3CCN(C4CC4)CC3)c3cc(NS(=O)(=O)C4CC4)ncn3)s2)n1] | | |
| I-250 | (S)-N-(1-(6-(cyclopropanesulfonamido)pyrimidin-4-yl)-2- | 545 | 1.245 |
| | (pyrrolidin-1-yl)ethyl)-5-(6-ethoxypyrazin-2-yl)thiazole-2- | | LCMS-AP |
| | carboxamide | | |
| | 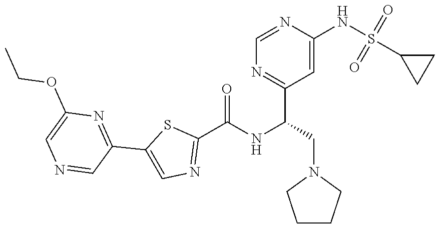 [SMILES: CCOc1cncc(-c2cnc(C(=O)N[C@@H](CN3CCCC3)c3cc(NS(=O)(=O)C4CC4)ncn3)s2)n1] | | |
| I-251 | (R)-N-(1-(6-(cyclopropanesulfonamido)pyrimidin-4-yl)-2- | 545 | 1.231 |
| | (pyrrolidin-1-yl)ethyl)-5-(6-ethoxypyrazin-2-yl)thiazole-2- | | LCMS-AP |
| | carboxamide | | |
| | 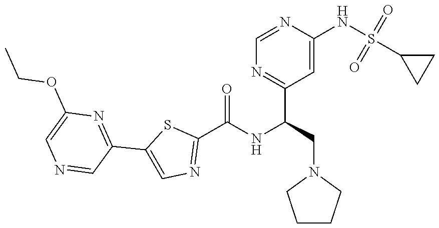 [SMILES: CCOc1cncc(-c2cnc(C(=O)N[C@H](CN3CCCC3)c3cc(NS(=O)(=O)C4CC4)ncn3)s2)n1] | | |
| I-252 | N-((S)-2-((1R,4R)-2-oxa-5-azabicyclo[2.2.1]heptan-5-yl)-1-(6- | 573 | 0.988 |
| | (cyclopropanesulfonamido)pyrazin-2-yl)ethyl)-5-(6- | | LCMS-P |
| | ethoxypyrazin-2-yl)thiazole-2-carboxamide, formic acid salt | | |
| | 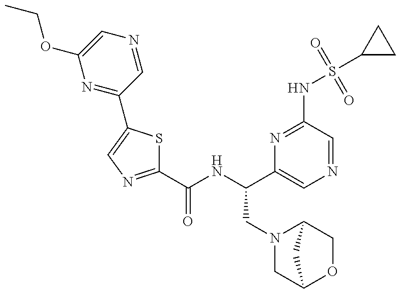 [SMILES: CCOc1cncc(-c2cnc(C(=O)N[C@@H](CN3C[C@H]4C[C@@H]3CO4)c3cncc(NS(=O)(=O)C4CC4)n3)s2)n1] | | |
| I-253 | N-((R)-2-((1R,4R)-2-oxa-5-azabicyclo[2.2.1]heptan-5-yl)-1-(6- | 573 | 1.593 |
| | (cyclopropanesulfonamido)pyrazin-2-yl)ethyl)-5-(6- | | LCMS-Q |
| | ethoxypyrazin-2-yl)thiazole-2-carboxamide, formic acid salt | | |
| | 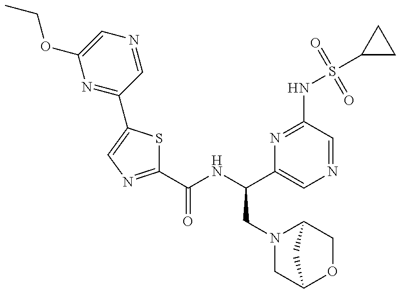 [SMILES: CCOc1cncc(-c2cnc(C(=O)N[C@H](CN3C[C@H]4C[C@@H]3CO4)c3cncc(NS(=O)(=O)C4CC4)n3)s2)n1] | | |
| I-254 | (S)-N-((6-(cyclopropanesulfonamido)pyrimidin-4- | 546 | 1.088 |
| | yl)(tetrahydro-2H-pyran-4-yl)methyl)-5-(6-ethoxypyrazin-2- | | LCMS-AI |
| | yl)thiazole-2-carboxamide | | |
| | 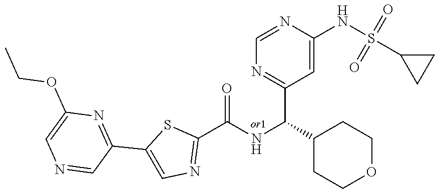 [SMILES: CCOc1cncc(-c2cnc(C(=O)N[C@H](c3cc(NS(=O)(=O)C4CC4)ncn3)C3CCOCC3)s2)n1] | | |
| I-255 | (R)-N-((6-(cyclopropanesulfonamido)pyrimidin-4- | 546 | 1.095 |
| | yl)(tetrahydro-2H-pyran-4-yl)methyl)-5-(6-ethoxypyrazin-2- | | LCMS-AI |
| | yl)thiazole-2-carboxamide | | |
| | 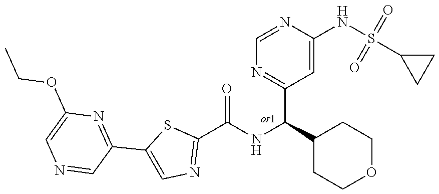 [SMILES: CCOc1cncc(-c2cnc(C(=O)N[C@@H](c3cc(NS(=O)(=O)C4CC4)ncn3)C3CCOCC3)s2)n1] | | |
| I-256 | N-((S)-(6-(cyclopropanesulfonamido)pyrimidin-4-yl)((S)- | 532 | 0.695 |
| | tetrahydrofuran-3-yl)methyl)-5-(6-ethoxypyrazin-2-yl)thiazole- | | LCMS-BB |
| | 2-carboxamide | | |
| | 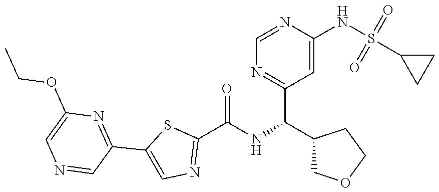 [SMILES: CCOc1cncc(-c2cnc(C(=O)N[C@H](c3cc(NS(=O)(=O)C4CC4)ncn3)[C@@H]3CCOC3)s2)n1] | | |
| I-257 | N-((R)-(6-(cyclopropanesulfonamido)pyrimidin-4-yl)((S)- | 532 | 0.685 |
| | tetrahydrofuran-3-yl)methyl)-5-(6-ethoxypyrazin-2-yl)thiazole- | | LCMS-BB |
| | 2-carboxamide | | |
| | 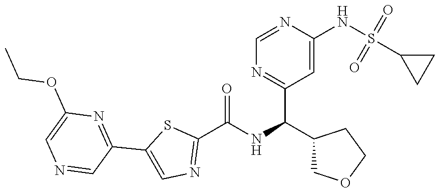 [SMILES: CCOc1cncc(-c2cnc(C(=O)N[C@@H](c3cc(NS(=O)(=O)C4CC4)ncn3)[C@@H]3CCOC3)s2)n1] | | |
| I-258 | N-((R)-(6-(cyclopropanesulfonamido)pyrimidin-4-yl)((R)- | 532 | 0.686 |
| | tetrahydrofuran-3-yl)methyl)-5-(6-ethoxypyrazin-2-yl)thiazole- | | LCMS-BB |
| | 2-carboxamide | | |
| | 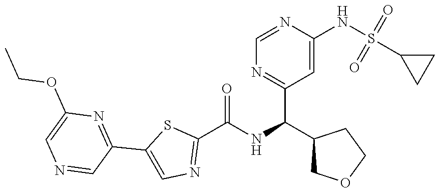 [SMILES: CCOc1cncc(-c2cnc(C(=O)N[C@@H](c3cc(NS(=O)(=O)C4CC4)ncn3)[C@H]3CCOC3)s2)n1] | | |
| I-259 | N-((S)-(6-(cyclopropanesulfonamido)pyrimidin-4-yl)((R)- | 532 | 1.388 |
| | tetrahydrofuran-3-yl)methyl)-5-(6-ethoxypyrazin-2-yl)thiazole- | | LCMS-BD |
| | 2-carboxamide | | |
| | 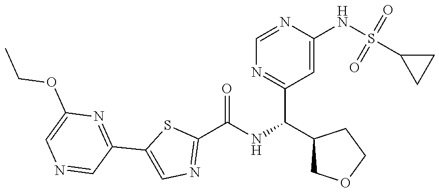 [SMILES: CCOc1cncc(-c2cnc(C(=O)N[C@H](c3cc(NS(=O)(=O)C4CC4)ncn3)[C@H]3CCOC3)s2)n1] | | |
| I-260 | 5-(5-chloropyrazolo[1,5-a]pyridin-3-yl)-N-((S)-(2- | 560/562 | 0.815 |
| | (cyclopropanesulfonamido)pyrimidin-4-yl)((R)-tetrahydrofuran- | | LCMS-AU |
| | 3-yl)methyl)thiazole-2-carboxamide | | |
| | 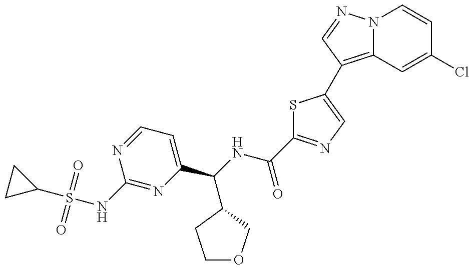 [SMILES: O=C(N[C@H](c1ccnc(NS(=O)(=O)C2CC2)n1)[C@H]1CCOC1)c1ncc(-c2cnn3ccc(Cl)cc23)s1] | | |
| I-261 | 5-(5-chloropyrazolo[1,5-a]pyridin-3-yl)-N-((S)-(2- | 560/562 | 0.815 |
| | (cyclopropanesulfonamido)pyrimidin-4-yl)((S)-tetrahydrofuran- | | LCMS-AV |
| | 3-yl)methyl)thiazole-2-carboxamide | | |
| | 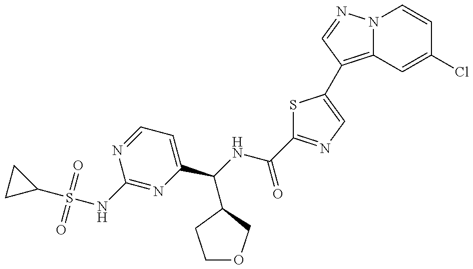 [SMILES: O=C(N[C@H](c1ccnc(NS(=O)(=O)C2CC2)n1)[C@@H]1CCOC1)c1ncc(-c2cnn3ccc(Cl)cc23)s1] | | |
| I-262 | (R)-N-(1-(4-(cyclopropanesulfonamido)pyridin-2-yl)-2- | 491 | 1.455 |
| | hydroxyethyl)-5-(6-ethoxypyrazin-2-yl)thiazole-2-carboxamide | | LCMS-BC |
| | 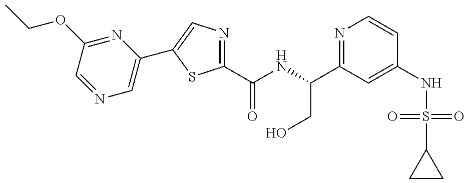 [SMILES: CCOc1cncc(-c2cnc(C(=O)N[C@@H](CO)c3cc(NS(=O)(=O)C4CC4)ccn3)s2)n1] | | |
| I-263 | (S)-N-(1-(4-(cyclopropanesulfonamido)pyridin-2-yl)-2- | 491 | 0.744 |
| | hydroxyethyl)-5-(6-ethoxypyrazin-2-yl)thiazole-2-carboxamide | | LCMS-BB |
| | 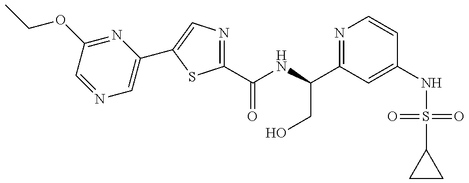 [SMILES: CCOc1cncc(-c2cnc(C(=O)N[C@H](CO)c3cc(NS(=O)(=O)C4CC4)ccn3)s2)n1] | | |
| I-264 | N-((1R,2R)-1-(4-(cyclopropanesulfonamido)pyridin-2-yl)-2- | 505 | 1.365 |
| | hydroxypropyl)-5-(6-ethoxypyrazin-2-yl)thiazole-2- | | LCMS-G |
| | carboxamide | | |
| | 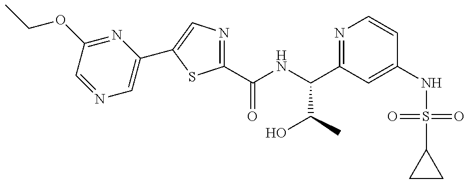 [SMILES: CCOc1cncc(-c2cnc(C(=O)N[C@H](c3cc(NS(=O)(=O)C4CC4)ccn3)[C@@H](C)O)s2)n1] | | |
| I-265 | N-((1S,2R)-1-(4-(cyclopropanesulfonamido)pyridin-2-yl)-2- | 505 | 1.567 |
| | hydroxypropyl)-5-(6-ethoxypyrazin-2-yl)thiazole-2- | | LCMS-T |
| | carboxamide | | |
| | 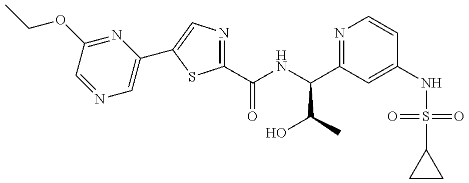 [SMILES: CCOc1cncc(-c2cnc(C(=O)N[C@@H](c3cc(NS(=O)(=O)C4CC4)ccn3)[C@@H](C)O)s2)n1] | | |
| I-266 | N-((1R,2S)-1-(4-(cyclopropanesulfonamido)pyridin-2-yl)-2- | 505 | 0.84 |
| | hydroxypropyl)-5-(6-ethoxypyrazin-2-yl)thiazole-2- | | LCMS-AU |
| | carboxamide | | |
| | 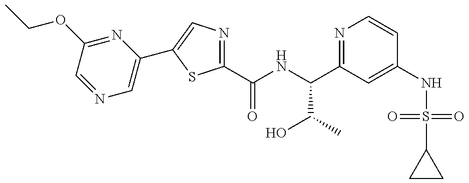 [SMILES: CCOc1cncc(-c2cnc(C(=O)N[C@H](c3cc(NS(=O)(=O)C4CC4)ccn3)[C@H](C)O)s2)n1] | | |
| I-267 | N-((1R,2R)-1-(4-(cyclopropanesulfonamido)pyridin-2-yl)-2- | 505 | 0.84 |
| | hydroxypropyl)-5-(6-ethoxypyrazin-2-yl)thiazole-2- | | LCMS-AU |
| | carboxamide | | |
| | 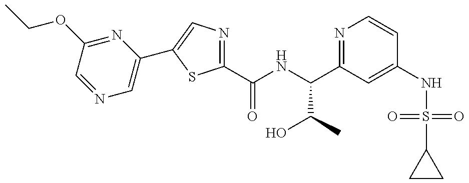 [SMILES: CCOc1cncc(-c2cnc(C(=O)N[C@H](c3cc(NS(=O)(=O)C4CC4)ccn3)[C@@H](C)O)s2)n1] | | |
| I-269 | N-((1S,2R)-1-(4-(cyclopropanesulfonamido)pyridin-2-yl)-2- | 505 | 0.99 |
| | hydroxypropyl)-5-(6-ethoxypyrazin-2-yl)thiazole-2- | | LCMS-P |
| | carboxamide | | |
| | 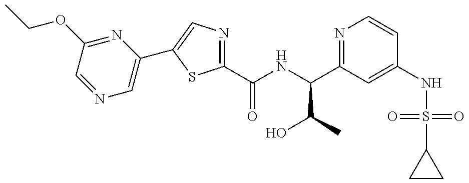 [SMILES: CCOc1cncc(-c2cnc(C(=O)N[C@@H](c3cc(NS(=O)(=O)C4CC4)ccn3)[C@@H](C)O)s2)n1] | | |
| I-270 | N-((1S,2S)-1-(4-(cyclopropanesulfonamido)pyridin-2-yl)-2- | 505 | 1.011 |
| | hydroxypropyl)-5-(6-ethoxypyrazin-2-yl)thiazole-2- | | LCMS-P |
| | carboxamide | | |
| | 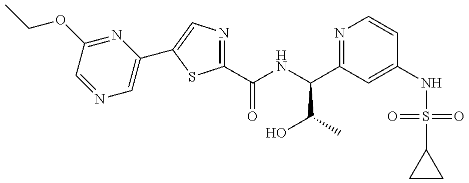 [SMILES: CCOc1cncc(-c2cnc(C(=O)N[C@@H](c3cc(NS(=O)(=O)C4CC4)ccn3)[C@H](C)O)s2)n1] | | |
| I-271 | 5-(5-chloropyrazolo[1,5-a]pyridin-3-yl)-N-((1R,2R)-1-(4- | 533/535 | 1.441 |
| | (cyclopropanesulfonamido)pyridin-2-yl)-2- | | LCMS-G |
| | hydroxypropyl)thiazole-2-carboxamide | | |
| | 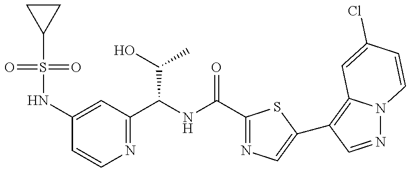 [SMILES: C[C@@H](O)[C@H](NC(=O)c1ncc(-c2cnn3ccc(Cl)cc23)s1)c1cc(NS(=O)(=O)C2CC2)ccn1] | | |
| I-272 | 5-(5-chloropyrazolo[1,5-a]pyridin-3-yl)-N-((1R,2S)-1-(4- | 533/535 | 1.427 |
| | (cyclopropanesulfonamido)pyridin-2-yl)-2- | | LCMS-G |
| | hydroxypropyl)thiazole-2-carboxamide | | |
| | 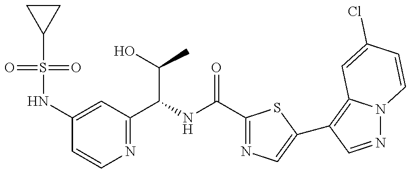 [SMILES: C[C@H](O)[C@H](NC(=O)c1ncc(-c2cnn3ccc(Cl)cc23)s1)c1cc(NS(=O)(=O)C2CC2)ccn1] | | |
| I-273 | 5-(5-chloropyrazolo[1,5-a]pyridin-3-yl)-N-((1S,2S)-1-(4- | 533/535 | 1.447 |
| | (cyclopropanesulfonamido)pyridin-2-yl)-2- | | LCMS-G |
| | hydroxypropyl)thiazole-2-carboxamide | | |
| | 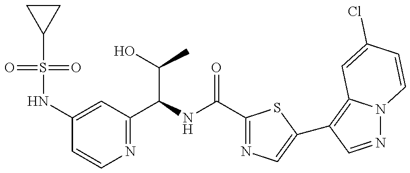 [SMILES: C[C@H](O)[C@@H](NC(=O)c1ncc(-c2cnn3ccc(Cl)cc23)s1)c1cc(NS(=O)(=O)C2CC2)ccn1] | | |
| I-274 | 5-(5-chloropyrazolo[1,5-a]pyridin-3-yl)-N-((1S,2R)-1-(4- | 533/535 | 1.008 |
| | (cyclopropanesulfonamido)pyridin-2-yl)-2- | | LCMS-L |
| | hydroxypropyl)thiazole-2-carboxamide | | |
| | 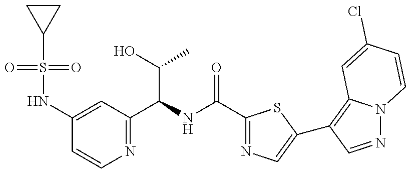 [SMILES: C[C@@H](O)[C@@H](NC(=O)c1ncc(-c2cnn3ccc(Cl)cc23)s1)c1cc(NS(=O)(=O)C2CC2)ccn1] | | |
| I-275 | (S)-N-(1-(4-(cyclopropanesulfonamido)pyridin-2-yl)-2- | 519 | 1.022 |
| | hydroxy-2-methylpropyl)-5-(6-ethoxypyrazin-2-yl)thiazole-2- | | LCMS-P |
| | carboxamide | | |
| | 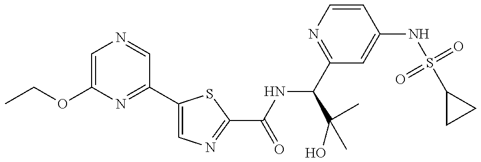 [SMILES: CCOc1cncc(-c2cnc(C(=O)N[C@@H](c3cc(NS(=O)(=O)C4CC4)ccn3)C(C)(C)O)s2)n1] | | |
| I-276 | (R)-N-(1-(4-(cyclopropanesulfonamido)pyridin-2-yl)-2- | 519 | 0.882 |
| | hydroxy-2-methylpropyl)-5-(6-ethoxypyrazin-2-yl)thiazole-2- | | LCMS-AV |
| | carboxamide | | |
| | 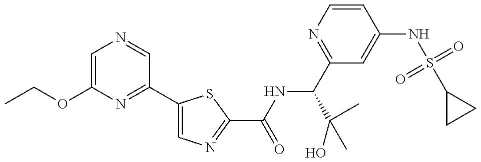 [SMILES: CCOc1cncc(-c2cnc(C(=O)N[C@H](c3cc(NS(=O)(=O)C4CC4)ccn3)C(C)(C)O)s2)n1] | | |
| I-277 | (R)-5-(5-chloropyrazolo[1,5-a]pyridin-3-yl)-N-(1-(4- | 547/549 | 1.837 |
| | (cyclopropanesulfonamido)pyridin-2-yl)-2-hydroxy-2- | | LCMS-G |
| | methylpropyl)thiazole-2-carboxamide | | |
| | 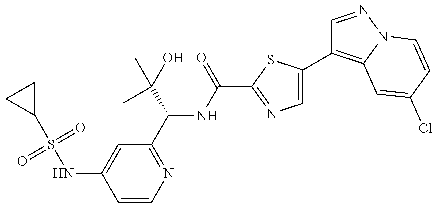 [SMILES: CC(C)(O)[C@H](NC(=O)c1ncc(-c2cnn3ccc(Cl)cc23)s1)c1cc(NS(=O)(=O)C2CC2)ccn1] | | |
| I-278 | (S)-5-(5-chloropyrazolo[1,5-a]pyridin-3-yl)-N-(1-(4- | 547/549 | 1.833 |
| | (cyclopropanesulfonamido)pyridin-2-yl)-2-hydroxy-2- | | LCMS-G |
| | methylpropyl)thiazole-2-carboxamide | | |
| | 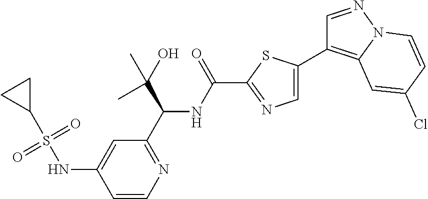 [SMILES: CC(C)(O)[C@@H](NC(=O)c1ncc(-c2cnn3ccc(Cl)cc23)s1)c1cc(NS(=O)(=O)C2CC2)ccn1] | | |
| I-279 | (R)-N-(1-(4-(cyclopropanesulfonamido)pyridin-2-yl)-2- | 498 | 1.065 |
| | methoxyethyl)-4-(6-ethoxypyrazin-2-yl)benzamide | | LCMS-AN |
| | 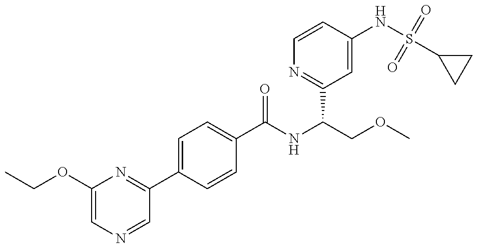 [SMILES: CCOc1cncc(-c2ccc(C(=O)N[C@@H](COC)c3cc(NS(=O)(=O)C4CC4)ccn3)cc2)n1] | | |
| I-280 | (S)-N-(1-(4-(cyclopropanesulfonamido)pyridin-2-yl)-2- | 498 | 1.064 |
| | methoxyethyl)-4-(6-ethoxypyrazin-2-yl)benzamide | | LCMS-AN |
| | 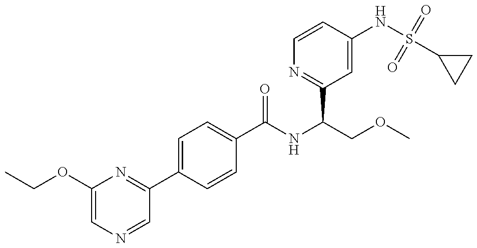 [SMILES: CCOc1cncc(-c2ccc(C(=O)N[C@H](COC)c3cc(NS(=O)(=O)C4CC4)ccn3)cc2)n1] | | |
| I-282 | (R)-N-(1-(4-(cyclopropanesulfonamido)pyridin-2-yl)-2- | 516 | 0.873 |
| | methoxyethyl)-4-(6-ethoxypyrazin-2-yl)-2-fluorobenzamide | | LCMS-AX |
| | 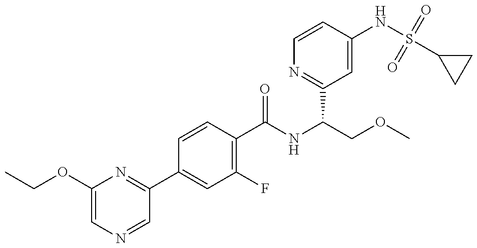 [SMILES: CCOc1cncc(-c2ccc(C(=O)N[C@@H](COC)c3cc(NS(=O)(=O)C4CC4)ccn3)c(F)c2)n1] | | |
| I-283 | (S)-N-(1-(4-(cyclopropanesulfonamido)pyridin-2-yl)-2- | 516 | 0.873 |
| | methoxyethyl)-4-(6-ethoxypyrazin-2-yl)-2-fluorobenzamide | | LCMS-AX |
| | 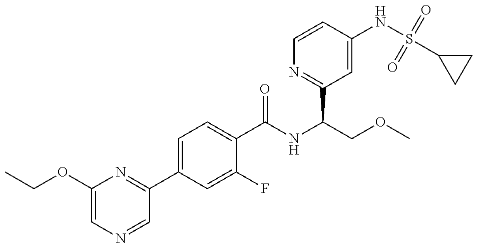 [SMILES: CCOc1cncc(-c2ccc(C(=O)N[C@H](COC)c3cc(NS(=O)(=O)C4CC4)ccn3)c(F)c2)n1] | | |
| I-284 | (R)-4-(5-chloropyrazolo[1,5-a]pyridin-3-yl)-N-(1-(4- | 544/546 | 1.693 |
| | (cyclopropanesulfonamido)pyridin-2-yl)-2-methoxyethyl)-2- | | LCMS-H |
| | fluorobenzamide | | |
| | 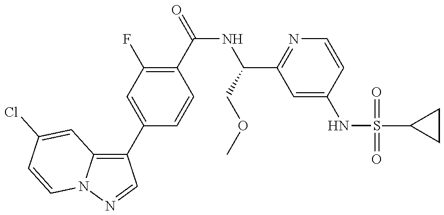 [SMILES: COC[C@H](NC(=O)c1ccc(-c2cnn3ccc(Cl)cc23)cc1F)c1cc(NS(=O)(=O)C2CC2)ccn1] | | |
| I-285 | (S)-4-(5-chloropyrazolo[1,5-a]pyridin-3-yl)-N-(1-(4- | 544/546 | 1.697 |
| | (cyclopropanesulfonamido)pyridin-2-yl)-2-methoxyethyl)-2- | | LCMS-H |
| | fluorobenzamide | | |
| | 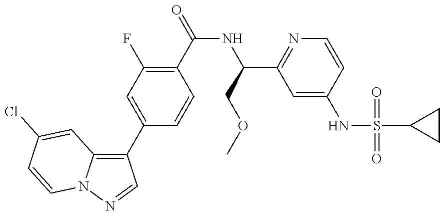 [SMILES: COC[C@@H](NC(=O)c1ccc(-c2cnn3ccc(Cl)cc23)cc1F)c1cc(NS(=O)(=O)C2CC2)ccn1] | | |
| I-286 | N-((1R,2R)-1-(3-((difluoromethyl)sulfonamido)phenyl)-2- | 514 | 1.195 |
| | hydroxypropyl)-5-(6-ethoxypyrazin-2-yl)thiazole-2- | | LCMS-P |
| | carboxamide | | |
| | 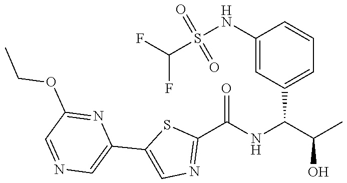 [SMILES: CCOc1cncc(-c2cnc(C(=O)N[C@H](c3cccc(NS(=O)(=O)C(F)F)c3)[C@@H](C)O)s2)n1] | | |
| I-287 | N-((1S,2R)-1-(3-((difluoromethyl)sulfonamido)phenyl)-2- | 514 | 1.195 |
| | hydroxypropyl)-5-(6-ethoxypyrazin-2-yl)thiazole-2- | | LCMS-P |
| | carboxamide | | |
| | 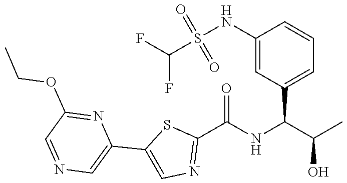 [SMILES: CCOc1cncc(-c2cnc(C(=O)N[C@@H](c3cccc(NS(=O)(=O)C(F)F)c3)[C@@H](C)O)s2)n1] | | |
| I-288 | N-((1S,2S)-1-(3-((difluoromethyl)sulfonamido)phenyl)-2- | 514 | 1.195 |
| | hydroxypropyl)-5-(6-ethoxypyrazin-2-yl)thiazole-2- | | LCMS-P |
| | carboxamide | | |
| | 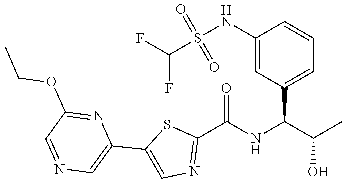 [SMILES: CCOc1cncc(-c2cnc(C(=O)N[C@@H](c3cccc(NS(=O)(=O)C(F)F)c3)[C@H](C)O)s2)n1] | | |
| I-289 | N-((1R,2S)-1-(3-((difluoromethyl)sulfonamido)phenyl)-2- | 514 | 1.195 |
| | hydroxypropyl)-5-(6-ethoxypyrazin-2-yl)thiazole-2- | | LCMS-P |
| | carboxamide | | |
| | 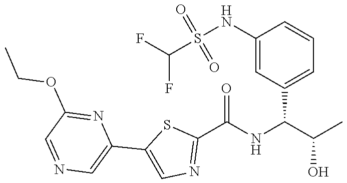 [SMILES: CCOc1cncc(-c2cnc(C(=O)N[C@H](c3cccc(NS(=O)(=O)C(F)F)c3)[C@H](C)O)s2)n1] | | |
| I-290 | (S)-N-(1-(3-((difluoromethyl)sulfonamido)phenyl)-2-hydroxy- | 528 | 1.748 |
| | 2-methylpropyl)-5-(6-ethoxypyrazin-2-yl)thiazole-2- | | LCMS-N |
| | carboxamide | | |
| | 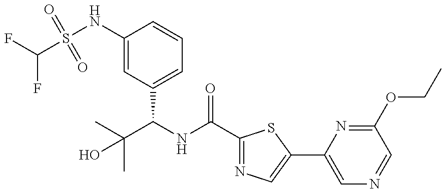 [SMILES: CCOc1cncc(-c2cnc(C(=O)N[C@@H](c3cccc(NS(=O)(=O)C(F)F)c3)C(C)(C)O)s2)n1] | | |
| I-291 | (R)-N-(1-(3-((difluoromethyl)sulfonamido)phenyl)-2-hydroxy- | 528 | 1.754 |
| | 2-methylpropyl)-5-(6-ethoxypyrazin-2-yl)thiazole-2- | | LCMS-N |
| | carboxamide | | |
| | 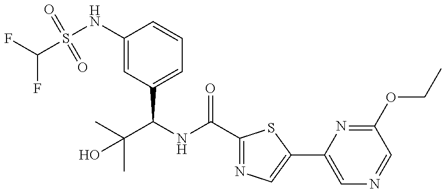 [SMILES: CCOc1cncc(-c2cnc(C(=O)N[C@H](c3cccc(NS(=O)(=O)C(F)F)c3)C(C)(C)O)s2)n1] | | |
| I-292 | (S)-N-(1-(3-((difluoromethyl)sulfonamido)phenyl)-2- | 514 | 1.492 |
| | methoxyethyl)-5-(6-ethoxypyrazin-2-yl)thiazole-2-carboxamide | | LCMS-BF |
| | 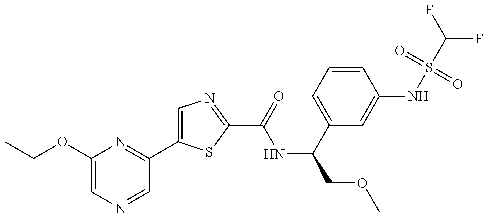 [SMILES: CCOc1cncc(-c2cnc(C(=O)N[C@H](COC)c3cccc(NS(=O)(=O)C(F)F)c3)s2)n1] | | |
| I-293 | (R)-N-(1-(3-((difluoromethyl)sulfonamido)phenyl)-2- | 514 | 1.488 |
| | methoxyethyl)-5-(6-ethoxypyrazin-2-yl)thiazole-2-carboxamide | | LCMS-BF |
| | 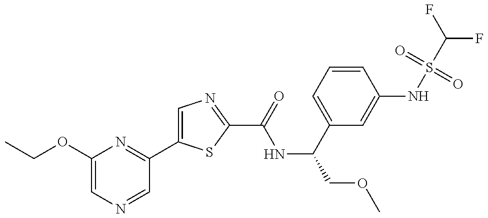 [SMILES: CCOc1cncc(-c2cnc(C(=O)N[C@@H](COC)c3cccc(NS(=O)(=O)C(F)F)c3)s2)n1] | | |
| I-281 | (R)-N-(1-(2-(cyclopropanesulfonamido)pyrimidin-4-yl)-2- | 506 | 0.828 |
| | methoxyethyl)-5-(6-ethoxypyrazin-2-yl)thiazole-2-carboxamide | | LCMS-AU |
| | 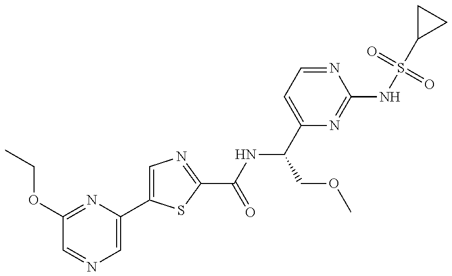 [SMILES: CCOc1cncc(-c2cnc(C(=O)N[C@@H](COC)c3ccnc(NS(=O)(=O)C4CC4)n3)s2)n1] | | |
| I-268 | (S)-N-(1-(2-(cyclopropanesulfonamido)pyrimidin-4-yl)-2- | 506 | 0.815 |
| | methoxyethyl)-5-(6-ethoxypyrazin-2-yl)thiazole-2-carboxamide | | LCMS-AV |
| | 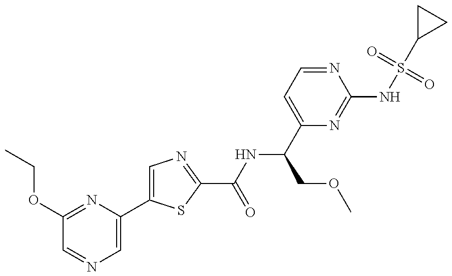 [SMILES: CCOc1cncc(-c2cnc(C(=O)N[C@H](COC)c3ccnc(NS(=O)(=O)C4CC4)n3)s2)n1] | | |
| I-98 | (R)-N-(1-(4-(cyclopropanesulfonamido)pyrimidin-2-yl)-2- | 506 | 1.304 |
| | methoxyethyl)-5-(6-ethoxypyrazin-2-yl)thiazole-2-carboxamide | | LCMS-AB |
| | 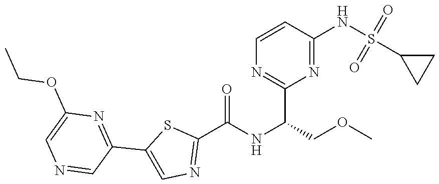 [SMILES: CCOc1cncc(-c2cnc(C(=O)N[C@@H](COC)c3nccc(NS(=O)(=O)C4CC4)n3)s2)n1] | | |
| I-21 | (S)-N-(1-(4-(cyclopropanesulfonamido)pyrimidin-2-yl)-2- | 506 | 1.301 |
| | methoxyethyl)-5-(6-ethoxypyrazin-2-yl)thiazole-2-carboxamide | | LCMS-AB |
| | 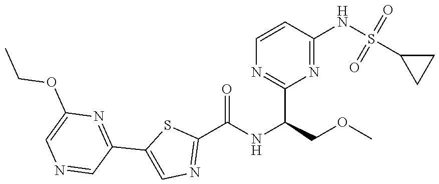 [SMILES: CCOc1cncc(-c2cnc(C(=O)N[C@H](COC)c3nccc(NS(=O)(=O)C4CC4)n3)s2)n1] | | |

TABLE 4
additional compounds

| | Name/Structure | MS (ES): | LCMS Rt |
|---|---|---|---|
| | Stereochemistry alpha to central amide | m/z | (mins) |
| Cmpd. No. | arbitrary assigned | [M + H]+ | Method |
| II-1 | (R)-5-(5-chloropyrazolo[1,5-a]pyridin-3-yl)-N- | 551/553 | 0.74 |
| | (1-(4-(cyclopropanesulfonamido)-3- | | LCMS-BB |
| | fluoropyridin-2-yl)-2-methoxyethyl)thiazole-2- | | |
| | carboxamide | | |
| | 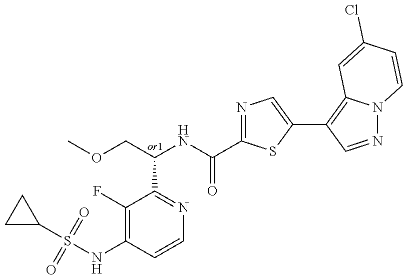 [SMILES: COC[C@H](NC(=O)c1ncc(-c2cnn3ccc(Cl)cc23)s1)c1nccc(NS(=O)(=O)C2CC2)c1F] | | |
| II-2 | (S)-5-(5-chloropyrazolo[1,5-a]pyridin-3-yl)-N- | 551/553 | 0.746 |
| | (1-(4-(cyclopropanesulfonamido)-3- | | LCMS-BB |
| | fluoropyridin-2-yl)-2-methoxyethyl)thiazole-2- | | |
| | carboxamide | | |
| | 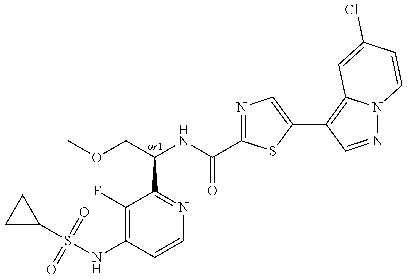 [SMILES: COC[C@@H](NC(=O)c1ncc(-c2cnn3ccc(Cl)cc23)s1)c1nccc(NS(=O)(=O)C2CC2)c1F] | | |
| II-3 | 5-(5-chloropyrazolo[1,5-a]pyridin-3-yl)-N- | 577/579 | 0.712 |
| | ((S)-(4-(cyclopropanesulfonamido)-3- | | LCMS-BB |
| | fluoropyridin-2-yl)((R)-tetrahydrofuran-3- | | |
| | yl)methyl)thiazole-2-carboxamide | | |
| | 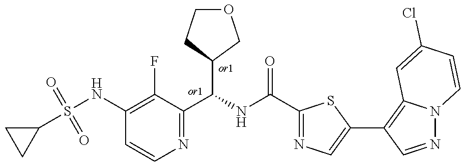 [SMILES: O=C(N[C@H](c1nccc(NS(=O)(=O)C2CC2)c1F)[C@H]1CCOC1)c1ncc(-c2cnn3ccc(Cl)cc23)s1] | | |
| II-4 | 5-(5-chloropyrazolo[1,5-a]pyridin-3-yl)-N- | 577/579 | 1.362 |
| | ((R)-(4-(cyclopropanesulfonamido)-3- | | LCMS-BC |
| | fluoropyridin-2-yl)((S)-tetrahydrofuran-3- | | |
| | yl)methyl)thiazole-2-carboxamide | | |
| | 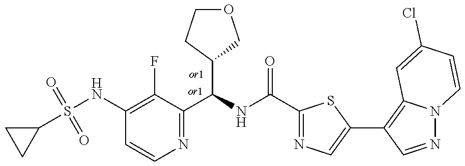 [SMILES: O=C(N[C@@H](c1nccc(NS(=O)(=O)C2CC2)c1F)[C@@H]1CCOC1)c1ncc(-c2cnn3ccc(Cl)cc23)s1] | | |
| II-5 | 5-(5-chloropyrazolo[1,5-a]pyridin-3-yl)-N- | 577/579 | 0.709 |
| | ((R)-(4-(cyclopropanesulfonamido)-3- | | LCMS-BB |
| | fluoropyridin-2-yl)((R)-tetrahydrofuran-3- | | |
| | yl)methyl)thiazole-2-carboxamide | | |
| | 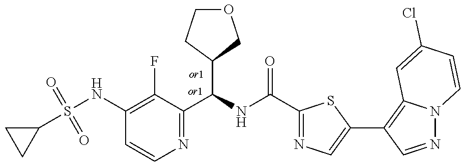 [SMILES: O=C(N[C@@H](c1nccc(NS(=O)(=O)C2CC2)c1F)[C@H]1CCOC1)c1ncc(-c2cnn3ccc(Cl)cc23)s1] | | |
| II-6 | 5-(5-chloropyrazolo[1,5-a]pyridin-3-yl)-N- | 577/579 | 1.336 |
| | ((S)-(4-(cyclopropanesulfonamido)-3- | | LCMS-BC |
| | fluoropyridin-2-yl)((S)-tetrahydrofuran-3- | | |
| | yl)methyl)thiazole-2-carboxamide | | |
| | 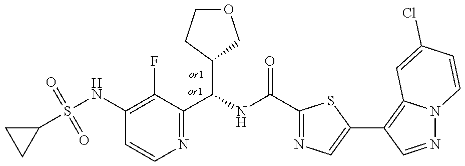 [SMILES: O=C(N[C@H](c1nccc(NS(=O)(=O)C2CC2)c1F)[C@@H]1CCOC1)c1ncc(-c2cnn3ccc(Cl)cc23)s1] | | |
| II-7 | (R)-N-(1-(6- | 506 | 0.921 |
| | (cyclopropanesulfonamido)pyrimidin-4-yl)-2- | | LCMS-AM |
| | methoxyethyl)-5-(6-ethoxypyrazin-2- | | |
| | yl)thiazole-2-carboxamide | | |
| | 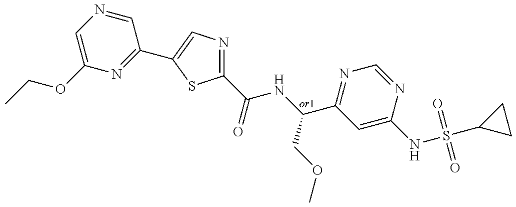 [SMILES: CCOc1cncc(-c2cnc(C(=O)N[C@@H](COC)c3cc(NS(=O)(=O)C4CC4)ncn3)s2)n1] | | |
| II-8 | (S)-N-(1-(6- | 506 | 0.921 |
| | (cyclopropanesulfonamido)pyrimidin-4-yl)-2- | | LCMS-AM |
| | methoxyethyl)-5-(6-ethoxypyrazin-2- | | |
| | yl)thiazole-2-carboxamide | | |
| | 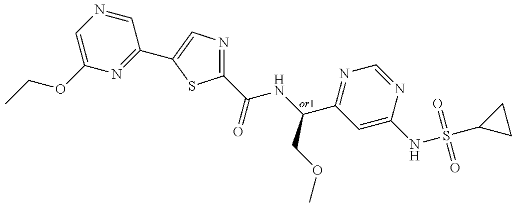 [SMILES: CCOc1cncc(-c2cnc(C(=O)N[C@H](COC)c3cc(NS(=O)(=O)C4CC4)ncn3)s2)n1] | | |
| II-9 | (R)-5-(5-chloropyrazolo[1,5-a]pyridin-3-yl)-N- | 534/536 | 0.696 |
| | (1-(4-(cyclopropanesulfonamido)pyrimidin-2- | | LCMS-BB |
| | yl)-2-methoxyethyl)thiazole-2-carboxamide | | |
| | 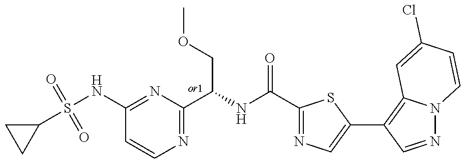 [SMILES: COC[C@H](NC(=O)c1ncc(-c2cnn3ccc(Cl)cc23)s1)c1nccc(NS(=O)(=O)C2CC2)n1] | | |
| II-10 | (S)-5-(5-chloropyrazolo[1,5-a]pyridin-3-yl)-N- | 534/536 | 0.693 |
| | (1-(4-(cyclopropanesulfonamido)pyrimidin-2- | | LCMS-BB |
| | yl)-2-methoxyethyl)thiazole-2-carboxamide | | |
| | 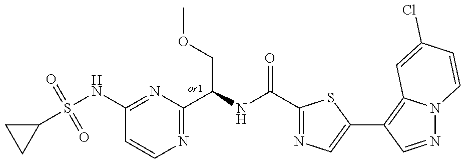 [SMILES: COC[C@@H](NC(=O)c1ncc(-c2cnn3ccc(Cl)cc23)s1)c1nccc(NS(=O)(=O)C2CC2)n1] | | |
| II-11 | N-((1R,2S)-1-(3- | 595 | 1.802 |
| | ((difluoromethyl)sulfonamido)phenyl)-2- | | LCMS-AE |
| | methyl-3-(piperidin-1-yl)propyl)-5-(6- | | |
| | ethoxypyrazin-2-yl)thiazole-2-carboxamide | | |
| | 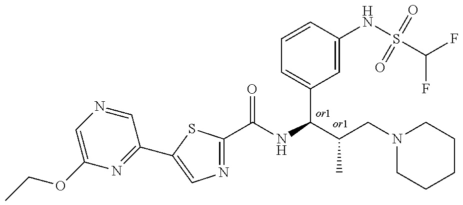 [SMILES: CCOc1cncc(-c2cnc(C(=O)N[C@@H](c3cccc(NS(=O)(=O)C(F)F)c3)[C@@H](C)CN3CCCCC3)s2)n1] | | |
| II-12 | N-((1R,2R)-1-(3- | 595 | 1.809 |
| | ((difluoromethyl)sulfonamido)phenyl)-2- | | LCMS-AE |
| | methyl-3-(piperidin-1-yl)propyl)-5-(6- | | |
| | ethoxypyrazin-2-yl)thiazole-2-carboxamide | | |
| | 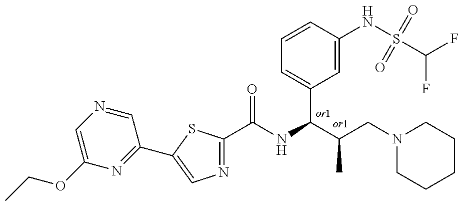 [SMILES: CCOc1cncc(-c2cnc(C(=O)N[C@@H](c3cccc(NS(=O)(=O)C(F)F)c3)[C@H](C)CN3CCCCC3)s2)n1] | | |
| 11-13 | N-((1S,2R)-1-(3- | 595 | 1.327 |
| | ((difluoromethyl)sulfonamido)phenyl)-2- | | LCMS-AB |
| | methyl-3-(piperidin-1-yl)propyl)-5-(6- | | |
| | ethoxypyrazin-2-yl)thiazole-2-carboxamide | | |
| | 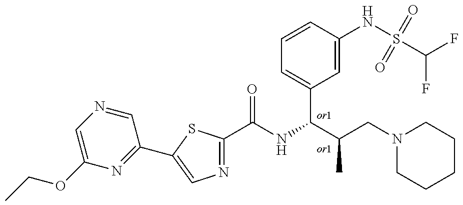 [SMILES: CCOc1cncc(-c2cnc(C(=O)N[C@H](c3cccc(NS(=O)(=O)C(F)F)c3)[C@H](C)CN3CCCCC3)s2)n1] | | |
| II-14 | N-((1S,2S)-1-(3- | 595 | 1.542 |
| | ((difluoromethyl)sulfonamido)phenyl)-2- | | LCMS-AG |
| | methyl-3-(piperidin-1-yl)propyl)-5-(6- | | |
| | ethoxypyrazin-2-yl)thiazole-2-carboxamide | | |
| | 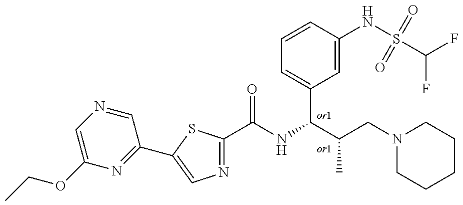 [SMILES: CCOc1cncc(-c2cnc(C(=O)N[C@H](c3cccc(NS(=O)(=O)C(F)F)c3)[C@@H](C)CN3CCCCC3)s2)n1] | | |
| II-15 | (S)-5-(5-chloropyrazolo[1,5-a]pyridin-3-yl)-N- | 574/576 | 1.586 |
| | ((4-(cyclopropanesulfonamido)pyridin-2- | | LCMS-G |
| | yl)(tetrahydro-2H-pyran-4-yl)methyl)-1,3,4- | | |
| | thiadiazole-2-carboxamide | | |
| | 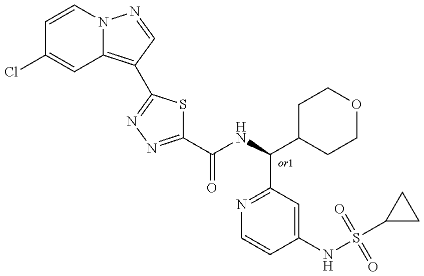 [SMILES: O=C(N[C@H](c1cc(NS(=O)(=O)C2CC2)ccn1)C1CCOCC1)c1nnc(-c2cnn3ccc(Cl)cc23)s1] | | |
| II-16 | (R)-5-(5-chloropyrazolo[1,5-a]pyridin-3-yl)-N- | 574/576 | 1.582 |
| | ((4-(cyclopropanesulfonamido)pyridin-2- | | LCMS-G |
| | yl)(tetrahydro-2H-pyran-4-yl)methyl)-1,3,4- | | |
| | thiadiazole-2-carboxamide | | |
| | 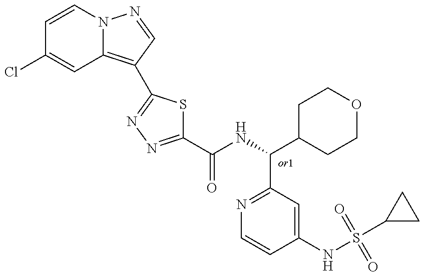 [SMILES: O=C(N[C@@H](c1cc(NS(=O)(=O)C2CC2)ccn1)C1CCOCC1)c1nnc(-c2cnn3ccc(Cl)cc23)s1] | | |
| II-17 | N-((S)-(4-(cyclopropanesulfonamido)-3- | 549 | 2.04 |
| | fluoropyridin-2-yl)((R)-tetrahydrofuran-3- | | LCMS-AK |
| | yl)methyl)-5-(6-ethoxypyrazin-2-yl)thiazole-2- | | |
| | carboxamide | | |
| | 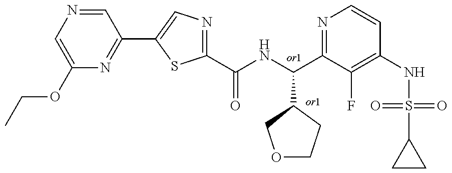 [SMILES: CCOc1cncc(-c2cnc(C(=O)N[C@H](c3nccc(NS(=O)(=O)C4CC4)c3F)[C@H]3CCOC3)s2)n1] | | |
| II-18 | N-((S)-(4-(cyclopropanesulfonamido)-3- | 549 | 2.04 |
| | fluoropyridin-2-yl)((S)-tetrahydrofuran-3- | | LCMS-AK |
| | yl)methyl)-5-(6-ethoxypyrazin-2-yl)thiazole-2- | | |
| | carboxamide | | |
| | 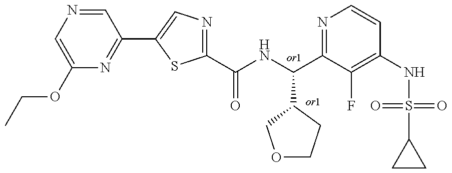 [SMILES: CCOc1cncc(-c2cnc(C(=O)N[C@H](c3nccc(NS(=O)(=O)C4CC4)c3F)[C@@H]3CCOC3)s2)n1] | | |
| II-19 | N-((R)-(4-(cyclopropanesulfonamido)-3- | 549 | 1.111 |
| | fluoropyridin-2-yl)((S)-tetrahydrofuran-3- | | LCMS-AS |
| | yl)methyl)-5-(6-ethoxypyrazin-2-yl)thiazole-2- | | |
| | carboxamide | | |
| | 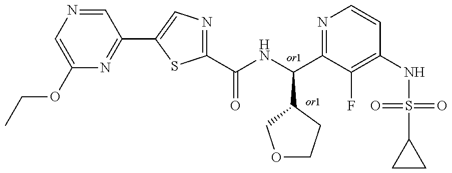 [SMILES: CCOc1cncc(-c2cnc(C(=O)N[C@@H](c3nccc(NS(=O)(=O)C4CC4)c3F)[C@@H]3CCOC3)s2)n1] | | |
| II-20 | N-((R)-(4-(cyclopropanesulfonamido)-3- | 549 | 1.103 |
| | fluoropyridin-2-yl)((R)-tetrahydrofuran-3- | | LCMS-AS |
| | yl)methyl)-5-(6-ethoxypyrazin-2-yl)thiazole-2- | | |
| | carboxamide | | |
| | 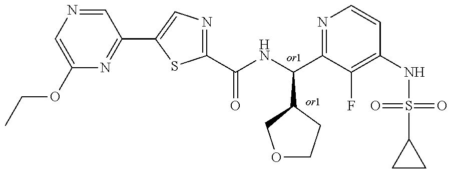 [SMILES: CCOc1cncc(-c2cnc(C(=O)N[C@@H](c3nccc(NS(=O)(=O)C4CC4)c3F)[C@H]3CCOC3)s2)n1] | | |
| II-21 | N-((4-(cyclopropanesulfonamido)-3- | 479 | 1.44 |
| | fluoropyridin-2-yl)methyl)-5-(6- | | LCMS-AF |
| | ethoxypyrazin-2-yl)thiazole-2-carboxamide | | |
| | 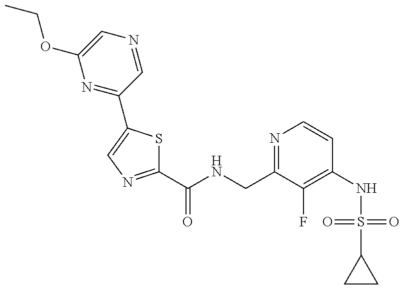 [SMILES: CCOc1cncc(-c2cnc(C(=O)NCc3nccc(NS(=O)(=O)C4CC4)c3F)s2)n1] | | |

### Example 12: ADP-Glo™ Assay

The enzyme inhibitory activities of the compounds of the invention against the target of interest were determined using the ADP-Glo™ assay (Promega). Assays for human CTPS1 and CTPS2 were performed in 1× assay buffer containing 50 mM HEPES (Life Technologies), 10 mM Mg2+, 5 mM KCl, 2 mM DTT, 0.01% F-127, pH to 7.4 accordingly. All reagents are from Sigma-Aldrich, unless specified otherwise. Purified human full length active N-terminal FLAG-8×HIS-TEV tagged CTPS1 (UniprotKB—P17812, MDYKDDDDKGTHHHHHHHHENLYFQGS-CTPS1[1-591]) was generated using a mammalian expression system at Viva Biotech (Shanghai) Ltd.

Purified human CTPS1 protein was prepared in 1× assay buffer to the final working protein concentration required for the reaction. A 2.5 μL volume per well of human CTPS 1 protein was mixed with 0.1 μL per well of test compound dissolved in DMSO and pre-incubated at 25 degrees C. for 10 minutes. 2.5 μL per well of the reaction precursors ATP (UltraPure ATP from ADP-Glo™ kit) and UTP were then added and pre-incubated for an additional 10 minutes at 25 degrees C. Finally, the reaction was initiated by the addition of 5 μL of the reaction precursors L-glutamine and GTP. The final concentration of all reaction components in the assay: ATP (120 μM), UTP (160 μM), GTP (60 μM), L-Glutamine (100 μM), DMSO (1%), hCTPS1 (25 nM). This mixture was incubated for an appropriate amount of time within the determined linear phase of the reaction at 25 degrees C. under sealed plate conditions with constant agitation at 500 revolutions per minute (rpm). ADP-Glo™ reagent was added for 60 minutes (10 μL per well) and subsequently ADP-Glo™ development reagent was added for 60 minutes (20 μL per well) prior to signal detection in a microplate reader (Envision Multilabel Reader, Perkin Elmer). Following each reagent addition over the course of the assay, assay plates were pulse centrifuged for 1 minute at 1000 rpm.

The enzyme converts ATP to ADP and the ADP-Glo™ reagent subsequently depletes any remaining ATP in the reaction system. The ADP-Glo™ detection reagents converts the ADP that has been produced by the enzyme back into ATP, which is then utilized as a substrate together with luciferin for the enzyme luciferase. Light generated by this chemiluminescent reaction is quantified and is directly proportional to the amount of ADP produced by the CTPS1 enzyme reaction. A reduction in this signal by compound treatment demonstrates enzyme inhibition, and the percentage inhibition produced by each concentration of compound was calculated as: % inhibition=1−((Meanₘᵢₙ−Meanᵢₙₕ)/(Meanₘᵢₙ−Meanₘₐₓ))×100. The data for all compounds tested are presented below in Table 5 and Table 6.

Compounds with an IC₅₀ less than or equal to 0.005 μM are designated as “A.”

Compounds with an IC₅₀ greater than 0.005 μM and less than or equal to 0.025 μM are designated as “B.”

Compounds with an IC₅₀ greater than 0.025 μM and less than or equal to 0.05 μM are designated as “C.”

Compounds with an IC₅₀ greater than 0.05 μM or equal to 0.1 μM are designated as “D.”

Compounds with an IC₅₀ greater than 0.1 μM are designated as “E.”

TABLE 4

| | ADP- |
|---|---|
| | Glo_WT_hCTPS1_{—} |
| Cmpd. | 25 nM_IC50 (IC50) |
| No. | [μM] |
| I-11 | B |
| I-12 | B |
| I-255 | B |
| I-19 | B |
| I-20 | B |
| I-13 | D |
| I-14 | B |
| I-5 | B |
| I-15 | D |
| I-16 | B |
| I-17 | A |
| I-18 | E |
| I-28 | A |
| I-29 | E |
| I-32 | C |
| I-34 | B |
| I-35 | A |
| I-33 | B |
| I-26 | E |
| I-27 | B |
| I-36 | D |
| I-37 | B |
| I-30 | C |
| I-31 | B |
| I-9 | E |
| I-10 | B |
| I-1 | B |
| I-2 | B |
| I-6 | E |
| I-7 | B |
| I-8 | C |
| I-44 | B |
| I-45 | B |
| I-46 | B |
| I-47 | B |
| I-93 | B |
| I-94 | E |
| I-95 | B |
| I-96 | B |
| I-97 | B |
| I-134 | B |
| I-185 | D |
| I-186 | B |
| I-123 | D |
| I-124 | C |
| I-135 | A |
| I-136 | B |
| I-51 | B |
| I-52 | D |
| I-53 | B |
| I-54 | B |
| I-125 | C |
| I-126 | B |
| I-125 | C |
| I-102 | D |
| I-104 | A |
| I-127 | B |
| I-129 | D |
| I-130 | B |
| I-131 | B |
| I-132 | B |
| I-48 | B |
| I-103 | E |
| I-115 | D |
| I-116 | C |
| I-117 | B |
| I-118 | B |
| I-143 | D |
| I-144 | B |
| I-107 | D |
| I-108 | B |
| I-109 | B |
| I-110 | A |
| I-226 | B |
| I-227 | A |
| I-228 | B |
| I-229 | A |
| I-139 | C |
| I-140 | B |
| I-49 | D |
| I-50 | B |
| I-137 | C |
| I-138 | B |
| I-279 | B |
| I-280 | C |
| I-282 | C |
| I-283 | D |
| I-172 | C |
| I-173 | B |
| I-174 | B |
| I-175 | B |
| I-42 | D |
| I-43 | B |
| I-147 | B |
| I-148 | B |
| I-67 | C |
| I-68 | C |
| I-69 | C |
| I-70 | C |
| I-149 | D |
| I-150 | B |
| I-264 | B |
| I-265 | B |
| I-171 | E |
| I-171 | D |
| I-112 | B |
| I-113 | B |
| I-114 | B |
| I-63 | E |
| I-64 | D |
| I-65 | E |
| I-66 | C |
| I-141 | D |
| I-142 | E |
| I-151 | D |
| I-152 | B |
| I-204 | B |
| I-205 | C |
| I-105 | B |
| I-106 | B |
| I-153 | C |
| I-154 | B |
| I-155 | C |
| I-156 | B |
| I-157 | C |
| I-158 | B |
| I-38 | B |
| I-39 | C |
| I-206 | B |
| I-207 | C |
| I-208 | B |
| I-209 | B |
| I-246 | B |
| I-247 | B |
| I-187 | B |
| I-188 | B |
| I-189 | A |
| I-190 | B |
| I-292 | B |
| I-293 | B |
| I-176 | B |
| I-177 | B |
| I-286 | B |
| I-287 | B |
| I-288 | B |
| I-289 | B |
| I-233 | B |
| I-255 | B |
| I-159 | B |
| I-266 | B |
| I-267 | B |
| I-269 | A |
| I-270 | A |
| I-167 | C |
| I-168 | B |
| I-222 | B |
| I-224 | B |
| I-262 | C |
| I-263 | B |
| I-166 | D |
| I-169 | C |
| I-71 | B |
| I-72 | B |
| I-73 | B |
| I-74 | B |
| I-75 | B |
| I-76 | A |
| I-180 | B |
| I-59 | C |
| I-60 | B |
| I-61 | B |
| I-62 | B |
| I-119 | C |
| I-120 | C |
| I-121 | B |
| I-122 | B |
| I-275 | C |
| I-276 | B |
| I-91 | B |
| I-92 | C |
| I-40 | C |
| I-41 | B |
| I-160 | B |
| I-161 | A |
| I-191 | C |
| I-192 | B |
| I-178 | C |
| I-87 | A |
| I-193 | D |
| I-145 | B |
| I-146 | B |
| I-22 | B |
| I-23 | B |
| I-24 | B |
| I-25 | B |
| I-230 | A |
| I-231 | B |
| I-232 | B |
| I-233 | B |
| I-281 | B |
| I-268 | C |
| I-210 | B |
| I-211 | D |
| I-81 | A |
| I-82 | A |
| I-83 | B |
| I-84 | A |
| I-170 | E |
| I-242 | A |
| I-243 | B |
| I-245 | A |
| I-88 | A |
| I-179 | B |
| I-3 | B |
| I-4 | B |
| I-238 | C |
| I-239 | B |
| I-240 | B |
| I-241 | B |
| I-252 | C |
| I-253 | D |
| I-212 | C |
| I-213 | B |
| I-194 | B |
| I-195 | B |
| I-183 | E |
| I-184 | B |
| I-97 | C |
| I-99 | B |
| I-100 | B |
| I-101 | B |
| I-244 | A |
| I-271 | B |
| I-272 | B |
| I-273 | B |
| I-274 | B |
| I-234 | A |
| I-235 | B |
| I-236 | A |
| I-237 | A |
| I-214 | B |
| I-215 | B |
| I-216 | A |
| I-217 | B |
| I-290 | B |
| I-291 | B |
| I-198 | B |
| I-199 | B |
| I-200 | B |
| I-201 | B |
| I-181 | C |
| I-182 | B |
| I-85 | C |
| I-86 | B |
| I-98 | C |
| I-21 | B |
| I-248 | C |
| I-249 | C |
| I-250 | C |
| I-251 | C |
| I-89 | B |
| I-90 | B |
| I-162 | D |
| I-163 | C |
| I-260 | B |
| I-261 | B |
| I-254 | B |
| I-256 | B |
| I-257 | B |
| I-258 | B |
| I-259 | B |
| I-77 | B |
| I-78 | B |
| I-79 | B |
| I-80 | B |
| I-218 | C |
| I-219 | B |
| I-164 | D |
| I-165 | B |
| I-196 | B |
| I-197 | B |
| I-277 | D |
| I-278 | D |
| I-202 | B |
| I-203 | B |
| I-55 | D |
| I-56 | B |
| I-57 | B |
| I-58 | D |
| I-220 | C |
| I-221 | C |
| I-284 | E |
| I-285 | D |

TABLE 6
Additional compounds

| | ADP- |
|---|---|
| | Glo_WT_hCTPS1_{—} |
| | 25 nM_IC50 |
| | (IC50) [uM] |
| II-1 | B |
| II-2 | B |
| II-3 | B |
| II-4 | B |
| II-5 | B |
| II-6 | B |
| II-7 | B |
| II-8 | B |
| II-9 | B |
| II-10 | C |
| II-11 | C |
| II-12 | B |
| II-13 | E |
| II-14 | D |
| II-15 | B |
| II-16 | B |
| II-17 | B |
| II-18 | B |
| II-19 | B |

We claim:

1. A compound of formula I: 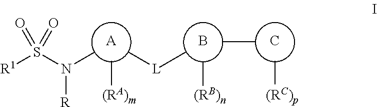
or a pharmaceutically acceptable salt thereof, wherein:
R¹ is selected from C₁₋₆ aliphatic; a 3-7 membered saturated or partially unsaturated monocyclic carbocyclic ring; and a 3-7 membered saturated or partially unsaturated monocyclic heterocyclic ring having 1-2 heteroatoms independently selected from nitrogen, oxygen, and sulfur; each of which is substituted with q instances of R^{A};
Ring A is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur;
L is 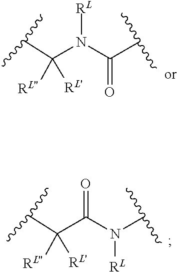
wherein R^{L} is hydrogen or optionally substituted C₁₋₆ aliphatic and one of R^{L′} or R^{L″} is hydrogen and the other of R^{L′} or R^{L″} is -L²-R², wherein:
R² is hydrogen, halogen, —OR, —NR₂, an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 7-12 membered saturated or partially unsaturated spirocyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—; or
R^{L} is hydrogen or optionally substituted C₁₋₆ aliphatic and an R^{L′} and R^{L″} group are taken together with the atom to which they are attached, to form an optionally substituted 3-7 membered saturated or partially unsaturated monocyclic carbocyclic ring; or a 3-7 membered optionally substituted saturated or partially unsaturated monocyclic heterocyclic ring having 1-2 heteroatoms independently selected from nitrogen, oxygen, and sulfur;
Ring B is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-3 heteroatoms independently selected from nitrogen, oxygen, and sulfur; a 5-11 membered saturated or partially unsaturated fused, bridged, or spiro, bicyclic carbocyclic ring; a 7-11 membered fused bicyclic aryl ring; a 7-12 membered saturated or partially unsaturated bicyclic heterocyclic ring that is optionally bridged bicyclic (having 1-4 heteroatoms independently selected from nitrogen, oxygen, and sulfur), a 7-12 membered saturated or partially unsaturated spirocyclic heterocyclic ring (having 1-4 heteroatoms independently selected from nitrogen, oxygen, and sulfur); and a 7-11 membered fused bicyclic heteroaryl ring having 1-4 heteroatoms independently selected from nitrogen, oxygen, and sulfur;
Ring C is selected from a phenyl, 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 7-11 membered fused bicyclic heteroaryl ring having 1-4 heteroatoms independently selected from nitrogen, oxygen, and sulfur;
each instance of R^{A}, R^{B}, and R^{C} is independently optionally substituted C₁4 aliphatic, oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR, or —P(O)R₂;
each R is independently hydrogen, —CN, halogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 9-12 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring; or
two R groups are taken together with the atoms to which each R is attached, to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and
m is 0, 1, or 2;
n is 0, 1, or 2;
p is 0, 1, or 2; and
q is 0, 1, 2, 3, or 4.

2. The compound of claim 1, wherein R¹ is C₁₋₆ aliphatic or a 3-7 membered saturated or partially unsaturated monocyclic carbocyclic ring; each of which is substituted with q instances of R^{A}.

3. The compound of claim 1, wherein R¹ is selected from C₁₋₆ haloalkyl or cyclopropyl; each of which is substituted with q instances of R^{A}.

4. The compound of claim 1, wherein R¹

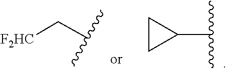

5. The compound of claim 1, wherein Ring A is phenyl or a 5-6 membered monocyclic heteroaryl ring having 1-4 heteroatoms independently selected from nitrogen, oxygen, and sulfur.

6. The compound of claim 1, wherein Ring A is phenyl, pyrazinyl, pyrimidinyl, or pyridinyl.

7. The compound of claim 1, wherein Ring A is

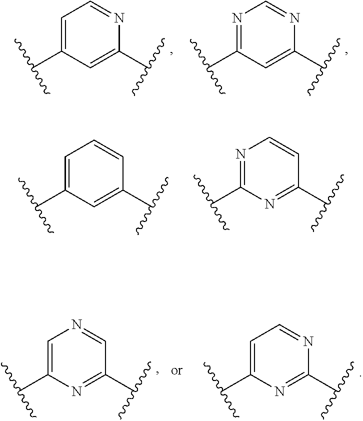 [SMILES: *c1ccnc(*)c1 ; *c1cc(*)ncn1 ; *c1cccc(*)c1 ; *c1ccnc(*)n1 ; *c1cncc(*)n1 ; *c1ccnc(*)n1]

8. The compound of claim 1, wherein L is

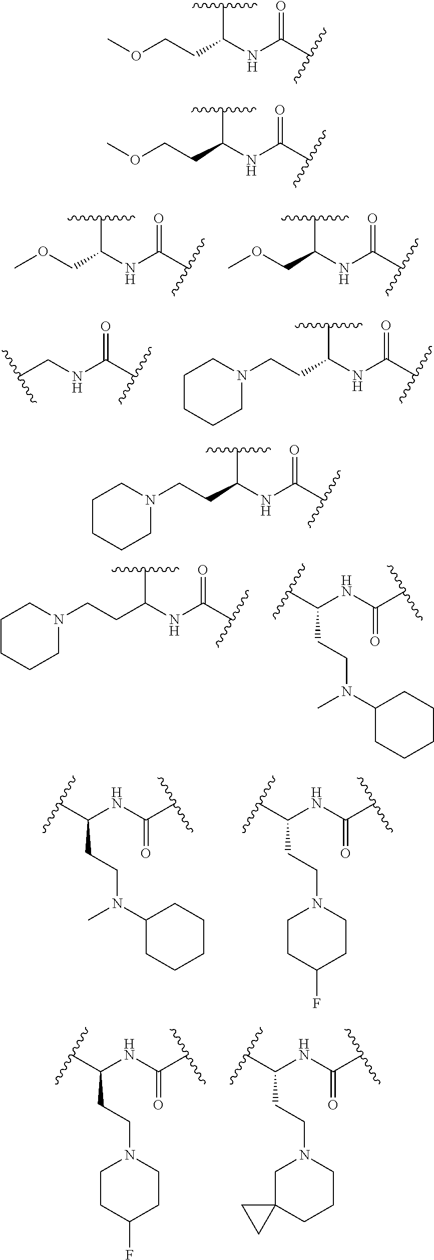

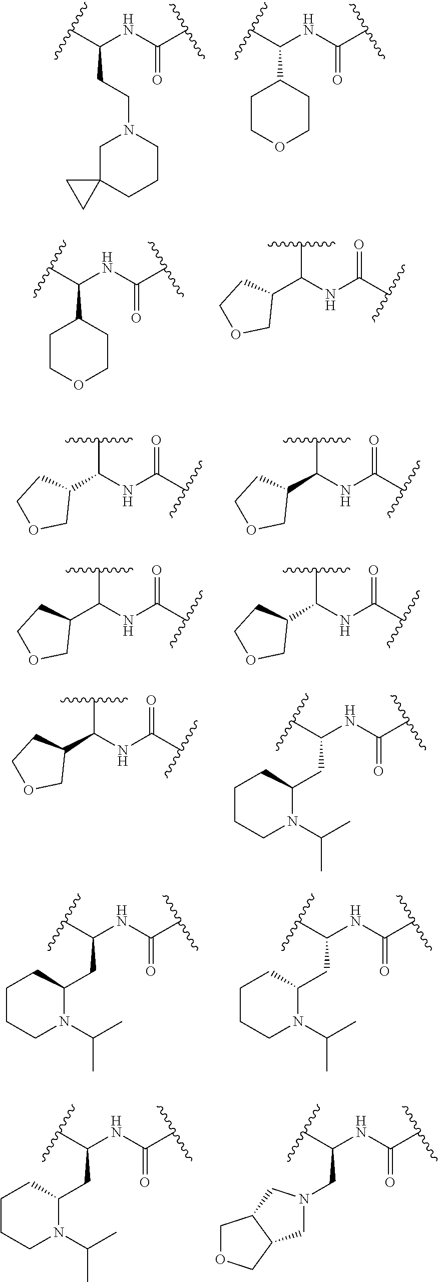

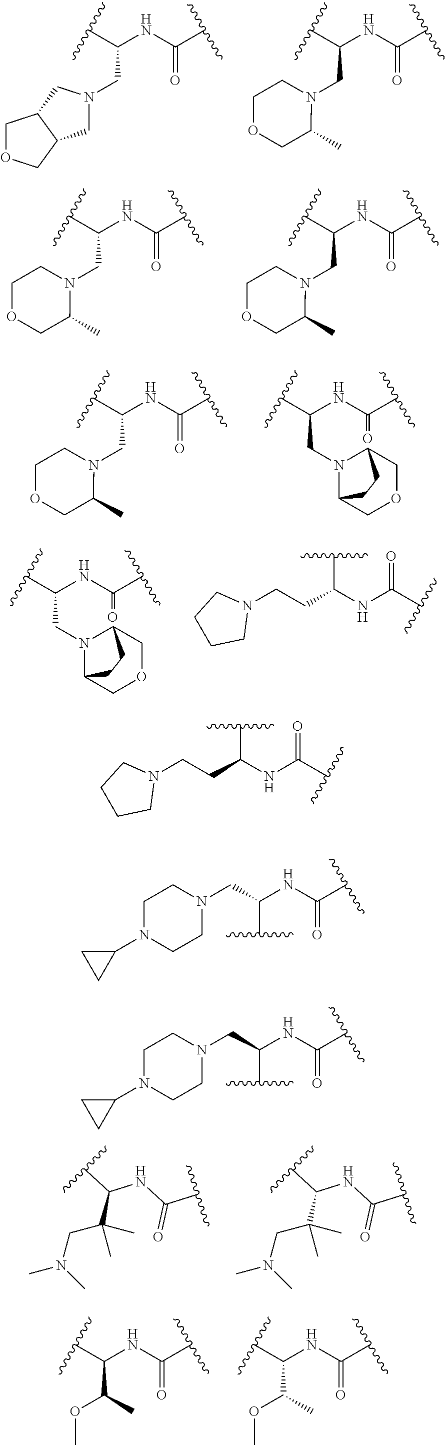

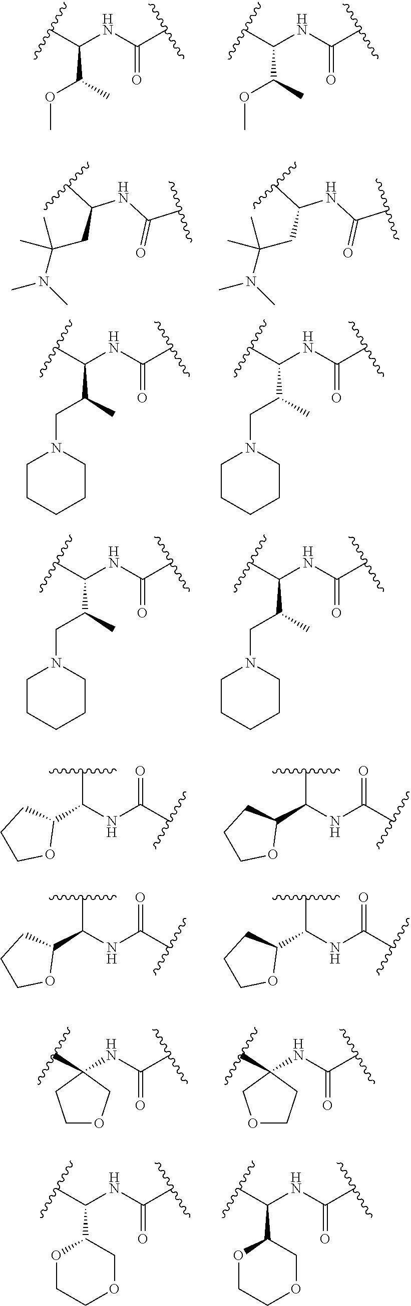

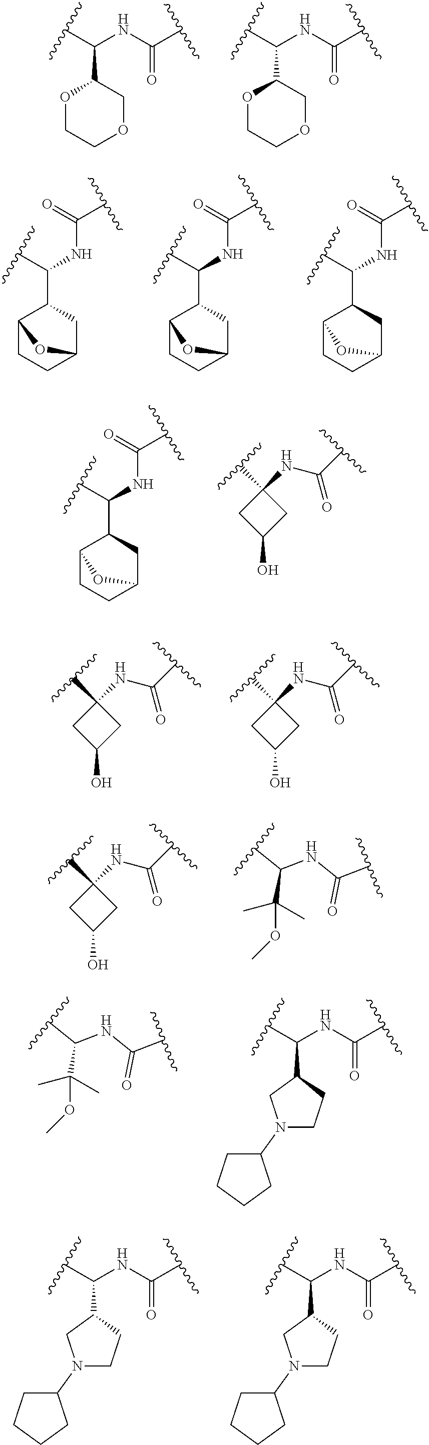

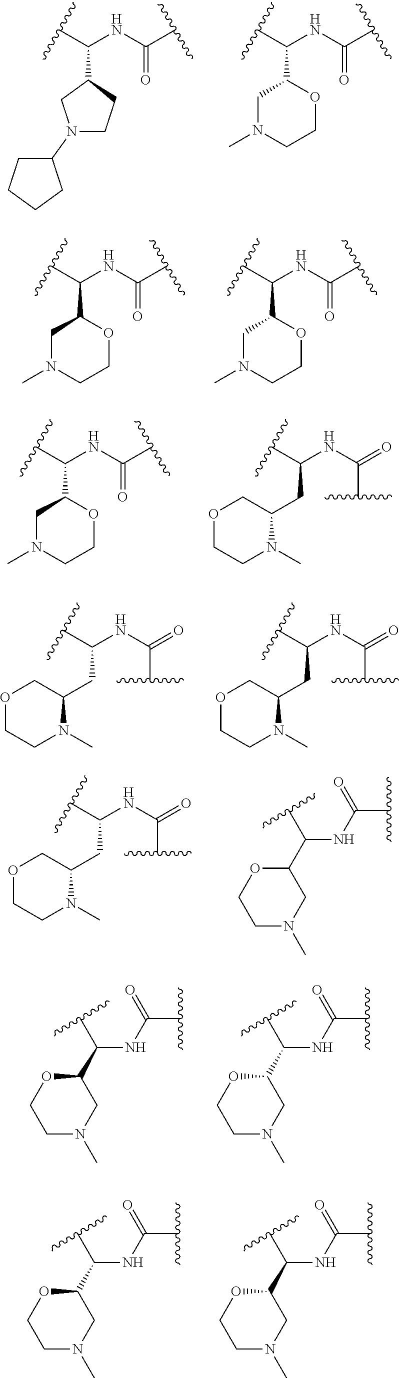

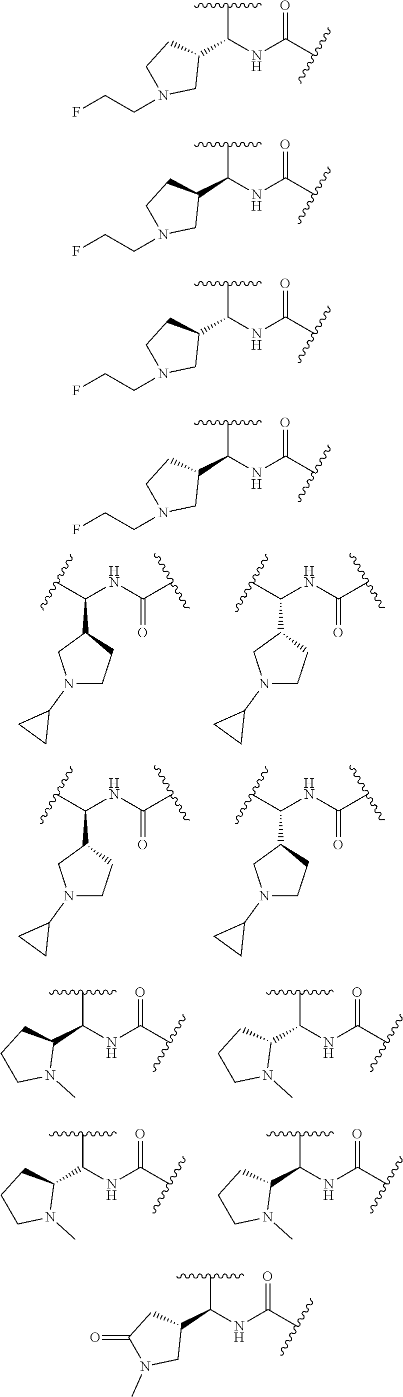

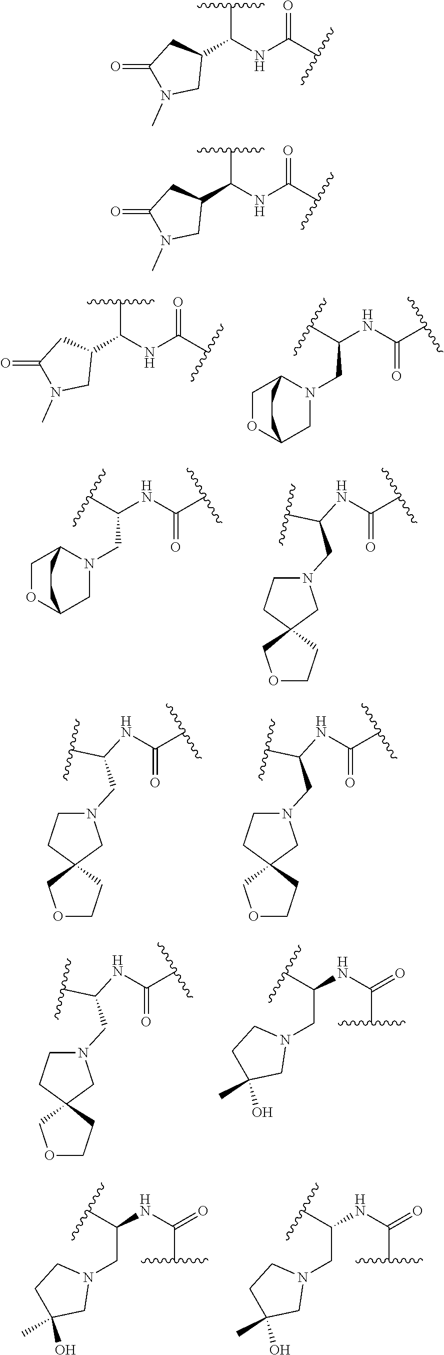

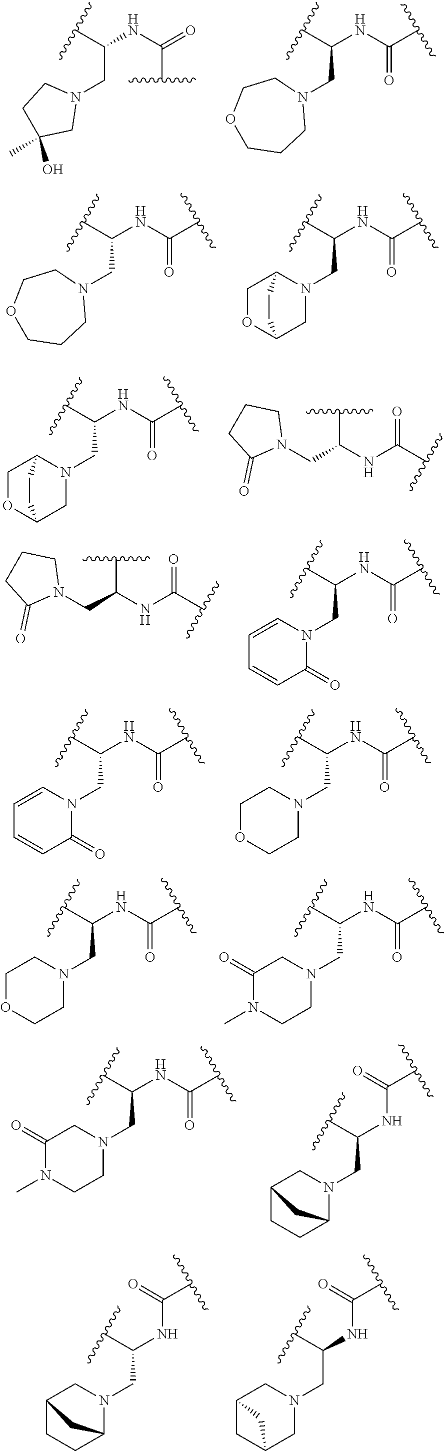

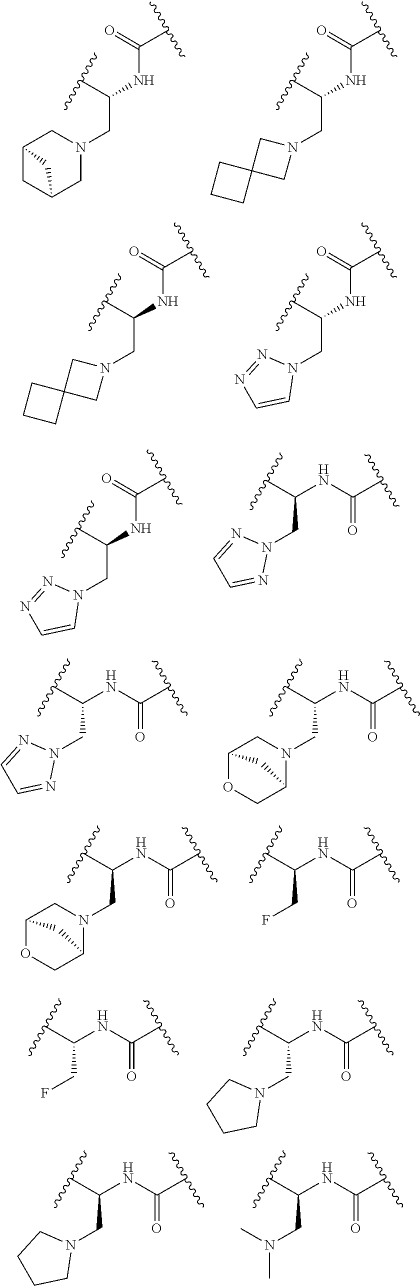

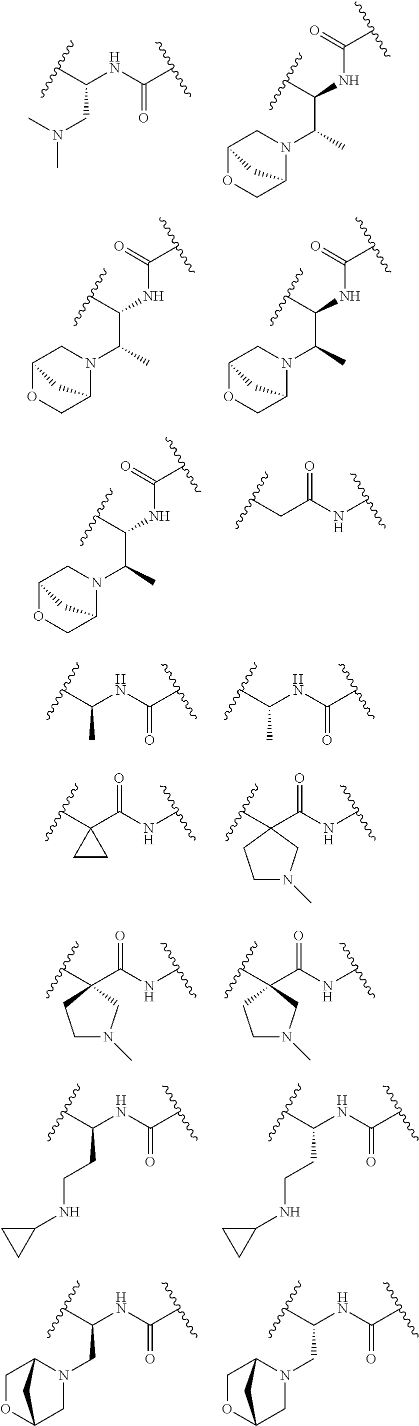

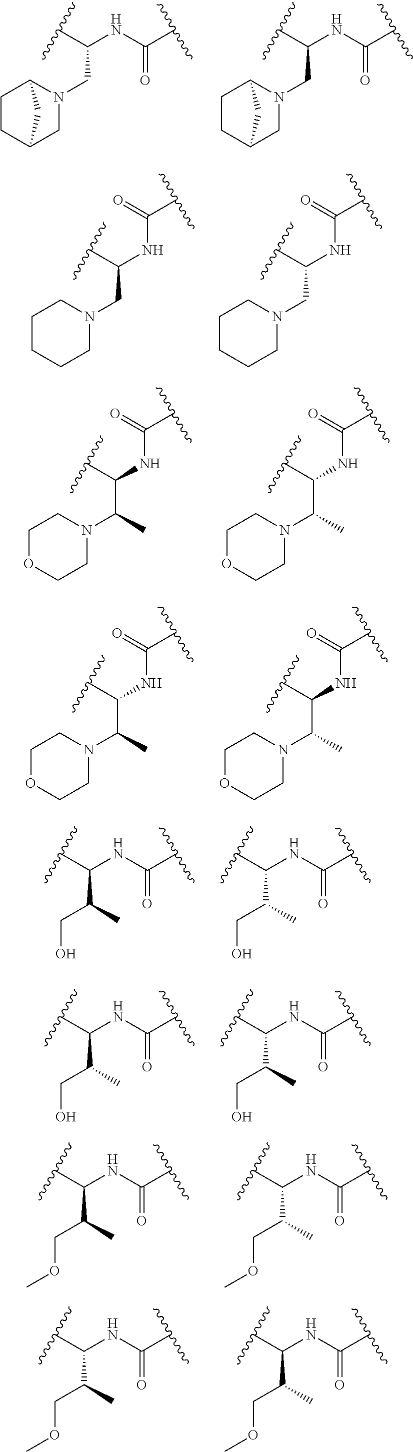

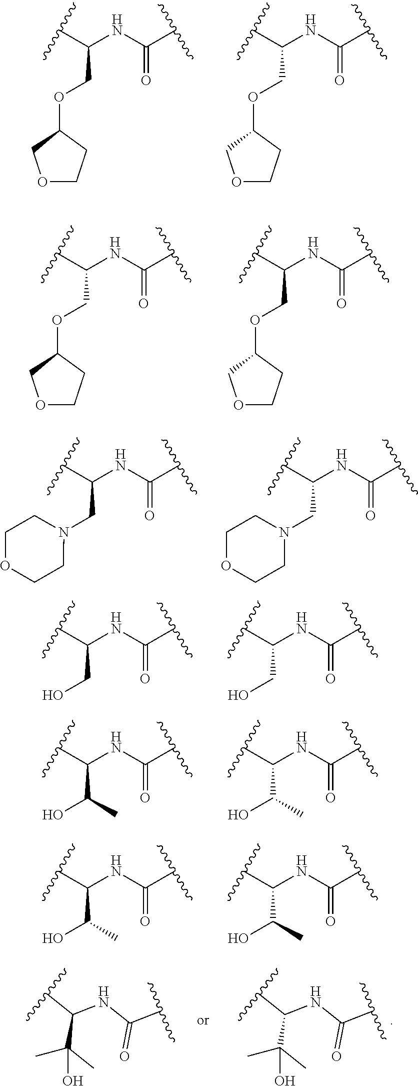

9. The compound of claim 1, wherein one of R^{L′} and R^{L″} is hydrogen and the other one of R^{L′} and R^{L″} is not hydrogen.

10. The compound of claim 1, wherein Ring B is phenyl or a 5-6 membered monocyclic heteroaryl ring having 1-4 heteroatoms independently selected from nitrogen, oxygen, and sulfur.

11. The compound of claim 1, wherein Ring B is phenyl, thiazolyl, or pyridinyl.

12. The compound of claim 1, wherein Ring B is

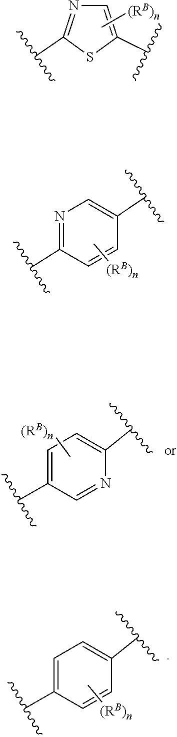

13. The compound of claim 1, wherein Ring B and R^{B} together are

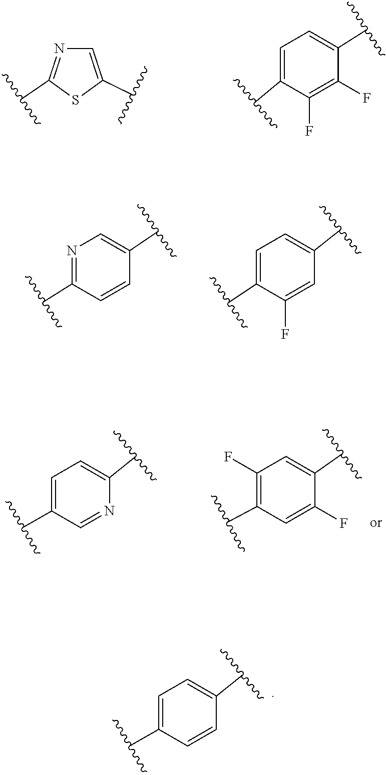

14. The compound of claim 1, wherein Ring B and R^{B} together are

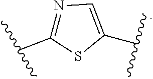 [SMILES: *c1cnc(*)s1]

15. The compound of claim 1, wherein Ring C is selected from a 6-membered monocyclic heteroaryl ring having 1-4 heteroatoms independently selected from nitrogen, oxygen, and sulfur and a 7-11 membered fused bicyclic heteroaryl ring having 1-4 heteroatoms independently selected from nitrogen, oxygen, and sulfur.

16. The compound of claim 1, wherein Ring C is pyrazinyl or pyrazolo[1,5-a]pyridinyl.

17. The compound of claim 1, wherein Ring C is

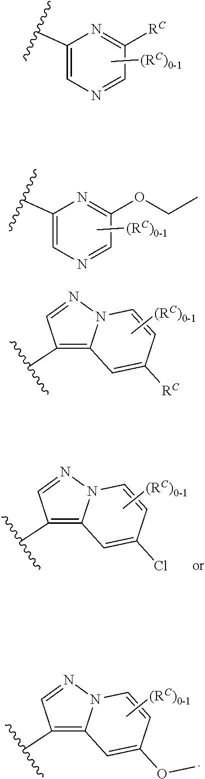

18. The compound of claim 1, wherein Ring C and its R^{C} substituents together are

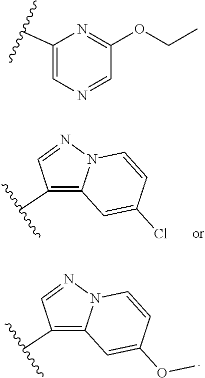

19. The compound of claim 1, having the formula of IV-a, IV-b, or IV-c: 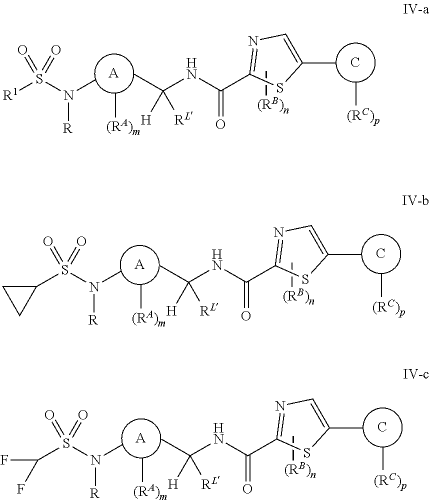
or a pharmaceutically acceptable salt thereof.

20. The compound of claim 1, having the formula of IX-a, IX-b, or IX-c: 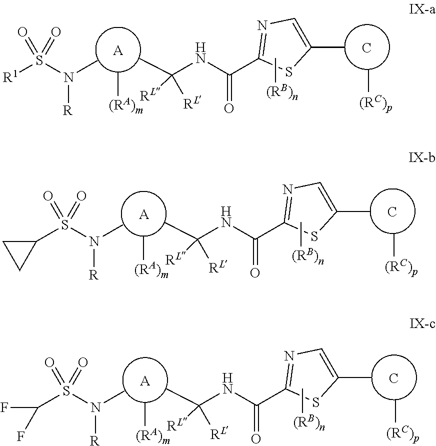
or a pharmaceutically acceptable salt thereof, wherein the R^{L′} and R^{L″} group are taken together with the atom to which they are attached, to form an optionally substituted 3-7 membered saturated or partially unsaturated monocyclic carbocyclic ring; or a 3-7 membered optionally substituted saturated or partially unsaturated monocyclic heterocyclic ring having 1-2 heteroatoms independently selected from nitrogen, oxygen, and sulfur.

21. The compound of claim 1, having the formula of V-a, V-b, V-c, V-d, V-e, or V-f: 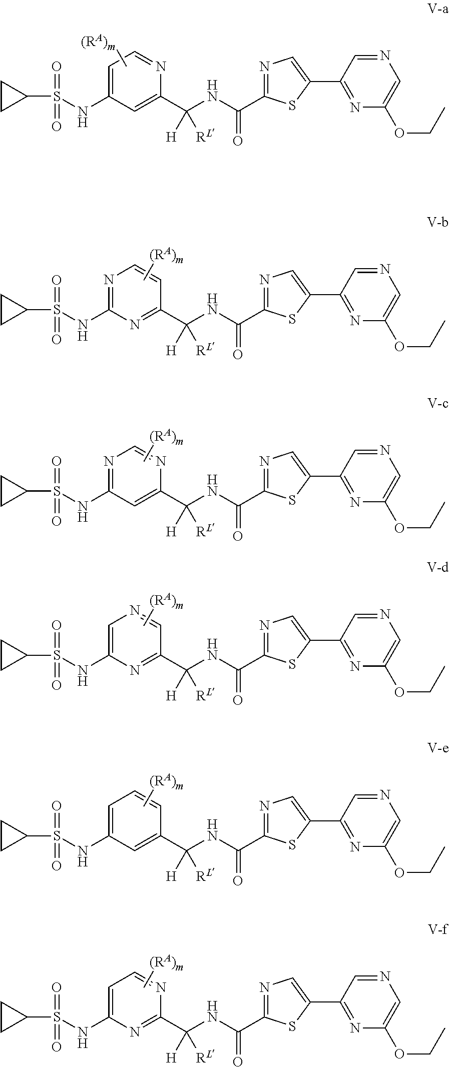
or a pharmaceutically acceptable salt thereof.

22. The compound of claim 1, having the formula of VT-a, VI-b, VI-c, VI-d, VI-e, or VI-f: 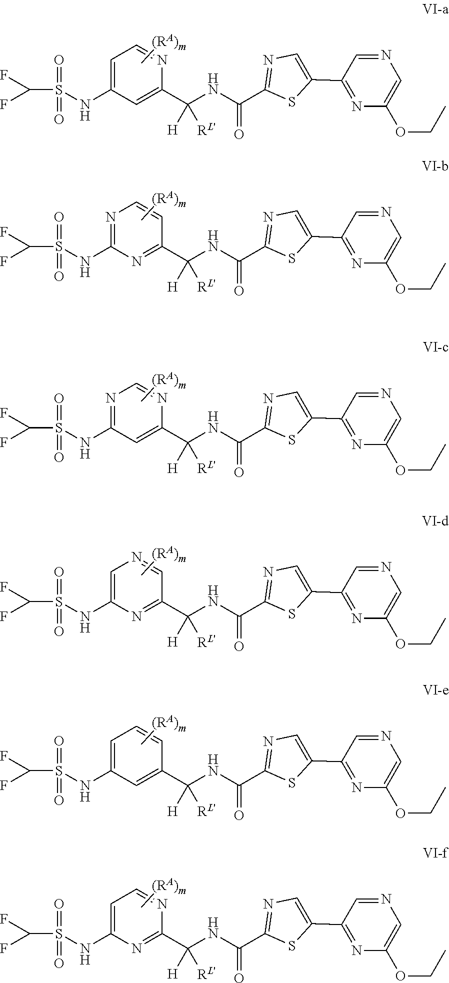
or a pharmaceutically acceptable salt thereof.

23. The compound of claim 1, having the formula of VII-a, VII-b, VII-c, VII-d, VII-e, or VII-f: 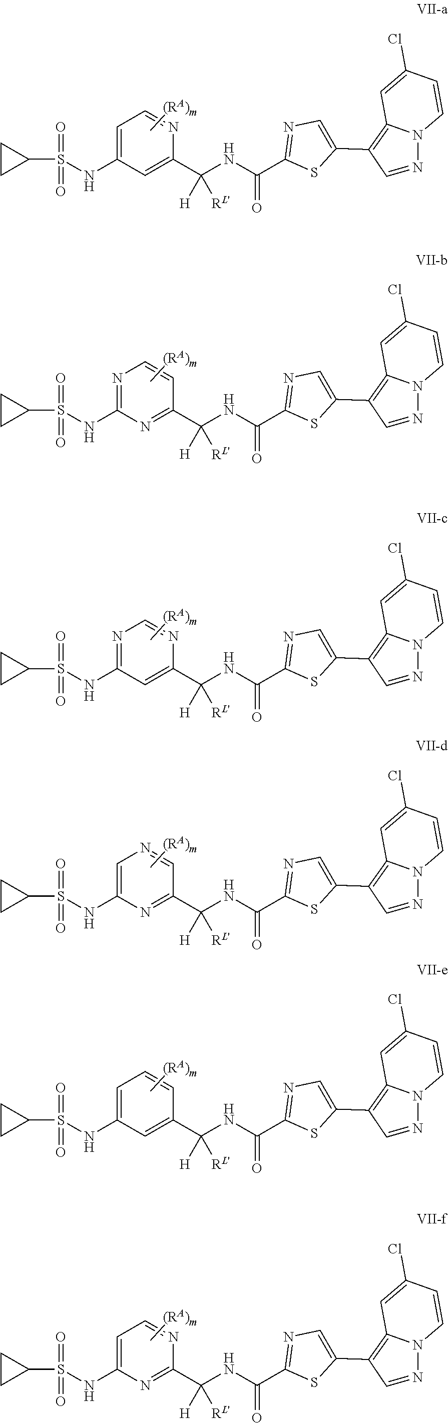
or a pharmaceutically acceptable salt thereof.

24. The compound of claim 1, having the formula of VIII-a, VIII-b, VIII-c, VIII-d, VIII-e, or VIII-f: 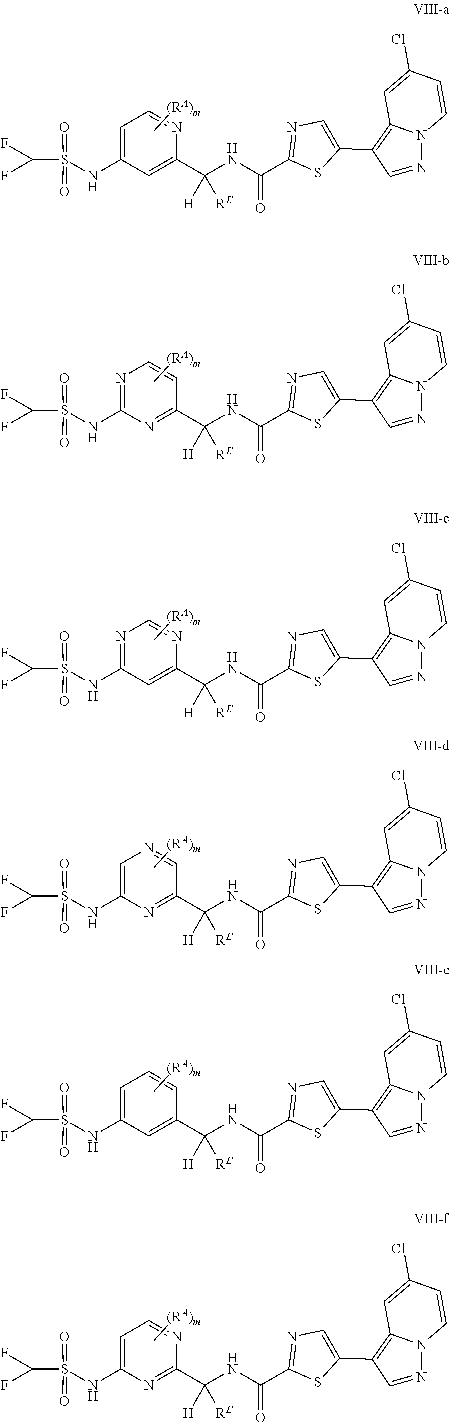
a pharmaceutically acceptable salt thereof.

25. The compound of claim 1, wherein the compound is selected from those depicted in Table 1, or a pharmaceutically acceptable salt thereof.

26. A pharmaceutical composition comprising a compound according to any one of claims 1-25, or a pharmaceutically acceptable salt thereof, and a pharmaceutically acceptable carrier, adjuvant, or vehicle.

27. The compound of any one of claims 1-25, or the pharmaceutical composition of claim 26, for use as a medicament.

28. A method of treating a CPTS1-mediated disorder, disease, or condition in a patient comprising administering to the patient the compound of any one of claims 1-25, or a pharmaceutically acceptable salt thereof, or the pharmaceutical composition of claim 26.

29. The method of claim 28, wherein the disorder, disease, or condition is selected from rejection of transplanted cells and tissues, Graft-related diseases or disorders, allergies, and autoimmune diseases.

30. The method of claim 29, wherein the disorder, disease, or condition is graft versus host disease (GVHD).

31. The method of claim 29, wherein the disorder, disease, or condition is Addison's Disease, Adult-onset Still's disease, Alopecia Areata, Alzheimer's disease, Anti-neutrophil Cytoplasmic Antibodies (ANCA)-Associated Vasculitis, Ankylosing Spondylitis, Anti-phospholipid Syndrome (Hughes Syndrome), Aplastic Anemia, Arthritis, Asthma, Atherosclerosis, Atherosclerotic plaque, Atopic Dermatitis, Autoimmune Hemolytic Anemia, Autoimmune Hepatitis, Autoimmune Hypophysitis (Lymphocytic Hypophysitis), Autoimmune Inner Ear Disease, Autoimmune Lymphoproliferative Syndrome, Autoimmune Myocarditis, Autoimmune Neutropenia, Autoimmune Oophoritis, Autoimmune Orchitis, Auto-Inflammatory Diseases requiring an immunosuppressive treatment, Azoospermia, Bechet's Disease, Berger's Disease, Bullous Pemphigoid, Cardiomyopathy, Cardiovascular disease, Celiac disease including Refractory Celiac Disease (type I and type II), Chronic Fatigue Immune Dysfunction Syndrome (CFIDS), Chronic Idiopathic Polyneuritis, Chronic Inflammatory Demyelinating Polyneuropathy (CIPD), Chronic Relapsing Polyneuropathy (Guillain-Barre syndrome), Churg-Strauss Syndrome (CSS), Cicatricial Pemphigoid, Cold Agglutinin Disease (CAD), chronic obstructive pulmonary disease (COPD), CREST Syndrome, Cryoglobulin Syndromes, Cutaneous Lupus, Dermatitis Herpetiformis, Dermatomyositis, Eczema, Epidermolysis Bullosa Acquisita, Essential Mixed Cryoglobulinemia, Evan's Syndrome, Exophthalmos, Fibromyalgia, Goodpasture's Syndrome, Grave's disease, Hemophagocytic Lymphohistiocytosis (HLH) (including Type 1 Hemophagocytic Lymphohistiocytosis), Histiocytosis/Histiocytic Disorders, Hashimoto's Thyroiditis, Idiopathic Pulmonary Fibrosis, Idiopathic Thrombocytopenia Purpura (ITP), IgA Nephropathy, Immunoproliferative Diseases or Disorders, Inflammatory Bowel Disease (IBD), Interstitial Lung Disease, Juvenile Arthritis, Juvenile Idiopathic Arthritis (JIA), Kawasaki's Disease, Lambert-Eaton Myasthenic Syndrome, Lichen Planus, Localized Scleroderma, Lupus Nephritis, Meniere's Disease, Microangiopathic Hemoytic Anemia, Microscopic Polyangiitis, Miller Fischer Syndrome/Acute Disseminated Encephalomyeloradiculopathy, Mixed Connective Tissue Disease, Multiple Sclerosis (MS), Muscular Rheumatism, Myalgic Encephalomyelitis (ME), Myasthenia Gravis, Ocular Inflammation, Pemphigus Foliaceus, Pemphigus Vulgaris, Pernicious Anemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune Cholangiopathy, Primary Glomerulonephritis, Primary Sclerosing Cholangitis, Psoriasis, Psoriatic Arthritis, Pure Red Cell Anemia, Raynaud's Phenomenon, Reiter's Syndrome/Reactive Arthritis, Relapsing Polychondritis, Restenosis, Rheumatic Fever, Rheumatic Disease, Rheumatoid Arthritis, Sarcoidosis, Schmidt's Syndrome, Scleroderma/Systemic Sclerosis, Sjorgen's Syndrome, Stiff-Man Syndrome, The Sweet Syndrome (Febrile Neutrophilic Dermatosis), Systemic Lupus Erythematosus (SLE), Systemic Scleroderma, Takayasu Arteritis, Temporal Arteritis/Giant Cell Arteritis, Thyroiditis, Type 1 diabetes, Type 2 diabetes, Uveitis, Vasculitis, Vitiligo, Wegener's Granulomatosis, or X-linked lymphoproliferative disease.
